SEQUENCE LISTING (19 sequences)

SEQ ID NO: 1 - DNA, 2604 bp, Artificial Sequence
  source: /note=“Description of Artificial Sequence Synthetic polynucleotide”
atgagagtgatggggacacagaagaattgtcaacaatggtggatatggggcatcttaggcttctggatgctaatgatttgtaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccaagcgccgcgtggtgcagcgcgagaagcgcgccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgaaggccatcgaggcccagcagcacatgctgcagctgaccgtgtggggcatcaagcagctgcaggcccgcgtgctggccatcgagcgctacctgaaggaccagcagctgctgggcatctggggctgcagcggccgcctgatctgcaccaccgccgtgccctggaacagcagctggagcaacaagagcgagaaggacatctgggacaacatgacctggatgcagtgggaccgcgagatcagcaactacaccggcctgatctacaacctgctggaggacagccagaaccagcaggagaagaacgagaaggacctgctggagctggacaagtggaacaacctgtggaactggttcgacatcagcaactggccctggtacatcaagatcttcatcatgatcgtgggcggcctgatcggcctgcgcatcatcttcgccgtgctgagcatcgtgaaccgcgtgcgccagggctacagccccctgagcttccagaccctgacccccagcccccgcggcctggaccgcctgggcggcatcgaggaggagggcggcgagcaggaccgcgaccgcagcatccgcctggtgagcggcttcctgagcctggcctgggacgacctgcgcaacctgtgcctgttcagctaccaccgcctgcgcgacttcatcctgatcgccgtgcgcgccgtggagctgctgggccacagcagcctgcgcggcctgcagcgcggctgggagatcctgaagtacctgggcagcctggtgcagtactggggcctggagctgaagaagagcgccatcagcctgctggacaccatcgccatcaccgtggccgagggcaccgaccgcatcatcgagctggtgcagcgcatctgccgcgccatcctgaacatcccccgccgcatccgccagggcttcgaggccgccctgctgtaa

SEQ ID NO: 2 - protein, 867 aa, Artificial Sequence
  source: /note=“Description of Artificial Sequence Synthetic polypeptide”
MRVMGTQKNCQQWWIWGILGFWMLMICNTEDLWVTVYYGVPVWRDAKTTLFCASDAKAYETEVHNVWATHACVPTDPNPQEIVLGNVTENFNMWKNDMADQMHEDVISLWDQSLKPCVKLTPLCVTLNCTDTNVTGNRTVTGNSTNNTNGTGIYNIEEMKNCSFNATTELRDKKHKEYALFYRLDIVPLNENSDNFTYRLINCNTSTITQACPKVSFDPIPIHYCAPAGYAILKCNNKTFNGTGPCYNVSTVQCTHGIKPVVSTQLLLNGSLAEEGIIIRSENLTENTKTIIVHLNESVEINCTRPNNNTRKSVRIGPGQAFYATNDVIGNIRQAHCNISTDRWNKTLQQVMKKLGEHFPNKTIQFKPHAGGDLEITMHSFNCRGEFFYCNTSNLFNSTYHSNNGTYKYNGNSSSPITLQCKIKQIVRMWQGVGQATYAPPIAGNITCRSNITGILLTRDGGFNTTNNTETFRPGGGDMRDNWRSELYKYKVVEIKPLGIAPTKAKRRVVQREKRAVGIGAVFLGFLGAAGSTMGAASITLTVQARQLLSGIVQQQSNLLKAIEAQQHMLQLTVWGIKQLQARVLAIERYLKDQQLLGIWGCSGRLICTTAVPWNSSWSNKSEKDIWDNMTWMQWDREISNYTGLIYNLLEDSQNQQEKNEKDLLELDKWNNLWNWFDISNWPWYIKIFIMIVGGLIGLRIIFAVLSIVNRVRQGYSPLSFQTLTPSPRGLDRLGGIEEEGGEQDRDRSIRLVSGFLSLAWDDLRNLCLFSYHRLRDFILIAVRAVELLGHSSLRGLQRGWEILKYLGSLVQYWGLELKKSAISLLDTIAITVAEGTDRIIELVQRICRAILNIPRRIRQGFEAALL

SEQ ID NO: 3 - DNA, 2046 bp, Human immunodeficiency virus 1
atggatgcaatgaagagagggctctgctgtgtgctgctgctgtgtggagcagtcttcgtttcgcccaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccatctcctccgtggtgcagagcgagaagagcgccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgaaggccatcgaggcccagcagcacatgctgcagctgaccgtgtggggcatcaagcagctgcaggcccgcgtgctggccatcgagcgctacctgaaggaccagcagctgctgggcatctggggctgcagcggccgcctgatctgcaccaccgccgtgccctggaacagcagctggagcaacaagagcgagaaggacatctgggacaacatgacctggatgcagtgggaccgcgagatcagcaactacaccggcctgatctacaacctgctggaggacagccagaaccagcaggagaagaacgagaaggacctgctggagctggacaagtggaacaacctgtggaactggttcgacatcagcaactggccctggtacatctaa

SEQ ID NO: 4 - protein, 681 aa, Human immunodeficiency virus 1
MDAMKRGLCCVLLLCGAVFVSPNTEDLWVTVYYGVPVWRDAKTTLFCASDAKAYETEVHNVWATHACVPTDPNPQEIVLGNVTENFNMWKNDMADQMHEDVISLWDQSLKPCVKLTPLCVTLNCTDTNVTGNRTVTGNSTNNTNGTGIYNIEEMKNCSFNATTELRDKKHKEYALFYRLDIVPLNENSDNFTYRLINCNTSTITQACPKVSFDPIPIHYCAPAGYAILKCNNKTFNGTGPCYNVSTVQCTHGIKPVVSTQLLLNGSLAEEGIIIRSENLTENTKTIIVHLNESVEINCTRPNNNTRKSVRIGPGQAFYATNDVIGNIRQAHCNISTDRWNKTLQQVMKKLGEHFPNKTIQFKPHAGGDLEITMHSFNCRGEFFYCNTSNLFNSTYHSNNGTYKYNGNSSSPITLQCKIKQIVRMWQGVGQATYAPPIAGNITCRSNITGILLTRDGGFNTTNNTETFRPGGGDMRDNWRSELYKYKVVEIKPLGIAPTKAISSVVQSEKSAVGIGAVFLGFLGAAGSTMGAASITLTVQARQLLSGIVQQQSNLLKAIEAQQHMLQLTVWGIKQLQARVLAIERYLKDQQLLGIWGCSGRLICTTAVPWNSSWSNKSEKDIWDNMTWMQWDREISNYTGLIYNLLEDSQNQQEKNEKDLLELDKWNNLWNWFDISNWPWYI

SEQ ID NO: 5 - DNA, 2604 bp, Human immunodeficiency virus 1
atgcgcgtgatgggcacccagaagaactgccagcagtggtggatctggggcatcctgggcttctggatgctgatgatctgcaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccaagcgccgcgtggtgcagcgcgagaagcgcgccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgaaggccatcgaggcccagcagcacatgctgcagctgaccgtgtggggcatcaagcagctgcaggcccgcgtgctggccatcgagcgctacctgaaggaccagcagctgctgggcatctggggctgcagcggccgcctgatctgcaccaccgccgtgccctggaacagcagctggagcaacaagagcgagaaggacatctgggacaacatgacctggatgcagtgggaccgcgagatcagcaactacaccggcctgatctacaacctgctggaggacagccagaaccagcaggagaagaacgagaaggacctgctggagctggacaagtggaacaacctgtggaactggttcgacatcagcaactggccctggtacatcaagatcttcatcatgatcgtgggcggcctgatcggcctgcgcatcatcttcgccgtgctgagcatcgtgaaccgcgtgcgccagggctacagccccctgagcttccagaccctgacccccagcccccgcggcctggaccgcctgggcggcatcgaggaggagggcggcgagcaggaccgcgaccgcagcatccgcctggtgagcggcttcctgagcctggcctgggacgacctgcgcaacctgtgcctgttcagctaccaccgcctgcgcgacttcatcctgatcgccgtgcgcgccgtggagctgctgggccacagcagcctgcgcggcctgcagcgcggctgggagatcctgaagtacctgggcagcctggtgcagtactggggcctggagctgaagaagagcgccatcagcctgctggacaccatcgccatcaccgtggccgagggcaccgaccgcatcatcgagctggtgcagcgcatctgccgcgccatcctgaacatcccccgccgcatccgccagggcttcgaggccgccctgctgtaa

SEQ ID NO: 6 - protein, 867 aa, Human immunodeficiency virus 1; identical to SEQ ID NO: 2

SEQ ID NO: 7 - DNA, 1533 bp, Human immunodeficiency virus 1
atggatgcaatgaagagagggctctgctgtgtgctgctgctgtgtggagcagtcttcgtttcgcccaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccaagcgccgcgtggtgcagcgcgagaagcgctaa

SEQ ID NO: 8 - protein, 510 aa, Human immunodeficiency virus 1
MDAMKRGLCCVLLLCGAVFVSPNTEDLWVTVYYGVPVWRDAKTTLFCASDAKAYETEVHNVWATHACVPTDPNPQEIVLGNVTENFNMWKNDMADQMHEDVISLWDQSLKPCVKLTPLCVTLNCTDTNVTGNRTVTGNSTNNTNGTGIYNIEEMKNCSFNATTELRDKKHKEYALFYRLDIVPLNENSDNFTYRLINCNTSTITQACPKVSFDPIPIHYCAPAGYAILKCNNKTFNGTGPCYNVSTVQCTHGIKPVVSTQLLLNGSLAEEGIIIRSENLTENTKTIIVHLNESVEINCTRPNNNTRKSVRIGPGQAFYATNDVIGNIRQAHCNISTDRWNKTLQQVMKKLGEHFPNKTIQFKPHAGGDLEITMHSFNCRGEFFYCNTSNLFNSTYHSNNGTYKYNGNSSSPITLQCKIKQIVRMWQGVGQATYAPPIAGNITCRSNITGILLTRDGGFNTTNNTETFRPGGGDMRDNWRSELYKYKVVEIKPLGIAPTKAKRRVVQREKR

SEQ ID NO: 9 - DNA, 30 bp, Artificial Sequence
  source: /note=“Description of Artificial Sequence Synthetic oligonucleotide”
aaaaaaaaaaaaaaaaaaaaaaaaaaaaaa

SEQ ID NO: 10 - DNA, 13538 bp, Artificial Sequence
  source: /note=“Description of Artificial Sequence Synthetic polynucleotide”
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctcctcaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagagggatcacgggagaaaccgtgggatacgcggttacacacaatagcgagggcttcttgctatgcaaagttactgacacagtaaaaggagaacgggtatcgttccctgtgtgcacgtacatcccggccaccataaactcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacctaagaattcgccaccatggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgcgccgtgaccctgggcgccatgttcctgggcttcctgggcgccgccggcagcaccatgggcgcccgcagcctgaccctgaccgtgcaggcccgccagctgctgagcggcatcgtgcagcagcagaacaacctgctgcgcgccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccaacctgatctacaccctgatcgaggagagccagaaccagcaggagaagaacgagcaggagctgctggagctggacaagtgggccagcctgtggaactggttcgacatcagcaagtggctgtggtacatcaagatcttcatcatgatcgtgggcggcctggtgggcctgcgcatcgtgttcaccgtgctgagcatcgtgaaccgcgtgcgccagggctacagccccctgagcttccagacccgcttccccgccccccgcggccccgaccgccccgagggcatcgaggaggagggcggcgagcgcgaccgcgaccgcagcagccccctggtgcacggcctgctggccctgatctgggacgacctgcgcagcctgtgcctgttcagctaccaccgcctgcgcgacctgatcctgatcgccgcccgcatcgtggagctgctgggccgccgcggctgggaggccctgaagtactggggcaacctgctgcagtactggatccaggagctgaagaacagcgccgtgagcctgttcgacgccatcgccatcgccgtggccgagggcaccgaccgcatcatcgaggtggcccagcgcatcggccgcgccttcctgcacatcccccgccgcatccgccagggcttcgagcgcgccctgctgtaactcgagcaagtctagacggcgcgcccacccagcggccgccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgagctcctgtttaaaccagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggcccacgcgtaatacgactcactatag

SEQ ID NO: 11 - protein, 842 aa, Artificial Sequence
  source: /note=“Description of Artificial Sequence Synthetic polypeptide”
MDAMKRGLCCVLLLCGAVFVSPSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIKIFIMIVGGLVGLRIVFTVLSIVNRVRQGYSPLSFQTRFPAPRGPDRPEGIEEEGGERDRDRSSPLVHGLLALIWDDLRSLCLFSYHRLRDLILIAARIVELLGRRGWEALKYWGNLLQYWIQELKNSAVSLFDAIAIAVAEGTDRIIEVAQRIGRAFLHIPRRIRQGFERALL

SEQ ID NO: 12 - DNA, 13566 bp, Artificial Sequence
  source: /note=“Description of Artificial Sequence Synthetic polynucleotide”
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctcctcaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagagggatcacgggagaaaccgtgggatacgcggttacacacaatagcgagggcttcttgctatgcaaagttactgacacagtaaaaggagaacgggtatcgttccctgtgtgcacgtacatcccggccaccataaactcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacgccaccatgagagtgcgcggcatccccagaaactggccccagtggtggatctggggcatcctgggcttttggatgatcatcatctgccgggtggtcggcaacctggacctgtgggtgaccgtgtactacggcgtgcccgtgtggaaagaggccaagaccaccctgttctgcgccagcgacgccaaggcctacgacaaagaggtgcacaacgtctgggccacacacgcctgcgtgcccaccgaccccaaccctcaggaaatcgtcctggaaaacgtgaccgagaacttcaacatgtggaagaacgacatggtggaccagatgcacgaggacatcatcagcctgtgggaccagagcctgaagccctgcgtgaagctgacccccctgtgcgtgaccctgaactgcaagaacgtgaacatcagcgccaacgccaatgccacagccacactgaacagcagcatgaacggcgagatcaagaactgcagcttcaacaccaccaccgagctgcgggacaagaaacagaaggtgtacgccctgttctacaagcccgacgtggtgcctctgaacggcggcgagcacaacgagacaggcgagtacatcctgatcaactgcaacagctccaccatcacccaggcctgccccaaggtgtccttcgaccccatccccatccactattgcgcccctgccggctacgccatcctgaagtgcaacaacaagaccttcaacggcaccggcccctgcaacaacgtgtccaccgtgcagtgcacccacggcatcaagcccgtggtgtccacccagctgctgctgaatggcagcctggccgaggaagagatcatcgtccgcagcgagaacctgaccaacaacattaagaccatcatcgtgcacctgaacaagagcgtggagatcaagtgcacccggcccaacaacaacacccggaagtccgtgagaatcggccctggccagaccttctacgccactggcgagatcatcggcgacatccgggaggcccactgcaacatcagccgggagacatggaacagcaccctgatccaggtcaaagagaagctgcgggagcactacaataagaccatcaagttcgagcccagcagcggaggcgatctggaagtgaccacccacagcttcaactgcagaggcgagttcttctactgcgacaccaccaagctgttcaacgagacaaagctgtttaatgagagcgagtacgtggacaacaagacaatcatcctgccctgccggatcaagcagatcatcaatatgtggcaggaagtgggcagagccatgtacgcccctcccatcgagggcaacatcacatgcaagagcaacatcaccggcctgctgctgacctgggatggcggcgagaatagcaccgagggcgtgttcagacccggcggaggcaacatgaaggacaactggcggagcgagctgtacaagtacaaggtggtggagattaagcccctgggcgtggctcccaccaagagcaagcggaaggtggtcggccgggagaaaagagccgtgggcctgggagccgtgctgctgggatttctgggcgctgccggctctacaatgggggctgccagcatcacactgaccgtgcaggctagacagctgctgtccgggattgtgcagcagcagagcaacctgctgagagccattgaggcccagcagcatctgctgcagctgaccgtgtggggcatcaagcagctgcagacccgggtgctggccatcgagagatacctgaaggaccagcagctgctcgggctgtggggctgtagcggcaagctgatctgcgccacagccgtgccctggaacagcagctggtccaacaagagcctgggcgacatctgggacaacatgacctggatgcagtgggaccgggagatcagcaactacaccaacaccatcttcagactgctggaagatagccagaaccagcaggaaaagaacgagaaggacctgctggctctggacagctggaagaacctgtggaattggttcgacatcaccaactggctgtggtacatcaagatcttcatcatgatcgtgggcggcctgatcggcctgcggatcatcttcggcgtgctggctatcgtgaagagagtgcggcagggctacagccccctgagcttccagaccctgatccccaaccccagaggccccgacagactgggcagaatcgaggaagagggcggcgaacaggacaaggaccggtctatccggctggtgtccggatttctggccctggcctgggacgatctgcggagcctgtgcctgttcagctatcaccagctgagagacttcatcctgaccgccgctagagccgctgaactgctgggcagaagcagcctgagaggcctgcagaggggctgggaggtgctgaagtacctgggcaatctggtgcagtactggggcctggaactgaagcggagcgccatcaacctgttcgacacaatcgccattgccgtggccgagggcaccgaccggatcatcgaagtgatccagcggatctgccgggccatccggtacatccccacccggatcagacagggcttcgaggccgctctgctgtaatctagacggcgcgcccacccagcggccgccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgagctcctgtttaaaccagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggcccacgcgtaatacgactcactatag

SEQ ID NO: 13 - protein, 858 aa, Artificial Sequence
  source: /note=“Description of Artificial Sequence Synthetic polypeptide”
MRVRGIPRNWPQWWIWGILGFWMIIICRVVGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 14 - DNA, 12782 bp, Artificial Sequence
  source: /note=“Description of Artificial Sequence Synthetic polynucleotide”
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacgccaccatggatgcaatgaagagagggctctgctgtgtgctgctgctgtgtggagcagtcttcgtttcgcccaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccatctcctccgtggtgcagagcgagaagagcgccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgaaggccatcgaggcccagcagcacatgctgcagctgaccgtgtggggcatcaagcagctgcaggcccgcgtgctggccatcgagcgctacctgaaggaccagcagctgctgggcatctggggctgcagcggccgcctgatctgcaccaccgccgtgccctggaacagcagctggagcaacaagagcgagaaggacatctgggacaacatgacctggatgcagtgggaccgcgagatcagcaactacaccggcctgatctacaacctgctggaggacagccagaaccagcaggagaagaacgagaaggacctgctggagctggacaagtggaacaacctgtggaactggttcgacatcagcaactggccctggtacatctaatctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttaaaccagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggcccacgcgtaatacgactcactatag

SEQ ID NO: 15 - protein, 681 aa, Artificial Sequence; identical to SEQ ID NO: 4
  source: /note=“Description of Artificial Sequence Synthetic polypeptide”

SEQ ID NO: 16 - DNA, 6860 bp, Artificial Sequence
  source: /note=“Description of Artificial Sequence Synthetic polynucleotide”
gccgcggaatttcgactctaggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtccatttattatttacaaattcacatatacaacaacgccgtcccccgtgcccgcagtttttattaaacatagcgtgggatctccgacatctcgggtacgtgttccggacatgggctcttctccggtagcggcggagcttccacatccgagccctggtcccatccgtccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcacctggacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacctaagaattcgccaccatggatgcaatgaagagagggctctgctgtgtgctgctgctgtgtggagcagtcttcgtttcgcccagcgccgtggagaagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgcactgcaccaacctgaagaacgccaccaacaccaagagcagcaactggaaggagatggaccgcggcgagatcaagaactgcagcttcaaggtgaccaccagcatccgcaacaagatgcagaaggagtacgccctgttctacaagctggacgtggtgcccatcgacaacgacaacaccagctacaagctgatcaactgcaacaccagcgtgatcacccaggcctgccccaaggtgagcttcgagcccatccccatccactactgcgcccccgccggcttcgccatcctgaagtgcaacgacaagaagttcaacggcagcggcccctgcaccaacgtgagcaccgtgcagtgcacccacggcatccgccccgtggtgagcacccagctgctgctgaacggcagcctggccgaggagggcgtggtgatccgcagcgagaacttcaccgacaacgccaagaccatcatcgtgcagctgaaggagagcgtggagatcaactgcacccgccccaacaacaacacccgcaagagcatcaccatcggccccggccgcgccttctacgccaccggcgacatcatcggcgacatccgccaggcccactgcaacatcagcggcgagaagtggaacaacaccctgaagcagatcgtgaccaagctgcaggcccagttcggcaacaagaccatcgtgttcaagcagagcagcggcggcgaccccgagatcgtgatgcacagcttcaactgcggcggcgagttcttctactgcaacagcacccagctgttcaacagcacctggaacaacaccatcggccccaacaacaccaacggcaccatcaccctgccctgccgcatcaagcagatcatcaaccgctggcaggaggtgggcaaggccatgtacgccccccccatccgcggccagatccgctgcagcagcaacatcaccggcctgctgctgacccgcgacggcggcaaggagatcagcaacaccaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgcgccgtgaccctgggcgccatgttcctgggcttcctgggcgccgccggcagcaccatgggcgcccgcagcctgaccctgaccgtgcaggcccgccagctgctgagcggcatcgtgcagcagcagaacaacctgctgcgcgccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccaacctgatctacaccctgatcgaggagagccagaaccagcaggagaagaacgagcaggagctgctggagctggacaagtgggccagcctgtggaactggttcgacatcagcaagtggctgtggtacatcaagatcttcatcatgatcgtgggcggcctggtgggcctgcgcatcgtgttcaccgtgctgagcatcgtgaaccgcgtgcgccagggctacagccccctgagcttccagacccgcttccccgccccccgcggccccgaccgccccgagggcatcgaggaggagggcggcgagcgcgaccgcgaccgcagcagccccctggtgcacggcctgctggccctgatctgggacgacctgcgcagcctgtgcctgttcagctaccaccgcctgcgcgacctgatcctgatcgccgcccgcatcgtggagctgctgggccgccgcggctgggaggccctgaagtactggggcaacctgctgcagtactggatccaggagctgaagaacagcgccgtgagcctgttcgacgccatcgccatcgccgtggccgagggcaccgaccgcatcatcgaggtggcccagcgcatcggccgcgccttcctgcacatcccccgccgcatccgccagggcttcgagcgcgccctgctgtaactcgagcaagtctagaaagccatggatatcggatccactacgcgttagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctgtcaattccgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcggatcagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggcggttttttgtttgcaagcagcagattacgcgcagaaaaaaaggatctcaagaagatcctttgatcttttctactgaacggtgatccccaccggaattgcg

SEQ ID NO: 17 - protein, 842 aa, Artificial Sequence; identical to SEQ ID NO: 11
  source: /note=“Description of Artificial Sequence Synthetic polypeptide”

SEQ ID NO: 18 - DNA, 6357 bp, Artificial Sequence
  source: /note=“Description of Artificial Sequence Synthetic polynucleotide”
gccgcggaatttcgactctaggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtccatttattatttacaaattcacatatacaacaacgccgtcccccgtgcccgcagtttttattaaacatagcgtgggatctccgacatctcgggtacgtgttccggacatgggctcttctccggtagcggcggagcttccacatccgagccctggtcccatccgtccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcacctggacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacgccaccatggatgcaatgaagagagggctctgctgtgtgctgctgctgtgtggagcagtcttcgtttcgcccaacaccgaggacctgtgggtgaccgtgtactacggcgtgcccgtgtggcgcgacgccaagaccaccctgttctgcgccagcgacgccaaggcctacgagaccgaggtgcacaacgtgtgggccacccacgcctgcgtgcccaccgaccccaacccccaggagatcgtgctgggcaacgtgaccgagaacttcaacatgtggaagaacgacatggccgaccagatgcacgaggacgtgatcagcctgtgggaccagagcctgaagccctgcgtgaagctgacccccctgtgcgtgaccctgaactgcaccgacaccaacgtgaccggcaaccgcaccgtgaccggcaacagcaccaacaacaccaacggcaccggcatctacaacatcgaggagatgaagaactgcagcttcaacgccaccaccgagctgcgcgacaagaagcacaaggagtacgccctgttctaccgcctggacatcgtgcccctgaacgagaacagcgacaacttcacctaccgcctgatcaactgcaacaccagcaccatcacccaggcctgccccaaggtgagcttcgaccccatccccatccactactgcgcccccgccggctacgccatcctgaagtgcaacaacaagaccttcaacggcaccggcccctgctacaacgtgagcaccgtgcagtgcacccacggcatcaagcccgtggtgagcacccagctgctgctgaacggcagcctggccgaggagggcatcatcatccgcagcgagaacctgaccgagaacaccaagaccatcatcgtgcacctgaacgagagcgtggagatcaactgcacccgccccaacaacaacacccgcaagagcgtgcgcatcggccccggccaggccttctacgccaccaacgacgtgatcggcaacatccgccaggcccactgcaacatcagcaccgaccgctggaacaagaccctgcagcaggtgatgaagaagctgggcgagcacttccccaacaagaccatccagttcaagccccacgccggcggcgacctggagatcaccatgcacagcttcaactgccgcggcgagttcttctactgcaacaccagcaacctgttcaacagcacctaccacagcaacaacggcacctacaagtacaacggcaacagcagcagccccatcaccctgcagtgcaagatcaagcagatcgtgcgcatgtggcagggcgtgggccaggccacctacgccccccccatcgccggcaacatcacctgccgcagcaacatcaccggcatcctgctgacccgcgacggcggcttcaacaccaccaacaacaccgagaccttccgccccggcggcggcgacatgcgcgacaactggcgcagcgagctgtacaagtacaaggtggtggagatcaagcccctgggcatcgcccccaccaaggccatctcctccgtggtgcagagcgagaagagcgccgtgggcatcggcgccgtgttcctgggcttcctgggcgccgccggcagcaccatgggcgccgccagcatcaccctgaccgtgcaggcccgccagctgctgagcggcatcgtgcagcagcagagcaacctgctgaaggccatcgaggcccagcagcacatgctgcagctgaccgtgtggggcatcaagcagctgcaggcccgcgtgctggccatcgagcgctacctgaaggaccagcagctgctgggcatctggggctgcagcggccgcctgatctgcaccaccgccgtgccctggaacagcagctggagcaacaagagcgagaaggacatctgggacaacatgacctggatgcagtgggaccgcgagatcagcaactacaccggcctgatctacaacctgctggaggacagccagaaccagcaggagaagaacgagaaggacctgctggagctggacaagtggaacaacctgtggaactggttcgacatcagcaactggccctggtacatctaatctagaaagccatggatatcggatccactacgcgttagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctgtcaattccgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcggatcagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggcggttttttgtttgcaagcagcagattacgcgcagaaaaaaaggatctcaagaagatcctttgatcttttctactgaacggtgatccccaccggaattgcg

SEQ ID NO: 19 - protein, 681 aa, Artificial Sequence; identical to SEQ ID NO: 4
  source: /note=“Description of Artificial Sequence Synthetic polypeptide”

(19) United States
(12) Patent Application Publication
(10) Pub. No.: US 2015/0140068 A1
(43) Pub. Date: May 21, 2015

(54) IMMUNOGENIC COMPOSITIONS AND USES THEREOF

(71) Applicant: Novartis AG, Basel (CH)

(72) Inventors: Susan Barnett, San Francisco, CA (US); Kaustuv Bannerjee, Cambridge, MA (US); Gillis Otten, Rowley, MA (US); Andrew Geall, Littleton, MA (US)

(21) Appl. No.: 14/410,728

(22) PCT Filed: Jun. 29, 2013
(86) PCT No.: PCT/EP2013/006749

§ 371 Date: Dec. 23, 2014

Related U.S. Application Data
(60) Provisional application No. 61/669,010, filed on Jul. 6, 2012.
(60) Provisional application No. 61/698,971, filed on Sep. 10, 2012.

(51) Int. Cl. A61K 39/21; C12N 7/00

(52) U.S. Cl. CPC A61K 39/21; C12N 7/00; A61K 2039/53
USPC 424/450; 424/208.1

(57) ABSTRACT

This invention generally relates to immunogenic compositions that comprise an HIV RNA component and a HIV polypeptide component. Immunogenic compositions that deliver antigenic epitopes in two different forms—a first epitope from human immunodeficiency virus (HIV), in RNA-coded form; and a second epitope from HIV, in polypeptide form—are effective in inducing immune response to HIV. The invention also relates to a kit comprising an HIV RNA-based priming composition and an HIV polypeptide-based boosting composition. The kit may be used for sequential administration of the priming and the boosting compositions.

### GOVERNMENT FUNDING

This invention was made with government support under HIVRAD Grant No. 5 PO1AI066287 awarded by the National Institutes of Health; HIV DDT ADB Contract No. NO1-AI-50007 awarded by the National Institutes of Health; and HIV DDT Contract No. HHSN266200500007C awarded by the National Institutes of Health. The government has certain rights in the invention.

This application claims the benefit of U.S. Provisional Application No. 61/669,010, filed Jul. 6, 2012 and U.S. Provisional Application No. 61/698,971 filed Sep. 10, 2012, the complete contents of the foregoing applications are hereby incorporated herein by reference for all purposes.

### BACKGROUND OF THE INVENTION

The human immunodeficiency virus (HIV-1, also referred to as HTLV-III, LAV or HTLV-III/LAV) is the etiological agent of the acquired immune deficiency syndrome (AIDS) and related disorders (see, e.g., Barre-Sinoussi et al. (1983) Science 220:868-871; Gallo et al. (1984) Science 224:500-503; Levy et al. (1984) Science 225:840-842; Siegal et al. (1981) N. Engl. J. Med. 305:1439-1444). There are several known strains of HIV including HIV-1, a collective term referring to several strains isolated in Europe or America, and HIV-2, a strain endemic in many West African countries. HIV-1 is classified by phylogenetic analysis into three groups, group M (major), group O (outlier) and a variant of HIV-1, designated group N, that has been identified with its epicenter in Cameroon (Simon et al. (1998) Nat. Med. 4: 1032-1037). All three HIV-1 groups cause AIDS.

AIDS patients usually have a long asymptomatic period followed by the progressive degeneration of the immune system and the central nervous system. Replication of the virus is highly regulated, and both latent and lytic infection of the CD4 positive helper subset of T-lymphocytes occur in tissue culture (Zagury et al. (1986) Science 231:850-853). Molecular studies of HIV-1 show that it encodes a number of genes (Ratner et al. (1985) Nature 313:277-284; Sanchez-Pescador et al. (1985) Science 227:484-492), including three structural genes—gag, pol and env—that are common to all retroviruses. Nucleotide sequences from viral genomes of other retroviruses, particularly HIV-2 and simian immunodeficiency viruses, SIV (previously referred to as STLV-III), also contain these structural genes (Guyader et al. (1987) Nature 326:662-669).

The envelope protein of HIV-1, HTV-2 and SIV is a glycoprotein of about 160 kd (gp160). During virus infection of the host cell, gp160 is cleaved by host cell proteases to form gp120 and the integral membrane protein, gp41. The gp41 portion is anchored in the membrane bilayer of virion, while the gp120 segment protrudes into the surrounding environment. gp120 and gp41 are more covalently associated and free gp120 can be released from the surface of virions and infected cells. Furthermore, upon binding to its receptor, CD4, the Env polypeptide undergoes a significant structural rearrangement. After this conformational change a CCR5 or other chemokine binding co-receptor binding site is exposed. Exposure of this chemokine receptor binding site, in turn, mediates viral entry into the host cell. See, e.g., Wyatt, R. et al. (1998) Nature 393:705-711; Kwong, P. et al. (1998) Nature 393:648-659.

Vaccines for immunizing patients against HIV infection have been under development for two decades, but with limited success. Many approaches to immunization have focused on the HIV envelope glycoprotein, although there has also been interest in other antigens such as tat or gag.

There remains a need for compositions that can elicit an immunological response (e.g., neutralizing and/or protective antibodies) in a subject against various HIV strains and subtypes, for example when administered as a vaccine or immunogenic composition. There also remains a need for improved ways of immunizing against HIV.

### SUMMARY OF THE INVENTION

Certain terms that are used to describe the invention in this are defined and explained herein in Section 6.

This invention generally relates to immunogenic compositions that comprise an HIV RNA component and an HIV polypeptide component. Immunogenic compositions that deliver antigenic epitopes in two different forms—a first epitope from HIV, in RNA-coded form; and a second epitope from HIV, in polypeptide form—can enhance the immune response to HIV, as compared to immunization with RNA alone, or polypeptide alone. Preferably, the first epitope and the second epitope are the same epitope.

The invention also relates to a kit comprising an HIV RNA-based priming composition and an HIV polypeptide-based boosting composition for sequential administration. The kit is suitable for, for example, a “RNA prime, protein boost” immunization regimen to generate an immune response to HIV.

The invention also relates to methods for treating or preventing HIV infection, methods for inducing an immune response (e.g., a humoral response such as a neutralizing antibody response and/or a cellular immune response), and methods of vaccinating a subject, by co-delivery of an HIV RNA molecule and an HIV polypeptide molecule (co-administration).

The invention also relates to methods for treating or preventing an HIV, methods for inducing an immune response, or methods of vaccinating a subject, by sequential administration of an HIV RNA molecule and an HIV polypeptide molecule (prime-boost).

### BRIEF DESCRIPTION OF THE DRAWINGS

FIG. 1 shows the immunization schedule for administering the HIV gp160/gp140 formulations of Example VI to BALB/c mice. “PRE” refers to a time point before a protein boost was administered; “POST” refers to a time point after a protein boost was administered.

FIG. 2 summarizes the adverse effects of the HIV gp160/gp140 formulations of Example I on the BALB/c mice. Co-delivery of RNA replicon and its encoded protein antigen showed no adverse effect.

FIG. 3A shows the HIV gp140-specific IgG antibody titers at various time points in the BALB/c mice that were administered with the HIV gp160/gp140 formulations of Example I. The numbers on top row of the graph refer to the number of animals showing detectable IgG response, out of the total number of animals examined in each group. FIG. 3B compares the anti-gp140 IgG titers in the same 5 mice before and after a boost (10 μg protein/MF59, see Table I-1) was administered. After a protein boost was administered, the IgG titers of the 1 μg RNA/Liposome primed group did not differ significantly from that of 15 μg DNA/Liposome primed group, VRP (1e7) primed group, or protein primed group.

FIGS. 4A-4C show the IgG1:IgG2a profiles of the immunized BALB/c mice. RNA/Liposome formulations induced a balanced IgG1:IgG2a subtype profile, similar to that of VRP. FIG. 4A shows the IgG1 and IgG2a titers in the BALB/c mice administered with the HIV gp160/gp140 formulations of Example I (see, Table I-1). FIG. 4B shows IgG1:IgG2a ratios in the BALB/c mice administered with the HIV gp160/gp140 formulations of Example I (see, Table I-1). FIG. 4C shows the IgG1 and IgG2a titers and IgG1:IgG2a ratios in the naked RNA primed group after the protein/MF59 boost (IgG titers were not detectable before the protein/MF59 boost).

FIG. 5 compares the immunogenicity of Clade C (DU422.1) gp160 antigen and Clade B (SF162) gp160 antigen, both delivered as liposome formulated RNA. Post-boost Th1 and Th2 type IgG responses showed a balanced profile for both Clade B and Clade C antigens.

FIGS. 6A-6B show the T-cell responses induced by the HIV gp160/gp140 formulations of Example I. FIG. 6A shows the CD4+ T-cell responses, as measured by the percentage of cytokine-secreting cells. FIG. 6B shows the CD8+ T-cell responses, as measured by the percentage of cytokine-secreting cells.

FIG. 7 shows the gp140-specific IgA antibody titers in vaginal washes of the BALB/c mice administered with the HIV gp160/gp140 formulations of Example I.

FIG. 8 shows the immunization schedule for administering various HIV gp140 formulations of Example II to BALB/c mice.

FIG. 9 summarizes the adverse effects of the HIV gp140 formulations of Example II on the BALB/c mice. Co-delivery of RNA replicon and its encoded protein antigen showed no adverse effect.

FIG. 10 shows the HIV gp140-specific IgG antibody titers in the BALB/c mice that were administered with the HIV gp140 formulations of Example II (pre-boost). Combining RNA replicon with gp140 protein induced a stronger immune response as compared to that of RNA replicon alone.

FIG. 11 shows the anti-gp140 IgG titers after a boost (10 μg protein/MF59) was administered.

FIGS. 12A and 12B show the IgG1:IgG2a profiles of the BALB/c mice that were administered with the HIV gp140 formulations of Example I. RNA/Liposome and RNA/Liposome/Protein formulations induced a balanced IgG1:IgG2a subtype profile, similar to that of VRP. FIG. 12A shows the IgG1 and IgG2a titers in the BALB/c mice administered with the HIV gp140 formulations of Example II. FIG. 12B shows IgG1:IgG2a ratios in the BALB/c mice administered with the HIVgp140 formulations of Example II. FIG. 12C shows the IgG1 and IgG2a titers in the naked RNA primed group after the protein/MF59 boost (IgG titers were not detectable before the protein/MF59 boost).

FIG. 13 shows the gp140-specific IgA antibody titers in vaginal washes of the BALB/c mice administered with the HIV gp140 formulations of Example II.

FIG. 14 shows the anti-Env IgG binding antibody titers in rabbits following RNA vaccination. Five rabbits per group were immunized intramuscularly with the respective vaccines at 0 and 4 weeks followed by two boosters with an MF59-adjuvanted-o-gp140 (TV1.C) (Env/MF59) vaccine at 12 and 24 weeks. The nucleic acid and VRP vaccines encoded the o-gp140 protein of TV1.C. Anti-Env binding antibody titers to TV1.C o-gp140 was determined using an ELISA. Sera were titrated from a dilution of 1:400 (dotted line). Geometric mean titers with SEM are shown.

FIG. 15 shows antibodies that neutralize MW965 Env pseudovirus are induced upon RNA vaccination. Sera from the 2wp2, 2wp3, and 2wp4 time-points were assayed for neutralization using an U87 CD4 CCR5 neutralization assay with the MW965 Env pseudovirus. Each symbol is the titer obtained for a rabbit with the horizontal bar showing the geometric mean titer. Numbers above the graph show the number of responders (titers at or above the serum titration start of 1:160; dotted line)/5 rabbits. Statistical analysis was carried out using a Kruskal-Wallis test with Dunns post test.

FIGS. 16A-B are graphs showing the total (A) and anti-Env (B) Ig titers in rabbit vaginal washes. Samples were titrated starting at 1:25 (total Ig; A) or neat (anti-Env Ig; B) on ELISA plates using an anti-rabbit Ig capture antibody (A) or coated Env protein (B). Cut-off at 2 for the Env-specific Ig graph (B) at bottom is arbitrary. Greater than 90% of pre-immune washes yield a titer between neat and 2 and therefore this was chosen as the cut-off titer. In a few instances, pre-immune washes (1-2 rabbits depending on group) yielded high non-specific titers (>2). Rabbits that these were harvested from were removed from the analysis for all time-points. Horizontal bar for each group shows the geometric mean titer.

FIGS. 17A-D show the anti-Env IgG binding antibody titers in rhesus macaques following RNA vaccination. Six macaques per group were immunized intramuscularly with the respective vaccines at 0, 4, and 12 weeks (solid black triangles on x-axis) followed by two boosters with an MF59-adjuvanted-o-gp140 (TV1.C) (Env/MF59) vaccine at 24 and 36 weeks (open triangles on x-axis). The nucleic acid and VRP vaccines encoded the o-gp140 protein of TV1.C. Anti-Env binding antibody titers to TV1.C o-gp140 was determined using an ELISA. Sera were titrated from a dilution of 1:25. Each symbol denotes the titer from one macaque and numbers above each graph denotes the number of responders (titers above 1:25)/6 macaques.

FIG. 18 shows anti-Env T-cell responses in rhesus macaques following RNA vaccination. PBMCs from each of the immunized macaques from the respective groups were re-stimulated with either a pool of the consensus Clade C gp120 peptide library (first column) or a pool of the consensus Clade C gp41 peptide library (middle column) or TV1.0 protein in an ELISPOT assay. Graphs show the T-cell response over time expressed as the number of IFNγ spot forming cells (SFC)/10⁶ PBMC for each individual macque/group. Arrows below the graphs show immunizations.

FIG. 19 shows the vector used to transcribe H351 [T7G-VCR-CHIM2.12-SF162gp160mod] RNA, the annotated sequence of the vector and the insert.

FIG. 20 shows the vector used to transcribe H350 [T7G-VCR-CHIM2.12-Du422.1 gp160mod] RNA, the annotated sequence of the vector and the insert.

FIG. 21 shows the vector used to transcribe H354 [T7(−G)-TV1c8.2 gp140mod UNC] RNA, the annotated sequence of the vector and the insert.

FIG. 22 shows the vector used to transcribe H412 [pCMV-KM2 SF162 TPA-gp160mod UNC] RNA, the annotated sequence of the vector and the insert; and the vector used to transcribe H425 [pCMV-KM2 TV1c8.2 TPA gp140mod UNC] RNA, the annotated sequence of the vector and the insert.

FIG. 23 is a graph showing the Env-specific binding IgG titers of rabbits following RNA, RNA and protein, or protein only vaccination. Rabbits (n=6) were immunized at 0 and 4 weeks with 25 μg of the HIV-SAM/CMF34 vaccine and/or 25 μg of the MF59- or alum-adjuvanted Env vaccine. For concurrent vaccination of the HIV-SAM/CMF34 and MF59- or alum-adjuvanted Env vaccine, animals either received the vaccines separated approximately by 3 cms in the same quadriceps muscle (same side, 2sites) or each vaccine was immunized in the quadriceps muscle of a leg (opp. Side—opposite side). Sera from 2w (2wp2) or 8w (8wp2) after the 2nd immunization were assayed by ELISA to estimate TV1, gp140 Env-specific binding IgG titers. Each symbol in the graph shows the titer for a rabbit with the horizontal line for a group denoting the geometric mean titer.

FIGS. 24A-C show vaccine induced antigen-specific T-cell responses in time. IFN-γ (FIG. 24A), IL2 (FIG. 24B) and IL4 (FIG. 24C) secretion by PBMC of all individual animals per group towards gp120 Consensus (Cons) C peptide pool (pp), gp41 Cons C pp, or recombinant TV 1 gp140 were measured by ELISpot assay.

FIG. 25 shows neutralization (IC₅₀) of sera taken at two weeks post 4th (wk 26) and two weeks post 5th (wk 38) immunization. Sera were evaluated against a clade C Tier 2 (SHIV1157ipd3N4) Pseudovirus, a Tier 1 (SHIV1157ipEL-p) PV, a Tier 1 HIV-1/TV1 PV and against a Tier 1 Clade B PV (SHIV SF162P4).

FIG. 26 shows neutralization (IC₅₀) of sera taken at two weeks post 5th (wk 38) immunization. Sera were evaluated against a clade C Tier 1 (MW965.26) in TZM-bl cells and Tier 2 viruses (TV1.21.LucR.T2A.ecto and Ce1176_A3.LucR.T2A.ecto) in A3R5.7 cells.

### DETAILED DESCRIPTION OF THE INVENTION

### 1. Overview

One particular advantage of an HIV RNA vaccine is that RNA molecules are self-adjuvanting. For example, the inventors observed that RNA molecules (formulated in liposomes) induced several serum cytokines, including IFN-α, IP-10 (CXCL-10), IL-6, KC (CXCL1), IL-5, IL-13, MCP-1, and MIP-α, within 24 hours of intramuscular injection into a mouse model. The cytokines can enhance the host immune response to the protein antigen that was encoded by the RNA molecule.

Vaccination strategies that combine an HIV RNA molecule and an HIV polypeptide molecule (e.g., administering an immunogenic composition that has an RNA component and a protein component; or sequential administration regimens such as “RNA prime, protein boost”) provide several benefits. For example, the polypeptide molecule can enhance total antibody titers in the host, while the RNA molecule can enhance the production of antibodies that recognize an antigen in its native structure. Thus the combination can induce an antibody response with an enhanced ratio of functional antibodies (e.g., neutralizing antibodies) to total antibodies. Furthermore, RNA molecules promote type 1 T helper responses (Th1, IFN-γ^{hi}, IL-4^{lo}), whereas protein molecules promote type 2 T helper responses. Thus, combining an RNA molecule and a polypeptide molecule can promote both T cell-mediated immunity as well as humoral immunity. In addition, RNAs molecule may be delivered to cells using delivery systems such as liposomes or oil-in-water emulsions. Liposomes and oil-in-water emulsions are also known to have adjuvant activities. Thus, the adjuvant activity of the RNA together with adjuvant activity of the delivery system can act synergistically to enhance the immune response to an antigen. Finally, multivalency may be achieved by combining a polypeptide antigen with an RNA that encodes a different antigen from the same pathogen.

### (A) Co-Administration of an RNA Molecule and a Polypeptide Molecule

In one aspect, the invention relates to immunogenic compositions that comprise an HIV RNA component and an HIV polypeptide component. Immunogenic compositions that deliver antigenic epitopes in two different forms—a first epitope from HIV, in RNA-coded form; and a second epitope from HIV, in polypeptide form—can enhance the immune response to HIV.

Preferably, the first epitope and the second epitope are the same epitope (i.e., the first antigen, in RNA-coded form, and the second antigen, in polypeptide form, share at least one common epitope). For example, the RNA component of the immunogenic composition can encode a protein that is substantially the same as the polypeptide component of the immunogenic composition (e.g., the amino acid sequence encoded by the RNA molecule and the polypeptide component of the immunogenic composition share at least about 90% sequence identity across the length of the shorter antigen). Alternatively, the two antigens have the same epitope, such as the same immunodominant epitope(s).

As described herein, the inventors have evaluated the efficacies of immunogenic compositions that comprise (i) a self-replicating RNA molecule that encodes an HIV antigen, and (ii) HIV antigen in polypeptide form. The results demonstrated that co-administering an RNA molecule that encodes an HIV antigen, together with the HIV antigen in polypeptide form, potentiated the immune response to the antigen, resulting in higher antibody titers as compared to administering the RNA molecule alone. In addition, co-administering an HIV antigen in RNA-coded form and in polypeptide form enhanced isotype switching from IgG₁ to IgG₂ₐ, producing a more balanced IgG₁:IgG₂ₐ subtype profile as compared to administering the polypeptide antigen alone. Finally, the studies disclosed herein also show that administrating an antigen in RNA-coded form and polypeptide form can enhance CD4+ and CD8+ T cell-mediated immunity.

The immunogenic compositions described herein can be formulated as a vaccine to induce or enhance the host immune response to HIV infection. Also provided herein are methods of using the immunogenic compositions of the invention to induce or enhance an immune response in a subject in need thereof.

### (B) Prime-Boost

In another aspect, the invention relates to a kit comprising: (i) a priming composition comprising a self-replicating RNA molecule that encodes an HIV polypeptide antigen that comprises a first epitope, and (ii) a boosting composition comprising an HIV polypeptide antigen that comprises a second epitope; wherein said first epitope and second epitope are the same epitope (i.e., the first antigen, in RNA-coded form, and the second antigen, in polypeptide form, share at least one common epitope). The kit may be used for sequential administration of the priming and the boosting compositions.

In another aspect, the invention relates to a method for treating or preventing an infectious disease, a method for inducing an immune response in a subject, or a method of vaccinating a subject, comprising: (i) administering to a subject in need thereof at least once a therapeutically effective amount of a priming composition comprising a self-replicating RNA molecule that encodes an HIV polypeptide antigen that comprises a first epitope, and (ii) subsequently administering to the subject at least once a therapeutically effective amount of a boosting composition comprising a polypeptide antigen that comprises a second epitope; wherein said first epitope and second epitope are the same epitope (i.e., the first antigen, in RNA-coded form, and the second antigen, in polypeptide form, share at least one common epitope).

As described herein, the inventors have evaluated RNA prime, protein boost vaccination strategies. These studies demonstrate several benefits of the RNA prime, protein boost strategy, as compared to a protein prime, protein boost strategy, including, for example, increased antibody titers, a more balanced IgG₁:IgG₂ₐ subtype profile, induction of T_{H}1 type, CD4+ T cell-mediated immune response that was similar to that of viral particles, and reduced production of non-neutralizing antibodies.

Preferably, the RNA molecule in the priming composition encodes an HIV protein that is substantially the same as the polypeptide molecule in the boosting composition (e.g., the amino acid sequence encoded by the RNA molecule in the priming composition and the polypeptide in the boosting composition share at least about 90% sequence identity across the length of the shorter antigen). Alternatively, the two antigens have the same epitope, such as the same immunodominant epitope(s).

The priming and boosting compositions described herein can be formulated as a vaccine to induce or enhance the immune response to a pathogen. Also provided herein are methods of using the priming and boosting compositions of the invention to induce or enhance an immune response in a subject in need thereof.

The invention also relates to immunogenic compositions, pharmaceutical compositions, or kits as described herein for use in therapy, and to the use of immunogenic compositions, pharmaceutical compositions, or kits as described herein for the manufacture of a medicament for inducing, enhancing or generating an immune response.

### 2. Immunogenic Compositions

In one aspect, the invention provides an immunogenic composition comprising an HIV RNA component and an HIV polypeptide component. The immunogenic composition comprises: (i) a self-replicating RNA molecule that encodes a first polypeptide antigen comprising a first epitope (the RNA component); and (ii) a second polypeptide antigen comprising a second epitope (the polypeptide component); wherein said first epitope and second epitope are epitopes from HIV.

The first epitope and second epitope can be the same epitope, or different epitopes if desired. The first epitope and second epitope can be from the same polypeptide of HIV, or different polypeptides of HIV. The first epitope and second epitope can also be epitopes which are highly conserved between different strains or subspecies of the pathogen, such as those epitopes with limited or no mutational variations.

In certain embodiments, the first polypeptide antigen and the second polypeptide antigen are derived from the same protein from HIV. For example, the RNA molecule may encode a first polypeptide antigen comprising a full-length protein from HIV, or an antigenic portion thereof, optionally fused with a heterologous sequence that may facilitate the expression, production, purification or detection of the viral protein encoded by the RNA. The second polypeptide antigen may be a recombinant protein comprising the full-length protein, or an antigenic portion thereof, optionally fused with a heterologous sequence (e.g., His-tag) that may facilitate the expression, production, purification or detection of the second polypeptide antigen or a truncated form (e.g., gp140 is a truncated form of gp160). Alternatively, the first polypeptide antigen, the second polypeptide antigen, or both, may comprise a mutation variant of a protein from HIV (e.g., a viral protein having amino acid substitution(s), addition(s), or deletion(s)).

Preferably, the amino acid sequence identity between the first polypeptide antigen and the second polypeptide antigen is at least about 40%, least about 50%, least about 60%, least about 65%, least about 70%, at least about 75%, at least about 80%, at least about 85%, at least about 90%, at least about 95%, at least about 96%, at least about 97%, at least about 98%, or at least about 99%. In certain embodiments, the first polypeptide antigen and the second polypeptide antigen are the same antigen.

In certain embodiments, the first polypeptide antigen and the second polypeptide antigen share at least 1, at least 2, at least 3, at least 4, or at least 5 common B-cell or T-cell epitopes. In certain embodiments, the first polypeptide antigen and the second polypeptide antigen have at least one common immunodominant epitope. In certain embodiments, the first polypeptide antigen and the second polypeptide antigen have the same immunodominant epitope(s), or the same primary immunodominant epitope.

In certain embodiments, the first polypeptide antigen is a soluble or membrane anchored polypeptide, and the second polypeptide antigen is a soluble polypeptide. For example, if the wild type viral protein is a transmembrane surface protein, the RNA molecule may comprise the full-length coding sequence to produce the first (membrane-anchored) antigen, while the transmembrane region of the viral protein may be deleted to produce the second polypeptide antigen (which is soluble).

In certain embodiments, the first antigen or the second antigen is a fusion polypeptide further comprising a third epitope. The third epitope may be from a pathogen other than HIV, or from a different HIV antigen.

### A. Antigens

Antigens suitable for inclusion in the immunogenic compositions described herein (either in RNA-coded form or in polypeptide form) may be derived from any pathogen (e.g., a bacterial pathogen, a viral pathogen, a fungal pathogen, a protozoan pathogen, or a multi-cellular parasitic pathogen), allergen or tumor.

### HIV

In certain embodiments, the first and second antigens are derived from HIV-1, including any HIV-1 strain, such as HIV-1_{CM235}, HIV-1_{US4}, HIV-1_{SF162}, HIV-1_{TV1}, HIV-1_{MJ4}, HIV-1 subtype (or clade), such as A, B, C, D, F, G, H, J. K, and O, and HIV-1 circulating recombinant forms (CRFs), including, A/B, A/E, A/G, A/G/I, etc.

In certain embodiments, the first and second antigens are independently derived from one or more of the following proteins: gag (p24gag, p55gag), pol, env (gp160, gp140, gp120, gp41), tax, tat, rex, rev, nef, vif, vpu, or vpr. In certain embodiments, the first and second antigens are HIV Env polypeptides, such as gp160, gp140 or gp120. The Env polypeptides can be monomers or oligomers, for example a gp120 monomer, or homo- or hetero-oligomers of gp140 and gp160.

In certain embodiments, the HIV antigen suitable for inclusion in the immunogenic compositions described herein is derived from HIV (e.g., HIV-1) Env protein (including, e.g., gp120, gp140, and gp160).

The nucleic acid sequences encoding, and the amino acid sequences of, Env proteins from many HIV isolates are well known in the art. For example, the amino acid sequences of Env protein (gp160 precursors) from HIV-1 Bru, HIV-1 MN, HIV-1 ELI, HIV-1 RF, HIV-1 SF2C and HIV-1 SC, are disclosed as SEQ ID NOS; 1-6 in U.S. Pat. No. 6,284,248.

It is well-known that Env is synthesized first as a gp160 polyprotein precursor in the endoplasmic reticulum, which is cleaved to form gp120 and gp41, or truncated to form gp140. gp120 corresponds to the N-terminal end of the gp160 without the oligomerization domain or transmembrane domain, gp140 corresponds to the N-terminal end of the gp160 without the transmembrane domain, but retains the oligomerization domain. See, Morikawa et al., J. Virol, 67:3601-3604 (1993); Richmond et al., J. Virol., 72:9092-9100 (1998); Earl et al. J. Virol., 75:645-653 (2001).

The gp160 polyprotein precursor is cleaved, at a major cleavage site and/or minor cleavage site, to form gp120. If desired one or both cleavage sites can be mutated to prevent processing of gp160 into gp120. A number of suitable mutations are well known in the art and are described, for example, in U.S. Pat. No. 6,284,248, and U.S. Patent Application Publication No. 2010/0316698.

An exemplary gp140 sequence is set forth as SEQ ID NO: [______]. An exemplary gp120 sequence is set forth as SEQ ID NO: [______]. An exemplary gp160 sequence is set forth as SEQ ID NO: [______]. The invention may use an HIV Env antigen comprising SEQ ID NOs: ______, ______, or ______, or comprising an amino acid sequence that is at least 75% identical to SEQ ID NOs: ______, ______, or ______, (e.g., at least 80%, at least 85%, at least 90%, at least 95%, at least 97%, at least 98%, at least 99%, or 100% identical to SEQ ID NOs: ______, ______ or ______).

AX456011 nucleotide seq (SEQ ID NO:___):
atga gagtgatggg gacacagaag aattgtcaac aatggtggat atggggcatc
ttaggcttct ggatgctaat gatttgtaac accgaggacc tgtgggtgac cgtgtactac
ggcgtgcccg tgtggcgcga cgccaagacc accctgttct gcgccagcga cgccaaggcc
tacgagaccg aggtgcacaa cgtgtgggcc acccacgcct gcgtgcccac cgaccccaac
ccccaggaga tcgtgctggg caacgtgacc gagaacttca acatgtggaa gaacgacatg
gccgaccaga tgcacgagga cgtgatcagc ctgtgggacc agagcctgaa gccctgcgtg
aagctgaccc ccctgtgcgt gaccctgaac tgcaccgaca ccaacgtgac cggcaaccgc
accgtgaccg gcaacagcac caacaacacc aacggcaccg gcatctacaa catcgaggag
atgaagaact gcagcttcaa cgccaccacc gagctgcgcg acaagaagca caaggagtac
gccctgttct accgcctgga catcgtgccc ctgaacgaga acagcgacaa cttcacctac
cgcctgatca actgcaacac cagcaccatc acccaggcct gccccaaggt gagcttcgac
cccatcccca tccactactg cgcccccgcc ggctacgcca tcctgaagtg caacaacaag
accttcaacg gcaccggccc ctgctacaac gtgagcaccg tgcagtgcac ccacggcatc
aagcccgtgg tgagcaccca gctgctgctg aacggcagcc tggccgagga gggcatcatc
atccgcagcg agaacctgac cgagaacacc aagaccatca tcgtgcacct gaacgagagc
gtggagatca actgcacccg ccccaacaac aacacccgca agagcgtgcg catcggcccc
ggccaggcct tctacgccac caacgacgtg atcggcaaca tccgccaggc ccactgcaac
atcagcaccg accgctggaa caagaccctg cagcaggtga tgaagaagct gggcgagcac
ttccccaaca agaccatcca gttcaagccc cacgccggcg gcgacctgga gatcaccatg
cacagcttca actgccgcgg cgagttcttc tactgcaaca ccagcaacct gttcaacagc
acctaccaca gcaacaacgg cacctacaag tacaacggca acagcagcag ccccatcacc
ctgcagtgca agatcaagca gatcgtgcgc atgtggcagg gcgtgggcca ggccacctac
gcccccccca tcgccggcaa catcacctgc cgcagcaaca tcaccggcat cctgctgacc
cgcgacggcg gcttcaacac caccaacaac accgagacct tccgccccgg cggcggcgac
atgcgcgaca actggcgcag cgagctgtac aagtacaagg tggtggagat caagcccctg
ggcatcgccc ccaccaaggc caagcgccgc gtggtgcagc gcgagaagcg cgccgtgggc
atcggcgccg tgttcctggg cttcctgggc gccgccggca gcaccatggg cgccgccagc
atcaccctga ccgtgcaggc ccgccagctg ctgagcggca tcgtgcagca gcagagcaac
ctgctgaagg ccatcgaggc ccagcagcac atgctgcagc tgaccgtgtg gggcatcaag
cagctgcagg cccgcgtgct ggccatcgag cgctacctga aggaccagca gctgctgggc
atctggggct gcagcggccg cctgatctgc accaccgccg tgccctggaa cagcagctgg
agcaacaaga gcgagaagga catctgggac aacatgacct ggatgcagtg ggaccgcgag
atcagcaact acaccggcct gatctacaac ctgctggagg acagccagaa ccagcaggag
aagaacgaga aggacctgct ggagctggac aagtggaaca acctgtggaa ctggttcgac
atcagcaact ggccctggta catcaagatc ttcatcatga tcgtgggcgg cctgatcggc
ctgcgcatca tcttcgccgt gctgagcatc gtgaaccgcg tgcgccaggg ctacagcccc
ctgagcttcc agaccctgac ccccagcccc cgcggcctgg accgcctggg cggcatcgag
gaggagggcg gcgagcagga ccgcgaccgc agcatccgcc tggtgagcgg cttcctgagc
ctggcctggg acgacctgcg caacctgtgc ctgttcagct accaccgcct gcgcgacttc
atcctgatcg ccgtgcgcgc cgtggagctg ctgggccaca gcagcctgcg cggcctgcag
cgcggctggg agatcctgaa gtacctgggc agcctggtgc agtactgggg cctggagctg
aagaagagcg ccatcagcct gctggacacc atcgccatca ccgtggccga gggcaccgac
cgcatcatcg agctggtgca gcgcatctgc cgcgccatcc tgaacatccc ccgccgcatc
cgccagggct tcgaggccgc cctgctgtaa
AX456011 AA seq (SEQ ID NO:___):
MRVMGTQKNCQQWWIWGILGFWMLMICNTEDLWVTVYYGVPVWRDAKTTLFCASDAKAYETEVHN
VWATHACVPTDPNPQEIVLGNVTENFNMWKNDMADQMHEDVISLWDQSLKPCVKLTPLCVTLNCT
DTNVTGNRTVTGNSTNNTNGTGIYNIEEMKNCSFNATTELRDKKHKEYALFYRLDIVPLNENSDN
FTYRLINCNTSTITQACPKVSFDPIPIHYCAPAGYAILKCNNKTFNGTGPCYNVSTVQCTHGIKP
VVSTQLLLNGSLAEEGIIIRSENLTENTKTIIVHLNESVEINCTRPNNNTRKSVRIGPGQAFYAT
NDVIGNIRQAHCNISTDRWNKTLQQVMKKLGEHFPNKTIQFKPHAGGDLEITMHSFNCRGEFFYC
NTSNLFNSTYHSNNGTYKYNGNSSSPITLQCKIKQIVRMWQGVGQATYAPPIAGNITCRSNITGI
LLTRDGGFNTTNNTETFRPGGGDMRDNWRSELYKYKVVEIKPLGIAPTKAKRRVVQREKRAVGIG
AVFLGFLGAAGSTMGAASITLTVQARQLLSGIVQQQSNLLKAIEAQQHMLQLTVWGIKQLQARVL
AIERYLKDQQLLGIWGCSGRLICTTAVPWNSSWSNKSEKDIWDNMTWMQWDREISNYTGLIYNLL
EDSQNQQEKNEKDLLELDKWNNLWNWFDISNWPWYIKIFIMIVGGLIGLRIIFAVLSIVNRVRQG
YSPLSFQTLTPSPRGLDRLGGIEEEGGEQDRDRSIRLVSGFLSLAWDDLRNLCLFSYHRLRDFIL
IAVRAVELLGHSSLRGLQRGWEILKYLGSLVQYWGLELKKSAISLLDTIAITVAEGTDRIIELVQ
RICRAILNIPRRIRQGFEAALL*
gp140 nucleotide sequence (SEQ ID NO:___):
atggatgcaatgaagagagggctctgctgtgtgctgctgctgtgtggagcagtcttcgtttcgcc
caacaccgaggacctgtgggtgaccgtgtactacggcgtgcccgtgtggcgcgacgccaagacca
ccctgttctgcgccagcgacgccaaggcctacgagaccgaggtgcacaacgtgtgggccacccac
gcctgcgtgcccaccgaccccaacccccaggagatcgtgctgggcaacgtgaccgagaacttcaa
catgtggaagaacgacatggccgaccagatgcacgaggacgtgatcagcctgtgggaccagagcc
tgaagccctgcgtgaagctgacccccctgtgcgtgaccctgaactgcaccgacaccaacgtgacc
ggcaaccgcaccgtgaccggcaacagcaccaacaacaccaacggcaccggcatctacaacatcga
ggagatgaagaactgcagcttcaacgccaccaccgagctgcgcgacaagaagcacaaggagtacg
ccctgttctaccgcctggacatcgtgcccctgaacgagaacagcgacaacttcacctaccgcctg
atcaactgcaacaccagcaccatcacccaggcctgccccaaggtgagcttcgaccccatccccat
ccactactgcgcccccgccggctacgccatcctgaagtgcaacaacaagaccttcaacggcaccg
gcccctgctacaacgtgagcaccgtgcagtgcacccacggcatcaagcccgtggtgagcacccag
ctgctgctgaacggcagcctggccgaggagggcatcatcatccgcagcgagaacctgaccgagaa
caccaagaccatcatcgtgcacctgaacgagagcgtggagatcaactgcacccgccccaacaaca
acacccgcaagagcgtgcgcatcggccccggccaggccttctacgccaccaacgacgtgatcggc
aacatccgccaggcccactgcaacatcagcaccgaccgctggaacaagaccctgcagcaggtgat
gaagaagctgggcgagcacttccccaacaagaccatccagttcaagccccacgccggcggcgacc
tggagatcaccatgcacagcttcaactgccgcggcgagttcttctactgcaacaccagcaacctg
ttcaacagcacctaccacagcaacaacggcacctacaagtacaacggcaacagcagcagccccat
caccctgcagtgcaagatcaagcagatcgtgcgcatgtggcagggcgtgggccaggccacctacg
ccccccccatcgccggcaacatcacctgccgcagcaacatcaccggcatcctgctgacccgcgac
ggcggcttcaacaccaccaacaacaccgagaccttccgccccggcggcggcgacatgcgcgacaa
ctggcgcagcgagctgtacaagtacaaggtggtggagatcaagcccctgggcatcgcccccacca
aggccatctcctccgtggtgcagagcgagaagagcgccgtgggcatcggcgccgtgttcctgggc
ttcctgggcgccgccggcagcaccatgggcgccgccagcatcaccctgaccgtgcaggcccgcca
gctgctgagcggcatcgtgcagcagcagagcaacctgctgaaggccatcgaggcccagcagcaca
tgctgcagctgaccgtgtggggcatcaagcagctgcaggcccgcgtgctggccatcgagcgctac
ctgaaggaccagcagctgctgggcatctggggctgcagcggccgcctgatctgcaccaccgccgt
gccctggaacagcagctggagcaacaagagcgagaaggacatctgggacaacatgacctggatgc
agtgggaccgcgagatcagcaactacaccggcctgatctacaacctgctggaggacagccagaac
cagcaggagaagaacgagaaggacctgctggagctggacaagtggaacaacctgtggaactggtt
cgacatcagcaactggccctggtacatctaa
gp140 AA sequence (SEQ ID NO:___):
MDAMKRGLCCVLLLCGAVFVSPNTEDLWVTVYYGVPVWRDAKTTLFCASDAKAYETEVHNVWATH
ACVPTDPNPQEIVLGNVTENFNMWKNDMADQMHEDVISLWDQSLKPCVKLTPLCVTLNCTDTNVT
GNRTVTGNSTNNTNGTGIYNIEEMKNCSFNATTELRDKKHKEYALFYRLDIVPLNENSDNFTYRL
INCNTSTITQACPKVSFDPIPIHYCAPAGYAILKCNNKTFNGTGPCYNVSTVQCTHGIKPVVSTQ
LLLNGSLAEEGIIIRSENLTENTKTIIVHLNESVEINCTRPNNNTRKSVRIGPGQAFYATNDVIG
NIRQAHCNISTDRWNKTLQQVMKKLGEHFPNKTIQFKPHAGGDLEITMHSFNCRGEFFYCNTSNL
FNSTYHSNNGTYKYNGNSSSPITLQCKIKQIVRMWQGVGQATYAPPIAGNITCRSNITGILLTRD
GGFNTTTNNTETFRPGGGDMRDNWRSELYKYKVVEIKPLGIAPTKAISSVVQSEKSAVGIGAVFL
GFLGAAGSTMGAASITLTVQARQLLSGIVQQQSNLLKAIEAQQHMLQLTVWGIKQLQARVLAIER
YLKDQQLLGIWGCSGRLICTTAVPWNSSWSNKSEKDIWDNMTWMQWDREISNYTGLIYNLLEDSQ
NQQEKNEKDLLELDKWNNLWNWFDISNWPWYI*
TV1
Gp160 nt seq (SEQ ID NO:___):
atgcgcgtgatgggcacccagaagaactgccagcagtggtggatctggggcatcctgggcttctg
gatgctgatgatctgcaacaccgaggacctgtgggtgaccgtgtactacggcgtgcccgtgtggc
gcgacgccaagaccaccctgttctgcgccagcgacgccaaggcctacgagaccgaggtgcacaac
gtgtgggccacccacgcctgcgtgcccaccgaccccaacccccaggagatcgtgctgggcaacgt
gaccgagaacttcaacatgtggaagaacgacatggccgaccagatgcacgaggacgtgatcagcc
tgtgggaccagagcctgaagccctgcgtgaagctgacccccctgtgcgtgaccctgaactgcacc
gacaccaacgtgaccggcaaccgcaccgtgaccggcaacagcaccaacaacaccaacggcaccgg
catctacaacatcgaggagatgaagaactgcagcttcaacgccaccaccgagctgcgcgacaaga
agcacaaggagtacgccctgttctaccgcctggacatcgtgcccctgaacgagaacagcgacaac
ttcacctaccgcctgatcaactgcaacaccagcaccatcacccaggcctgccccaaggtgagctt
cgaccccatccccatccactactgcgcccccgccggctacgccatcctgaagtgcaacaacaaga
ccttcaacggcaccggcccctgctacaacgtgagcaccgtgcagtgcacccacggcatcaagccc
gtggtgagcacccagctgctgctgaacggcagcctggccgaggagggcatcatcatccgcagcga
gaacctgaccgagaacaccaagaccatcatcgtgcacctgaacgagagcgtggagatcaactgca
cccgccccaacaacaacacccgcaagagcgtgcgcatcggccccggccaggccttctacgccacc
aacgacgtgatcggcaacatccgccaggcccactgcaacatcagcaccgaccgctggaacaagac
cctgcagcaggtgatgaagaagctgggcgagcacttccccaacaagaccatccagttcaagcccc
acgccggcggcgacctggagatcaccatgcacagcttcaactgccgcggcgagttcttctactgc
aacaccagcaacctgttcaacagcacctaccacagcaacaacggcacctacaagtacaacggcaa
cagcagcagccccatcaccctgcagtgcaagatcaagcagatcgtgcgcatgtggcagggcgtgg
gccaggccacctacgccccccccatcgccggcaacatcacctgccgcagcaacatcaccggcatc
ctgctgacccgcgacggcggcttcaacaccaccaacaacaccgagaccttccgccccggcggcgg
cgacatgcgcgacaactggcgcagcgagctgtacaagtacaaggtggtggagatcaagcccctgg
gcatcgcccccaccaaggccaagcgccgcgtggtgcagcgcgagaagcgcgccgtgggcatcggc
gccgtgttcctgggcttcctgggcgccgccggcagcaccatgggcgccgccagcatcaccctgac
cgtgcaggcccgccagctgctgagcggcatcgtgcagcagcagagcaacctgctgaaggccatcg
aggcccagcagcacatgctgcagctgaccgtgtggggcatcaagcagctgcaggcccgcgtgctg
gccatcgagcgctacctgaaggaccagcagctgctgggcatctggggctgcagcggccgcctgat
ctgcaccaccgccgtgccctggaacagcagctggagcaacaagagcgagaaggacatctgggaca
acatgacctggatgcagtgggaccgcgagatcagcaactacaccggcctgatctacaacctgctg
gaggacagccagaaccagcaggagaagaacgagaaggacctgctggagctggacaagtggaacaa
cctgtggaactggttcgacatcagcaactggccctggtacatcaagatcttcatcatgatcgtgg
gcggcctgatcggcctgcgcatcatcttcgccgtgctgagcatcgtgaaccgcgtgcgccagggc
tacagccccctgagcttccagaccctgacccccagcccccgcggcctggaccgcctgggcggcat
cgaggaggagggcggcgagcaggaccgcgaccgcagcatccgcctggtgagcggcttcctgagcc
tggcctgggacgacctgcgcaacctgtgcctgttcagctaccaccgcctgcgcgacttcatcctg
atcgccgtgcgcgccgtggagctgctgggccacagcagcctgcgcggcctgcagcgcggctggga
gatcctgaagtacctgggcagcctggtgcagtactggggcctggagctgaagaagagcgccatca
gcctgctggacaccatcgccatcaccgtggccgagggcaccgaccgcatcatcgagctggtgcag
cgcatctgccgcgccatcctgaacatcccccgccgcatccgccagggcttcgaggccgccctgct
gtaa
Gp160 AA seq (SEQ ID NO:___):
MRVMGTQKNCQQWWIWGILGFWMLMICNTEDLWVTVYYGVPVWRDAKTTLFCASDAKAYETEVHN
VWATHACVPTDPNPQEIVLGNVTENFNMWKNDMADQMHEDVISLWDQSLKPCVKLTPLCVTLNCT
DTNVTGNRTVTGNSTNNTNGTGIYNIEEMKNCSFNATTELRDKKHKEYALFYRLDIVPLNENSDN
FTYRLINCNTSTITQACPKVSFDPIPIHYCAPAGYAILKCNNKTFNGTGPCYNVSTVQCTHGIKP
VVSTQLLLNGSLAEEGIIIRSENLTENTKTIIVHLNESVEINCTRPNNNTRKSVRIGPGQAFYAT
NDVIGNIRQAHCNISTDRWNKTLQQVMKKLGEHFPNKTIQFKPHAGGDLEITMHSFNCRGEFFYC
NTSNLFNSTYHSNNGTYKYNGNSSSPITLQCKIKQIVRMWQGVGQATYAPPIAGNITCRSNITGI
LLTRDGGFNTTNNTETFRPGGGDMRDNWRSELYKYKVVEIKPLGIAPTKAKRRVVQREKRAVGIG
AVFLGFLGAAGSTMGAASITLTVQARQLLSGIVQQQSNLLKAIEAQQHMLQLTVWGIKQLQARVL
AIERYLKDQQLLGIWGCSGRLICTTAVPWNSSWSNKSEKDIWDNMTWMQWDREISNYTGLIYNLL
EDSQNQQEKNEKDLLELDKWNNLWNWFDISNWPWYIKJFIMIVGGLIGLRIIFAVLSIVNRVRQG
YSPLSFQTLTPSPRGLDRLGGIEEEGGEQDRDRSIRLVSGFLSLAWDDLRNLCLFSYHRLRDFIL
IAVRAVELLGHSSLRGLQRGWEILKYLGSLVQYWGLELKKSAISLLDTIAITVAEGTDRIIELVQ
RICRAILNIPRRIRQGFEAALL*
Gp120 nt seq (SEQ ID NO:___):
atggatgcaatgaagagagggctctgctgtgtgctgctgctgtgtggagcagtttcgtttcgccc
aacaccgaggacctgtgggtgaccgtgtactacggcgtgcccgtgtggcgcgacgccaagaccac
cctgttctgcgccagcgacgccaaggcctacgagaccgaggtgcacaacgtgtgggccacccacg
cctgcgtgcccaccgaccccaacccccaggagatcgtgctgggcaacgtgaccgagaacttcaac
atgtggaagaacgacatggccgaccagatgcacgaggacgtgatcagcctgtgggaccagagcct
gaagccctgcgtgaagctgacccccctgtgcgtgaccctgaactgcaccgacaccaacgtgaccg
gcaaccgcaccgtgaccggcaacagcaccaacaacaccaacggcaccggcatctacaacatcgag
gagatgaagaactgcagcttcaacgccaccaccgagctgcgcgacaagaagcacaaggagtacgc
cctgttctaccgcctggacatcgtgcccctgaacgagaacagcgacaacttcacctaccgcctga
tcaactgcaacaccagcaccatcacccaggcctgccccaaggtgagcttcgaccccatccccatc
cactactgcgcccccgccggctacgccatcctgaagtgcaacaacaagaccttcaacggcaccgg
cccctgctacaacgtgagcaccgtgcagtgcacccacggcatcaagcccgtggtgagcacccagc
tgctgctgaacggcagcctggccgaggagggcatcatcatccgcagcgagaacctgaccgagaac
accaagaccatcatcgtgcacctgaacgagagcgtggagatcaactgcacccgccccaacaacaa
cacccgcaagagcgtgcgcatcggccccggccaggccttctacgccaccaacgacgtgatcggca
acatccgccaggcccactgcaacatcagcaccgaccgctggaacaagaccctgcagcaggtgatg
aagaagctgggcgagcacttccccaacaagaccatccagttcaagccccacgccggcggcgacct
ggagatcaccatgcacagcttcaactgccgcggcgagttcttctactgcaacaccagcaacctgt
tcaacagcacctaccacagcaacaacggcacctacaagtacaacggcaacagcagcagccccatc
accctgcagtgcaagatcaagcagatcgtgcgcatgtggcagggcgtgggccaggccacctacgc
cccccccatcgccggcaacatcacctgccgcagcaacatcaccggcatcctgctgacccgcgacg
gcggcttcaacaccaccaacaacaccgagaccttccgccccggcggcggcgacatgcgcgacaac
tggcgcagcgagctgtacaagtacaaggtggtggagatcaagcccctgggcatcgcccccaccaa
ggccaagcgccgcgtggtgcagcgcgagaagcgctaa
Gp120 AA seq (SEQ ID NO:___):
MDAMKRGLCCVLLLCGAVFVSPNTEDLWVTVYYGVPVWRDAKTTLFCASDAKAYETEVHNVWATH
ACVPTDPNPQEIVLGNVTENFNMWKNDMADQMHEDVISLWDQSLKPCVKLTPLCVTLNCTDTNVT
GNRTVTGNSTNNTNGTGIYNIEEMKNCSFNATTELRDKKHKEYALFYRLDIVPLNENSDNFTYRL
INCNTSTITQACPKVSFDPIPIHYCAPAGYAILKCNNKTFNGTGPCYNVSTVQCTHGIKPVVSTQ
LLLNGSLAEEGIIIRSENLTENTKTIIVHLNESVEINCTRPNNNTRKSVRIGPGQAFYATNDVIG
NIRQAHCNISTDRWNKTLQQVMKKLGEHFPNKTIQFKPHAGGDLEITMHSFNCRGEFFYCNTSNL
FNSTYHSNNGTYKYNGNSSSPITLQCKIKQIVRMWQGVGQATYAPPIAGNITCRSNITGILLTRD
GGFNTTNNTETFRPGGGDMRDNWRSELYKYKVVEIKPLGIAPTKAKRRVVQREKR*

The Env antigen can a soluble protein, formed, for example, by deletion of the transmembrane region of gp160. This transmembrane region is located in the zone corresponding to the gp41, from the amino acid residue at approximately position 659 to the amino acid residue at approximately position 680. Optionally, another hydrophobic region, from the amino acid residue at approximately position 487 to the amino acid residue at approximately position 514, could also be deleted.

At least three domains in gp160 contain sequences that are hypervariable from one gp160 to another. These three domains are commonly referred to as the V₁, V₂ and V₃ domains (or loops). The first two domains, V₁ and V₂, are located between the cysteine residue at approximately position 96 and the cysteine residue at approximately position 171, while the third domain, V₃, is located from the cysteine residue at approximately position 271 to the cysteine residue at approximately position 306. There is also a final domain exhibiting some degree of variability, albeit considered to be lesser. This is the site of binding to the CD4 receptor of T-helper lymphocytes; it being located approximately from the amino acid residue at position 340 to the amino acid residue at approximately position 440. It is believed that the first and the second hypervariable domains, as well as the CD4 receptor binding site, have an influence on the degree of immunity that could be obtained.

In certain embodiments, the Env antigen may contain modifications, such as deletion of variable regions V₁ and/or V₂ in gp160, gp140, or gp120.

The HIV antigen may also be a fusion polypeptide. For example, the antigen may comprise a first domain and a second domain, wherein (i) the first domain comprises an HIV Env polypeptide (e.g. gp160, gp140, gp120, or an antigenic fragment thereof), and (ii) the second domain comprises another viral protein (e.g., another HIV antigen such as, gag, vif, vpr, tat, rev, vpu, nef, or an antigenic fragment thereof).

### B. The RNA Molecule

The immunogenic composition described herein comprises an RNA component and a polypeptide component. Preferably, the RNA is a self-replicating RNA.

The composition can contain more than one RNA molecule encoding an antigen, e.g., two, three, five, ten or more RNA molecules. Alternatively or in addition, one RNA molecule may also encode more than one antigen, e.g., a bicistronic, or tricistronic RNA molecule that encodes different or identical antigens.

The sequence of the RNA molecule may be codon optimized or deoptimized for expression in a desired host, such as a human cell.

The sequence of the RNA molecule may be modified if desired, for example to increase the efficacy of expression or replication of the RNA, or to provide additional stability or resistance to degradation. For example, the RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy.

If desired, the RNA molecule can comprise one or more modified nucleotides in addition to any 5′ cap structure. There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g. from U.S. Pat. Nos. 4,373,071, 4,458,066, 4,500,707, 4,668,777, 4,973,679, 5,047,524, 5,132,418, 5,153,319, 5,262,530, 5,700,642 all of which are incorporated by reference in their entirety herein, and many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers. See, e.g., WO 2011/005799 which is incorporated herein by reference.

If desired, the RNA molecule can contain phosphoramidate, phosphorothioate, and/or methylphosphonate linkages.

In some embodiments, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine. In other embodiments, the RNA may include a 5′ cap comprising a 7′-methylguanosine, and the first 1, 2 or 3 5′ ribonucleotides may be methylated at the 2′ position of the ribose.

Self-Replicating RNA

In some aspects, the immunogenic composition contains a self-replicating RNA molecule. In certain embodiments, the self-replicating RNA molecule is derived from or based on an alphavirus.

Self-replicating RNA molecules are well known in the art and can be produced by using replication elements derived from, e.g., alphaviruses, and substituting the structural viral proteins with a nucleotide sequence encoding a protein of interest. Cells transfected with self-replicating RNA briefly produce of antigen before undergoing apoptotic death. This death is a likely result of requisite double-stranded (ds) RNA intermediates, which also have been shown to super-activate Dendritic Cells. Thus, the enhanced immunogenicity of self-replicating RNA may be a result of the production of pro-inflammatory dsRNA, which mimics an RNA-virus infection of host cells.

Advantageously, the cell's machinery is used by self-replicating RNA molecules to generate an exponential increase of encoded gene products, such as proteins or antigens, which can accumulate in the cells or be secreted from the cells. Overexpression of proteins or antigens by self-replicating RNA molecules takes advantage of the immunostimulatory adjuvant effects, including stimulation of toll-like receptors (TLR) 3, 7 and 8 and non TLR pathways (e.g, RIG-1, MD-5) by the products of RNA replication and amplification, and translation which induces apoptosis of the transfected cell.

The self-replicating RNA generally contains at least one or more genes selected from the group consisting of viral replicases, viral proteases, viral helicases and other nonstructural viral proteins, and also comprise 5′- and 3′-end cis-active replication sequences, and if desired, a heterologous sequence that encodes a desired amino acid sequence (e.g., an antigen of interest). A subgenomic promoter that directs expression of the heterologous sequence can be included in the self-replicating RNA. If desired, the heterologous sequence (e.g., an antigen of interest) may be fused in frame to other coding regions in the self-replicating RNA and/or may be under the control of an internal ribosome entry site (IRES).

In certain embodiments, the self-replicating RNA molecule is not encapsulated in a virus-like particle. Self-replicating RNA molecules of the invention can be designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alpha virus, such as Sinebis virus (SIN), Semliki forest virus and Venezuelan equine encephalitis virus (VEE), one or more genes encoding viral structural proteins, such as capsid and/or envelope glycoproteins, can be omitted.

If desired, self-replicating RNA molecules of the invention can also be designed to induce production of infectious viral particles that are attenuated or virulent, or to produce viral particles that are capable of a single round of subsequent infection.

When delivered to a vertebrate cell, a self-replicating RNA molecule can lead to the production of multiple daughter RNAs by transcription from itself (or from an antisense copy of itself). The self-replicating RNA can be directly translated after delivery to a cell, and this translation provides a RNA-dependent RNA polymerase which then produces transcripts from the delivered RNA. Thus the delivered RNA leads to the production of multiple daughter RNAs. These transcripts are antisense relative to the delivered RNA and may be translated themselves to provide in situ expression of a gene product, or may be transcribed to provide further transcripts with the same sense as the delivered RNA which are translated to provide in situ expression of the gene product.

One suitable system for achieving self-replication is to use an alphavirus-based RNA replicon. Alphaviruses comprise a set of genetically, structurally, and serologically related arthropod-borne viruses of the Togaviridae family. Twenty-six known viruses and virus subtypes have been classified within the alphavirus genus, including, Sindbis virus, Semliki Forest virus, Ross River virus, and Venezuelan equine encephalitis virus. As such, the self-replicating RNA of the invention may incorporate a RNA replicase derived from semliki forest virus (SFV), sindbis virus (SIN), Venezuelan equine encephalitis virus (VEE), Ross-River virus (RRV), or other viruses belonging to the alphavirus family.

An alphavirus-based “replicon” expression vector can be used in the invention. Replicon vectors may be utilized in several formats, including DNA, RNA, and recombinant replicon particles. Such replicon vectors have been derived from alphaviruses that include, for example, Sindbis virus (Xiong et al. (1989) Science 243:1188-1191; Dubensky et al., (1996) J. Virol. 70:508-519; Hariharan et al. (1998) J. Virol. 72:950-958; Polo et al. (1999) PNAS 96:4598-4603), Semliki Forest virus (Liljestrom (1991) Bio/Technology 9:1356-1361; Berglund et al. (1998) Nat. Biotech. 16:562-565), and Venezuelan equine encephalitis virus (Pushko et al. (1997) Virology 239:389-401). Alphavirus-derived replicons are generally quite similar in overall characteristics (e.g., structure, replication), individual alphaviruses may exhibit some particular property (e.g., receptor binding, interferon sensitivity, and disease profile) that is unique. Therefore, chimeric alphavirus replicons made from divergent virus families may also be useful.

Alphavirus-based replicons are (+)-stranded replicons that can be translated after delivery to a cell to give of a replicase (or replicase-transcriptase). The replicase is translated as a polyprotein which auto-cleaves to provide a replication complex which creates genomic (−)-strand copies of the +-strand delivered RNA. These (−)-strand transcripts can themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) a polypeptide antigen. The polymerase can be an alphavirus replicase e.g. comprising alphavirus protein nsP4.

Whereas natural alphavirus genomes encode structural virion proteins in addition to the non-structural replicase, it is preferred that an alphavirus based self-replicating RNA molecule of the invention does not encode alphavirus structural proteins. Thus the self-replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the invention and their place is taken by gene(s) encoding the desired gene product, such that the subgenomic transcript encodes the desired gene product rather than the structural alphavirus virion proteins.

Thus a self-replicating RNA molecule useful with the invention may have two open reading frames. The first (5′) open reading frame encodes a replicase; the second (3′) open reading frame encodes a polypeptide antigen. In some embodiments the RNA may have additional (downstream) open reading frames e.g. that encode another desired gene product. A self-replicating RNA molecule can have a 5′ sequence which is compatible with the encoded replicase.

In other aspects, the self-replicating RNA molecule is derived from or based on a virus other than an alphavirus, preferably, a positive-stranded RNA virus, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The self-replicating RNA molecules of the invention are larger than other types of RNA (e.g. mRNA). Typically, the self-replicating RNA molecules of the invention contain at least about 4 kb. For example, the self-replicating RNA can contain at least about 5 kb, at least about 6 kb, at least about 7 kb, at least about 8 kb, at least about 9 kb, at least about 10 kb, at least about 11 kb, at least about 12 kb or more than 12 kb. In certain examples, the self-replicating RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

The self-replicating RNA molecules of the invention may comprise one or more modified nucleotides (e.g., pseudouridine, N6-methyladenosine, 5-methylcytidine, 5-methyluridine).

The self-replicating RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-replicating RNA may then be produced as a fusion polypeptide or engineered in such a manner to result in separate polypeptide or peptide sequences.

The self-replicating RNA of the invention may encode one or more polypeptide antigens that contain a range of epitopes. Preferably epitopes capable of eliciting either a helper T-cell response or a cytotoxic T-cell response or both.

The self-replicating RNA molecules described herein may be engineered to express multiple nucleotide sequences, from two or more open reading frames, thereby allowing co-expression of proteins, such as two or more antigens together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

The self-replicating RNA molecules of the invention can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses.

Suitable synthetic methods can be used alone, or in combination with one or more other methods (e.g., recombinant DNA or RNA technology), to produce a self-replicating RNA molecule of the invention. Suitable methods for de novo synthesis are well-known in the art and can be adapted for particular applications. Exemplary methods include, for example, chemical synthesis using suitable protecting groups such as CEM (Masuda et al., (2007) Nucleic Acids Symposium Series 51:3-4), the β-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedron Lett 27:4055-8; Gaffney B L et al. (1988) Tetrahedron Lett 29:2619-22). These chemistries can be performed or adapted for use with automated nucleic acid synthesizers that are commercially available. Additional suitable synthetic methods are disclosed in Uhlmann et al. (1990) Chem Rev 90:544-84, and Goodchild J (1990) Bioconjugate Chem 1: 165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation and expression of nucleic acid sequences are known and conventional in the art. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

The presence and/or quantity of one or more modified nucleotides in a self-replicating RNA molecule can be determined using any suitable method. For example, a self-replicating RNA can be digested to monophosphates (e.g., using nuclease P1) and dephosphorylated (e.g., using a suitable phosphatase such as CIAP), and the resulting nucleosides analyzed by reversed phase HPLC (e.g., usings a YMC Pack ODS-AQ column (5 micron, 4.6×250 mm) and eluted using a gradient, 30% B (0-5 min) to 100% B (5-13 min) and at 100% B (13-40) min, flow Rate (0.7 ml/min), UV detection (wavelength: 260 nm), column temperature (30° C.). Buffer A (20 mM acetic acid—ammonium acetate pH 3.5), buffer B (20 mM acetic acid—ammonium acetate pH 3.5/methanol[90/10])).

Optionally, the self-replicating RNA molecules of the invention may include one or more modified nucleotides so that the self-replicating RNA molecule will have less immunomodulatory activity upon introduction or entry into a host cell (e.g., a human cell) in comparison to the corresponding self-replicating RNA molecule that does not contain modified nucleotides.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self replicating RNA molecule that encodes a polypeptide antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode a polypeptide antigen can also be tested for ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays that can be used to characterize the self-replicating RNA molecules of the invention can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

The self-replicating RNA of the invention may be delivered by a variety of methods, such as naked RNA delivery or in combination with lipids, polymers or other compounds that facilitate entry into the cells. The RNA molecules of the present invention can be introduced into target cells or subjects using any suitable technique, e.g., by direct injection, microinjection, electroporation, lipofection, biolystics, and the like.

### C. The Polypeptide Molecule

The immunogenic composition described herein comprises a polypeptide component and an RNA component. The polypeptide component encompasses multi-chain polypeptide structures, such as a polypeptide complex (e.g., a complex formed by two or more proteins), or a large polypeptide structure, such as VLP.

Suitable antigens that can be used as the polypeptide component (the “second polypeptide antigen”) of the immunogenic composition include proteins and peptides derived from HIV. The composition can contain more than one polypeptide antigen. Alternatively or in addition, the polypeptide may also be a fusion polypeptide comprising two or more epitopes from two different proteins of HIV.

The polypeptide antigen may include additional sequences, such as a sequence to facilitate expression, production, purification or detection (e.g., a poly-His sequence).

The polypeptide antigen will usually be isolated or purified. Thus, it will not be associated with molecules with which it is normally, if applicable, found in nature.

Polypeptides will usually be prepared by expression in a recombinant host system. Generally, they are produced by expression of recombinant constructs that encode the ecto-domains in suitable recombinant host cells, although any suitable methods can be used. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding a polypeptide can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

Polypeptides can be purified using any suitable methods. For example, methods for purifying polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the polypeptides can include a “tag” that facilitates purification, such as an epitope tag or a HIS tag. Such tagged polypeptides can conveniently be purified, for example from conditioned media, by chelating chromatography or affinity chromatography.

### D. Optional RNA Delivery Systems

In addition to the protein component and the RNA component, additional components, such as lipids, polymers or other compounds may be optionally included in the immunogenic composition as described herein to facilitate the entry of RNA into target cells.

Although RNA can be delivered as naked RNA (e.g. merely as an aqueous solution of RNA), to enhance entry into cells and also subsequent intercellular effects, the RNA molecule is preferably administered in combination with a delivery system, such as a particulate or emulsion delivery system. A large number of delivery systems are well known to those of skill in the art.

For example, the RNA molecule may be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor-binding moiety (e.g., a cyclic peptide having the sequence Arg-Gly-Asp).

The RNA molecule of the present invention can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

Three particularly useful delivery systems are (i) liposomes (ii) non-toxic and biodegradable polymer microparticles (iii) cationic submicron oil-in-water emulsions.

1. Liposomes

Various amphiphilic lipids can form bilayers in an aqueous environment to encapsulate a RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic. Suitable classes of phospholipid include, but are not limited to, phosphatidylethanolamines, phosphatidylcholines, phosphatidylserines, and phosphatidylglycerols, and some useful phospholipids are listed in Table 2. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

TABLE 2
Phospholipids

| | |
|---|---|
| DDPC | 1,2-Didecanoyl-sn-Glycero-3-phosphatidylcholine |
| DEPA | 1,2-Dierucoyl-sn-Glycero-3-Phosphate |
| DEPC | 1,2-Erucoyl-sn-Glycero-3-phosphatidylcholine |
| DEPE | 1,2-Dierucoyl-sn-Glycero-3-phosphatidylethanolamine |
| DEPG | 1,2-Dierucoyl-sn-Glycero-3[Phosphatidyl-rac-(1- |
| | glycerol . . . ) |
| DLOPC | 1,2-Linoleoyl-sn-Glycero-3-phosphatidylcholine |
| DLPA | 1,2-Dilauroyl-sn-Glycero-3-Phosphate |
| DLPC | 1,2-Dilauroyl-sn-Glycero-3-phosphatidylcholine |
| DLPE | 1,2-Dilauroyl-sn-Glycero-3-phosphatidylethanolamine |
| DLPG | 1,2-Dilauroyl-sn-Glycero-3[Phosphatidyl-rac-(1- |
| | glycerol . . . ) |
| DLPS | 1,2-Dilauroyl-sn-Glycero-3-phosphatidylserine |
| DMG | 1,2-Dimyristoyl-sn-glycero-3-phosphoethanolamine |
| DMPA | 1,2-Dimyristoyl-sn-Glycero-3-Phosphate |
| DMPC | 1,2-Dimyristoyl-sn-Glycero-3-phosphatidylcholine |
| DMPE | 1,2-Dimyristoyl-sn-Glycero-3-phosphatidylethanolamine |
| DMPG | 1,2-Myristoyl-sn-Glycero-3[Phosphatidyl-rac-(1- |
| | glycerol . . . ) |
| DMPS | 1,2-Dimyristoyl-sn-Glycero-3-phosphatidylserine |
| DOPA | 1,2-Dioleoyl-sn-Glycero-3-Phosphate |
| DOPC | 1,2-Dioleoyl-sn-Glycero-3-phosphatidylcholine |
| DOPE | 1,2-Dioleoyl-sn-Glycero-3-phosphatidylethanolamine |
| DOPG | 1,2-Dioleoyl-sn-Glycero-3[Phosphatidyl-rac-(1- |
| | glycerol . . . ) |
| DOPS | 1,2-Dioleoyl-sn-Glycero-3-phosphatidylserine |
| DPPA | 1,2-Dipalmitoyl-sn-Glycero-3-Phosphate |
| DPPC | 1,2-Dipalmitoyl-sn-Glycero-3-phosphatidylcholine |
| DPPE | 1,2-Dipalmitoyl-sn-Glycero-3-phosphatidylethanolamine |
| DPPG | 1,2-Dipalmitoyl-sn-Glycero-3[Phosphatidyl-rac-(1- |
| | glycerol . . . ) |
| DPPS | 1,2-Dipalmitoyl-sn-Glycero-3-phosphatidylserine |
| DPyPE | 1,2-diphytanoyl-sn-glycero-3-phosphoethanolamine |
| DSPA | 1,2-Distearoyl-sn-Glycero-3-Phosphate |
| DSPC | 1,2-Distearoyl-sn-Glycero-3-phosphatidylcholine |
| DSPE | 1,2-Diostearpyl-sn-Glycero-3-phosphatidylethanolamine |
| DSPG | 1,2-Distearoyl-sn-Glycero-3[Phosphatidyl-rac-(1- |
| | glycerol . . . ) |
| DSPS | 1,2-Distearoyl-sn-Glycero-3-phosphatidylserine |
| EPC | Egg-PC |
| HEPC | Hydrogenated Egg PC |
| HSPC | High purity Hydrogenated Soy PC |
| HSPC | Hydrogenated Soy PC |
| LYSOPC | 1-Myristoyl-sn-Glycero-3-phosphatidylcholine |
| MYRISTIC | |
| LYSOPC | 1-Palmitoyl-sn-Glycero-3-phosphatidylcholine |
| PALMITIC | |
| LYSOPC | 1-Stearoyl-sn-Glycero-3-phosphatidylcholine |
| STEARIC | |
| Milk | 1-Myristoyl,2-palmitoyl-sn-Glycero |
| Sphingomyelin | 3-phosphatidylcholine |
| MPPC | |
| MSPC | 1-Myristoyl,2-stearoyl-sn-Glycero-3-phosphatidylcholine |
| PMPC | 1-Palmitoyl,2-myristoyl-sn-Glycero-3- |
| | phosphatidylcholine |
| POPC | 1-Palmitoyl,2-oleoyl-sn-Glycero-3-phosphatidylcholine |
| POPE | 1-Palmitoyl-2-oleoyl-sn-Glycero-3- |
| | phosphatidylethanolamine |
| POPG | 1,2-Dioleoyl-sn-Glycero-3[Phosphatidyl-rac-(1- |
| | glycerol) . . . ] |
| PSPC | 1-Palmitoyl,2-stearoyl-sn-Glycero-3-phosphatidylcholine |
| SMPC | 1-Stearoyl,2-myristoyl-sn-Glycero-3-phosphatidylcholine |
| SOPC | 1-Stearoyl,2-oleoyl-sn-Glycero-3-phosphatidylcholine |
| SPPC | 1-Stearoyl,2-palmitoyl-sn-Glycero-3-phosphatidylcholine |

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all of the component lipids in the mixture need to be amphiphilic e.g. one or more amphiphilic lipids can be mixed with cholesterol.

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-87.

A mixture of DSPC, DlinDMA, PEG-DMPG and cholesterol is used in the examples. A separate aspect of the invention is a liposome comprising DSPC, DlinDMA, PEG-DMG and cholesterol. This liposome preferably encapsulates RNA, such as a self-replicating RNA e.g. encoding an immunogen.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter ≦50 nm, and LUVs have a diameter >50 nm. Liposomes useful with the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2.

Techniques for preparing suitable liposomes are well known in the art e.g. see Liposomes: Methods and Protocols, Volume 1: Pharmaceutical Nanocarriers: Methods and Protocols. (ed. Weissig). Humana Press, 2009. ISBN 160327359X; Liposome Technology, volumes I, II & III. (ed. Gregoriadis). Informa Healthcare, 2006; and Functional Polymer Colloids and Microparticles volume 4 (Microspheres, microcapsules & liposomes). (eds. Arshady & Guyot). Citus Books, 2002. One useful method involves mixing (i) an ethanolic solution of the lipids (ii) an aqueous solution of the nucleic acid and (iii) buffer, followed by mixing, equilibration, dilution and purification (Heyes et al. (2005) J Controlled Release 107:276-87.).

RNA is preferably encapsulated within the liposomes, and so the liposome forms a outer layer around an aqueous RNA-containing core. This encapsulation has been found to protect RNA from RNase digestion. The liposomes can include some external RNA (e.g. on the surface of the liposomes), but at least half of the RNA (and ideally all of it) is encapsulated.

2. Polymeric Microparticles

Various polymers can form microparticles to encapsulate or adsorb RNA. The use of a substantially non-toxic polymer means that a recipient can safely receive the particles, and the use of a biodegradable polymer means that the particles can be metabolised after delivery to avoid long-term persistence. Useful polymers are also sterilisable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 55:45, 60:40, 75:25. Useful PLG polymers include those having a molecular weight between, for example, 5,000-200,000 Da e.g. between 10,000-100,000, 20,000-70,000, 40,000-50,000 Da.

The microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g. see Functional Polymer Colloids and Microparticles volume 4 (Microspheres, microcapsules & liposomes). (eds. Arshady & Guyot). Citus Books, 2002; Polymers in Drug Delivery. (eds. Uchegbu & Schatzlein). CRC Press, 2006. (in particular chapter 7) and Microparticulate Systems for the Delivery of Proteins and Vaccines. (eds. Cohen & Bernstein). CRC Press, 1996. To facilitate adsorption of RNA, a microparticle may include a cationic surfactant and/or lipid e.g. as disclosed in O'Hagan et al. (2001) J Virology 75:9037-9043; and Singh et al. (2003) Pharmaceutical Research 20: 247-251. An alternative way of making polymeric microparticles is by molding and curing e.g. as disclosed in WO2009/132206.

Microparticles of the invention can have a zeta potential of between 40-100 mV.

RNA can be adsorbed to the microparticles, and adsorption is facilitated by including cationic materials (e.g. cationic lipids) in the microparticle.

3. Oil-in-Water Cationic Emulsions

Oil-in-water emulsions are known for adjuvanting influenza vaccines e.g. the MF59™ adjuvant in the FLUAD™ product, and the AS03 adjuvant in the PREPANDRIX™ product. RNA delivery according to the present invention can utilise an oil-in-water emulsion, provided that the emulsion includes one or more cationic molecules. For instance, a cationic lipid can be included in the emulsion to provide a positive droplet surface to which negatively-charged RNA can attach.

The emulsion comprises one or more oils. Suitable oil(s) include those from, for example, an animal (such as fish) or a vegetable source. The oil is ideally biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Squalene can also be obtained from yeast or other suitable microbes. In some embodiments, Squalene is preferably obtained from non-animal sources, such as from olives, olive oil or yeast. Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

Preferred emulsions comprise squalene, a shark liver oil which is a branched, unsaturated terpenoid (C₃₀H₅₀; [(CH₃)₂C[═CHCH₂CH₂C(CH₃)]₂═CHCH₂—]₂; 2,6,10,15,19,23-hexamethyl-2,6,10,14,18,22-tetracosahexaene; CAS RN 7683-64-9).

The oil in the emulsion may comprise a combination of oils e.g. squalene and at least one further oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. Preferably this lipid is a surfactant so that it can facilitate formation and stabilisation of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′-[N—(N′,N′-Dimethylaminoethane)-carbamoyl]Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), 1,2-dilinoleyloxy-3-dimethylaminopropane (DLinDMA). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), primary amines, secondary amines, tertiary amines, including but not limited to N,N′,N′-polyoxyethylene (10)-N-tallow-1,3-diaminopropane, other quaternary amine salts, including but not limited to dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C₁₂Me₆; C₁₂Bu₆), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (C₁₄GluCₙN⁺), cationic derivatives of cholesterol, including but not limited to cholesteryl-3 β-oxysuccinamidoethylenetrimethylammonium salt, cholesteryl-3 β-oxysuccinamidoethylene dimethylamine, cholesteryl-3 β-carboxyamidoethylenetrimethylammonium salt, cholesteryl-3 β-carboxyamidoethylenedimethylamine. Other useful cationic lipids are described in US 2008/0085870 and US 2008/0057080, which are incorporated herein by reference.

The cationic lipid is preferably biodegradable (metabolisable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, preferably 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and preferably at least 90%. Apparatuses for determining the average droplet size in an emulsion, and the size distribution, are commercially available. These these typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidisation. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidisation can be performed until an emulsion with a desired droplet size average and distribution are achieved.

As an alternative to microfluidisation, thermal methods can be used to cause phase inversion. These methods can also provide a submicron emulsion with a tight particle size distribution.

Preferred emulsions can be filter sterilised i.e. their droplets can pass through a 220 nm filter. As well as providing a sterilisation, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml, from about 0.6 mg/ml to about 25 mg/ml, from about 0.7 mg/ml to about 25 mg/ml, from about 0.8 mg/ml to about 25 mg/ml, from about 0.9 mg/ml to about 25 mg/ml, from about 1.0 mg/ml to about 25 mg/ml, from about 1.1 mg/ml to about 25 mg/ml, from about 1.2 mg/ml to about 25 mg/ml, from about 1.3 mg/ml to about 25 mg/ml, from about 1.4 mg/ml to about 25 mg/ml, from about 1.5 mg/ml to about 25 mg/ml, from about 1.6 mg/ml to about 25 mg/ml, from about 1.7 mg/ml to about 25 mg/ml, from about 0.5 mg/ml to about 24 mg/ml, from about 0.5 mg/ml to about 22 mg/ml, from about 0.5 mg/ml to about 20 mg/ml, from about 0.5 mg/ml to about 18 mg/ml, from about 0.5 mg/ml to about 15 mg/ml, from about 0.5 mg/ml to about 12 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1.9 mg/ml, from about 0.5 mg/ml to about 1.8 mg/ml, from about 0.5 mg/ml to about 1.7 mg/ml, from about 0.5 mg/ml to about 1.6 mg/ml, from about 0.6 mg/ml to about 1.6 mg/ml, from about 0.7 mg/ml to about 1.6 mg/ml, from about 0.8 mg/ml to about 1.6 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 12 mg/ml, about 18 mg/ml, about 20 mg/ml, about 21.8 mg/ml, about 24 mg/ml, etc. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.62 mg/ml to about 5 mg/ml, from about 1 mg/ml to about 5 mg/ml, from about 1.5 mg/ml to about 5 mg/ml, from about 2 mg/ml to about 5 mg/ml, from about 2.46 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, from about 0.1 mg/ml to about 4.92 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.46 mg/ml, from about 0.1 mg/ml to about 2 mg/ml, from about 0.1 mg/ml to about 1.5 mg/ml, from about 0.1 mg/ml to about 1 mg/ml, from about 0.1 mg/ml to about 0.62 mg/ml, about 0.15 mg/ml, about 0.3 mg/ml, about 0.6 mg/ml, about 0.62 mg/ml, about 0.9 mg/ml, about 1.2 mg/ml, about 2.46 mg/ml, about 4.92 mg/ml, etc. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 5 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.5 mg/ml, from about 0.1 mg/ml to about 2 mg/ml, from about 0.1 mg/ml to about 1.5 mg/ml, from about 0.1 mg/ml to about 1.45 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.6 mg/ml to about 5 mg/ml, from about 0.73 mg/ml to about 5 mg/ml, from about 0.8 mg/ml to about 5 mg/ml, from about 0.9 mg/ml to about 5 mg/ml, from about 1.0 mg/ml to about 5 mg/ml, from about 1.2 mg/ml to about 5 mg/ml, from about 1.45 mg/ml to about 5 mg/ml, from about 2 mg/ml to about 5 mg/ml, from about 2.5 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, about 1.2 mg/ml, about 1.45 mg/ml, etc. Alternatively, the cationic oil-in-water emulsion may comprise DDA at about 20 mg/ml, about 21 mg/ml, about 21.5 mg/ml, about 21.6 mg/ml, about 25 mg/ml. In an exemplary embodiment, the cationic oil-in-water emulsion comprises from about 0.73 mg/ml to about 1.45 mg/ml DDA, such as 1.45 mg/ml.

The RNA molecules of the invention can also be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have been transfected with the RNA molecule. The appropriate amount of cells to deliver to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

### E. Adjuvants

In certain embodiments, the immunogenic compositions provided herein include or optionally include one or more immunoregulatory agents such as adjuvants. Exemplary adjuvants include, but are not limited to, a TH1 adjuvant and/or a TH2 adjuvant, further discussed below. In certain embodiments, the adjuvants used in the immunogenic compositions provide herein include, but are not limited to:

### 1. Mineral-Containing Compositions;

### 2. Oil Emulsions;

### 3. Saponin Formulations;

### 4. Virosomes and Virus-Like Particles;

### 5. Bacterial or Microbial Derivatives;

### 6. Bioadhesives and Mucoadhesives;

### 7. Liposomes;

### 8. Polyoxyethylene Ether and Polyoxyethylene Ester Formulations;

### 9. Polyphosphazene (PCPP);

### 10. Muramyl Peptides;

### 11. Imidazoquinolone Compounds;

### 12. Thiosemicarbazone Compounds;

### 13. Tryptanthrin Compounds;

### 14. Human Immunomodulators;

### 15. Lipopeptides;

### 16. Benzonaphthyridines;

### 17. Microparticles

18. Immunostimulatory polynucleotide (such as RNA or DNA; e.g., CpG-containing
oligonucleotides)

1. Mineral Containing Compositions

Mineral containing compositions suitable for use as adjuvants include mineral salts, such as aluminum salts and calcium salts. The immunogenic composition may include mineral salts such as hydroxides (e.g., oxyhydroxides), phosphates (e.g., hydroxyphosphates, orthophosphates), sulfates, etc. (see, e.g., VACCINE DESIGN: THE SUBUNIT AND ADJUVANT APPROACH (Powell, M. F. and Newman, M J. eds.) (New York: Plenum Press) 1995, Chapters 8 and 9), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

In certain embodiments, the aluminum based adjuvant is alum (aluminum potassium sulfate (AlK(SO₄)₂), or an alum derivative, such as that formed in-situ by mixing an antigen in phosphate buffer with alum, followed by titration and precipitation with a base such as ammonium hydroxide or sodium hydroxide.

Another aluminum-based adjuvant suitable for use in vaccine formulations is aluminum hydroxide adjuvant (Al(OH)₃) or crystalline aluminum oxyhydroxide (AlOOH), which is an excellent adsorbant, having a surface area of approximately 500 m²/g. Alternatively, the aluminum based adjuvant can be aluminum phosphate adjuvant (AlPO₄) or aluminum hydroxyphosphate, which contains phosphate groups in place of some or all of the hydroxyl groups of aluminum hydroxide adjuvant. Preferred aluminum phosphate adjuvants provided herein are amorphous and soluble in acidic, basic and neutral media.

In certain embodiments, the adjuvant comprises both aluminum phosphate and aluminum hydroxide. In a more particular embodiment, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. In another embodiment, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep=4). Alternatively, pretreatment of aluminum hydroxide with phosphate lowers its isoelectric point, making it a preferred adjuvant for more basic antigens.

2. Oil-Emulsions

Oil-emulsion compositions and formulations suitable for use as adjuvants (with or without other specific immunostimulating agents such as muramyl peptides or bacterial cell wall components) include squalene-water emulsions, such as MF59 (5% Squalene, 0.5% Tween 80, and 0.5% Span 85, formulated into submicron particles using a microfluidizer). See WO 90/14837. See also, Podda (2001) VACCINE 19: 2673-2680; Frey et al. (2003) Vaccine 21:4234-4237. MF59 is used as the adjuvant in the FLUAD™ influenza virus trivalent subunit vaccine.

Particularly preferred oil-emulsion adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyethylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-SM-glycero-3-huydroxyphosphophoryloxy)-ethylamine (MTP-PE), for example, the submicron oil-in-water emulsion known as “MF59” (WO 90/14837; U.S. Pat. No. 6,299,884; U.S. Pat. No. 6,451,325; and Ott et al., “MF59—Design and Evaluation of a Safe and Potent Adjuvant for Human Vaccines” in Vaccine Design: The Subunit and Adjuvant Approach (Powell, M. F. and Newman, M J. eds.) (New York: Plenum Press) 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 11OY microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in WO 90/14837; U.S. Pat. No. 6,299,884; and U.S. Pat. No. 6,451,325.

Complete Freund's adjuvant (CFA) and incomplete Freund's adjuvant (IFA) may also be used as adjuvants in the invention.

3. Other Immunological Adjuvants

Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. Saponin adjuvant formulations include STIMULON® adjuvant (Antigenics, Inc., Lexington, Mass.).

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS 17, QS 18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Saponin formulations may include sterols, cholesterols and lipid formulations. Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of (an) additional detergent(s). See WO 00/07621.

A review of the development of saponin based adjuvants can be found in Barr et al. (1998) ADV. DRUG DEL. REV. 32:247-271. See also Sjolander et al. (1998) ADV. DRUG DEL. REV. 32:321-338.

Virosomes and Virus Like Particles (VLPs) generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein pi). VLPs are discussed further in WO 03/024480; WO 03/024481; Niikura et al. (2002) VIROLOGY 293:273-280; Lenz et al. (2001) J. ImmuNoL. 166(9):5346-5355′ Pinto et al. (2003) J. INFECT. DIS. 188:327-338; and Gerber et al. (200I) J. VIROL. 75(10):4752-4760. Virosomes are discussed further in, for example, Gluck et al. (2002) VACCINE 20:B10-B16. Immunopotentiating reconstituted influenza virosomes (IRIV) are used as the subunit antigen delivery system in the intranasal trivalent INFLEXAL™ product (Mischler and Metcalfe (2002) VACCINE 20 Suppl 5:B17-B23) and the INFLUVAC PLUS™ product.

Bacterial or microbial derivatives suitable for use as adjuvants include, but are not limited to:

(1) Non-toxic derivatives of enterobacterial lipopolysaccharide (LPS): Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives, e.g., RC-529. See Johnson et al. (1999) Bioorg. Med. Chem. Lett. 9:2273-2278.

(2) Lipid A Derivatives: Lipid A derivatives include derivatives of lipid A from Escherichia coli such as OM-174. OM-174 is described for example in Meraldi et al. (2003) Vaccine 21:2485-2491; and Pajak et al. (2003) Vaccine 21:836-842. Another exemplary adjuvant is the synthetic phospholipid dimer, E6020 (Eisai Co. Ltd., Tokyo, Japan), which mimics the physicochemical and biological properties of many of the natural lipid A's derived from Gram-negative bacteria.

(3) Immunostimulatory oligonucleotides: Immunostimulatory oligonucleotides or polymeric molecules suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory. The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla et al. (2003) Nucl. Acids Res. 31(9): 2393-2400; WO 02/26757; and WO 99/62923 for examples of possible analog substitutions. The adjuvant effect of CpG oligonucleotides is further discussed in Krieg (2003) Nat. Med. 9(7):831-835; McCluskie et al. (2002) FEMS Immunol. Med. Microbiol. 32: 179-185; WO 98/40100; U.S. Pat. No. 6,207,646; U.S. Pat. No. 6,239,116; and U.S. Pat. No. 6,429,199.

The CpG sequence may be directed to TLR9, such as the motif GTCGTT or TTCGTT. See Kandimalla et al. (2003) Biochem. Soc. Trans. 31 (part 3):654-658. The CpG sequence may be specific for inducing a ThI immune response, such as a CpG-A ODN, or it may be more specific for inducing a B cell response, such a CpG-B ODN. CpG-A and CpG-B ODNs are discussed in Blackwell et al. (2003) J. Immunol. 170(8):4061-4068; Krieg (2002) TRENDS Immunol. 23(2): 64-65; and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla et al. (2003) BBRC 306:948-953; Kandimalla et al. (2003) Biochem. Soc. Trans. 3 1(part 3):664-658’ Bhagat et al. (2003) BBRC 300:853-861; and WO03/035836.

Immunostimulatory oligonucleotides and polymeric molecules also include alternative polymer backbone structures such as, but not limited to, polyvinyl backbones (Pitha et al. (1970) Biochem. Biophys. Acta 204(1):39-48; Pitha et al. (1970) Biopolymers 9(8):965-977), and morpholino backbones (U.S. Pat. No. 5,142,047; U.S. Pat. No. 5,185,444). A variety of other charged and uncharged polynucleotide analogs are known in the art. Numerous backbone modifications are known in the art, including, but not limited to, uncharged linkages (e.g., methyl phosphonates, phosphotriesters, phosphoamidates, and carbamates) and charged linkages (e.g., phosphorothioates and phosphorodithioates).

Adjuvant IC31, Intercell AG, Vienna, Austria, is a synthetic formulation that contains an antimicrobial peptide, KLK, and an immunostimulatory oligonucleotide, ODNIa. The two component solution may be simply mixed with antigens (e.g., particles in accordance with the invention with an associated antigen), with no conjugation required.

ADP-ribosylating toxins and detoxified derivatives thereof: Bacterial ADP-ribosylating toxins and detoxified derivatives thereof may be used as adjuvants in the invention. Preferably, the protein is derived from E. coli (i.e., E. coli heat labile enterotoxin “LT”), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon et al. (2002) Infect. Immun. 70(6):3012-3019; Pizza et al. (2001) Vaccine 19:2534-2541; Pizza et al. (2000) J. Med. Microbiol. 290(4-5):455-461; Scharton-Kersten et al. (2000) Infect. Immun. 68(9):5306-5313′ Ryan et al. (1999) Infect. Immun. 67(12):6270-6280; Partidos et al. (1999) Immunol. Lett. 67(3):209-216; Peppoloni et al. (2003) Vaccines 2(2):285-293; and Pine et al. (2002) J. Control Release 85(1-3):263-270. Numerical reference for amino acid substitutions is preferably based on the alignments of the A and B subunits of ADP-ribosylating toxins set forth in Domenighini et al. (1995) MoI. Microbiol. 15(6): 1165-1167.

Bioadhesives and mucoadhesives may also be used as adjuvants. Suitable bioadhesives include esterified hyaluronic acid microspheres (Singh et al. (2001) J. Cont. Release 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention (see WO 99/27960).

Examples of liposome formulations suitable for use as adjuvants are described in U.S. Pat. No. 6,090,406; U.S. Pat. No. 5,916,588; and EP Patent Publication No. EP 0 626 169.

Adjuvants suitable for use in the invention include polyoxyethylene ethers and polyoxyethylene esters (see, e.g., WO 99/52549). Such formulations further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

PCPP formulations suitable for use as adjuvants are described, for example, in Andrianov et al. (1998) Biomaterials 19(1-3): 109-115; and Payne et al. (1998) Adv. Drug Del. Rev. 31(3): 185-196.

Examples of muramyl peptides suitable for use as adjuvants include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

Examples of imidazoquinoline compounds suitable for use as adjuvants include Imiquimod and its analogues, which are described further in Stanley (2002) Clin. Exp. Dermatol. 27(7):571-577; Jones (2003) Curr. Opin. Investig. Drugs 4(2):214-218; and U.S. Pat. Nos. 4,689,338; 5,389,640; 5,268,376; 4,929,624; 5,266,575; 5,352,784; 5,494,916; 5,482,936; 5,346,905; 5,395,937; 5,238,944; and 5,525,612.

Examples of thiosemicarbazone compounds suitable for use as adjuvants, as well as methods of formulating, manufacturing, and screening for such compounds, include those described in WO 04/60308. The thiosemicarbazones are particularly effective in the stimulation of human peripheral blood mononuclear cells for the production of cytokines, such as TNF-α.

Examples of tryptanthrin compounds suitable for use as adjuvants, as well as methods of formulating, manufacturing, and screening for such compounds, include those described in WO 04/64759. The tryptanthrin compounds are particularly effective in the stimulation of human peripheral blood mononuclear cells for the production of cytokines, such as TNF-α. examples of benzonaphthyridine compounds suitable for use as adjuvants include:

Examples of benzonaphthyridine compounds suitable for use as adjuvants, as well as methods of formulating and manufacturing, include those described in WO 2009/111337.

Lipopeptides suitable for use as adjuvants are described above. Other exemplary lipopeptides include, e.g., LP 40, which is an agonist of TLR2. See, e.g., Akdis, et al, EUR. J. IMMUNOLOGY, 33: 2717-26 (2003). Murein lipopeptides are lipopeptides derived from E. coli. See, Hantke, et al., Eur. J. Biochem., 34: 284-296 (1973). Murein lipopeptides comprise a peptide linked to N-acetyl muramic acid, and are thus related to Muramyl peptides, which are described in Baschang, et al., Tetrahedron, 45(20): 6331-6360 (1989).

The human immunomodulators suitable for use as adjuvants include, but are not limited to, cytokines, such as, by way of example only, interleukins (IL-1, IL-2, IL-4, IL-5, IL-6, IL-7, IL-12), interferons (such as, by way of example only, interferon-□), macrophage colony stimulating factor, and tumor necrosis factor.

Microparticles suitable for use as adjuvants include, but are not limited to, microparticles formed from materials that are biodegradable and non-toxic (e.g. a poly(.alpha.-hydroxy acid), a polyhydroxybutyric acid, a polyorthoester, a polyanhydride, a polycaprolactone, etc.), with poly(lactide-co-glycolide). In certain embodiments, such microparticles are treated to have a negatively-charged surface (e.g. with SDS) or a positively-charged surface (e.g. with a cationic detergent, such as CTAB). The microparticles suitable for use as adjuvants have a particle diameter of about 100 nm to about 150 μm in diameter. In certain embodiments, the particle diameter is about 200 nm to about 30 μm, and in other embodiments the particle diameter is about 500 nm to 10 μm.

### 3. Kits

### (A) Kits for Co-Administration of an RNA Molecule and a Polypeptide Molecule

The invention also provides kits, wherein an RNA molecule encoding a first polypeptide antigen (the RNA component); and a second polypeptide antigen (the polypeptide component), are in separate containers. For example, the kit can contain a first container comprising a composition comprising an RNA molecule encoding a first polypeptide antigen, and a second container comprising a composition comprising a second polypeptide antigen. The polypeptide or the RNA molecule can be in liquid form or can be in solid form (e.g., lyophilized).

The kits described may be used for co-delivery of the RNA component and the polypeptide component of the immunogenic compositions described herein (e.g., the RNA component and the polypeptide component are mixed prior to administration for simultaneous delivery, e.g., mixed within about 72 hours, about 48 hours, about 24 hours, about 12 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 15 minutes, about 10 minutes, or about 5 minutes prior to administration).

### (B) Kits for Prime-Boost

In another aspect, the invention provides a kit comprising: (i) a priming composition comprising a self-replicating RNA molecule that encodes a first polypeptide antigen that comprises a first epitope; and (ii) a boosting composition comprising a second polypeptide antigen that comprises a second epitope; wherein said first epitope and second epitope are the same epitope. The kits are suitable for sequential administration of the RNA and the polypeptide, such as a “RNA prime, protein boost” immunization regimen to generate an immune response to a pathogen.

Suitable antigens that can be used as the RNA-coded antigen (the first polypeptide antigen) for the priming composition, or the polypeptide antigen (the second polypeptide antigen) for the boosting composition include proteins and peptides derived from HIV.

The RNA molecule of the priming composition can be delivered as naked RNA (e.g. merely as an aqueous solution of RNA). Alternatively, to enhance entry into cells and also subsequent intercellular effects, the priming composition may optionally comprise a delivery system (such as a particulate or emulsion delivery system), so that the RNA molecule is administered in combination with the delivery system. Exemplary delivery systems are described above. The delivery system may be in the same container as the RNA molecule (e.g., pre-formulated), or in a different container from the RNA (e.g., the RNA and the delivery system are separately packaged, and may be combined, e.g., within about 72 hours, about 48 hours, about 24 hours, about 12 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 15 minutes, about 10 minutes, or about 5 minutes prior to administration).

The priming composition, the boosting composition, or both, may optionally include one or more immunoregulatory agents such as adjuvants, as described herein. The immunoregulatory agent may be in the same container as the priming or boosting composition, or in a separate contained that can be combined with the priming or boosting composition prior to administration.

The priming composition comprising the RNA molecule or the boosting composition comprising the polypeptide can be in liquid form or can be in solid form (e.g., lyophilized).

### (C) Other Components of the Kits

Suitable containers include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a third container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a fourth container comprising an adjuvant (such as an aluminum containing adjuvant or MF59).

The kit can also comprise a package insert containing written instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions, the priming compositions, or the boosting compositions described above.

### 4. Pharmaceutical Compositions

In one aspect, the invention relates to pharmaceutical compositions comprising an RNA component and a polypeptide component. The pharmaceutical composition comprises: (i) a self-replicating RNA molecule that encodes a first polypeptide antigen comprising a first epitope (the RNA component); and (ii) a second polypeptide antigen comprising a second epitope (the polypeptide component); wherein said first epitope and second epitope are epitopes from HIV; and (iii) a pharmaceutically acceptable carrier and/or a pharmaceutically acceptable vehicle.

In another aspect, the invention relates to a kit comprising: (i) a priming composition comprising a self-replicating RNA molecule that encodes a first polypeptide antigen that comprises a first epitope; and (ii) a boosting composition comprising a second polypeptide antigen that comprises a second epitope; wherein said first epitope and second epitope are the same epitope; and wherein the priming composition, the boosting composition, or both, comprise(s) a pharmaceutically acceptable carrier and/or a pharmaceutically acceptable vehicle.

The pharmaceutical compositions typically include a pharmaceutically acceptable carrier and/or a suitable delivery system as described herein (such as liposomes, nanoemulsions, PLG micro- and nanoparticles, lipoplexes, chitosan micro- and nanoparticles and other polyplexes for RNA delivery). If desired other pharmaceutically acceptable components can be included, such as excipients and adjuvants. These pharmaceutical compositions can be used as vaccines.

Pharmaceutically acceptable carriers are determined in part by the particular composition being administered, as well as by the particular method used to administer the composition. Accordingly, there is a wide variety of suitable formulations of pharmaceutical compositions of the present invention. A variety of aqueous carriers can be used. Suitable pharmaceutically acceptable carriers for use in the pharmaceutical compositions include plain water (e.g. w.f.i.) or a buffer e.g. a phosphate buffer, a Tris buffer, a borate buffer, a succinate buffer, a histidine buffer, or a citrate buffer. Buffer salts will typically be included in the 5-20 mM range.

The pharmaceutical compositions are preferably sterile, and may be sterilized by conventional sterilization techniques.

The compositions may contain pharmaceutically acceptable auxiliary substances as required to approximate physiological conditions such as pH adjusting and buffering agents, and tonicity adjusting agents and the like, for example, sodium acetate, sodium chloride, potassium chloride, calcium chloride, sodium lactate and the like.

Preferably, the pharmaceutical compositions of the invention may have a pH between 5.0 and 9.5, e.g. between 6.0 and 8.0.

Pharmaceutical compositions of the invention may include sodium salts (e.g. sodium chloride) to give tonicity. A concentration of 10±2 mg/ml NaCl is typical e.g. about 9 mg/ml.

Pharmaceutical compositions of the invention may have an osmolarity of between 200 mOsm/kg and 400 mOsm/kg, e.g. between 240-360 mOsm/kg, or between 290-310 mOsm/kg.

Pharmaceutical compositions of the invention may include one or more preservatives, such as thiomersal or 2-phenoxyethanol. Mercury-free compositions are preferred, and preservative-free vaccines can be prepared.

Pharmaceutical compositions of the invention are preferably non-pyrogenic e.g. containing <1 EU (endotoxin unit, a standard measure) per dose, and preferably <0.1 EU per dose. Pharmaceutical compositions of the invention are preferably gluten free.

The concentrations of the polypeptide molecule and/or the RNA molecule in the pharmaceutical compositions can vary, and will be selected based on fluid volumes, viscosities, body weight and other considerations in accordance with the particular mode of administration selected and the intended recipient's needs. However, the pharmaceutical compositions are formulated to provide an effective amount of RNA+polypeptide (either administered simultaneously, or administered sequentially, such as RNA prime, protein boost), such as an amount (either in a single dose or as part of a series) that is effective for treatment or prevention. This amount varies depending upon the health and physical condition of the individual to be treated, age, the taxonomic group of individual to be treated (e.g. non-human primate, primate, etc.), the capacity of the individual's immune system to react to the antigen encoded protein or peptide, the condition to be treated, and other relevant factors. It is expected that the amount will fall in a relatively broad range that can be determined through routine trials. The RNA content of compositions will generally be expressed in terms of the amount of RNA per dose. A preferred dose has ≧200 μg, <100 μg, ≦50 μg, or ≦10 μg RNA, and expression can be seen at much lower levels e.g. ≦1 μg/dose, ≦100 ng/dose, ≦10 ng/dose, ≦1 ng/dose, etc. The amount of polypeptide in each dose will generally comprise from about 0.1 to about 100 μg of polypeptide, with from about 5 to about 50 μg being preferred and from about 5 to about 25 μg/dose being alternatively preferred.

The amount of adjuvant, if any, will be an amount that will induce an immunomodulating response without significant adverse side effect. An optional amount for a particular vaccine can be ascertained by standard studies involving observation of a vaccine's antibody titers and their virus neutralization capabilities. The amount of adjuvant will be from about 1 to about 100 μg/dose, with from about 5 to about 50 μg/dose being preferred, and from about 20 to about 50 μg/dose being alternatively preferred.

Formulations suitable for parenteral administration, such as, for example, by intraarticular (in the joints), intravenous or intraperitoneal injection, and preferably by intramuscular, intradermal or subcutaneous injection, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials. Injection solutions and suspensions can be prepared from sterile powders, granules, and tablets. Cells transduced by the RNA molecules can also be administered intravenously or parenterally.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

It is recognized that polypeptide and RNA molecules, when administered orally, must be protected from digestion. Protection of polypeptide and RNA molecules can typically be accomplished either by complexing the RNA molecule or the polypeptide molecule with a composition to render the RNA/polypeptide resistant to acidic and enzymatic hydrolysis, or by packaging the RNA molecule or the polypeptide molecule in an appropriately resistant carrier such as a liposome. Means of protecting nucleic acids (such as RNA molecules) and polypeptides from digestion are well known in the art.

The pharmaceutical compositions can be encapsulated, e.g., in liposomes, or in a formulation that provides for slow release of the active ingredient. For example, the RNA molecule may be formulated as liposomes, then administered as a priming composition. Alternatively, liposome-formulated RNA may be mixed with the polypeptide molecule to produce the RNA+polypeptide immunogenic composition of the invention. Alternatively, the RNA molecule and the polypeptide molecule can be co-encapsulated in liposomes.

The compositions described herein (priming compositions, boosting compositions, or immunogenic compositions comprising an RNA and a polypeptide), alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable suppository formulations may contain the RNA, the polypeptide, or the polypeptide and RNA combination as described herein, and a suppository base. Suitable suppository bases include natural or synthetic triglycerides or paraffin hydrocarbons. It is also possible to use gelatin rectal capsules filled with the polypeptide and RNA molecules as described herein, and a suitable base, for example, liquid triglycerides, polyethylene glycols, and paraffin hydrocarbons.

### 5. Methods of Generating or Enhancing Immune Responses

### (A) Co-Administration of an RNA Molecule and a Polypeptide Molecule

In another aspect, the invention provides a method for inducing, generating or enhancing an immune response in a subject in need thereof, such as a human, comprising administering an effective amount of an immunogenic composition comprising an RNA component and a polypeptide component. The composition comprises: (i) a self-replicating RNA molecule that encodes a first polypeptide antigen comprising a first epitope (the RNA component); and (ii) a polypeptide antigen comprising a second epitope (the polypeptide component); wherein said first epitope and second epitope are epitopes from HIV. The immune response is preferably protective and preferably involves antibodies and/or cell-mediated immunity. The method may be used to induce a primary immune response and/or to boost an immune response.

In another aspect, the immunogenic compositions disclosed herein may be used in the manufacture of a medicament for inducing, generating, or enhancing an immune response in a subject in need thereof, such as a human.

In another aspect, the invention provides a method for treating or preventing an infectious disease in a subject (such as a human) in need thereof, comprising administering an effective amount of an immunogenic composition comprising an RNA component and a polypeptide component. The composition comprises: (i) a self-replicating RNA molecule that encodes a first polypeptide antigen comprising a first epitope(the RNA component); and (ii) a polypeptide antigen comprising a second epitope (the polypeptide component); wherein said first epitope and second epitope are epitopes from HIV.

In another aspect, the compositions disclosed herein may be used in the manufacture of a medicament for treating or preventing HIV in a subject in need thereof, such as a human.

In another aspect, the invention provides a method for vaccinating a subject, such as a human, or immunizing a subject against HIV, comprising administering to a subject in need thereof an effective amount of an immunogenic composition comprising an RNA component and a polypeptide component. The composition comprises: (i) a self-replicating RNA molecule that encodes a first polypeptide antigen comprising a first epitope (the RNA component); and (ii) a polypeptide antigen comprising a second epitope (the polypeptide component); wherein said first epitope and second epitope are epitopes from HIV.

In another aspect, the compositions disclosed herein may be used in the manufacture of a medicament for vaccinating a subject in need thereof, such as a human.

When the RNA molecule and the polypeptide molecule are co-administered, it may still be desirable to package the polypeptide molecule and RNA molecule separately. The two components may be combined, e.g., within about 72 hours, about 48 hours, about 24 hours, about 12 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 15 minutes, about 10 minutes, or about 5 minutes prior to administration. For example, the polypeptide molecule and RNA molecule can be combined at a patient's bedside.

### (B) Prime-Boost

One aspect of the invention relates to the “prime and boost” immunization regimes in which the immune response induced by a priming composition is boosted by a boosting composition. For example, following priming (at least once) with an antigen (e.g., a polypeptide antigen, an RNA-coded antigen, an attenuated pathogen, or a combination thereof), a boosting composition comprising substantially the same antigen in the same form (e.g., protein prime, protein boost; RNA prime, RNA boost; etc.), substantially the same antigen in a different form (e.g., RNA prime, protein boost; in which the RNA and the protein are directed to the same target antigen), or a different antigen in the same or a different form (e.g., RNA prime targeting antigen 1, protein boost targeting antigen 2, wherein antigen 1 and antigen 2 are different but share a common epitope), may be administered to boost the immune response in the primed host.

In another aspect, the invention provides a method for inducing, generating or enhancing an immune response in a subject in need thereof, such as a human, comprising: (i) administering to a subject in need thereof at least once a therapeutically effective amount of a priming composition comprising a self-replicating RNA molecule that encodes a first polypeptide antigen that comprises a first epitope; and (ii) subsequently administering the subject at least once a therapeutically effective amount of a boosting composition comprising a second polypeptide antigen that comprises a second epitope; wherein said first epitope and second epitope are the same epitope. The immune response is preferably protective and preferably involves antibodies and/or cell-mediated immunity.

In another aspect, the priming and boosting compositions disclosed herein may be used in the manufacture of a medicament for inducing, generating, or enhancing an immune response in a subject in need thereof, such as a human.

In another aspect, the invention provides a method for treating or preventing HIV in a subject in need thereof, such as a human, comprising: (i) administering to a subject in need thereof at least once a therapeutically effective amount of a priming composition comprising a self-replicating RNA molecule that encodes a first polypeptide antigen that comprises a first epitope; and (ii) subsequently administering the subject at least once a therapeutically effective amount of a boosting composition comprising a second polypeptide antigen that comprises a second epitope; wherein said first epitope and second epitope are the same epitope.

In another aspect, the priming and boosting compositions disclosed herein may be used in the manufacture of a medicament for treating or preventing HIV in a subject in need thereof, such as a human.

In another aspect, the invention provides a method for vaccinating a subject, such as a human, or immunizing a subject, such as a human, against HIV, comprising: (i) administering to a subject in need thereof at least once a therapeutically effective amount of a priming composition comprising a self-replicating RNA molecule that encodes a first polypeptide antigen that comprises a first epitope; and (ii) subsequently administering the subject at least once a therapeutically effective amount of a boosting composition comprising a second polypeptide antigen that comprises a second epitope; wherein said first epitope and second epitope are the same epitope.

In another aspect, the priming and boosting compositions disclosed herein may be used in the manufacture of a medicament for vaccinating a subject in need thereof, such as a human.

The priming composition and the boosting composition may be substantially the same (e.g., RNA+protein prime, RNA+protein boost), or may be different (e.g., RNA+protein prime, protein boost).

The antigens (either in polypeptide form or in RNA-coded form) to be included in the priming and boosting compositions need not be identical, but should share at least one common epitope (e.g., the priming composition comprising an RNA molecule that encodes a first polypeptide antigen that comprises a first epitope; the boosting composition comprising a second polypeptide antigen that comprises a second epitope; wherein said first epitope and second epitope are the same epitope).

One embodiment of the invention uses an “RNA prime, protein boost” immunization strategy. Following priming (at least once) with an RNA molecule, a polypeptide molecule is subsequently administered to boost the immune response in the primed host.

Another embodiment of the invention uses an “RNA+protein prime, protein boost” strategy. Following priming (at least once) with an immunogenic composition comprising an RNA molecule and a polypeptide molecule, a polypeptide molecule is subsequently administered to boost the immune response in the primed host.

The subject may be primed and/or boosted more than once. For example, the immunization strategy can be prime, prime, boost; or prime, boost, boost. In certain embodiment, the priming composition is administered as least twice, at least 3 times, at least 4 times, or at least 5 times. In certain embodiment, the boost composition is administered as least twice, at least 3 times, at least 4 times, or at least 5 times.

Administration of the boosting composition is generally weeks or months after administration of the priming composition, such as about 1 week, about 2 weeks, about 3 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1 month, about 2 months, about 3 months, about 4 months, about 5 months, about 6 months, about 7 months, about 8 months, about 9 months, about 10 months, about 11 months, about 12 months, about 18 months, about 2 years, about 3 years, about 4 years, about 5 years, about 6 years, about 7 years, about 8 years, about 9 years, or about 10 years after the priming composition is administered.

### (C) Additional Considerations for Administration

One way of checking efficacy of therapeutic treatment involves monitoring pathogen infection after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

Another way of assessing the immunogenicity of the compositions or vaccines disclosed herein where the nucleic acid molecule (e.g., the RNA) encodes a protein antigen is to express the protein antigen recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

The efficacy of the compositions can also be determined in vivo by challenging appropriate animal models of the pathogen of interest infection.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The compositions disclosed herein may be used to treat both children and adults. Thus a human subject may be less than 1 year old, 1-5 years old, 5-15 years old, 15-55 years old, or at least 55 years old.

Preferred routes of administration include, but are not limited to, intramuscular, intraperitoneal, intradermal, subcutaneous, intravenous, intraarterial, and intraoccular injection. Oral and transdermal administration, as well as administration by inhalation or suppository is also contemplated. Particularly preferred routes of administration include intramuscular, intradermal and subcutaneous injection. According to some embodiments of the present invention, the composition is administered to a host animal using a needleless injection device, which are well-known and widely available.

It is sometimes advantageous to employ a vaccine that targets a particular target cell type (e.g., an antigen presenting cell or an antigen processing cell).

Catheters or like devices may be used to deliver the composition of the invention, as polypeptide+naked RNA, polypeptide+RNA formulated with a delivery system (e.g., RNA encapsulated in liposomes), RNA only, or polypeptide only into a target organ or tissue. Suitable catheters are disclosed in, e.g., U.S. Pat. Nos. 4,186,745; 5,397,307; 5,547,472; 5,674,192; and 6,129,705, all of which are incorporated herein by reference. The RNA molecules of the invention can also be introduced directly into a tissue, such as muscle. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of RNA into cells of a mammal. These methods are useful not only for in vivo introduction of RNA into a mammal, but also for ex vivo modification of cells for reintroduction into a mammal.

The present invention includes the use of suitable delivery systems, such as liposomes, polymer microparticles or submicron emulsion microparticles with encapsulated or adsorbed RNA, or RNA+polypeptide, to deliver the RNA, or RNA+polypeptide, to elicit an immune response. The invention includes liposomes, microparticles, submicron emulsions, or combinations thereof, with adsorbed and/or encapsulated RNA, or RNA+polypeptide.

The compositions disclosed herein that include one or more antigens, or are used in conjunction with one or more antigens, may be administered to patients at substantially the same time as (e.g., during the same medical consultation or visit to a healthcare professional or vaccination centre) other vaccines, e.g., at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A C W135 Y vaccine), a respiratory syncytial virus vaccine, etc.

### 6. Definitions

The term “about”, as used here, refers to +/−10% of a value.

An “antigen” refers to a molecule containing one or more epitopes (either linear, conformational or both), that elicits an immunological response.

An “epitope” is a portion of an antigen that is recognized by the immune system (e.g., by an antibody, an immunoglobulin receptor, a B cell receptor, or a T cell receptor). An epitope can be linear or conformational. Commonly, an epitope is a polypeptide or polysaccharide in a naturally occurring antigen. In artificial antigens it can be a low molecular weight substance such as an arsanilic acid derivative.

T-cells and B-cells recognize antigens in different ways. T-cells recognize peptide fragments of proteins that are embedded in class-II or class-I MHC molecules at the surface of cells, whereas B-cells recognize surface features of an unprocessed antigen, via immunoglobulin-like cell surface receptors. The difference in antigen recognition mechanisms of T-cells and B-cells are reflected in the different natures of their epitopes. Thus, whereas B-cells recognize surface features of an antigen or a pathogen, T-cell epitopes (which comprise peptides of about 8-12 amino acids in length) can be “internal” as well as “surface” when viewed in the context of the three-dimensional structure of the antigen. Accordingly, a B-cell epitope is preferably exposed on the surface of the antigen or pathogen, and can be linear or conformational, whereas a T-cell epitope is typically linear but is not required to be available or on the surface of the antigen. Normally, a B-cell epitope will include at least about 5 amino acids but can be as small as 3-4 amino acids. A T-cell epitope, such as a CTL epitope, will typically include at least about 7-9 amino acids, and a helper T-cell epitope will typically include at least about 12-20 amino acids.

When an individual is immunized with a polypeptide antigen having multiple epitopes, in many instances the majority of responding T lymphocytes will be specific for one or a few linear epitopes from that antigen and/or a majority of the responding B lymphocytes will be specific for one or a few linear or conformational epitopes from that antigen. Such epitopes are typically referred to as “immunodominant epitopes.” In an antigen having several immunodominant epitopes, a single epitope may be most dominant, and is typically referred to as the “primary” immunodominant epitope. The remaining immunodominant epitopes are typically referred to as “secondary” immunodominant epitope(s).

The term “fusion polypeptide” refers to a single polypeptide in which the amino acid sequence is derived from at least two different naturally occurring proteins or polypeptide chains.

The term “naked” as used herein refers to nucleic acids that are substantially free of other macromolecules, such as lipids, polymers, and proteins. A “naked” nucleic acid, such as a self-replicating RNA, is not formulated with other macromolecules to improve cellular uptake. Accordingly, a naked nucleic acid is not encapsulated in, absorbed on, or bound to a liposome, a microparticle or nanoparticle, a cationic emulsion, and the like.

As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate.

As used herein, two epitopes are from the same pathogen when the two epitopes are from the same pathogen species, but not necessarily from the same strain, serotype, clade, etc. Therefore, the two epitopes can be from two different subspecies, strains, or serotypes of the same pathogen (e.g., one epitope from HIV-1 Clade B, the other epitope from HIV-1 Clade C; etc.).

As used herein, a “polypeptide antigen” refers to a polypeptide comprising one or more epitopes (either linear, conformational or both), that elicits an immunological response. Polypeptide antigens include, for example, a naturally-occurring protein, a mutational variant of a naturally-occurring protein (e.g., a protein that has amino acid substitution(s), addition(s), or deletion(s)), a truncated form of a naturally-occurring protein (e.g., an intracellular domain or extracellular domain of a membrane-anchored protein), as well as a fusion protein (a protein that is derived from at least two different naturally occurring proteins or polypeptide chains). In addition, polypeptide antigens also encompass polypeptides that comprise one or more amino acid stereoisomers, derivatives, or analogues. For example, amino acid derivatives include, e.g., chemical modifications of amino acids such as alkylation, acylation, carbamylation, iodination, etc. Amino acid analogues include, e.g., compounds that have the same basic chemical structure as a naturally occurring amino acid, such as homoserine, norleucine, methionine sulfoxide, methionine methyl sulfonium. Polypeptide antigens also encompass polypeptides that are modified post-translationally (such as acetylated, phosphorylated, or glycosylated polypeptides). Therefore, an epitope of a polypeptide antigen is not limited to a peptide. For example, an epitope of a glycosylated polypeptide may be a saccharide group that is attached to the polypeptide chain.

Two protein antigens are “substantially the same” if the amino acid sequence identify between the two antigens is at least about 90%, at least about 95%, at least about 96%, at least about 97%, at least about 98%, or at least about 99%, across the length of the shorter antigen.

The terms “treat,” “treating” or “treatment”, as used herein, include alleviating, abating or ameliorating disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments

The term “viral replicon particle” or “VRP” refers to recombinant infectious virions that cannot generate infectious progeny because of deletion of structural gene(s).

The term “virus-like particle” or “VLP” refers to a structure formed by viral coat proteins (e.g., a capsid) and optionally an evelope, but having no genetic material. A VLP resembles a viral particle.

### EXEMPLIFICATION

The invention now being generally described, it will be more readily understood by reference to the following examples, which are included merely for purposes of illustration of certain aspects and embodiments of the present invention, and are not intended to limit the invention.

### Methods:

### RNA Synthesis

Plasmid DNA encoding alphavirus replicons (see sequences, vA317, vA17, vA336, vA160, vA322, vA311, vA306, vA142, vA526, vA527, vA318, vA140, vA318, vA372, vA368, vA369) served as a template for synthesis of RNA in vitro. Replicons contain the genetic elements required for RNA replication but lack those encoding gene products necessary for particle assembly; the structural genes of the alphavirus genome are replaced by sequences encoding a heterologous protein. Upon delivery of the replicons to eukaryotic cells, the positive-stranded RNA is translated to produce four non-structural proteins, which together replicate the genomic RNA and transcribe abundant subgenomic mRNAs encoding the heterologous gene product. Due to the lack of expression of the alphavirus structural proteins, replicons are incapable of inducing the generation of infectious particles. A bacteriophage (T7 or SP6) promoter upstream of the alphavirus cDNA facilitates the synthesis of the replicon RNA in vitro and the hepatitis delta virus (HDV) ribozyme immediately downstream of the poly(A)-tail generates the correct 3′-end through its self-cleaving activity.

Following linearization of the plasmid DNA downstream of the HDV ribozyme with a suitable restriction endonuclease, run-off transcripts were synthesized in vitro using T7 or SP6 bacteriophage derived DNA-dependent RNA polymerase. Transcriptions were performed for 2 hours at 37° C. in the presence of 7.5 mM (T7 RNA polymerase) or 5 mM (SP6 RNA polymerase) of each of the nucleoside triphosphates (ATP, CTP, GTP and UTP) following the instructions provided by the manufacturer (Ambion, Austin, Tex.). Following transcription, the template DNA was digested with TURBO DNase (Ambion, Austin, Tex.). The replicon RNA was precipitated with LiCl and reconstituted in nuclease-free water. Uncapped RNA was capped post-transcripionally with Vaccinia Capping Enzyme (VCE) using the ScriptCap m⁷G Capping System (Epicentre Biotechnologies, Madison, Wis.) as outlined in the user manual. Post-transcriptionally capped RNA was precipitated with LiCl and reconstituted in nuclease-free water. The concentration of the RNA samples was determined by measuring the optical density at 260 nm. Integrity of the in vitro transcripts was confirmed by denaturing agarose gel electrophoresis.

### LNP Formulation

1,2-dilinoleyloxy-N,N-dimethyl-3-aminopropane (DlinDMA) was synthesized using a previously published procedure [Heyes, J., Palmer, L., Bremner, K., MacLachlan, I. Cationic lipid saturation influences intracellular delivery of encapsulated nucleic acids. Journal of Controlled Release, 107: 276-287 (2005)]. 1,2-Diastearoyl-sn-glycero-3-phosphocholine (DSPC) was purchased from Genzyme. Cholesterol was obtained from Sigma-Aldrich (St. Lois, Mo.). 1,2-dimyristoyl-sn-glycero-3-phosphoethanolamine-N-[methoxy(polyethylene glycol)-2000] (ammonium salt) (PEG DMG 2000), 1,2-dimyristoyl-sn-glycero-3-phosphoethanolamine-N-[methoxy(polyethylene glycol)-2000] (ammonium salt) (PEG DMG 1000) and 1,2-dimyristoyl-sn-glycero-3-phosphoethanolamine-N-[methoxy(polyethylene glycol)-2000] (ammonium salt) (PEG DMG 3000) were obtained from Avanti Polar Lipids (Alabaster, Ala.). 1,2-dioleoyl-3-trimethylammonium-propane (chloride salt) (DOTAP) and 3β-[N—(N′,N′-dimethylaminoethane)-carbamoyl] cholesterol hydrochloride (DC-chol) were obtained from Avanti Polar Lipids.

LNPs were Formulated Using Three Methods:

### Method A

(40 μg batch, no mustang, no second mixing, no TFF, with dialysis)

Fresh lipid stock solutions in ethanol were prepared. 37 mg of DlinDMA, 11.8 mg of DSPC, 27.8 mg of Cholesterol and 8.07 mg of PEG DMG 2000 were weighed and dissolved in 7.55 mL of ethanol. The freshly prepared lipid stock solution was gently rocked at 37° C. for about 15 min to form a homogenous mixture. Then, 120.9 μL of the stock was added to 1.879 mL ethanol to make a working lipid stock solution of 2 mL. This amount of lipids was used to form LNPs with 40 μg RNA at a 8:1 N:P (Nitrogen to Phosphate) ratio. The protonatable nitrogen on DlinDMA (the cationic lipid) and phosphates on the RNA are used for this calculation. Each μg of self-replicating RNA molecule was assumed to contain 3 nmoles of anionic phosphate, each μg of DlinDMA was assumed to contains 1.6 nmoles of cationic nitrogen. A 2 mL working solution of RNA was also prepared from a stock solution of ˜1 μg/μL in 100 mM citrate buffer (pH 6) (Teknova). Three 20 mL glass vials (with stir bars) were rinsed with RNase Away solution (Molecular BioProducts) and washed with plenty of MilliQ water before use to decontaminate the vials of RNAses. One of the vials was used for the RNA working solution and the others for collecting the lipid and RNA mixes (as described later). The working lipid and RNA solutions were heated at 37° C. for 10 min before being loaded into 3cc luer-lok syringes (BD Medical). 2 mL of citrate buffer (pH 6) was loaded in another 3 cc syringe. Syringes containing RNA and the lipids were connected to a T mixer (PEEK™ 500 μm ID junction) using FEP tubing([fluorinated ethylene-propylene] 2 mm ID×3 mm OD, Idex Health Science, Oak Harbor, Wash.). The outlet from the T mixer was also FEP tubing (2 mm ID×3 mm). The third syringe containing the citrate buffer was connected to a separate piece of tubing (2 mm ID×3 mm OD). All syringes were then driven at a flow rate of 7 mL/min using a syringe pump (from kdScientific, model no. KDS-220). The tube outlets were positioned to collect the mixtures in a 20 mL glass vial (while stirring). Next, LNPs were loaded into Pierce Slide-A-Lyzer Dialysis Cassettes (Thermo Scientific, extra strength, 0.5-3 mL capacity) and dialyzed against 400-500 mL of 1×PBS (diluted from 10× AccuGENE PBS, from Lonza) overnight at 4° C. in an autoclaved plastic container before recovering the final product. For in vitro and in vivo experiments, formulations were diluted to the required RNA concentration with 1×PBS (from Teknova).

pKas

Unless explicitly indicated otherwise, all pKas referred to herein are measured in water at standard temperature and pressure. Also, unless otherwise indicated, all references to pKa are references to pKa measured using the following technique. 2 mM solution of lipid in ethanol are prepared by weighing the lipid and then dissolving in ethanol. 0.3 mM solution of fluorescent probe TNS in ethanol:methanol 9:1 is prepared by first making 3 mM solution of TNS in methanol and then diluting to 0.3 mM with ethanol.

An aqueous buffer containing sodium phosphate, sodium citrate, sodium acetate and sodium chloride, at the concentrations 20 mM, 25 mM, 20 mM and 150 mM, respectively, is prepared. The buffer is split into eight parts and the pH adjusted either with 12N HCl or 6N NaOH to 4.44-4.52, 5.27, 6.15-6.21, 6.57, 7.10-7.20, 7.72-7.80, 8.27-8.33 and 10.47-11.12. 400 uL of 2 mM lipid solution and 800 uL of 0.3 mM TNS solution are mixed.

Using the Tecan Genesis RSP150 high throughput liquid handler and Gemini Software, 7.5 uL of probe/lipid mix are added to 242.5 uL of buffer in a 1 mL 96well plate (model NUNC 260252, Nalgae Nunc International). This is done with all eight buffers.

After mixing in 1 mL 96 well plate, 100 uL of each probe/lipid/buffer mixture is transferred to a 250 uL black with clear bottom 96 well plate (model COSTAR 3904, Corning). The fluorescence measurements are carried out on the SpectraMax M5 spectrophotometer using software SoftMax pro 5.2 and following parameters:

| | |
|---|---|
| Read Mode: | Fluorescence, Top read |
| Wavelengths: | Ex 322 nm, Em 431 nm, Auto Cutoff On 420 nm |
| Sensitivity: | Readings 6, PMT: Auto |
| Automix: | Before: Off |
| Autocalibrate: | On |
| Assay plate type: | 96 Well Standard clrbtm |
| Wells to read: | Read entire plate |
| Settling time: | Off |
| Column Wav. Priority: | Column priority |
| Carriage Speed: | Normal |
| Auto read: | Off |

After the measurement, the background fluorescence value of an empty well on the 96 well plate is subtracted from each probe/lipid/buffer mixture. The fluorescence intensity values are then normalized to the value at lowest pH. The normalized fluorescence intensity vs. pH chart is then plotted in the Microsoft Excel software. The eight points are connected with a smooth line.

The point on the line at which the normalized fluorescence intensity is equal to 0.5 is found. The pH corresponding to normalized fluorescence intensity equal to 0.5 is found and is considered the pKa of the lipid.

### Method B

(75 μg batch, PES hollow fibers and no mustang):

Fresh lipid stock solutions in ethanol were prepared. 37 mg of DlinDMA, 11.8 mg of DSPC, 27.8 mg of Cholesterol and 8.07 mg of PEG DMG 2000 were weighed and dissolved in 7.55 mL of ethanol. The freshly prepared lipid stock solution was gently rocked at 37° C. for about 15 min to form a homogenous mixture. Then, 226.7 μL of the stock was added to 1.773 mL ethanol to make a working lipid stock solution of 2 mL. This amount of lipids was used to form LNPs with 75 μg RNA at a 8:1 N:P (Nitrogen to Phosphate) ratio. The protonatable nitrogen on DlinDMA (the cationic lipid) and phosphates on the RNA are used for this calculation. Each μg of self-replicating RNA molecule was assumed to contain 3 nmoles of anionic phosphate, each μg of DlinDMA was assumed to contains 1.6 nmoles of cationic nitrogen. A 2 mL working solution of RNA was also prepared from a stock solution of ˜1 μg/μL in 100 mM citrate buffer (pH 6) (Teknova). Three 20 mL glass vials (with stir bars) were rinsed with RNase Away solution (Molecular BioProducts) and washed with plenty of MilliQ water before use to decontaminate the vials of RNAses. One of the vials was used for the RNA working solution and the others for collecting the lipid and RNA mixes (as described later). The working lipid and RNA solutions were heated at 37° C. for 10 min before being loaded into 3cc luer-lok syringes (BD Medical). 2 mL of citrate buffer (pH 6) was loaded in another 3 cc syringe. Syringes containing RNA and the lipids were connected to a T mixer (PEEK™ 500 μm ID junction) using FEP tubing([fluorinated ethylene-propylene] 2 mm ID×3 mm OD, Idex Health Science, Oak Harbor, Wash.). The outlet from the T mixer was also FEP tubing (2 mm ID×3 mm). The third syringe containing the citrate buffer was connected to a separate piece of tubing (2 mm ID×3 mm OD). All syringes were then driven at a flow rate of 7 mL/min using a syringe pump (from kdScientific, model no. KDS-220). The tube outlets were positioned to collect the mixtures in a 20 mL glass vial (while stirring). The stir bar was taken out and the ethanol/aqueous solution was allowed to equilibrate to room temperature for 1 h. Then the mixture was loaded in a 5 cc syringe (BD Medical), which was fitted to a piece of FEP tubing (2 mm ID×3 mm OD) and in another 5 cc syringe with equal length of FEP tubing, an equal volume of 100 mM citrate buffer (pH 6) was loaded. The two syringes were driven at 7 mL/min flow rate using a syringe pump and the final mixture collected in a 20 mL glass vial (while stirring). Next, LNPs were concentrated to 2 mL and dialyzed against 10-15 volumes of 1×PBS (from Teknova) using the Tangential Flow Filtration (TFF) system before recovering the final product. The TFF system and hollow fiber filtration membranes were purchased from Spectrum Labs and were used according to the manufacturer's guidelines. Polyethersulfone (PES) hollow fiber filtration membranes (part number P-C1-100E-100-01N) with a 100 kD pore size cutoff and 20 cm² surface area were used. For in vitro and in vivo experiments, formulations were diluted to the required RNA concentration with 1×PBS (from Teknova).

### Method C

(75 μg batch, with mustang and PES hollow fibers):

Fresh lipid stock solutions in ethanol were prepared. 37 mg of DlinDMA, 11.8 mg of DSPC, 27.8 mg of Cholesterol and 8.07 mg of PEG DMG 2000 were weighed and dissolved in 7.55 mL of ethanol. The freshly prepared lipid stock solution was gently rocked at 37° C. for about 15 min to form a homogenous mixture. Then, 226.7 μL of the stock was added to 1.773 mL ethanol to make a working lipid stock solution of 2 mL. This amount of lipids was used to form LNPs with 75 μg RNA at a 8:1 N:P (Nitrogen to Phosphate) ratio. The protonatable nitrogen on DlinDMA (the cationic lipid) and phosphates on the RNA are used for this calculation. Each μg of self-replicating RNA molecule was assumed to contain 3 nmoles of anionic phosphate, each μg of DlinDMA was assumed to contains 1.6 nmoles of cationic nitrogen. A 2 mL working solution of RNA was also prepared from a stock solution of ˜1 μg/μL in 100 mM citrate buffer (pH 6) (Teknova). Three 20 mL glass vials (with stir bars) were rinsed with RNase Away solution (Molecular BioProducts) and washed with plenty of MilliQ water before use to decontaminate the vials of RNAses. One of the vials was used for the RNA working solution and the others for collecting the lipid and RNA mixes (as described later). The working lipid and RNA solutions were heated at 37° C. for 10 min before being loaded into 3cc luer-lok syringes (BD Medical). 2 mL of citrate buffer (pH 6) was loaded in another 3 cc syringe. Syringes containing RNA and the lipids were connected to a T mixer (PEEK™ 500 μm ID junction) using FEP tubing([fluorinated ethylene-propylene] 2 mm ID×3 mm OD, Idex Health Science, Oak Harbor, Wash.). The outlet from the T mixer was also FEP tubing (2 mm ID×3 mm). The third syringe containing the citrate buffer was connected to a separate piece of tubing (2 mm ID×3 mm OD). All syringes were then driven at a flow rate of 7 mL/min using a syringe pump (from kdScientific, model no. KDS-220). The tube outlets were positioned to collect the mixtures in a 20 mL glass vial (while stirring). The stir bar was taken out and the ethanol/aqueous solution was allowed to equilibrate to room temperature for 1 h. Then the mixture was loaded in a 5 cc syringe (BD Medical), which was fitted to a piece of FEP tubing (2 mm ID×3 mm OD) and in another 5 cc syringe with equal length of FEP tubing, an equal volume of 100 mM citrate buffer (pH 6) was loaded. The two syringes were driven at 7 mL/min flow rate using a syringe pump and the final mixture collected in a 20 mL glass vial (while stirring). Next, the mixture collected from the second mixing step (LNPs) were passed through Mustang Q membrane (an anion-exchange support that binds and removes anionic molecules, obtained from Pall Corporation, AnnArbor, Mich., USA). Before passing the LNPs, 4 mL of 1 M NaOH, 4 mL of 1 M NaCl and 10 mL of 100 mM citrate buffer (pH 6) were successively passed through the Mustang membrane. LNPs were warmed for 10 min at 37° C. before passing through the mustang filter. Next, LNPs were concentrated to 2 mL and dialyzed against 10-15 volumes of 1×PBS (from Teknova) using the Tangential Flow Filtration (TFF) system before recovering the final product. The TFF system and hollow fiber filtration membranes were purchased from Spectrum Labs and were used according to the manufacturer's guidelines. Polyethersulfone (PES) hollow fiber filtration membranes (part number P-C1-100E-100-01N) with a 100 kD pore size cutoff and 20 cm² surface area were used. For in vitro and in vivo experiments, formulations were diluted to the required RNA concentration with 1×PBS (from Teknova).

### CNE Formulations

CNEs were prepared similar to charged MF59 as previously described (Ott et al., Journal of Controlled Release, volume 79, pages 1-5, 2002), with one major modification for CMF34. DOTAP was dissolved in the squalene directly, and no organic solvent was used. It was discovered that inclusion of a solvent in emulsions that contained greater than 1.6 mg/ml DOTAP produced a foamy feedstock that could not be microfluidized to produce an emulsion. Heating squalene to 37° C. allowed DOTAP to be directly dissolved in squalene, and then the oil phase could be successfully dispersed in the aqueous phase (e.g., by homogenization) to produce an emulsion.

| | Cationic | | | oil:Lipid | |
|---|---|---|---|---|---|
| | Lipid | | Squa- | ratio | Aqueous |
| CNE | mg/mL | Surfactant | lene | (mole:mole) | phase |
| CNE13 | DDA | 0.5% SPAN | 4.3% | 10 mM | DDA |
| | (in DCM) | 85 | | citrate buffer | (in DCM) |
| | 1.45 | 0.5% Tween | | pH 6.5 | |
| | | 80 | | | |
| CNE17 | DOTAP | 0.5% SPAN | 4.3% | 52.4:1 | 10 mM |
| | (in DCM) | 85 | | | citrate buffer |
| | 1.4 | 0.5% Tween | | | pH 6.5 |
| | | 80 | | | |
| CMF34 | DOTAP | 0.5% SPAN | 4.3% | 16.7:1 | 10 mM |
| | (no organic | 85 | | | citrate buffer |
| | solvent) | 0.5% Tween | | | pH 6.5 |
| | 4.4 | 80 | | | |

### RNA Complexation

The number of nitrogens in solution was calculated from the cationic lipid concentration, DOTAP for example has 1 nitrogen that can be protonated per molecule. The RNA concentration was used to calculate the amount of phosphate in solution using an estimate of 3 nmols of phosphate per microgram of RNA. By varying the amount of RNA:Lipid, the N/P ratio can be modified. RNA was complexed to the CNEs in a range of nitrogen/phosphate ratios (N/P). Calculation of the N/P ratio was done by calculating the number of moles of protonatable nitrogens in the emulsion per milliliter. To calculate the number of phosphates, a constant of 3 nmols of phosphate per microgram of RNA was used. N/P ratio was calculated using the formula:

$$N / P = \frac{( ( \frac{A}{C} ) \times D \times E )}{B \times 3}$$

A is the concentration (mg/ml) of cationic lipid, B is the amount of RNA (□g), C is the molecular weight of the cationic lipid, D is the volume of the emulsion to be complexed (ml), E is the number of protonizable nitrogen atoms in the cationic lipid. The constant 3 is the number of nmoles of phosphate per □g of RNA.

After the values were determined, the appropriate ratio of the emulsion was added to the RNA. Using these values, the RNA was diluted to the appropriate concentration and added directly into an equal volume of emulsion while vortexing lightly. The solution was allowed to sit at room temperature for approximately 2 hours. Once complexed the resulting solution was diluted to the appropriate concentration and used within 1 hour.

### Particle Size

Particle size was measured using a Zetasizer Nano ZS (Malvern Instruments, Worcestershire, UK) according to the manufacturer's instructions. Particle sizes are reported as the Z average with the polydispersity index (pdi). Liposomes were diluted in 1×PBS before measurement.

### Encapsulation Efficiency and RNA Concentration

The percentage of encapsulated RNA and RNA concentration were determined by Quant-iT RiboGreen RNA reagent kit (Invitrogen). Manufacturer's instructions were followed in the assay. The ribosomal RNA standard provided in the kit was used to generate a standard curve. LNPs were diluted ten fold or one hundred fold in 1×TE buffer (from kit), before addition of the dye. Separately, LNPs were diluted ten or 100 fold in 1×TE buffer containing 0.5% Triton X (Sigma-Aldrich), before addition of the dye. Thereafter an equal amount of dye was added to each solution and then ˜180 μL of each solution after dye addition was loaded in duplicate into a 96 well tissue culture plate (obtained from VWR, catalog #353072). The fluorescence (Ex 485 nm, Em 528 nm) was read on a microplate reader (from BioTek Instruments, Inc.).

Triton X was used to disrupt the LNPs, providing a fluorescence reading corresponding to the total RNA amount and the sample without Triton X provided fluorescence corresponding to the unencapsulated RNA. % RNA encapsulation was determined as follows: LNP RNA Encapsulation (%)=[(Fₜ−Fᵢ)/Fₜ]×100, where Fₜ is the fluorescence intensity of LNPs with triton X addition and Fᵢ is the fluorescence intensity of the LNP solution without detergent addition. These values (Fₜ and Fᵢ) were obtained after subtraction from blank (1×TE buffer) fluorescence intensity. The concentration of encapsulated RNA was obtained by comparing Fₜ-Fᵢ with the standard curve generated. All LNP formulations were dosed in vivo based on the encapsulated dose.

### Gel Electrophoresis

Denaturing gel electrophoresis was performed to evaluate the integrity of the RNA after the formulation process and to assess the RNAse protection of the encapsulated RNA. The gel was cast as follows: 0.4 g of agarose (Bio-Rad, Hercules, Calif.) was added to 36 ml of DEPC treated water and heated in a microwave until dissolved and then cooled until warm. 4 ml of 10× denaturing gel buffer (Ambion, Austin, Tex.), was then added to the agarose solution. The gel was poured and was allowed to set for at least 30 minutes at room temperature. The gel was then placed in a gel tank, and 1× Northernmax running buffer (Ambion, Austin, Tex.) was added to cover the gel by a few millimeters.

### RNase Protection Assay

RNase digestion was achieved by incubation with 3.8 mAU of RNase A per microgram of RNA (Ambion, Hercules, and CA) for 30 minutes at room temperature. RNase was inactivated with Protenase K (Novagen, Darmstadt, Germany) by incubating the sample at 55° C. for 10 minutes. Post RNase inactivation, a 1:1 v/v mixture of sample to 25:24:1 v/v/v, phenol:chloroform:isoamyl alcohol was added to extract the RNA from the lipids into the aqueous phase. Samples were mixed by vortexing for a few seconds and then placed on a centrifuge for 15 minutes at 12 k RPM. The aqueous phase (containing the RNA) was removed and used to analyze the RNA. Prior to loading (400 ng RNA per well) all the samples were incubated with formaldehyde loading dye, denatured for 10 minutes at 65° C. and cooled to room temperature. Ambion Millennium markers were used to approximate the molecular weight of the RNA construct. The gel was run at 90 V. The gel was stained using 0.1% SYBR gold according to the manufacturer's guidelines (Invitrogen, Carlsbad, Calif.) in water by rocking at room temperature for 1 hour. Gel images were taken on a Bio-Rad Chemidoc XRS imaging system (Hercules, Calif.).

### Secreted Alkaline Phosphatase (SEAP) Assay

To assess the kinetics and amount of antigen production in vivo, an RNA replicon encoding for SEAP was administered with and without formulation to mice via intramuscularly injection. Groups of 5 female BALB/c mice aged 8-10 weeks and weighing about 20 g were immunized with liposomes encapsulating RNA encoding for SEAP. Naked RNA was administered in RNase free 1×PBS. As a positive control, viral replicon particles (VRPs) at a dose of 5×10⁵ infectious units (IU) were also sometimes administered. A 100 μl dose was administered to each mouse (50 μl per site) in the quadriceps muscle. Blood samples were taken 1, 3, and 6 days post injection. Serum was separated from the blood immediately after collection, and stored at −30° C. until use.

A chemiluminescent SEAP assay Phospha-Light System (Applied Biosystems, Bedford, Mass.) was used to analyze the serum. Mouse sera were diluted 1:4 in 1× Phospha-Light dilution buffer. Samples were placed in a water bath sealed with aluminum sealing foil and heat inactivated for 30 minutes at 65° C. After cooling on ice for 3 minutes, and equilibrating to room temperature, 50 μL of Phospha-Light assay buffer was added to the wells and the samples were left at room temperature for 5 minutes. Then, 50 μL of reaction buffer containing 1:20 CSPD® (chemiluminescent alkaline phosphate substrate) substrate was added, and the luminescence was measured after 20 minutes of incubation at room temperature. Luminescence was measured on a Berthold Centro LB 960 luminometer (Oak Ridge, Tenn.) with a 1 second integration per well. The activity of SEAP in each sample was measured in duplicate and the mean of these two measurements taken.

### Viral Replicon Particles (VRP)

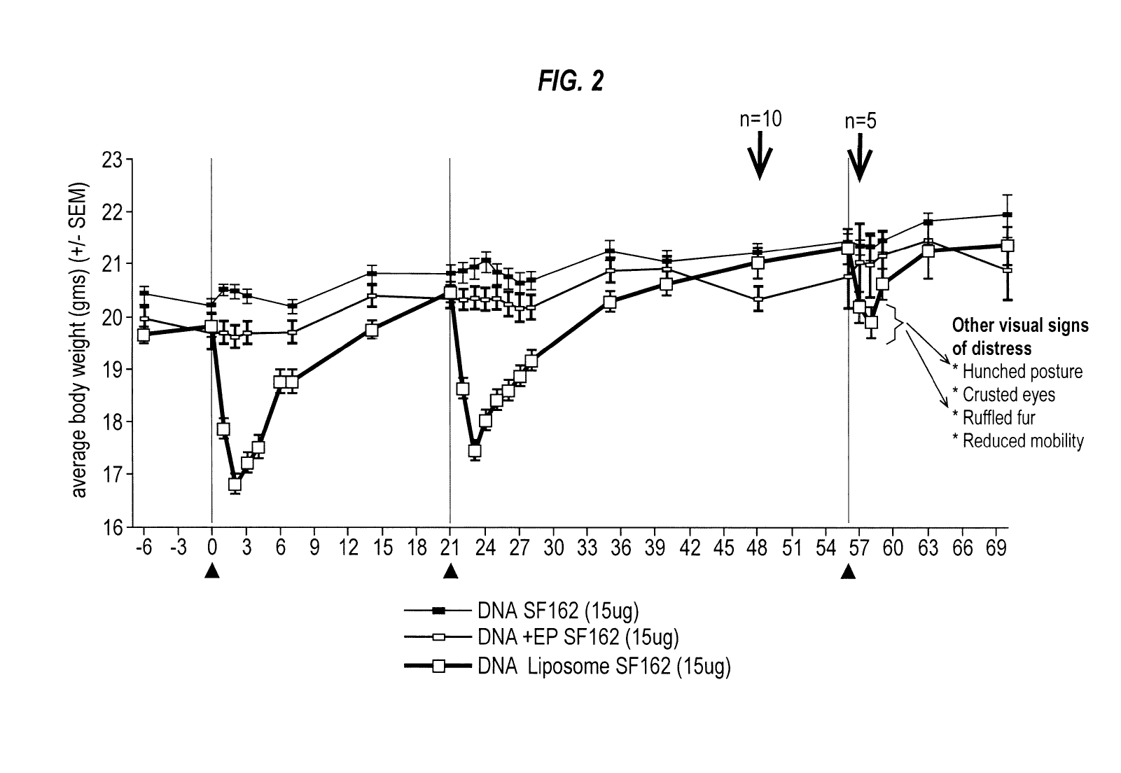

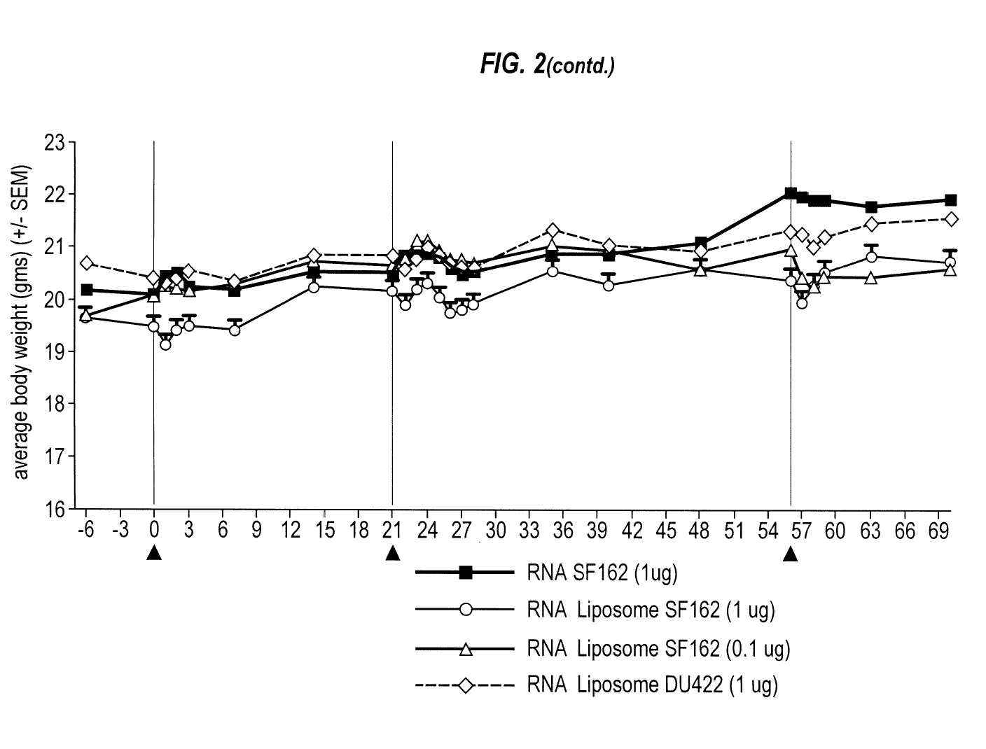

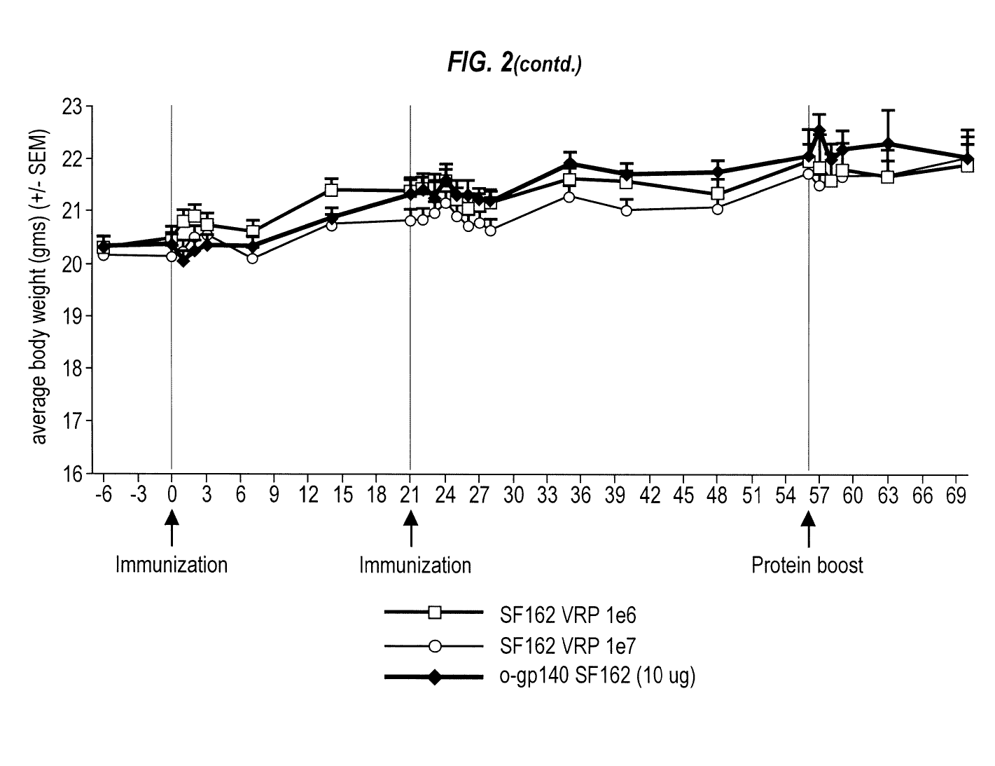

To compare RNA vaccines to traditional RNA-vectored approaches for achieving in vivo expression of reporter genes or antigens, we utilized viral replicon particles (VRPs) produced in BHK cells by the methods described by Perri et al. (2003) An alphavirus replicon particle chimera derived from venezuelan equine encephalitis and sindbis viruses is a potent gene-based vaccine delivery vector. J Virol 77: 10394-10403. In this system, the antigen (or reporter gene) replicons consisted of alphavirus chimeric replicons (VCR) derived from the genome of Venezuelan equine encephalitis virus (VEEV) engineered to contain the 3′ terminal sequences (3′ UTR) of Sindbis virus and a Sindbis virus packaging signal (PS) (see FIG. 2 of Perri et al). These replicons were packaged into VRPs by co-electroporating them into baby hamster kidney (BHK) cells along with defective helper RNAs encoding the Sindbis virus capsid and glycoprotein genes (see FIG. 2 of Perri et al). The VRPs were then harvested and titrated by standard methods and inoculated into animals in culture fluid or other isotonic buffers.

Perri S, Greer C E, Thudium K, Doe B, Legg H, Liu H, Romero R E, Tang Z, Bin Q, Dubensky T W, Jr. et al (2003) An alphavirus replicon particle chimera derived from venezuelan equine encephalitis and sindbis viruses is a potent gene-based vaccine delivery vector. J Virol 77: 10394-10403

### Example I

### HIV Envelop Proteins Study 1—Gp160/Gp140 (RNA Prime, Protein BOOST)

In this example, HIV envelop proteins gp160 and gp140 from HIV-1 Clade B (SF162), and from Clade C (DU422.1) were used as antigens. A “RNA prime, protein boost” regimen was used to assess the effect of sequential administration of (i) an RNA molecule that encodes HIV gp160, and (ii) a “cognate” polypeptide molecule, gp140. gp140 polypeptide corresponds to a truncated form of gp160 where the transmembrane spanning domain of gp160 has been deleted. Thus, the polypeptide antigen is a “cognate” antigen because it is a truncated form of and is substantially the same as the polypeptide encoded by the RNA molecule.

1. Study Design

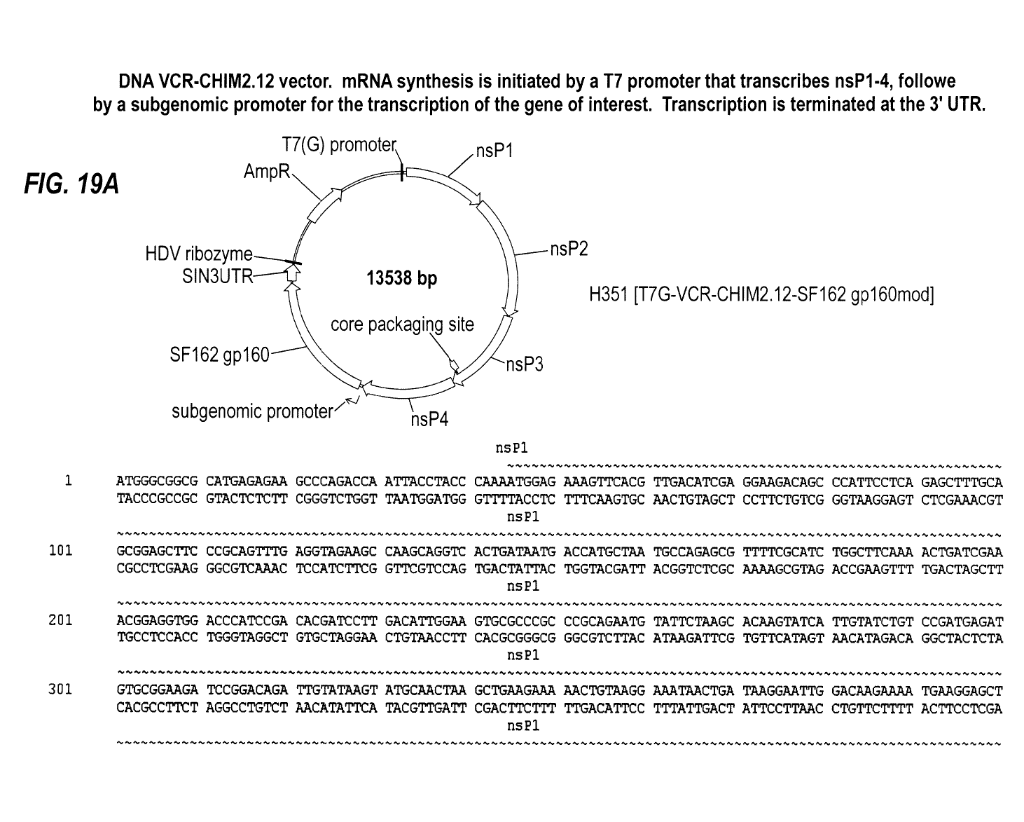

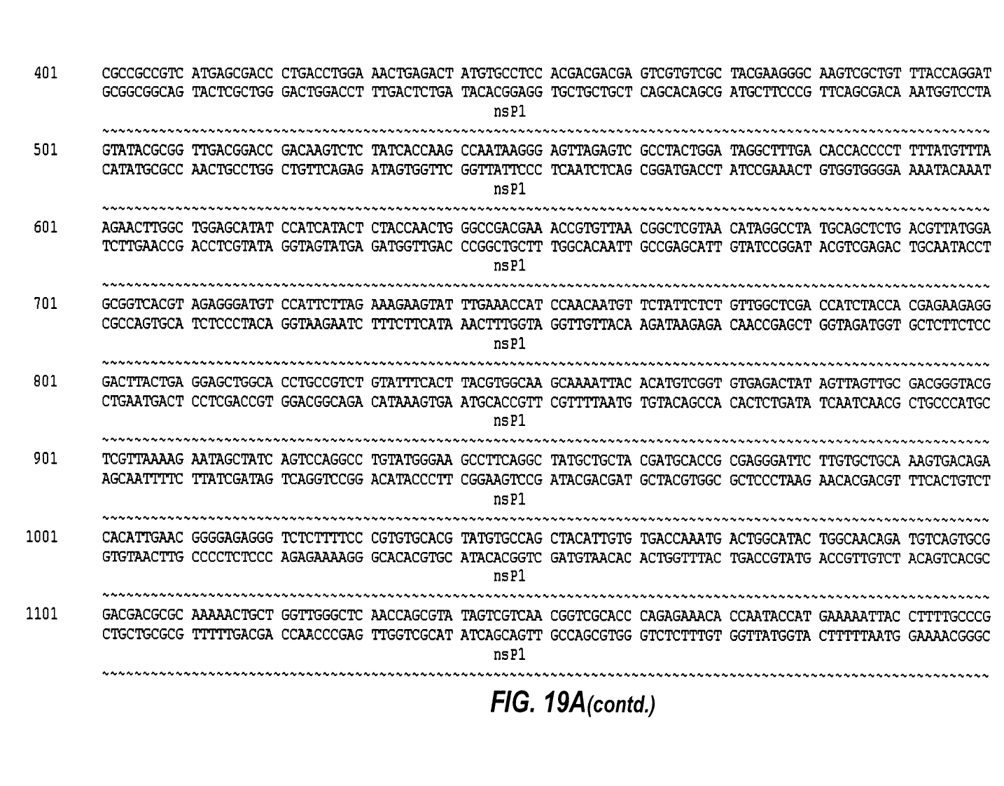

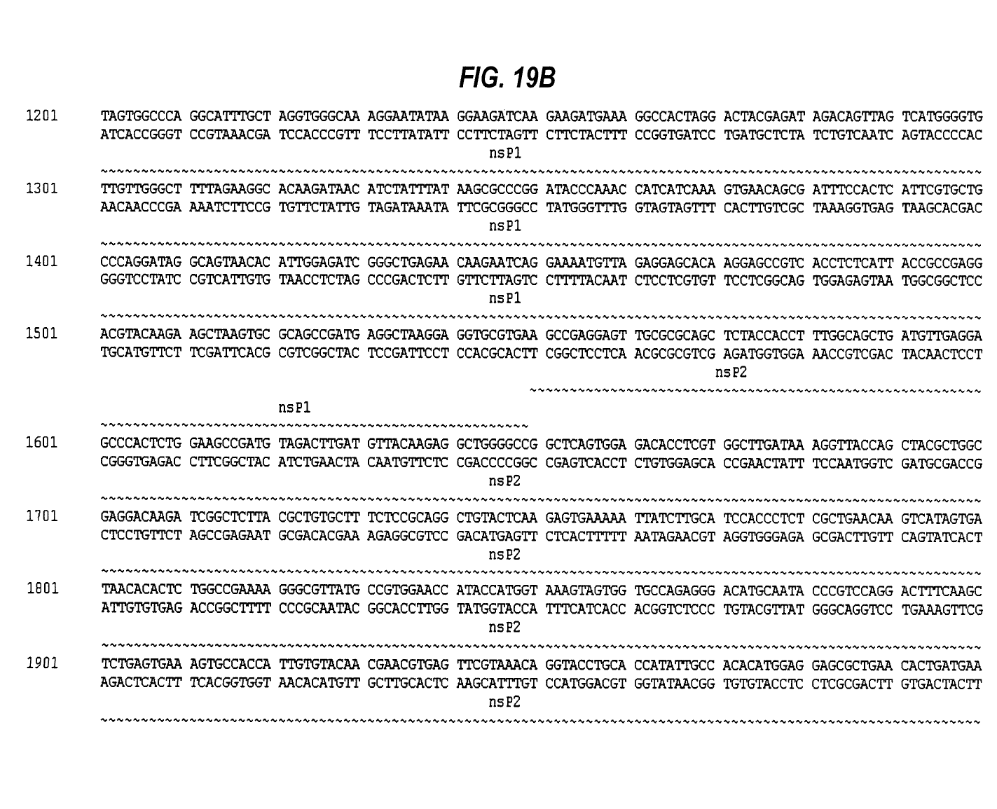

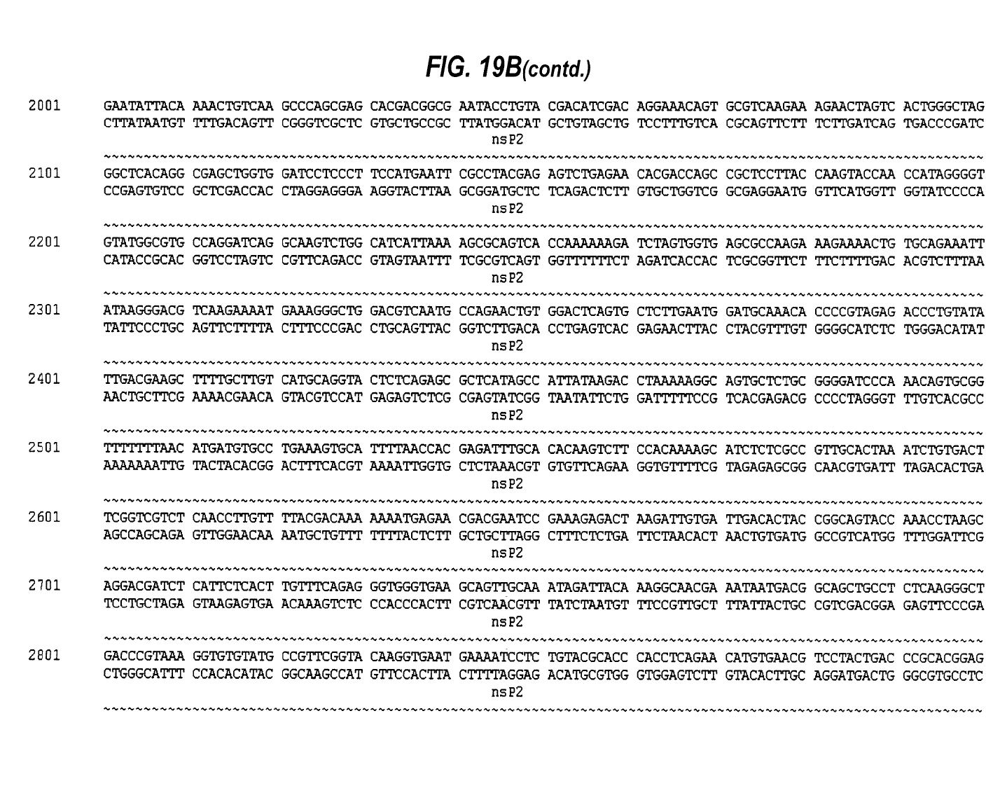

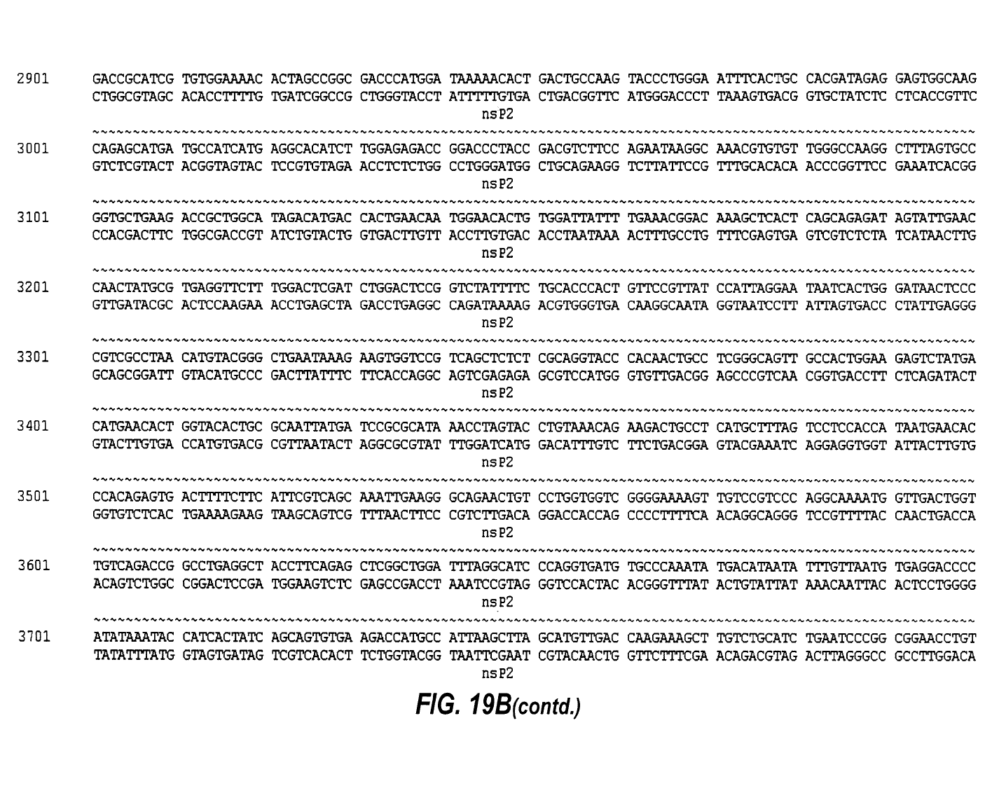

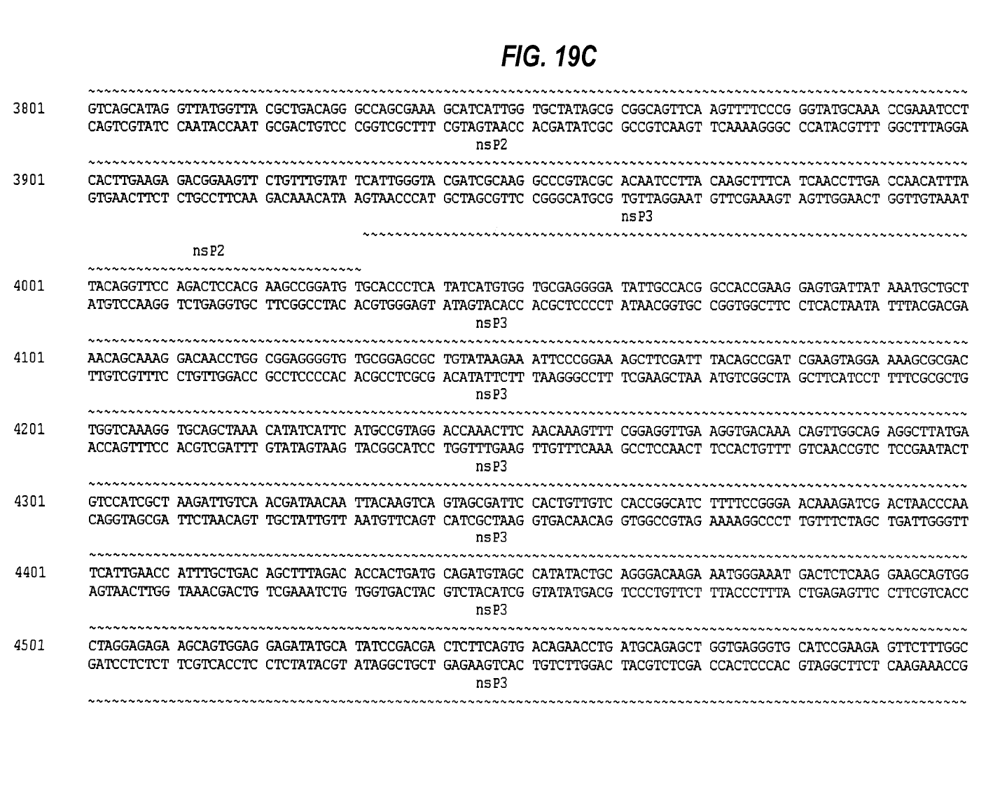

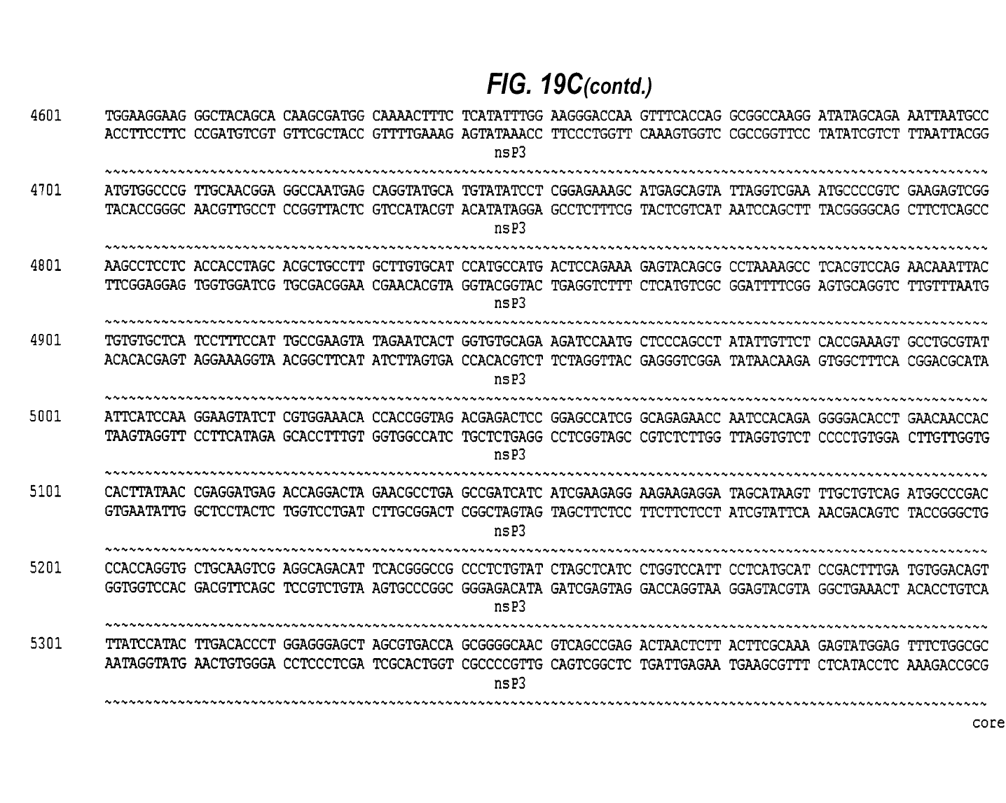

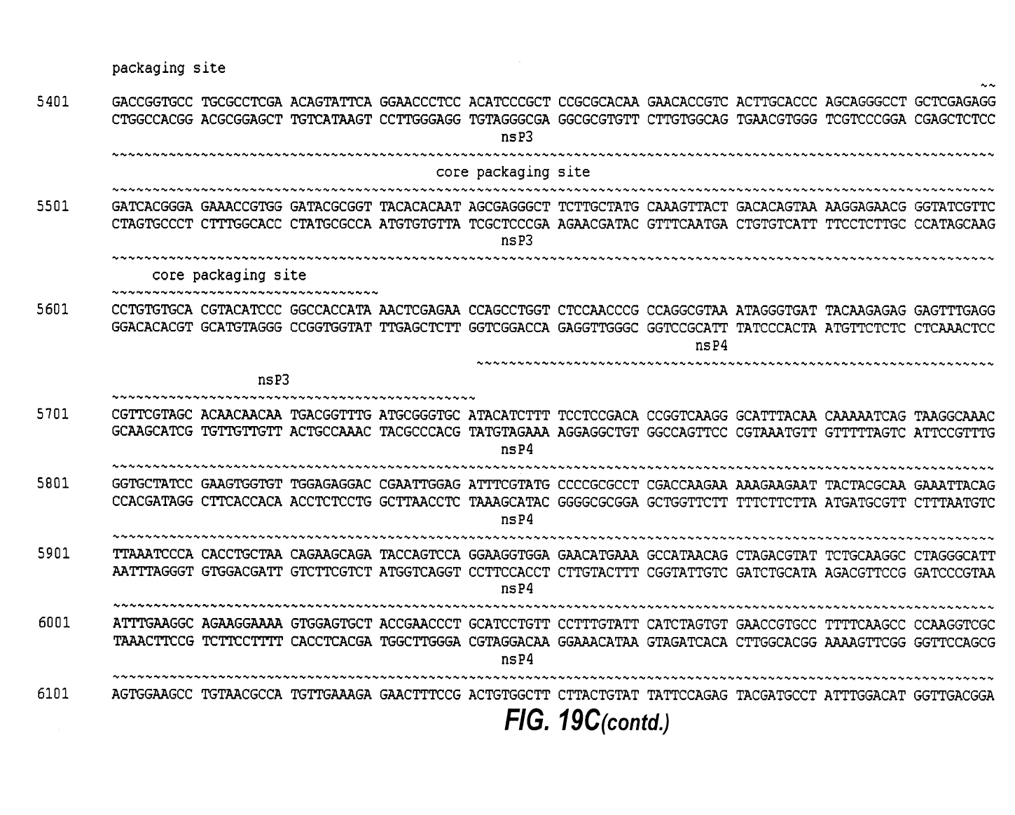

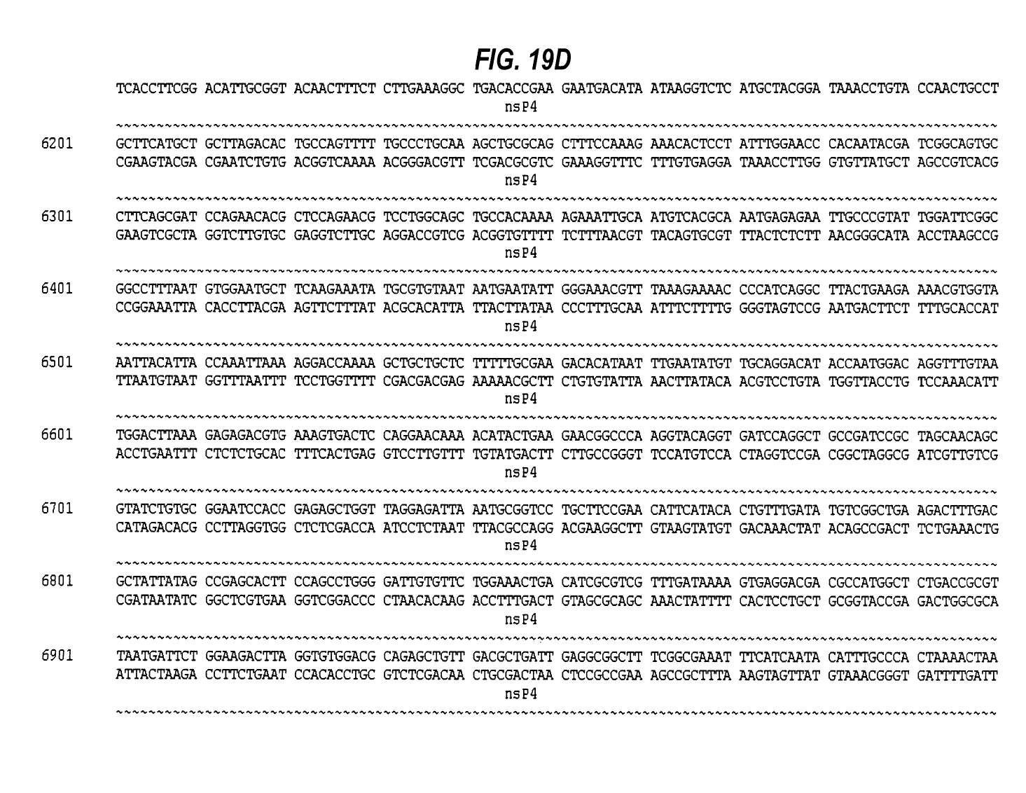

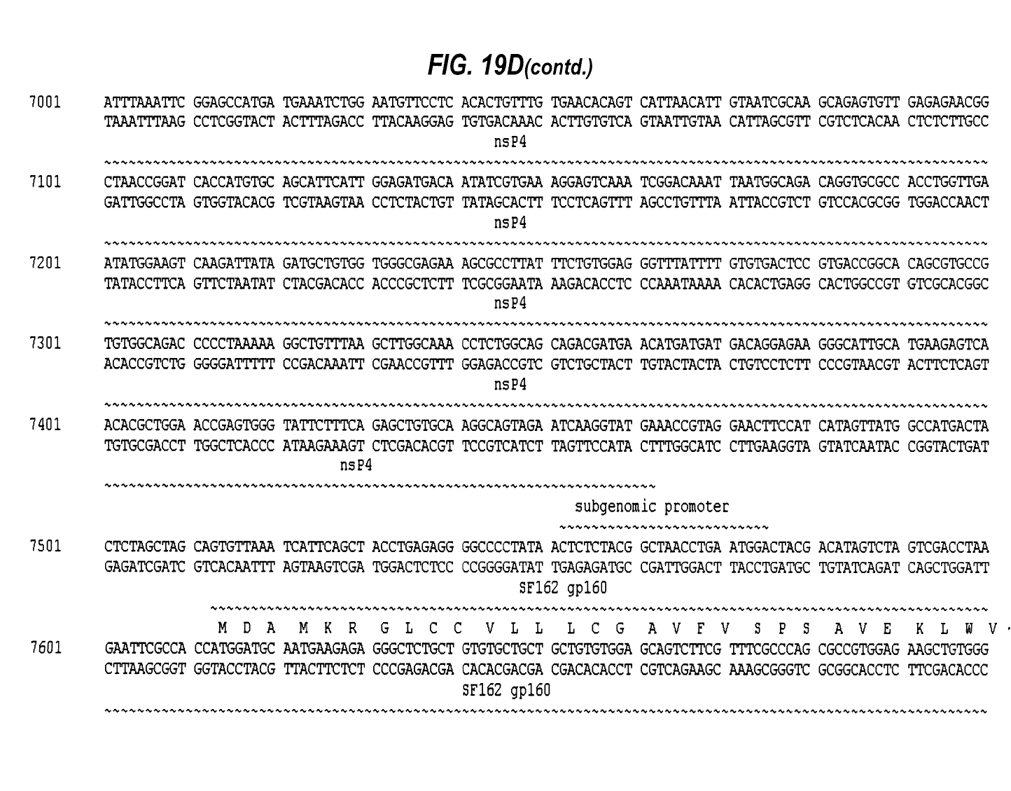

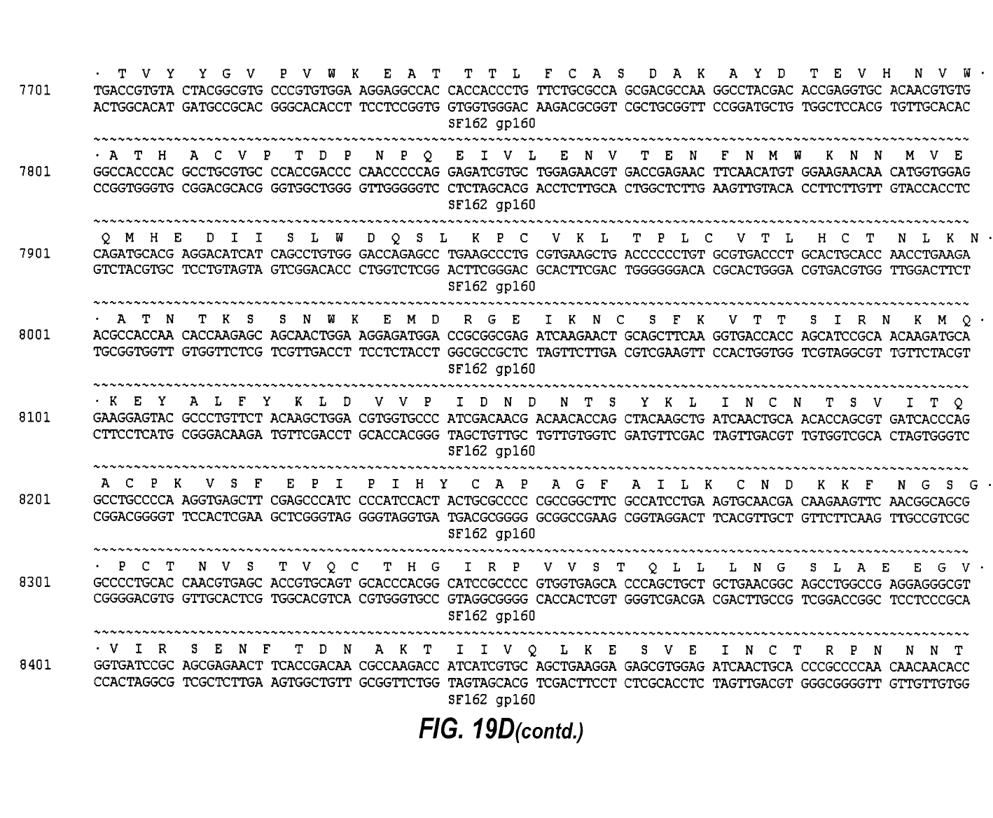

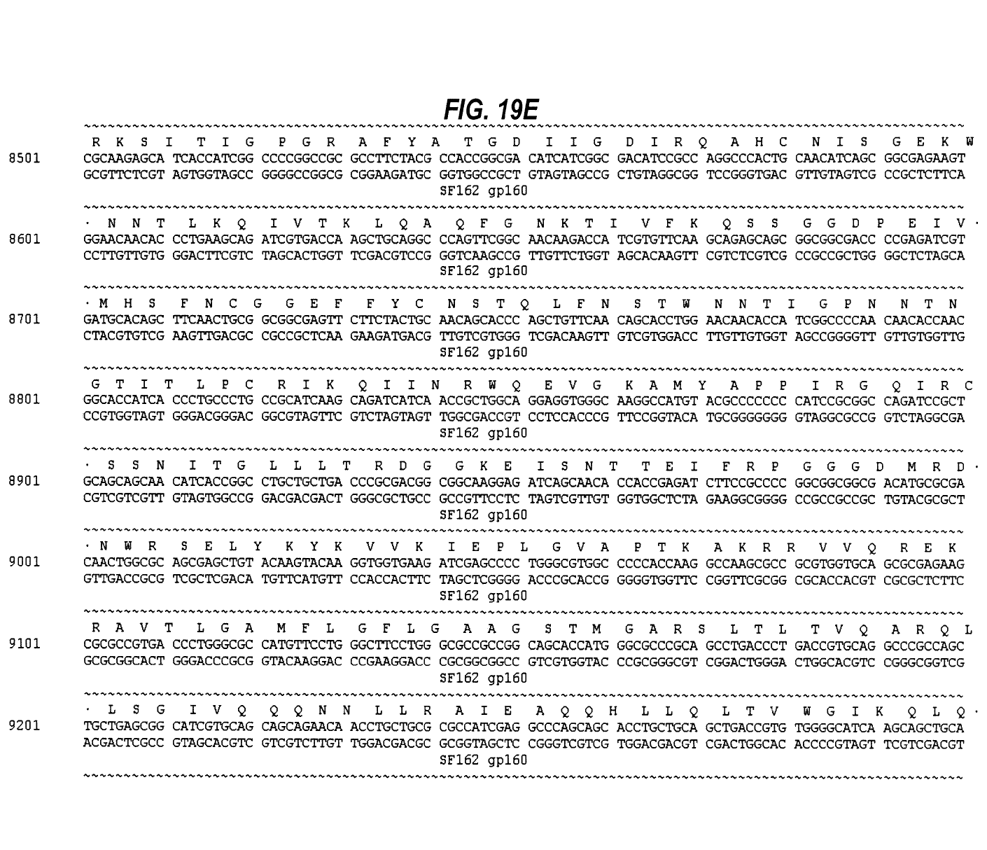

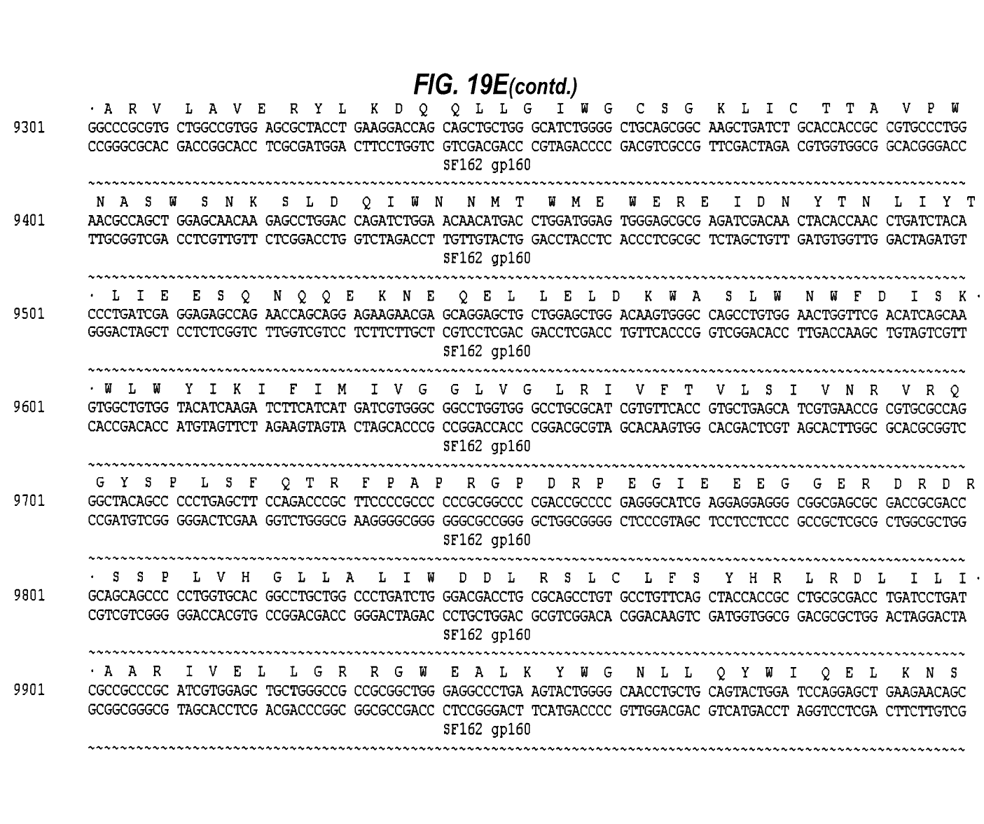

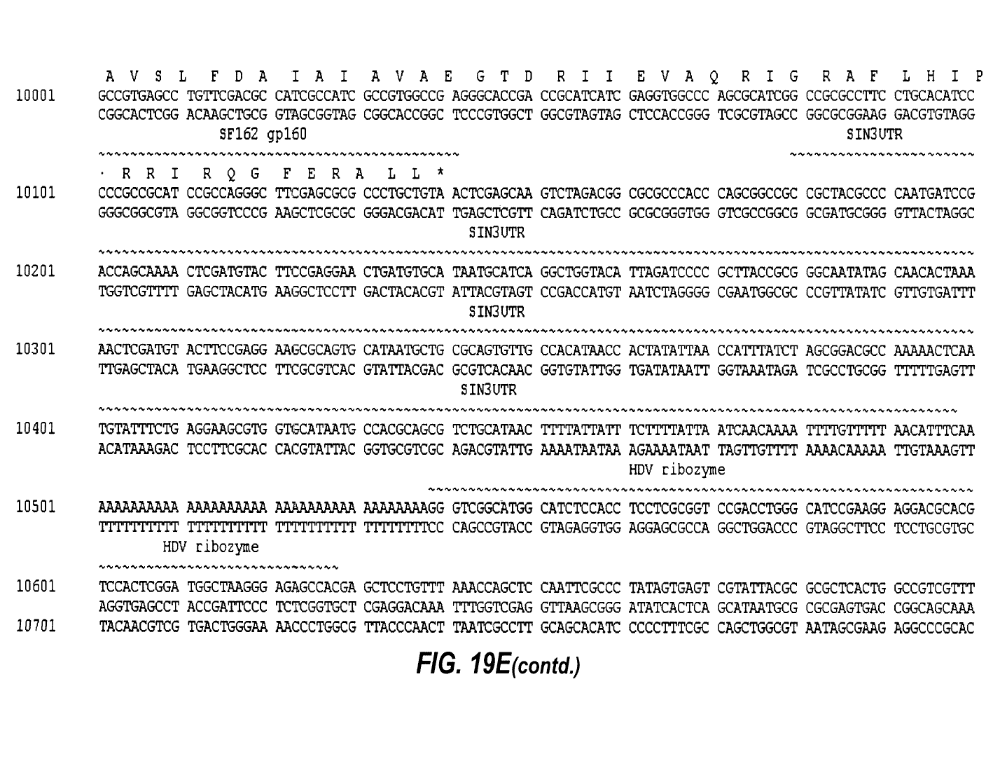

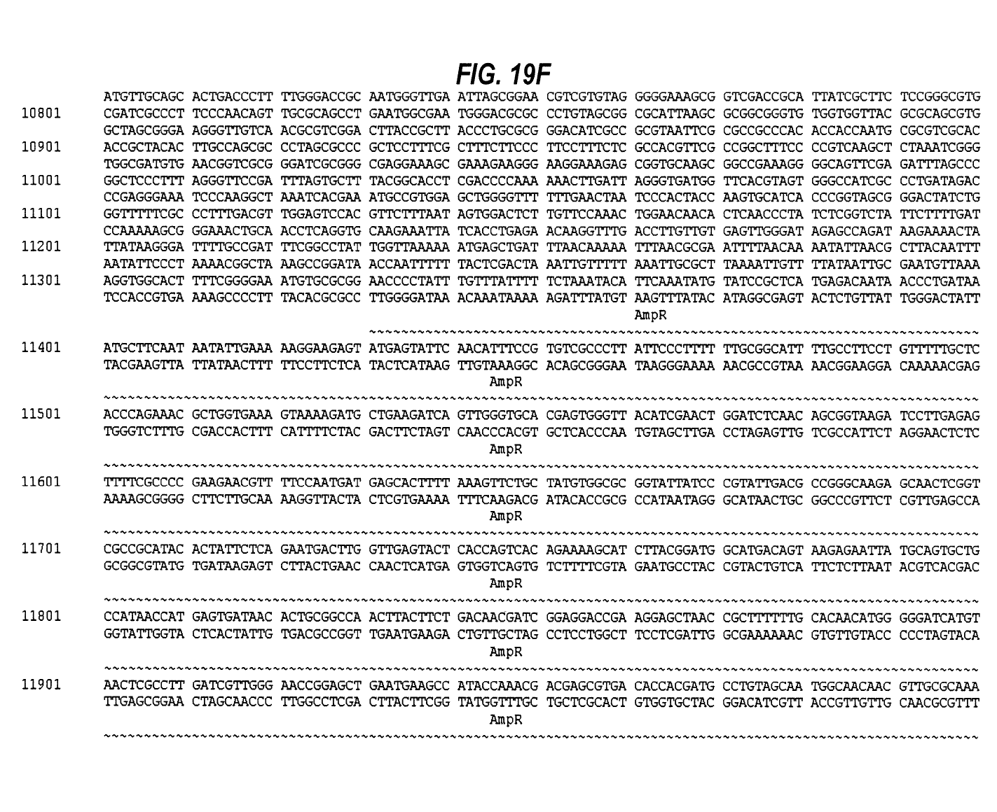

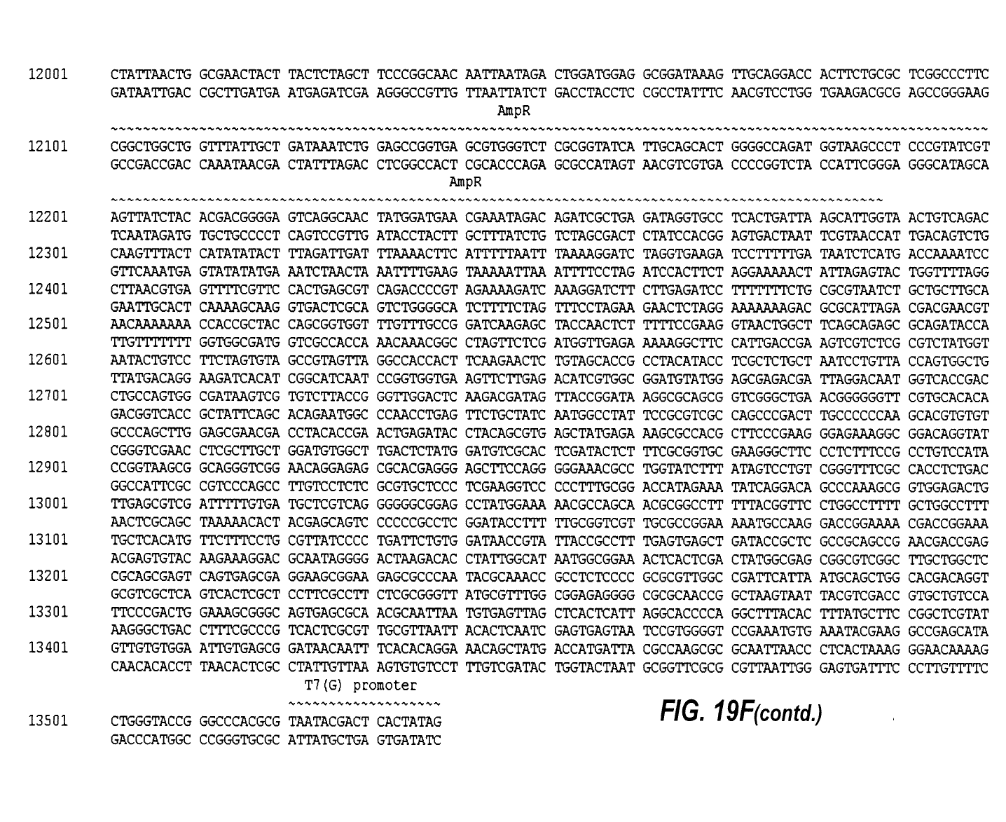

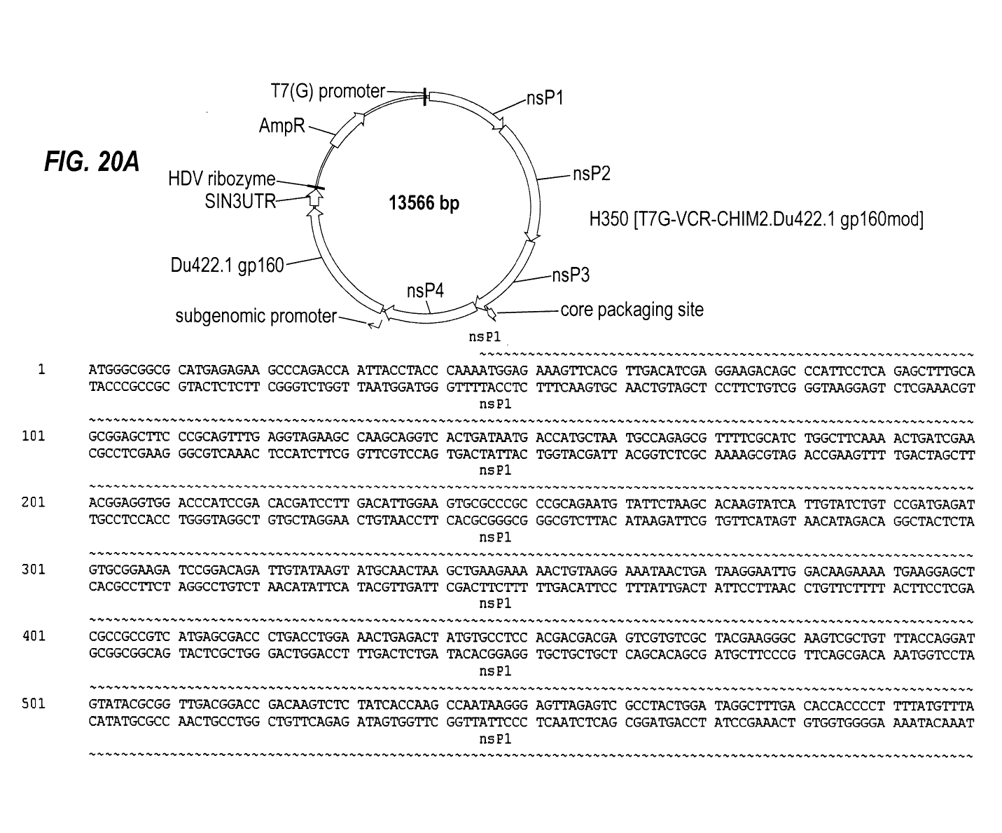

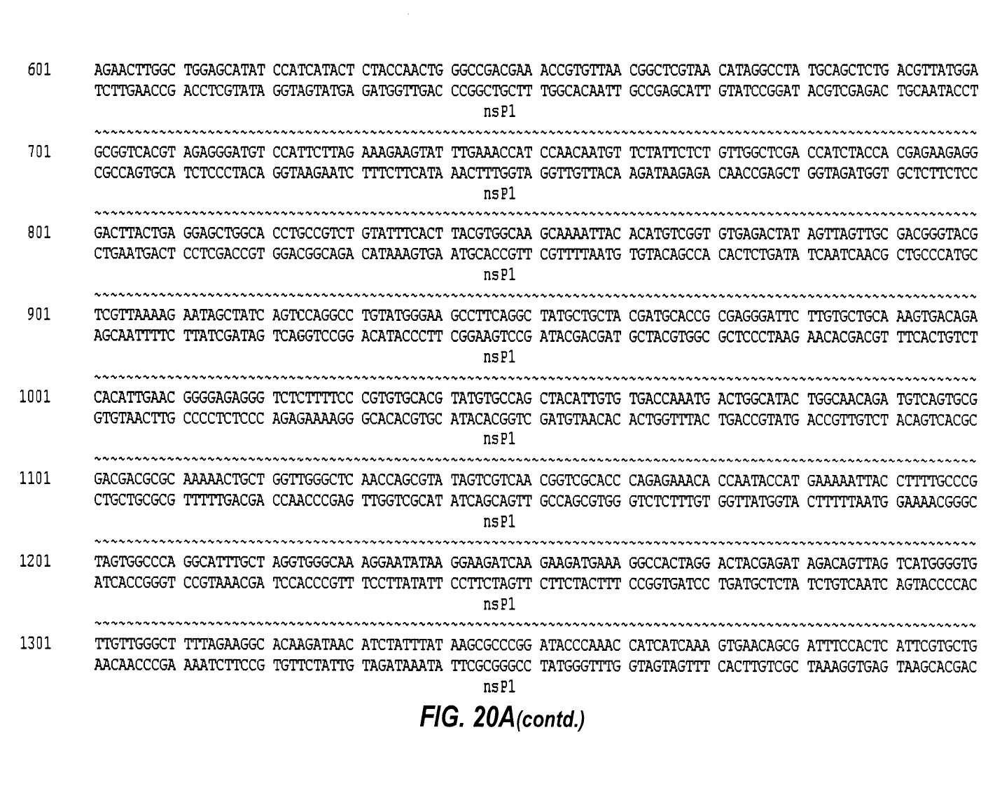

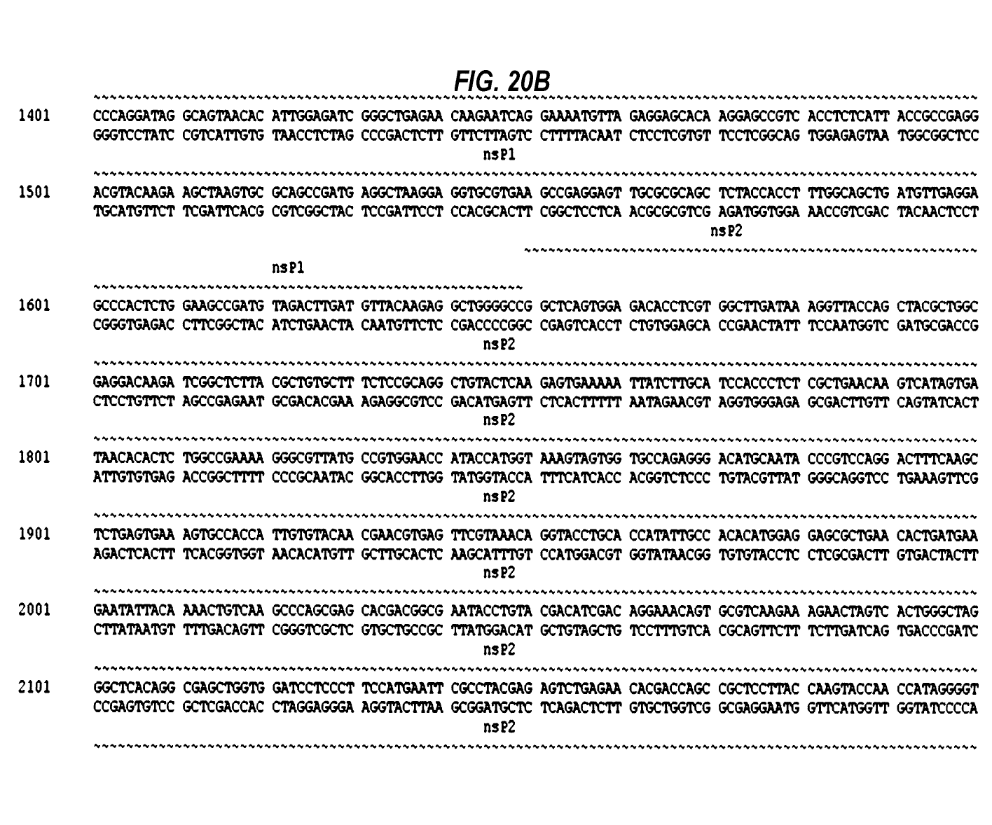

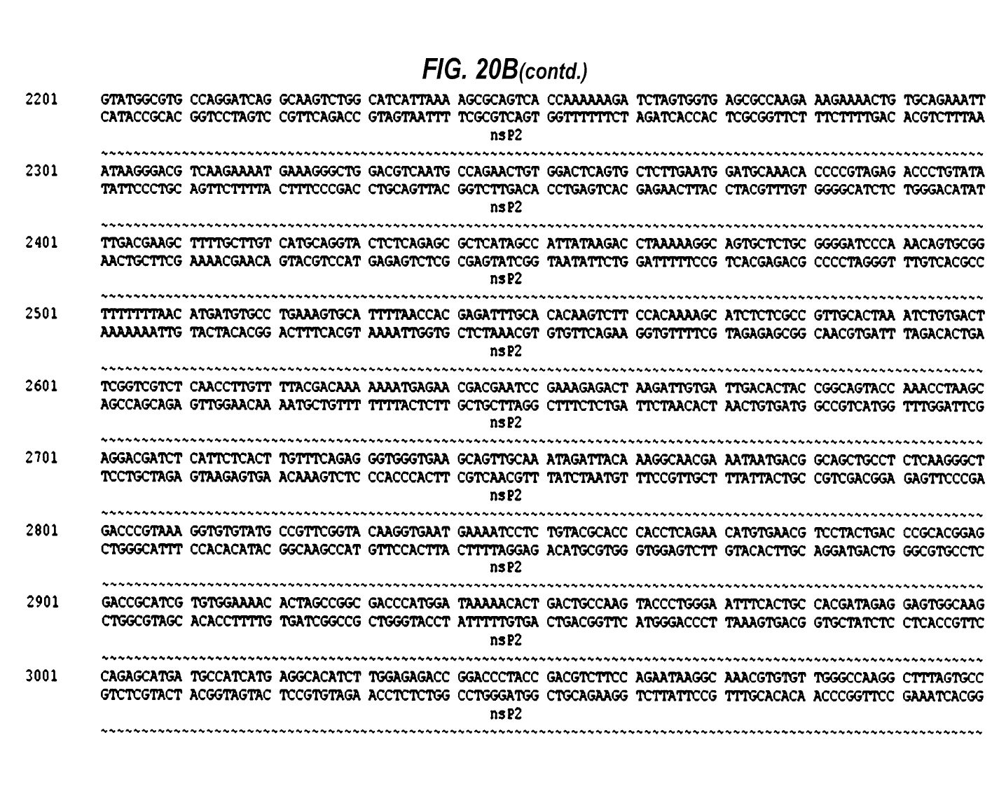

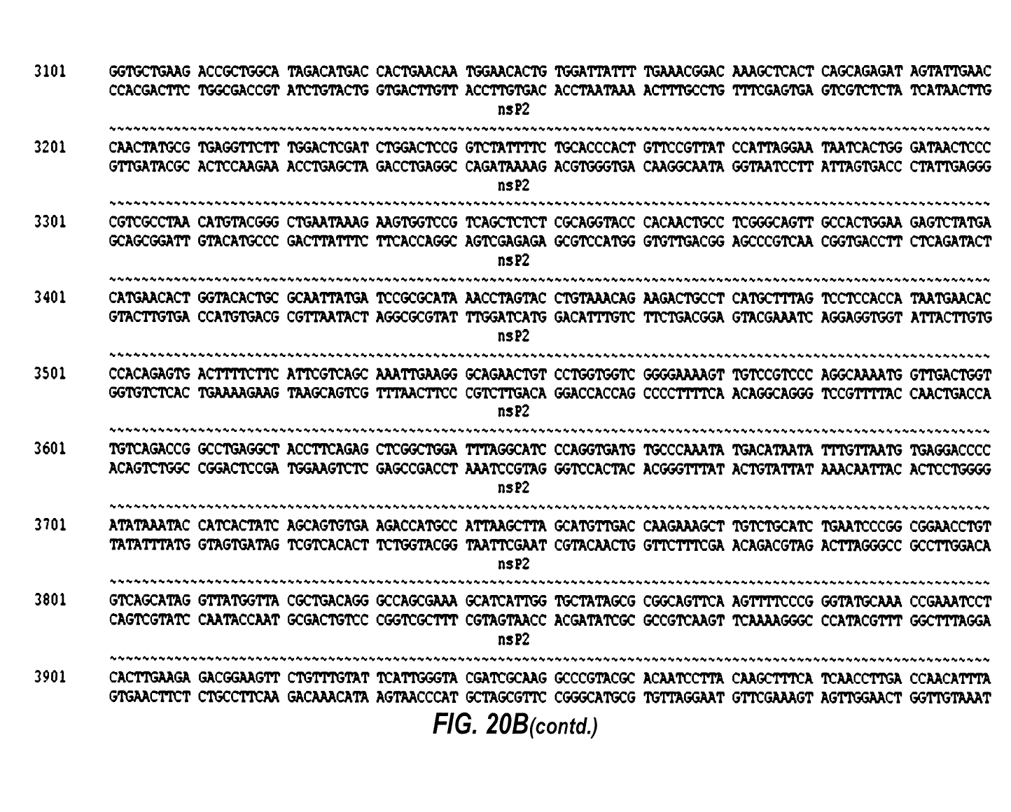

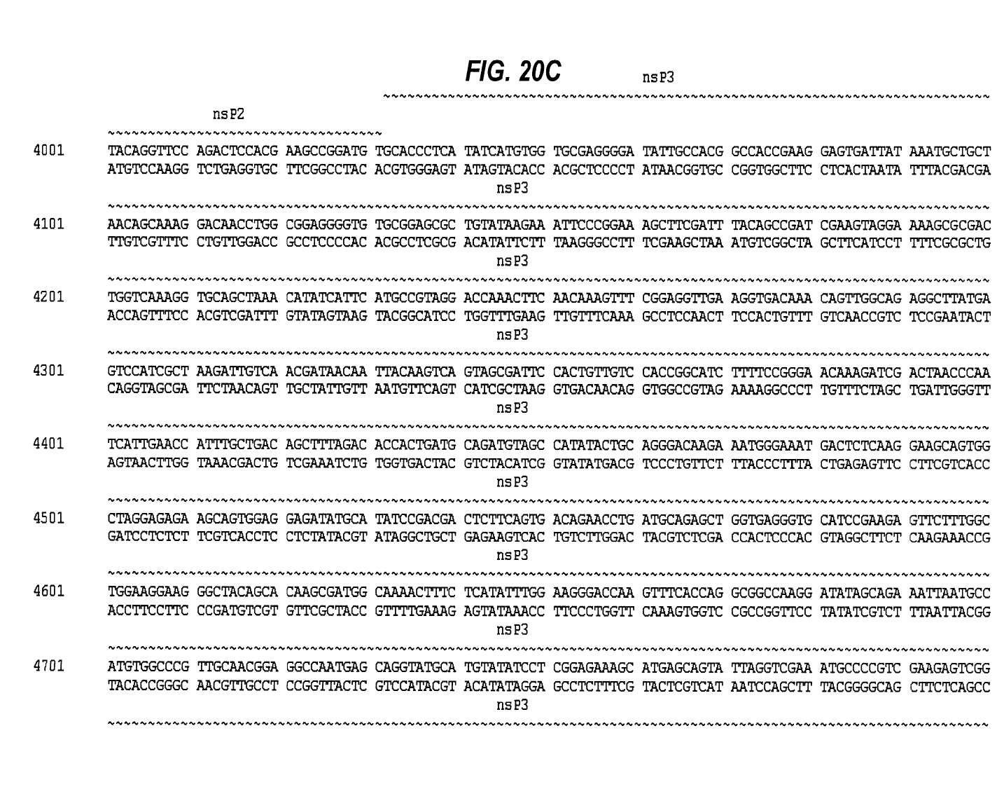

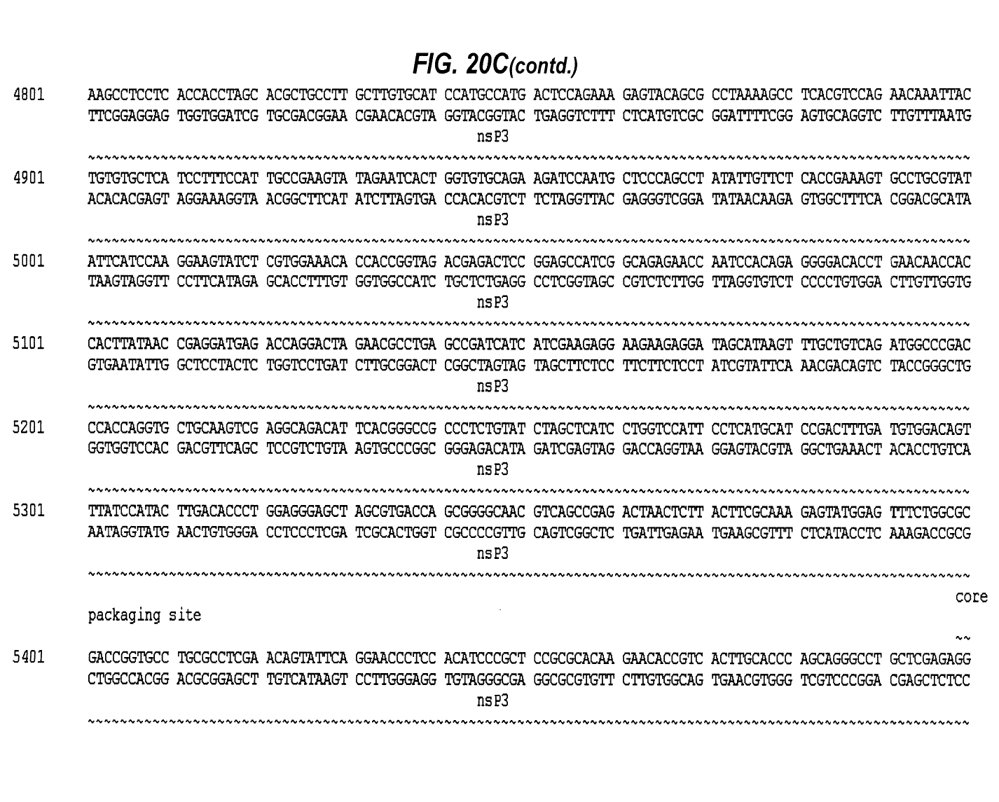

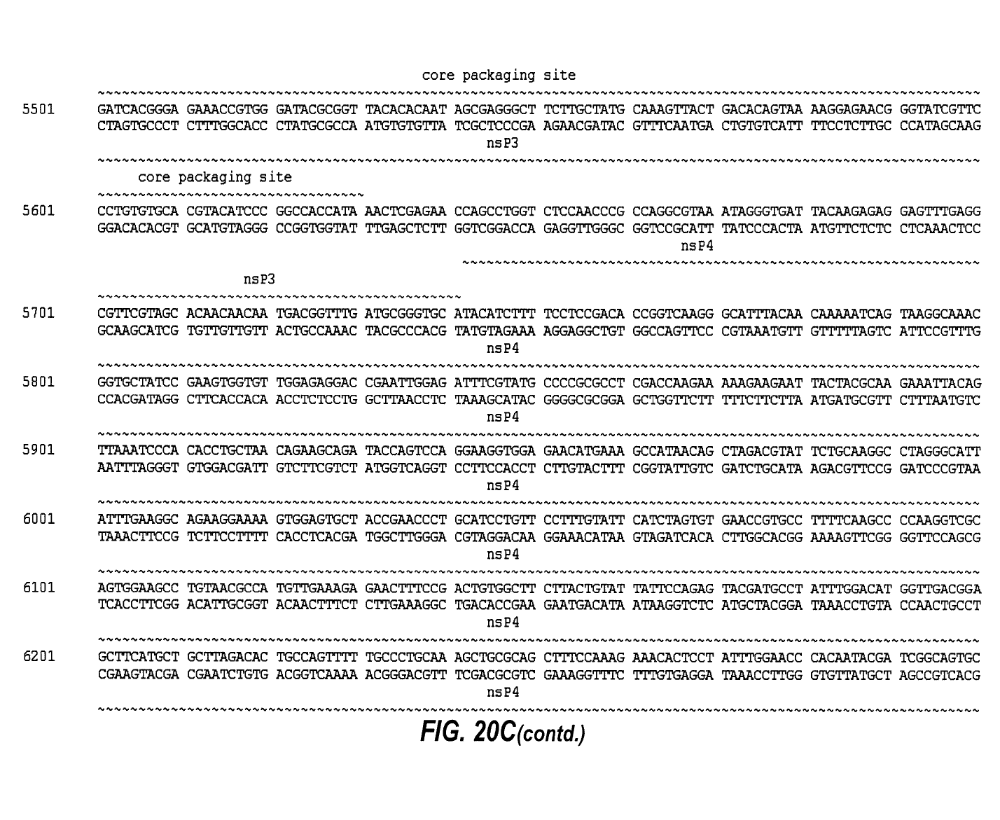

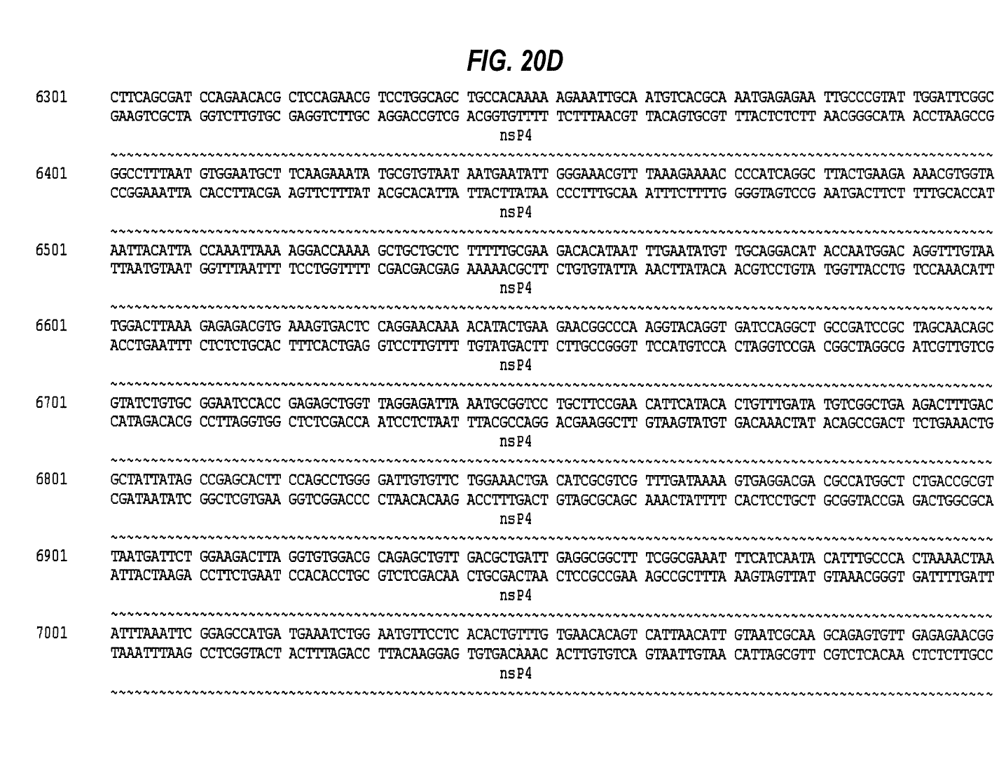

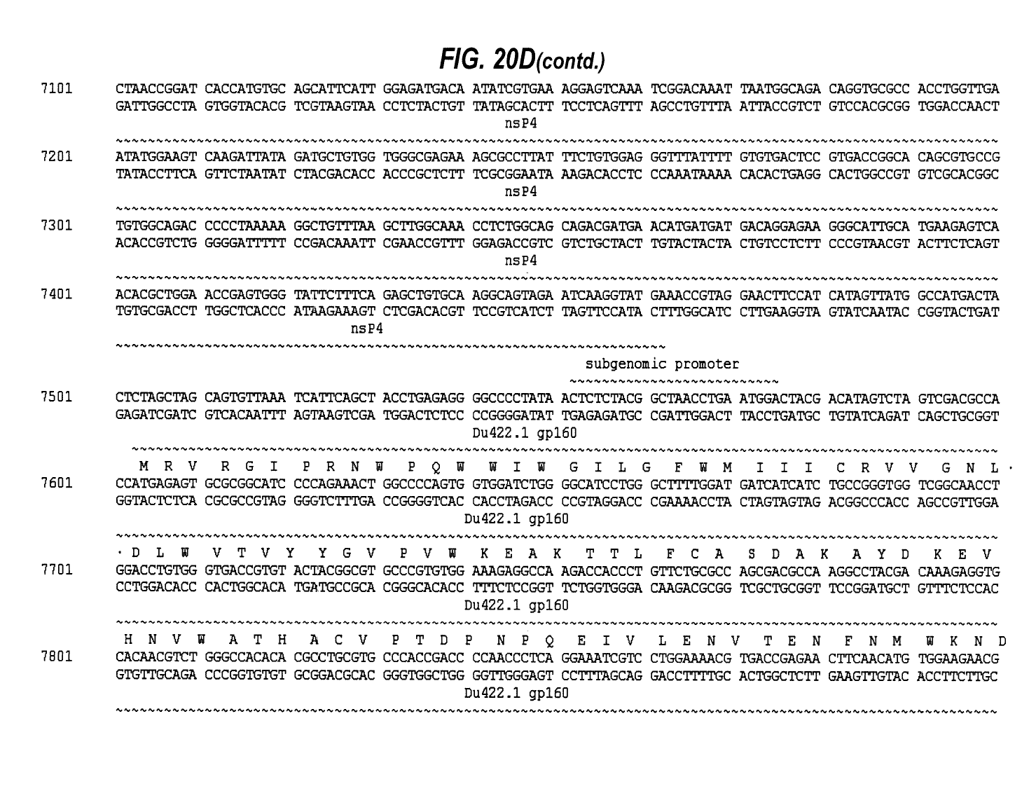

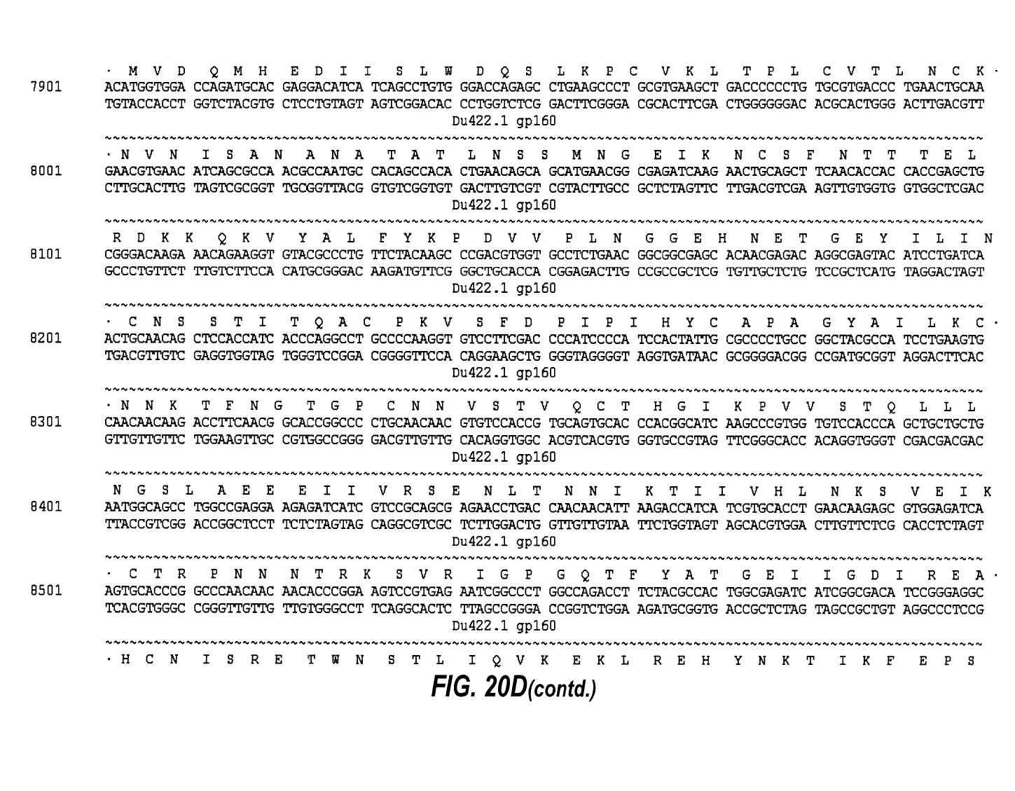

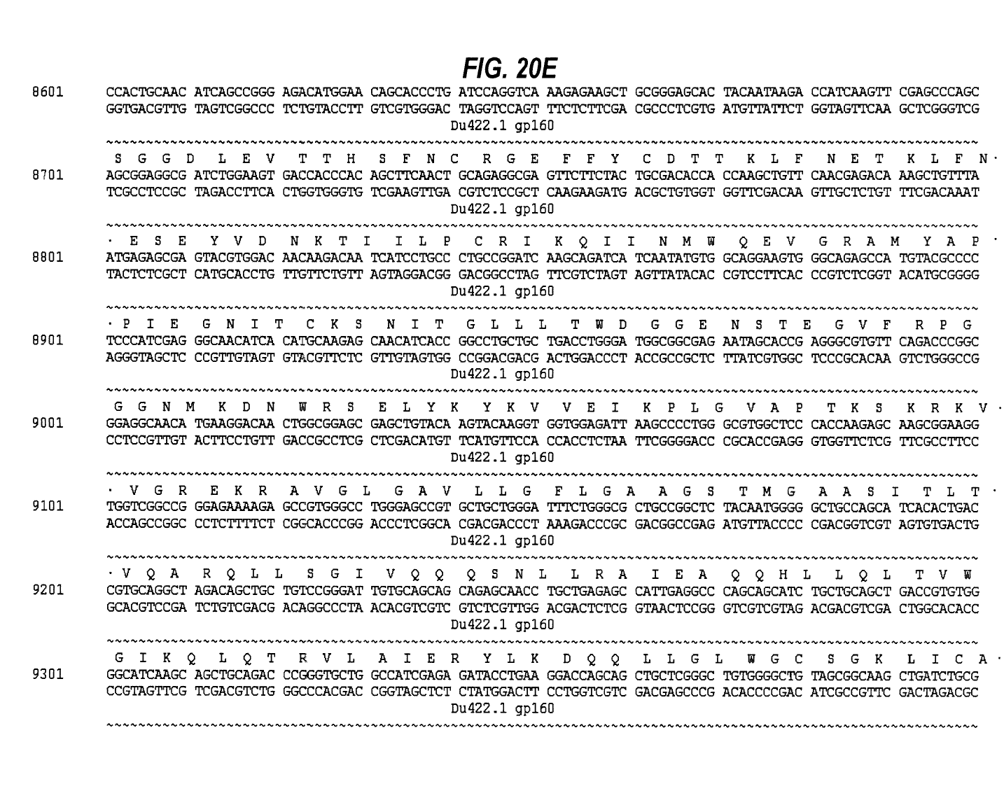

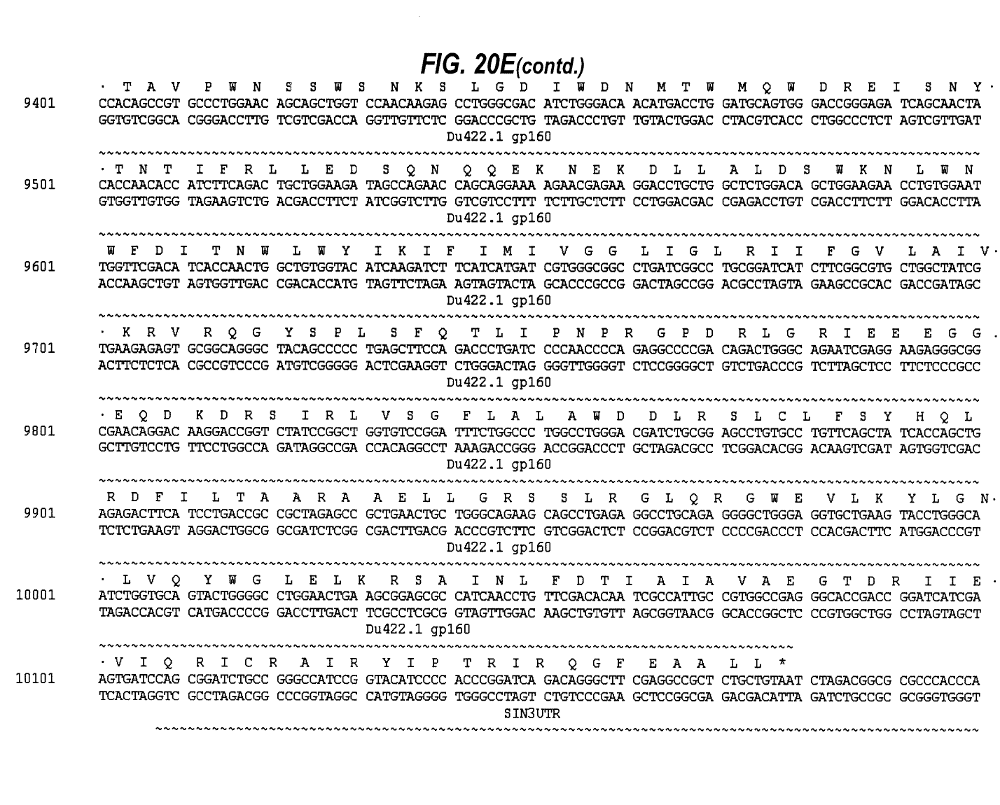

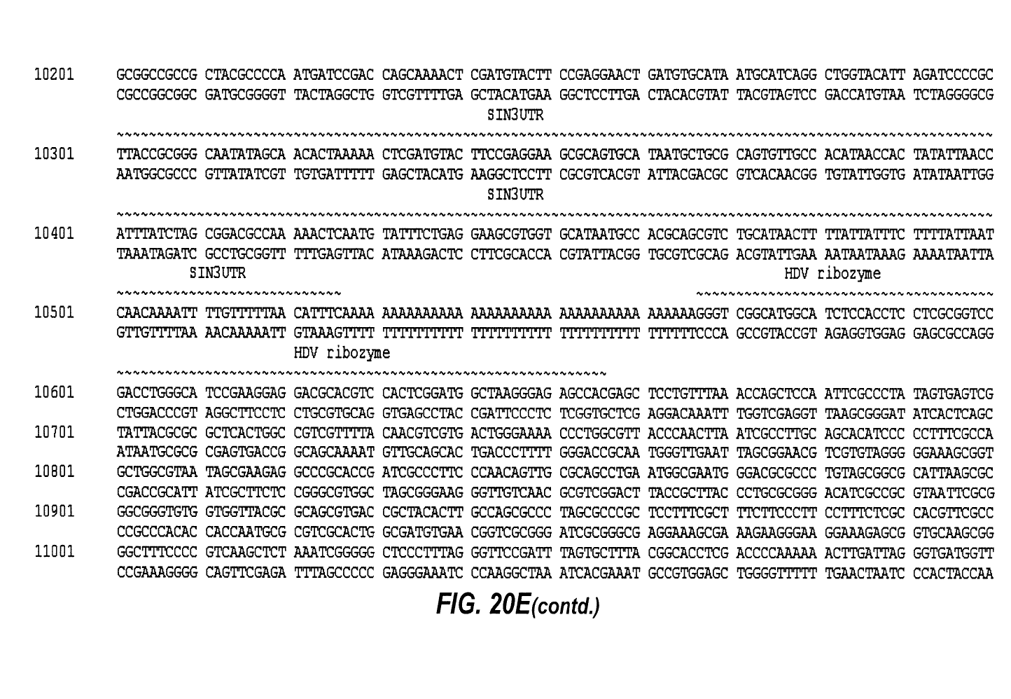

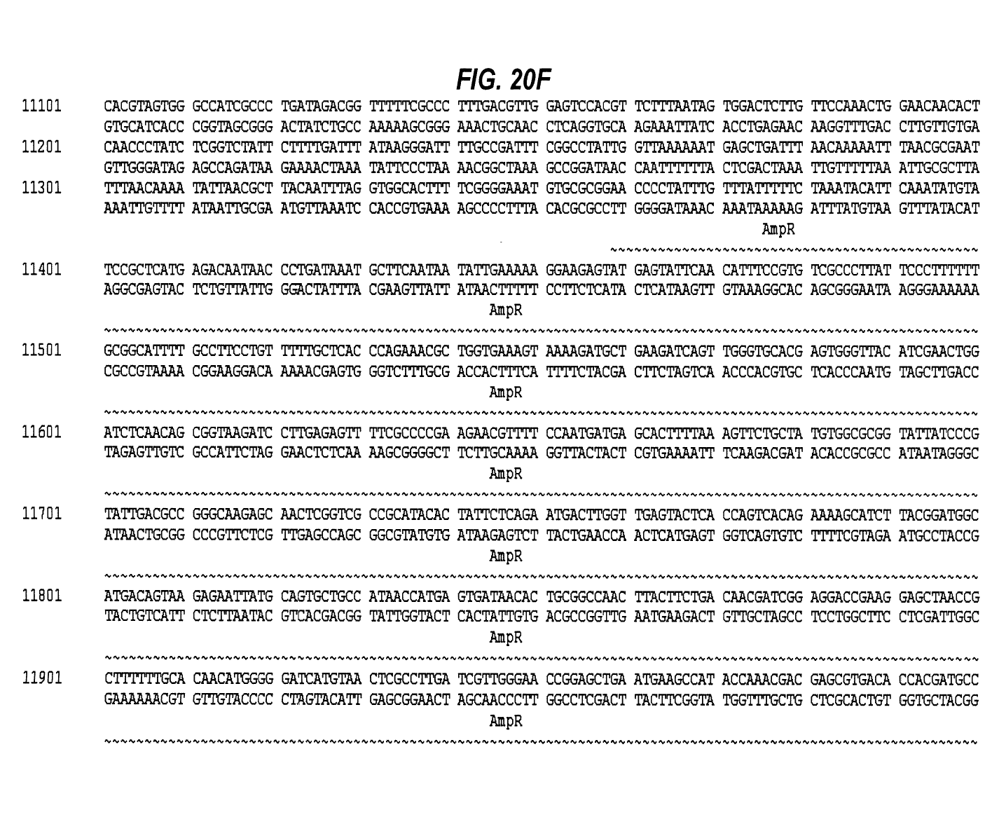

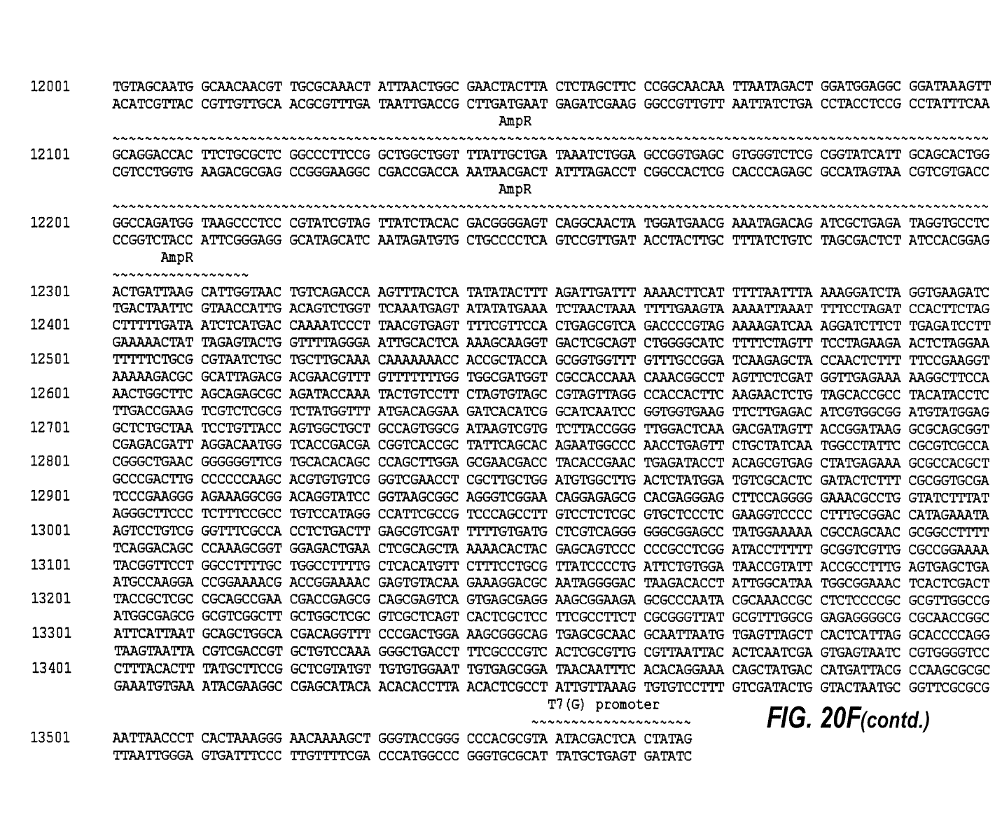

The following RNA replicons were used to prime mice
H351 T7G-VCR-CHIM2.12-SF162gp160mod—this RNA replicon expresses the gp160 envelope protein from the Clade B SF162 strain. The vector used to transcribe the RNA, the annotated sequence of the vector and the insert are shown in FIG. 19.
H350 T7G-VCR-CHIM2.12-DU422.1gp160mod—this RNA replicon expresses the gp160 envelope protein from the Clade C DU422.1 strain. The vector used to transcribe the RNA, the annotated sequence of the vector and the insert are shown in FIG. 20.

RNA production and purification—DNA was first linearized using PmeI and purified by phenol:chloroform extraction. RNA was in vitro transcribed using Ambion's MEGAscript T7 kit and purified by LiCl precipitation. Uncapped RNA was then 5′ capped using Cellscript's Scriptcap m⁷G Capping Enzyme System and purified by LiCl precipitation. RNA product was then visually confirmed by denaturing the RNA and running on an agarose gel.

The following DNA vector was used to prime mice

pCMV-KM2 gp160.SF162 mod—this DNA vector expresses the gp160 envelope protein from the Clade B SF162 strain. Gag and Env are cloned into the following eucaryotic expression vectors: pCMVKm2, for transient expression assays and DNA immunization studies, the pCMVKm2 vector is derived from pCMV6a (Chapman et al., Nuc. Acids Res. (1991) 19:3979-3986) and comprises a kanamycin selectable marker, a ColE1 origin of replication, a CMV promoter enhancer and Intron A, followed by an insertion site for the synthetic sequences described below followed by a polyadenylation signal derived from bovine growth hormone—the pCMVKm2 vector differs from the pCMV-link vector only in that a polylinker site is inserted into pCMVKm2 to generate pCMV-link; pESN2dhfr and pCMVPLEdhfr, for expression in Chinese Hamster Ovary (CHO) cells (See U.S. Pat. No. 7,943,375).

DNA production—plasmid DNA was used to transform Invitrogen Topten cells as per the protocol. Following 16-18 hours, a single colony was picked and used to inoculate 250 ml LB for 16-18 hours at 37° C. shaking at 225 rpm. Plasmid DNA was then purified from the culture using QIAGEN's EndoFree Plasmid Maxi Kit.

The following viral replicon particle (VRP) was used to prime mice

VRP gp140.dV2.SF162—this VRP expresses the gp140 envelope protein (variable loop 2 deleted) from the Clade B SF162 strain. See, e.g., Perri et al. (2003). J. Virol. 77(19): 10394-10403 regarding production and characterization of VRPs.

gp140 protein from Clade B SF162 strain—gp120 Env protein was expressed either from CHO stable cell lines or HEK293T transient transfections; in either case gp120 was expressed as a secreted, soluble protein. The conditioned medium was concentrated 10× and purified following a 2-step protocol including a Galanthus Nivalis lectin agarose capture step followed by cleaning using a DEAE column:
1. A Galanthus Nivalis lectin agarose (GNA) column was equilibrated with a buffer containing 20 mM Tris pH 8.0, 100 mM NaCl (column buffer).
2. Gp120 was captured on GNA column. After washing the column until A280 reading returns to baseline, the GNA column was connected in line with a DEAE column and a polymyxin column (to remove endotoxin) equilibrated with column buffer. Gp120 was eluted with column buffer with the addition of 500 mM MMP. Only contaminating proteins, but not gp120, bind to DEAE. Elution continues for about 7 column volumes or until A280 returns to baseline.
3. Using a stirred cell, purified gp120 was buffer exchanged with PBS and concentrated to about 1 mg/mL.
4. Concentration is determined by A280 and using the appropriate molar extinction coefficient. BCA assay was also used to confirm protein concentration.
5. Integrity of the protein was assessed by non reducing SDS-PAGE and SEC-HPLC.
6. Endotoxin content was measured using the Endosafe system
7. The final protein solution was frozen @−80 C

Nucleic acids (RNA or DNA) were encapsulated in liposome by combining lipids in ethanol with nucleic acids in sterile citrate buffer. Tangential Flow Filtration (TFF) was used to concentrate the liposomes and exchange the final buffer into PBS. Dynamic Light Scattering (DLS) determined the size distribution. To determine the nucleic acid encapsulation, Ribogreen and Picogreen assays were used to measure the total RNA and DNA content, respectively, after Triton-X treatment. The nucleic acid encapsulation (in μg/ml) was the total amount of nucleic acid after Triton-X treatment (disrupted liposomes) subtracted by the amount of RNA measured from undisrupted liposomes.

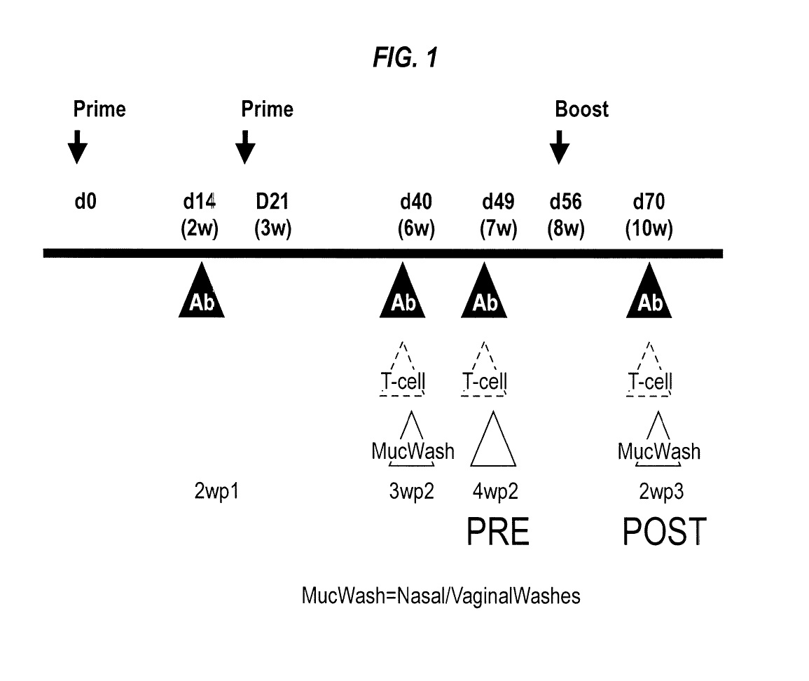

Various HIV gp160/gp140 formulations were administered to mice according to the schedule depicted in FIG. 1. Groups of 6-8 weeks BALB/c mice receiving the gp160/gp140 formulations are summarized in Table I-1 below.

TABLE I-1

| | No. | | | | |
|---|---|---|---|---|---|
| | of | | | Priming | |
| Group | mice | HIV gp160 formulation | | (2X) | Boost |
| Group 1 | | RNA gp160.SF162 | Naked RNA | 1 μg/ | 10 μg/mouse o- |
| | | | | mouse | gp140.SF162 |
| Group 2 | | RNA gp160.SF162 − | RNA in liposome | 1 μg/ | (with MF59) |
| | | Liposome | | mouse | |
| Group 3 | | RNA gp160.SF162 − | RNA in liposome | 0.1 μg/ | |
| | | Liposome | | mouse | |
| Group 4 | | DNA gp160.SF162 | Naked DNA | 15 μg/ | |
| | | | | mouse | |
| Group 5 | | DNA gp160.SF162 − | Naked DNA | 15 μg/ | |
| | | EP | delivered by | mouse | |
| | | | electroporation | | |
| Group 6 | | DNA gp160.SF162 − | DNA in liposome | 15 μg/ | |
| | | Liposome | | mouse | |
| Group 7 | | VRP gp140.SF162 | Virus replicon | 10⁶ | |
| | | | particles | IU/mouse | |
| Group 8 | | VRP gp140.SF162 | Virus replicon | 10⁷ | |
| | | | particles | IU/mouse | |
| Group 9 | | Protein o-gp140.SF162 | Protein (gp140) with | 10 μg/ | |
| | | | MF59 | mouse | |
| Group | | RNA gp160.DU422.1 − | RNA in liposome | 1 μg/ | 10 μg/mouse o- |
| 10 | | Liposome | | mouse | gp140.DU422.1 |
| | | | | | (with MF59) |

2 the “RNA Prime, Protein Boost” Regimen Induced a Robust and Balanced Immune Response.

First, the HIV gp160/gp140 formations described in Table I-1 were evaluated for potential adverse effects. FIG. 2 shows that administering liposome encapsulated RNA replicons showed no adverse effect. Transient loss of body weight and other visual signs of distress were observed after liposome encapsulated DNA formulation (at 15 μg dose) was administered, but there was no evidence of adverse effects with 0.1 μg RNA/Liposome, or 1 μg RNA/Liposome.

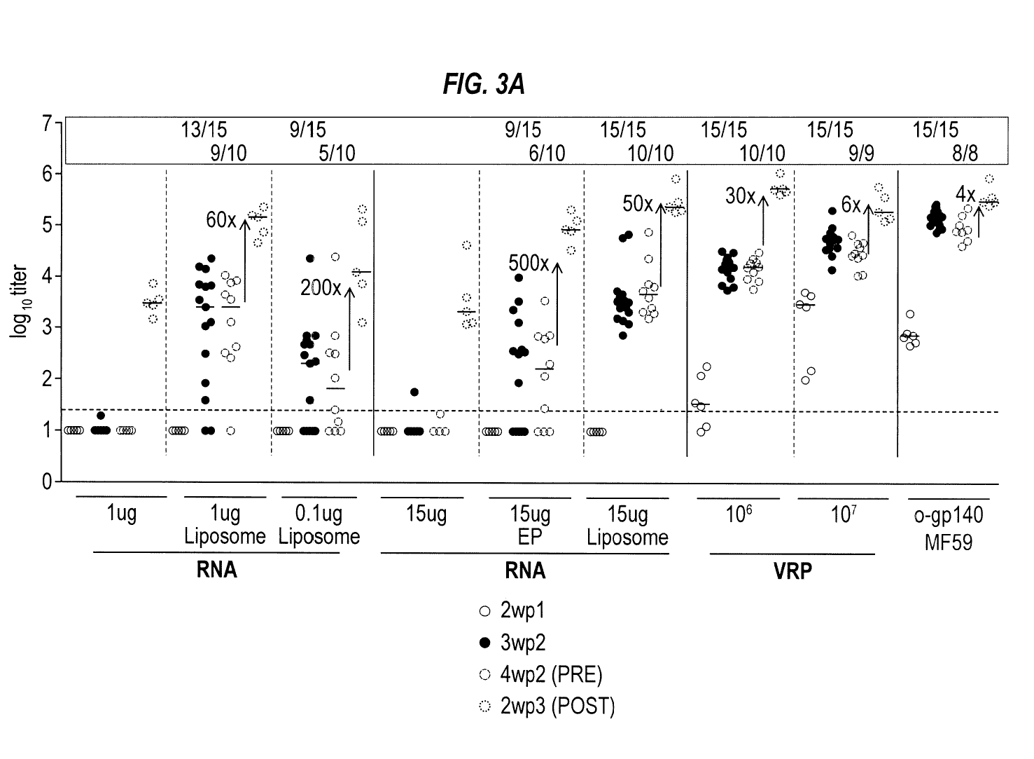

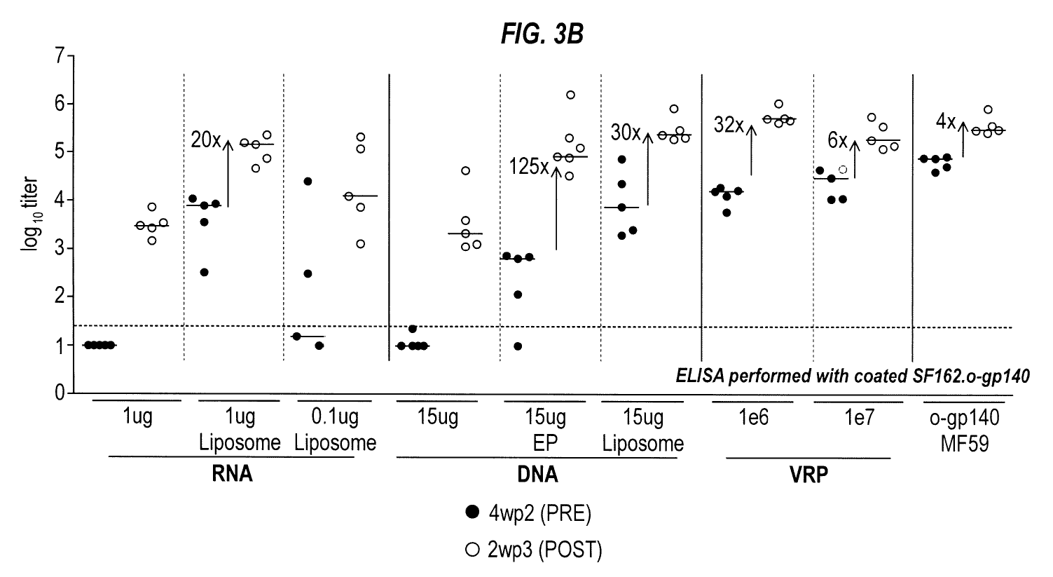

Second, anti-gp140 IgG antibody titers were measured to evaluate the immune response induced by the HIV gp160/gp140 formations described in Table I-1. As shown in FIGS. 3A and 3B, before the protein boost was administered, naked RNA induced no detectable IgG responses. RNA/Liposome formulations induced detectable IgG responses in 80-90% of the animals, and a dose-responsive effect was observed (compare the 1 μg dose versus 0.1 μg dose of RNA/Liposome in FIG. 3A). However, IgG titers in different animals showed significant variations. The median IgG titers induced by RNA/Liposome formulation at 1 μg were comparable to that of DNA/Liposome formulation at 15 μg, and were much higher than that of 15 μg of DNA delivered by electroporation.

A protein boost (10 μg protein/MF59, see Table I-1) resulted in a 20-fold increase of IgG titers in the 1 μg RNA/Liposome primed mice (FIG. 3B). After the protein boost was administered, the “1 μg RNA/Liposome prime, protein boost” regimen induced HIV-1 Env (SF162) specific IgG titers that were comparable to that of the “DNA/Liposome prime (15 μg), protein boost” regimen; and were also comparable to that of the “VRP (1e7) prime, protein boost,” or “protein prime, protein boost” regimens (less than a log lower) (FIGS. 3A and 3B). 1 μg RNA/Liposome prime, protein boost regimen also achieved superior results as compared to 10 μg DNA/Liposome prime, protein boost regimen (data not shown). IgG titers from the “naked RNA primed” group were also boosted and were similar to that of the protein/MF59 primed group at 2wp1 (see, FIG. 3A).

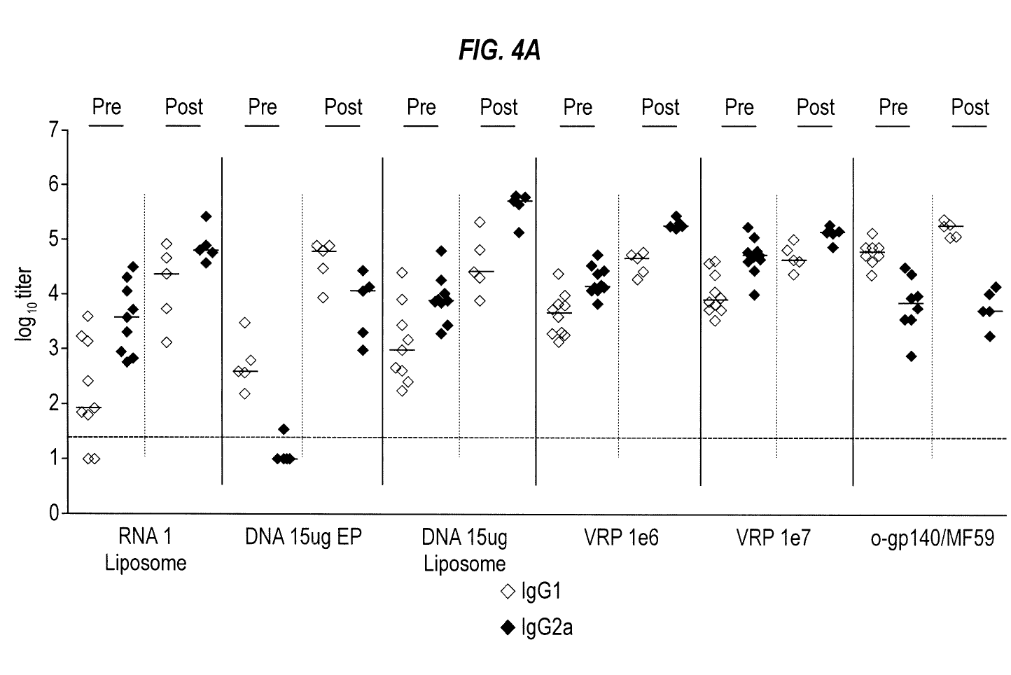

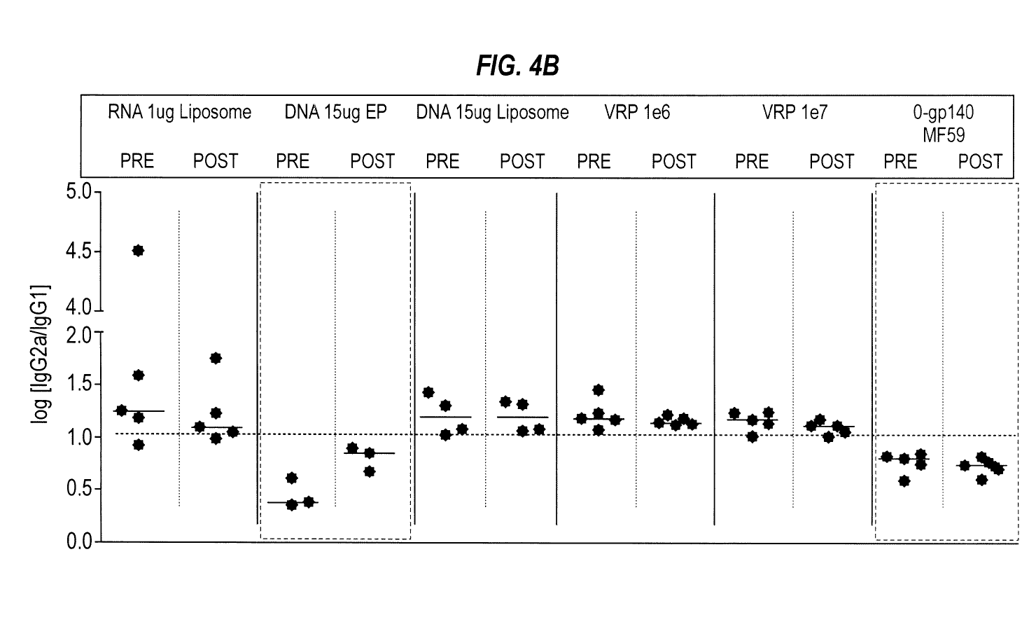

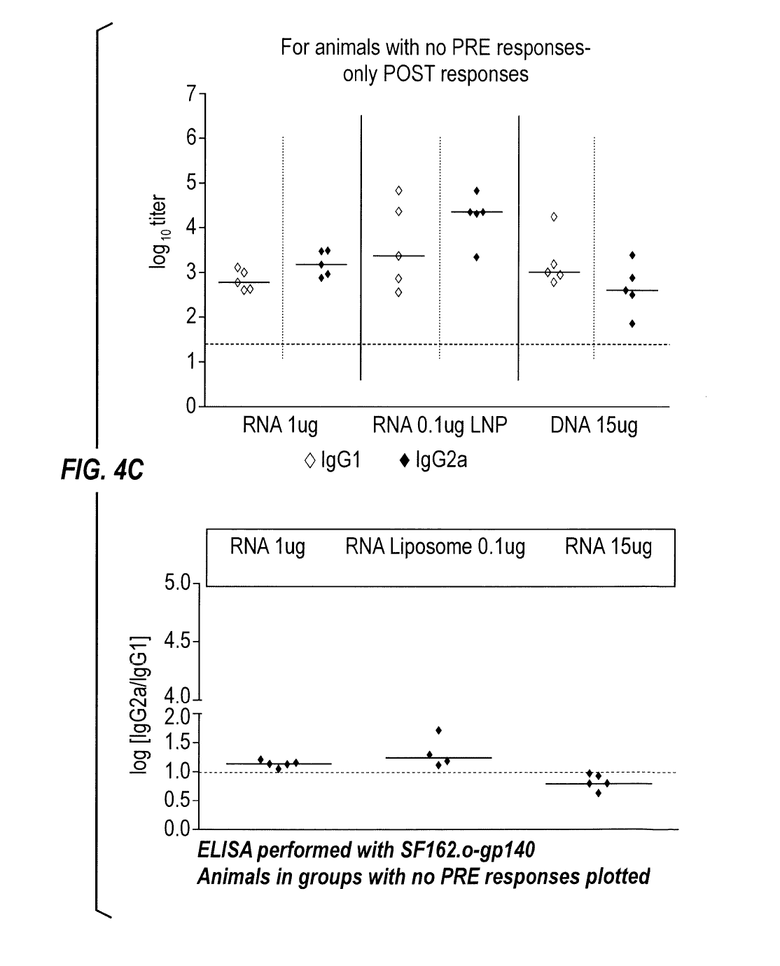

FIG. 4A shows that RNA/Liposome formulations induced a balanced IgG1:IgG2a subtype profile, similar to that of VRP. In contrast, in the protein/MF59 primed group, the IgG1 titers were significantly higher than IgG2a titers. IgG2a is considered as a surrogate of Th1 response, and IgG1 is considered as a surrogate of Th2 response. A balanced Th1:Th2 response is desirable. At both pre-boost and post-boost time points, the median IgG2a/IgG1 ratios in the RNA/Liposome primed group, DNA/Liposome primed group, and VRP primed group were higher than that of the DNA/electroporation primed group, or the protein/MF59 prime group (FIG. 4B). The “naked RNA” primed group, in which the IgG titers were not detectable before the protein/MF59 boost, also showed a balanced IgG1:IgG2a profile after boost (FIG. 4C).

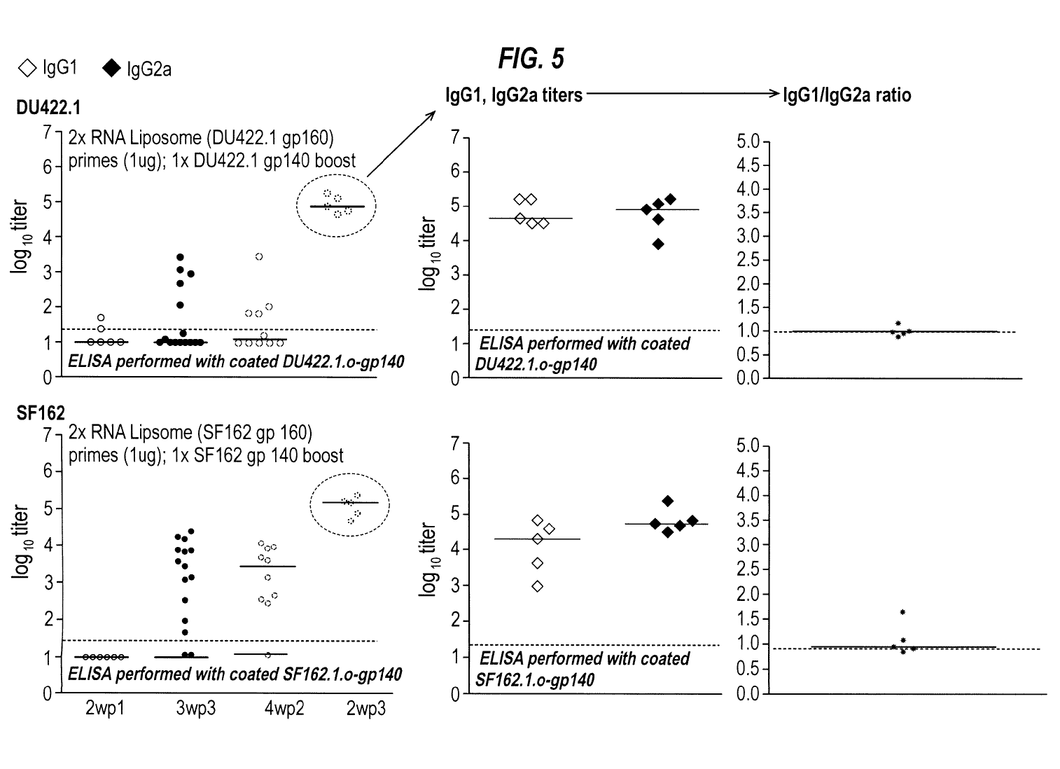

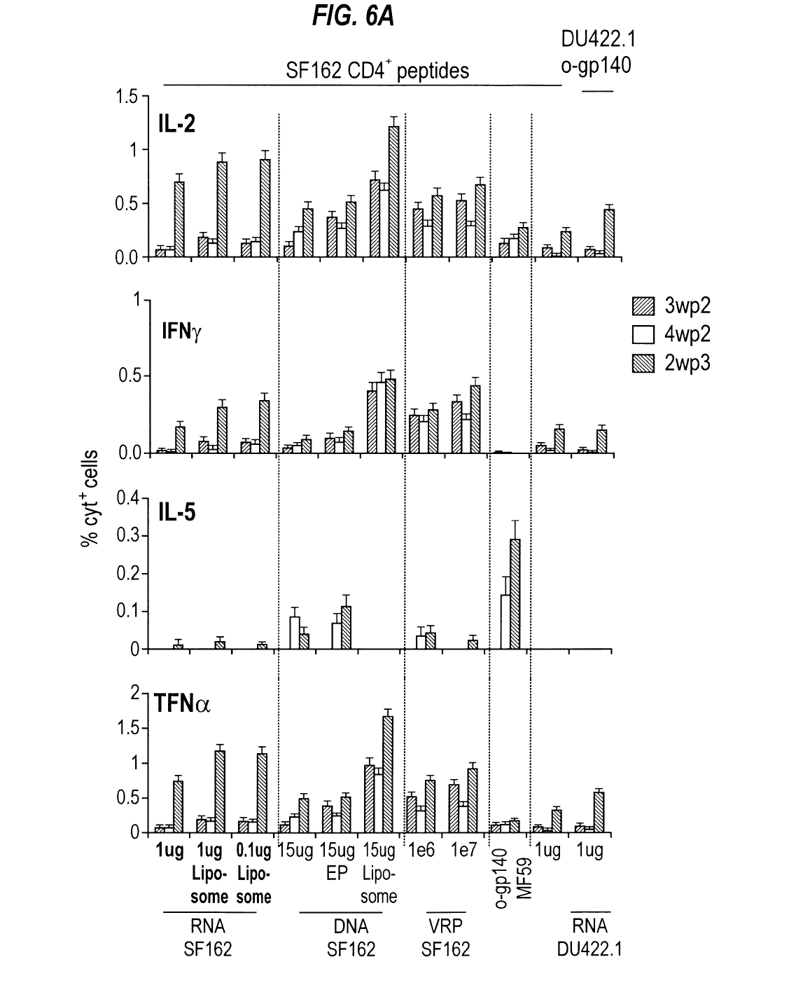

FIG. 5 compares the immunogenicity of Clade C (DU422.1) gp160 antigen and Clade B (SF162) gp160 antigen, both delivered as liposome formulated RNA. Clade C (DU422.1) gp160 antigen elicited a weaker IgG response before protein boost, as compared to Clade B (SF162) gp160 antigen. However, after the protein boost was administered, the total IgG titers for the two antigens were comparable. The IgG1:IgG2a profiles were similarly balanced for both Clade B and Clade C gp160 antigens.

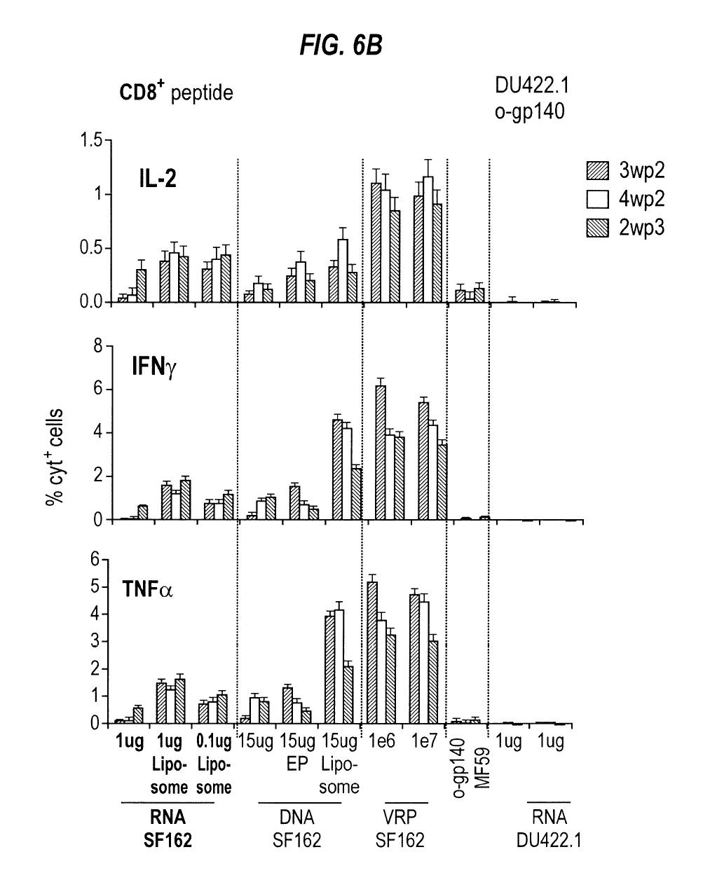

FIG. 6A shows that RNA/Liposome prime induced functional CD4+ T-cell-mediated immune responses, which were effectively boosted by the protein boost. CD4+ T cell responses were characterized by the increased levels of cytokine-secreting cells. As shown in FIG. 6A, before the protein boost was administered, the RNA/Liposome formulations (see Table I-1) induced detectable SF162 specific CD4+ T cell responses. The levels of cytokine-secreting CD4+ T cells in the RNA/Liposome primed groups were lower than that of the DNA/Liposome or VRP primed groups, but comparable to that of the protein/MF59 primed group.

After the protein boost was administered, the levels of cytokine-secreting CD4+ T cells were significantly increased in the RNA/Liposome primed groups (at either 0.1 or 1 μg priming doses, which were boosted equally). Protein boosting of CD4+ T-cell responses was more effective with RNA/Liposome priming than that seen with 15 μg DNA/electroporation priming; equal or more effective than that seen with the highest dose of VRP priming; and similar or slightly lower than that seen with 15 μg DNA/Liposome priming. CD4+ T-cell responses in the naked RNA primed group were also boosted.

IL-2-, IFNγ-, and TNFα-secreting cells in the RNA/Liposome prime, protein boost groups were higher than that of the group that received 3 doses of protein/MF59. IL-5 secretion from the CD4+ T-cells in the RNA/Liposome prime, protein-boost group was lower than that of the group that received 3 doses of protein/MF59. The results show that RNA priming initiated a T_{H}1 response (IL-2^{high}, IFNγ^{high}, TNFα^{high} IL-5⁻) that was sustained or elevated after a protein boost. Similar cytokine profiles were seen in the DNA/Liposome or VRP primed groups. The cytokine profile was in contrast to a T_{H}2 type (IL-2^{low}, IFNγ^{low}, TNFα^{low}, IL-5⁺) response that was seen in the protein prime, protein boost group.

FIG. 6B shows that RNA/Liposome prime induced functional CD8+ T-cell response, which was not affected by the protein boost. CD8+ T cell-mediated immune responses were characterized by the increased levels of cytokine-secreting cells. As shown in FIG. 6B, before the protein boost was administered, RNA/Liposome formulations induced detectable SF162 specific CD8+ T cells responses. The CD8+ T cells responses were lower than that of DNA/Liposome or VRP formulations but comparable to that of 15 μg of electroporated DNA.

After the protein boost was administered, the magnitude or quality of CD8+ T cell response in the RNA/Liposome primed groups was unaffected by the protein boost. For DNA and VRP primed groups, reduced frequency of CD8+ epitope specific T-cells (IFNγ- and TNFα-secreting cells) after the boost was evident at 4wp2 time point.

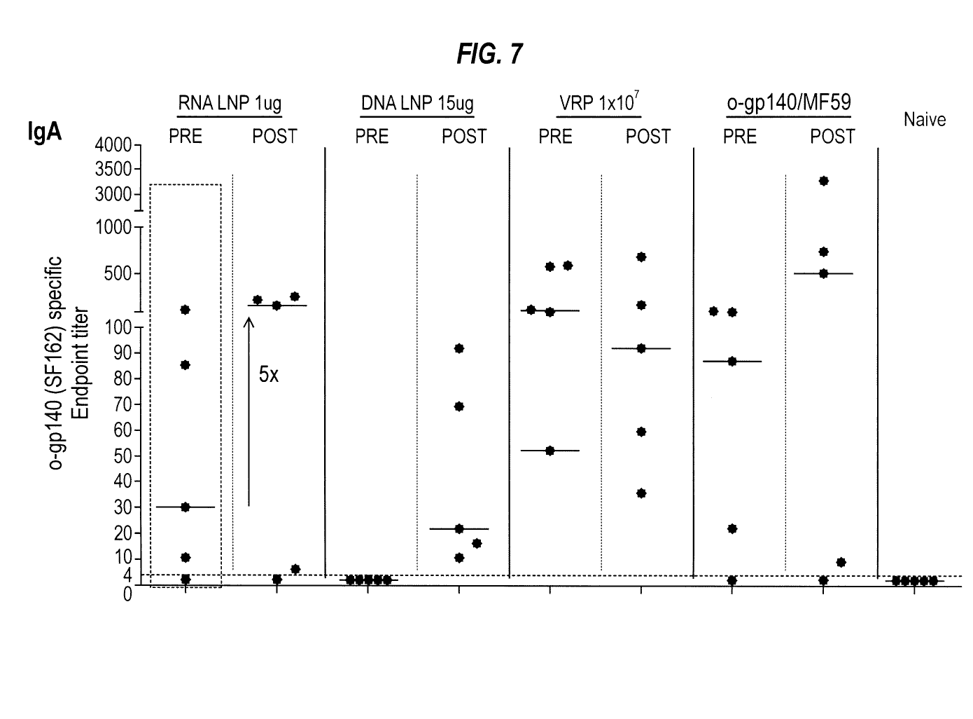

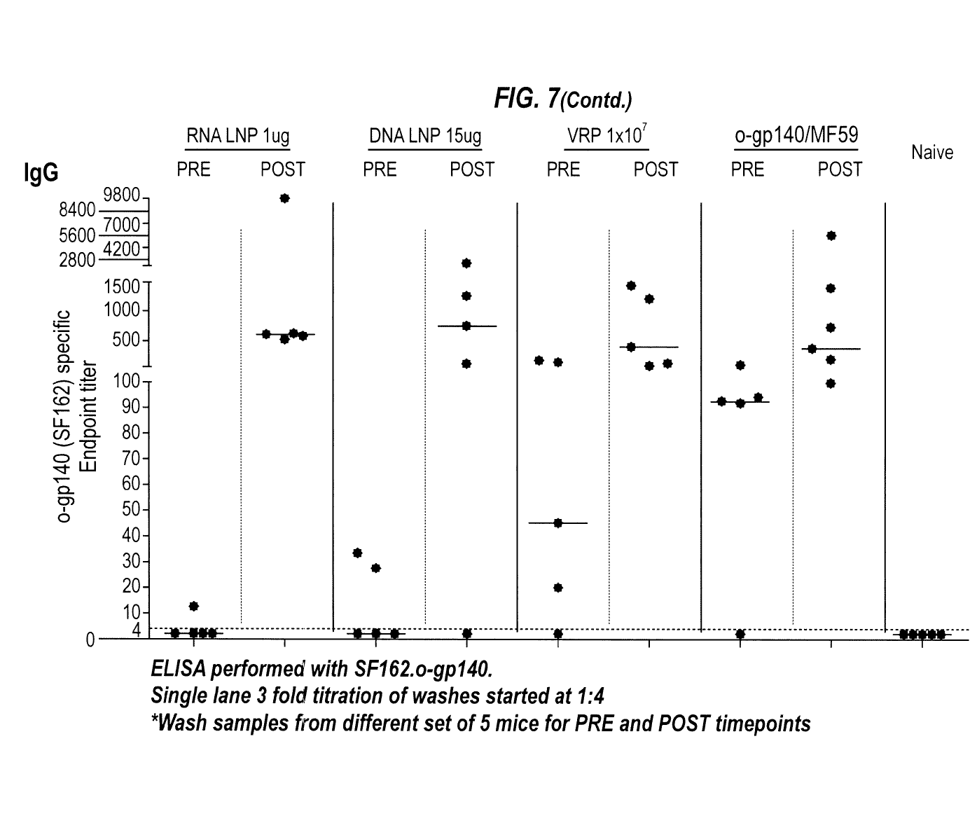

FIG. 7 shows the titers of gp140-specific IgA in vaginal washes of the mice administered the formulations shown in Table I-1. Before the protein boost was administered, priming the mice twice with the RNA/Liposome formulations induced detectable SF162 gp140-specific IgA antibodies in vaginal secretions. Secretion of anti-gp140 IgG antibody was not evident. Priming the mice twice with the VRP or protein/MF59 also induced SF162 gp140-specific IgA antibodies, with a median IgA titer higher than that of the RNA/Liposome group. SF162 gp140-specific IgA antibodies were not detectable in the DNA/Liposome primed (2× prime) group.

After the protein boost was administered, the IgA and IgG titers in the vaginal washes of a different set of 5 mice were measured. The median IgA titer of the RNA/Liposome prime, protein boost group was higher than that of the DNA/Liposome prime, protein boost group, and was comparable to that of VRP primed or protein primed groups. The median IgG titers post-protein boost were better clustered as compared to pre-boost. The median IgG titers were comparable for all groups.

### Example II

### HIV Envelop Protein Study 2—Gp140 (Co-Administration of RNA and Protein)

In this example, the HIV Clade C (TV1) envelop protein gp140 was used as the antigen. An RNA molecule encoding HIV gp140, and its encoded protein (gp140) were combined and co-administered, and the immunogenic effect of this combination was assessed.

### 1. Study Design

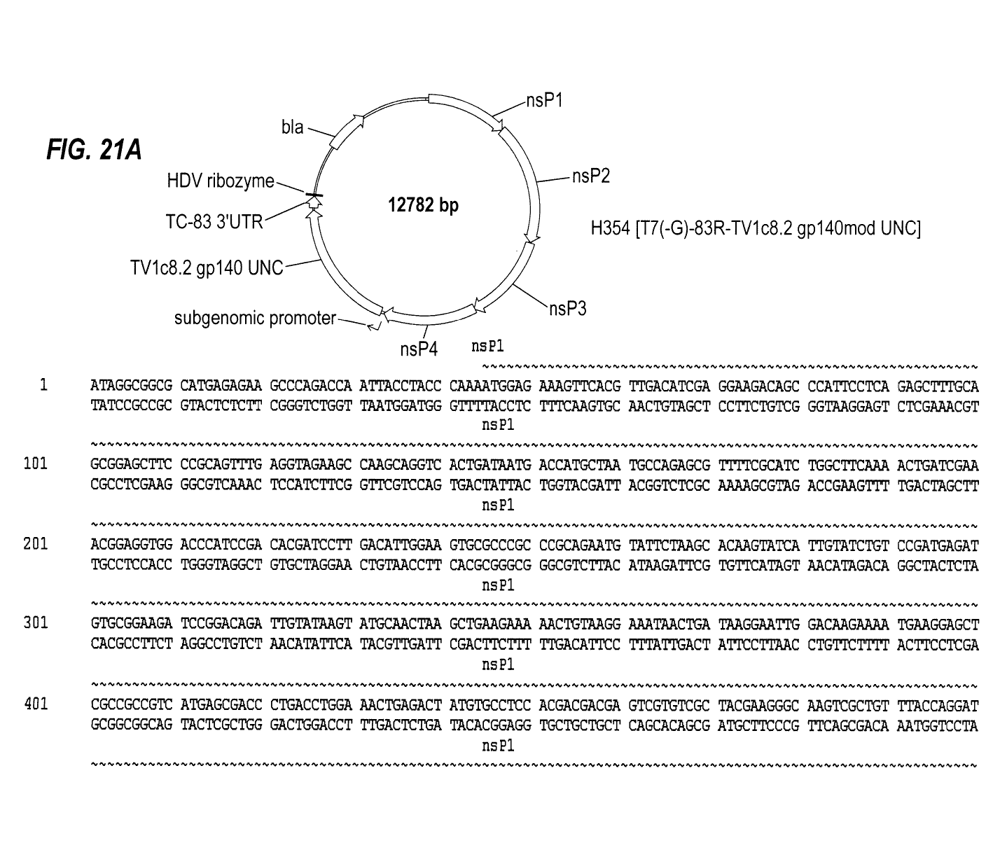

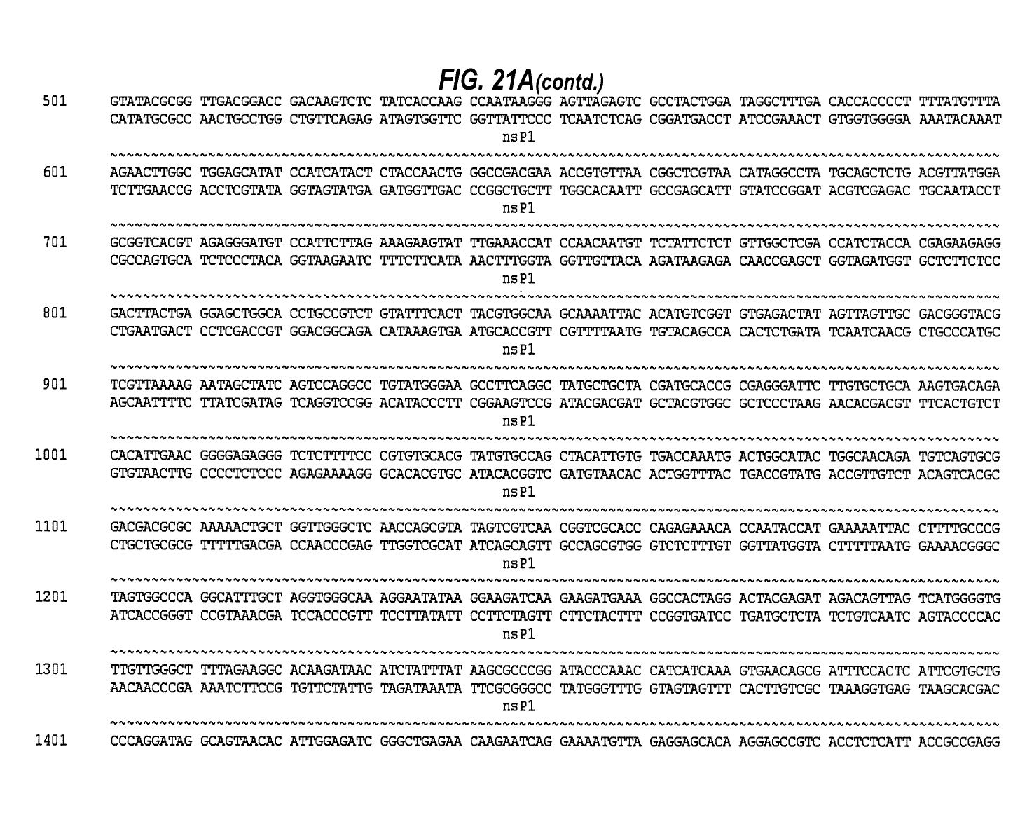

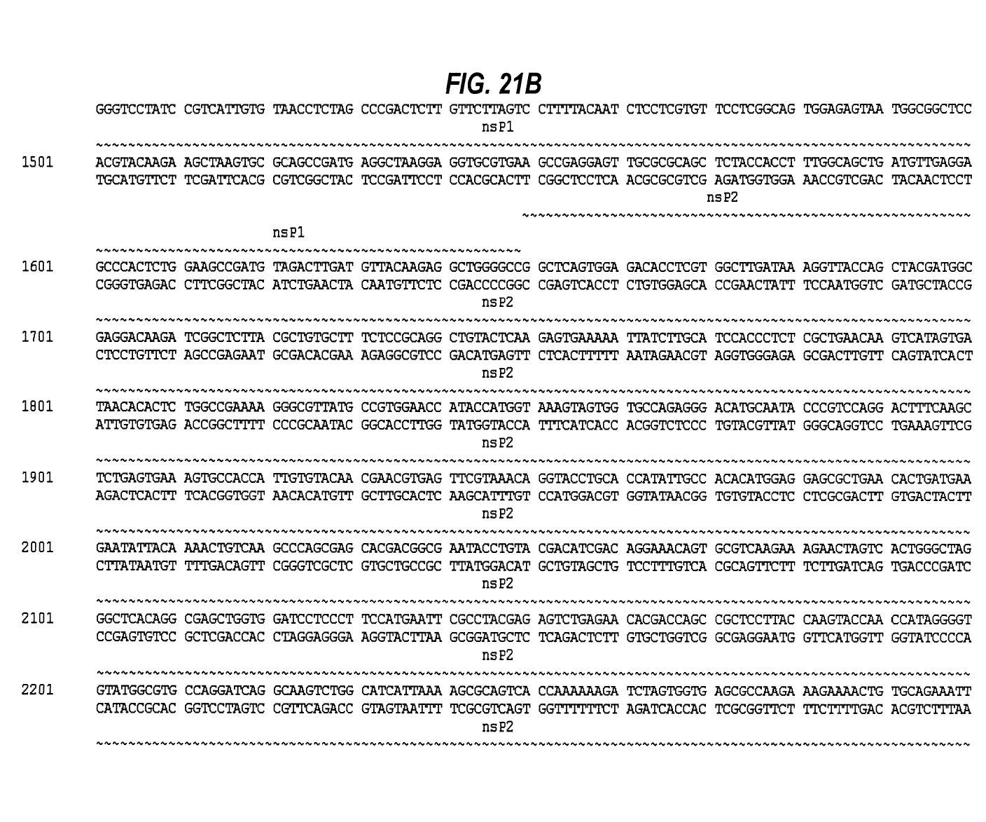

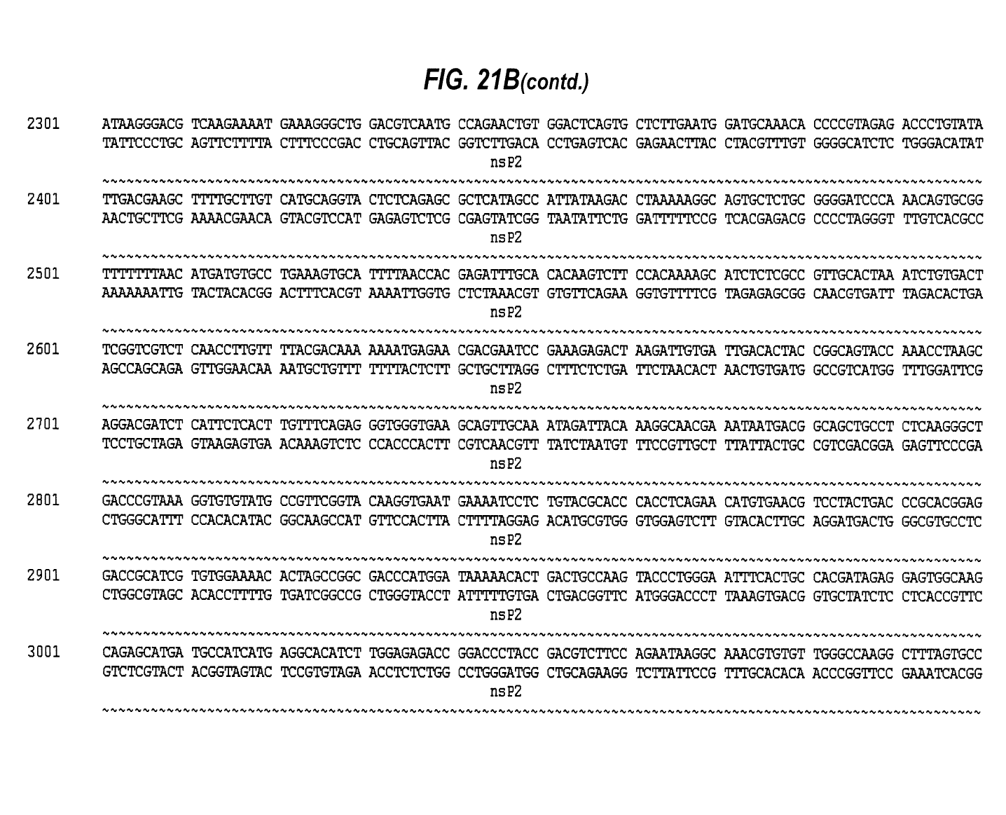

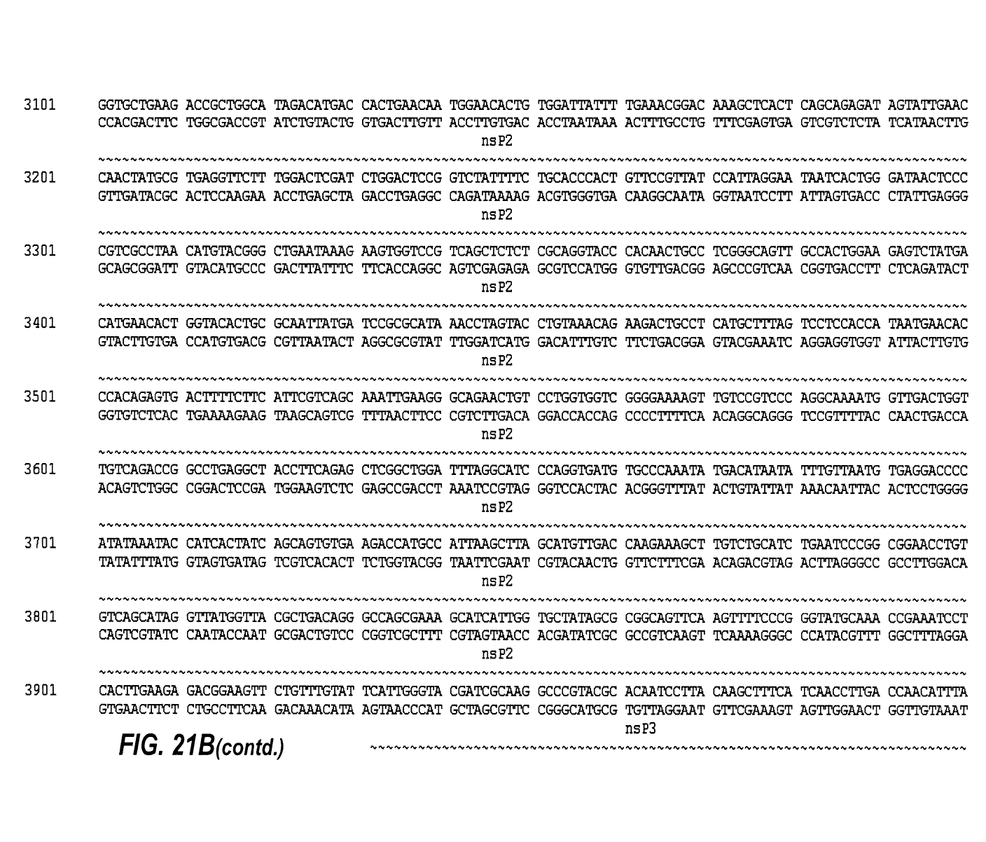

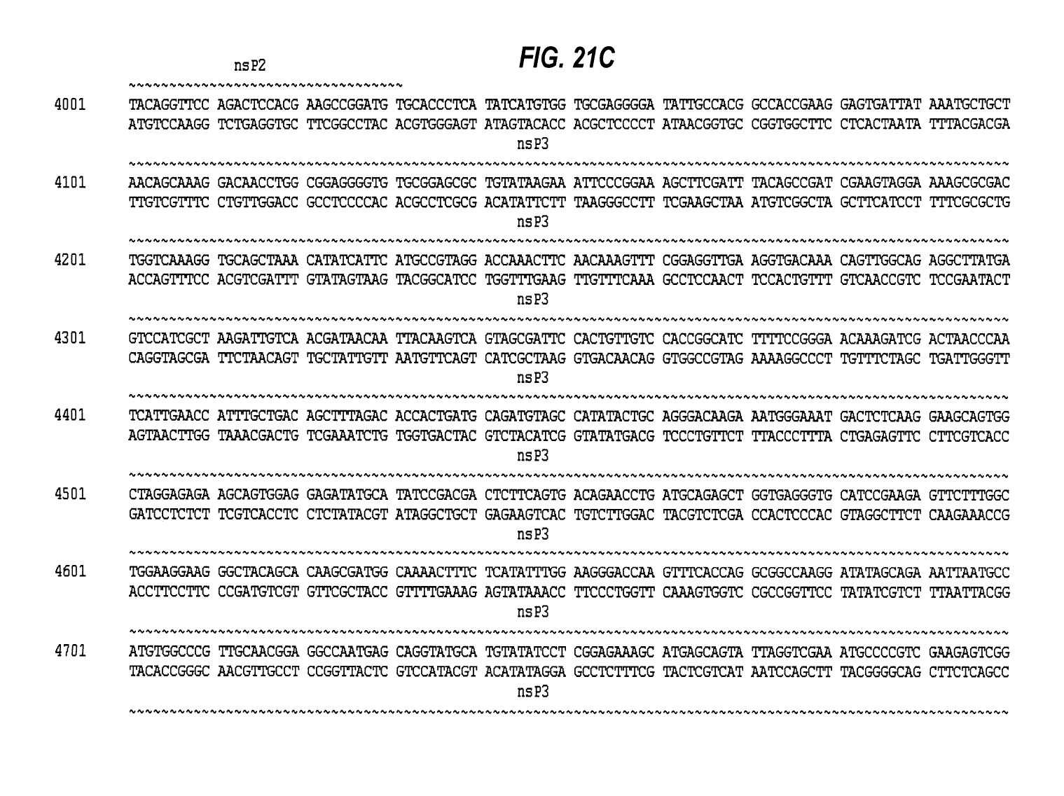

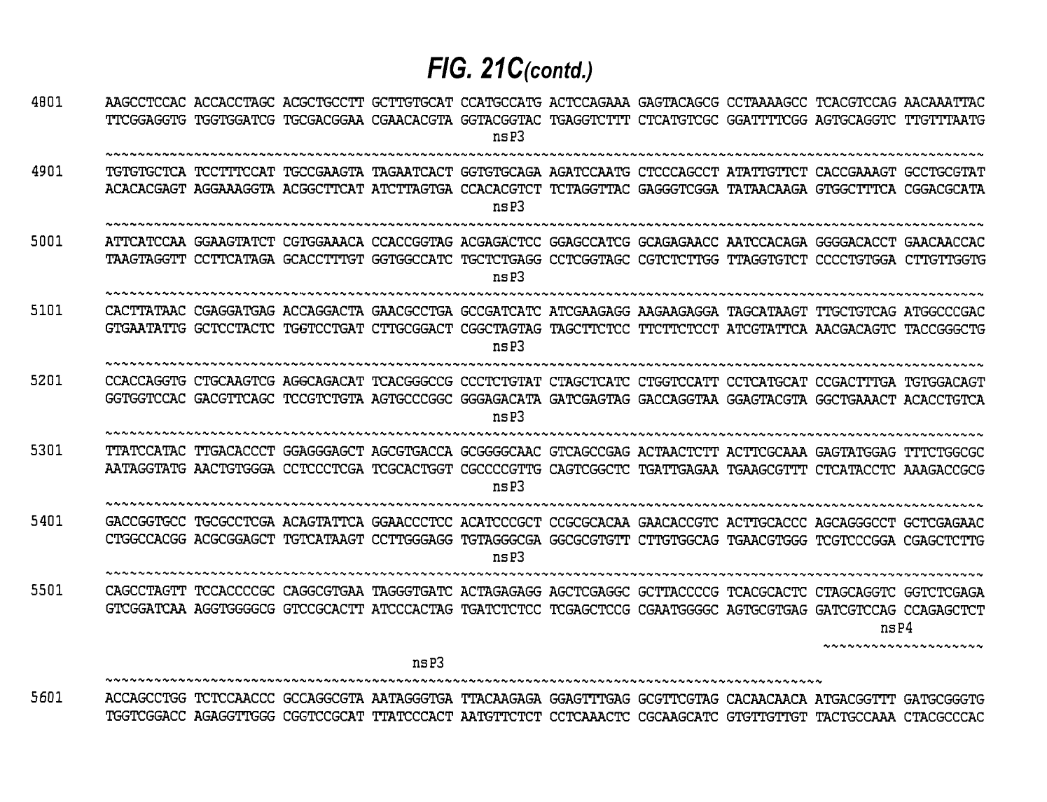

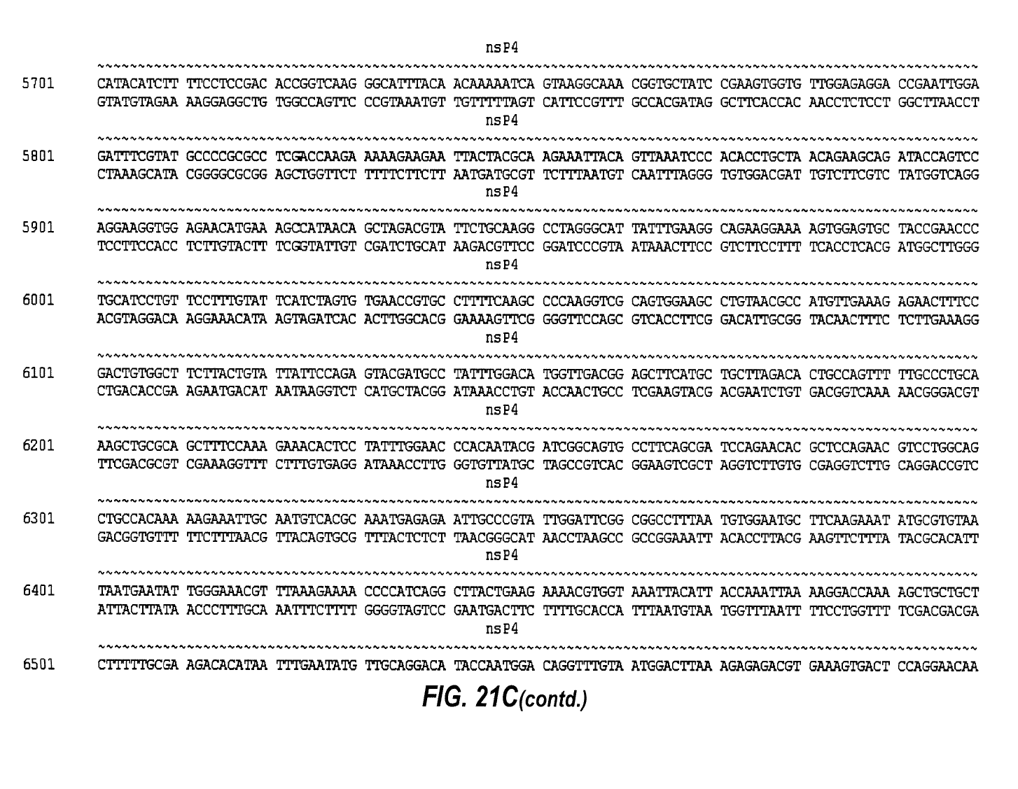

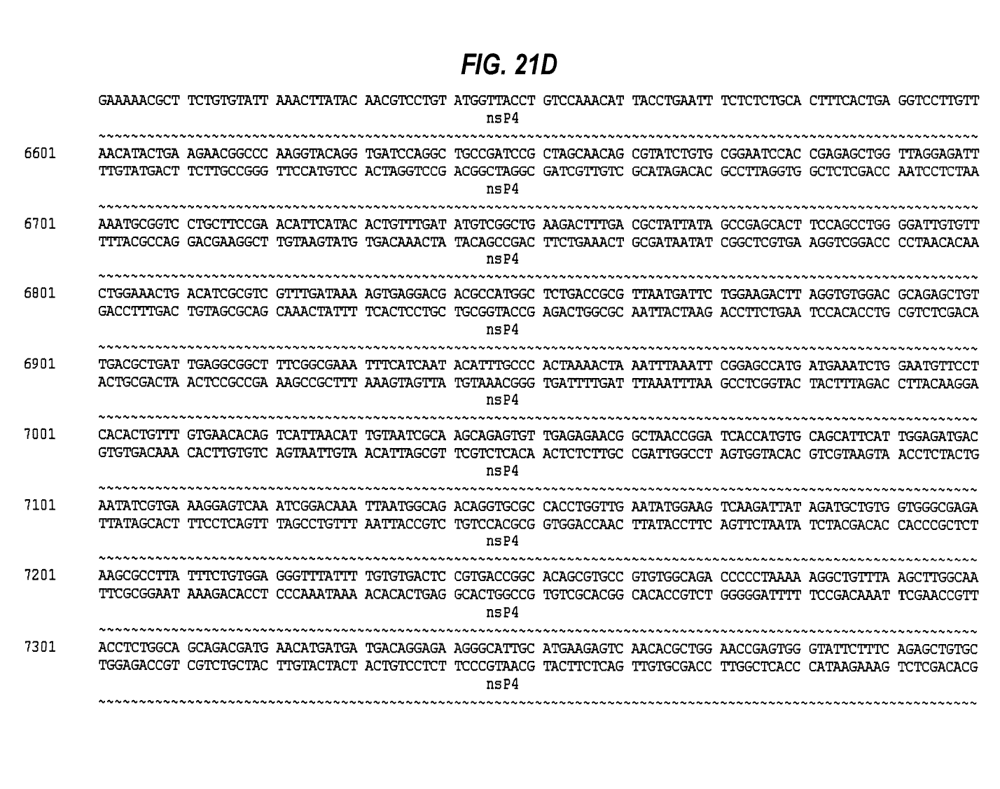

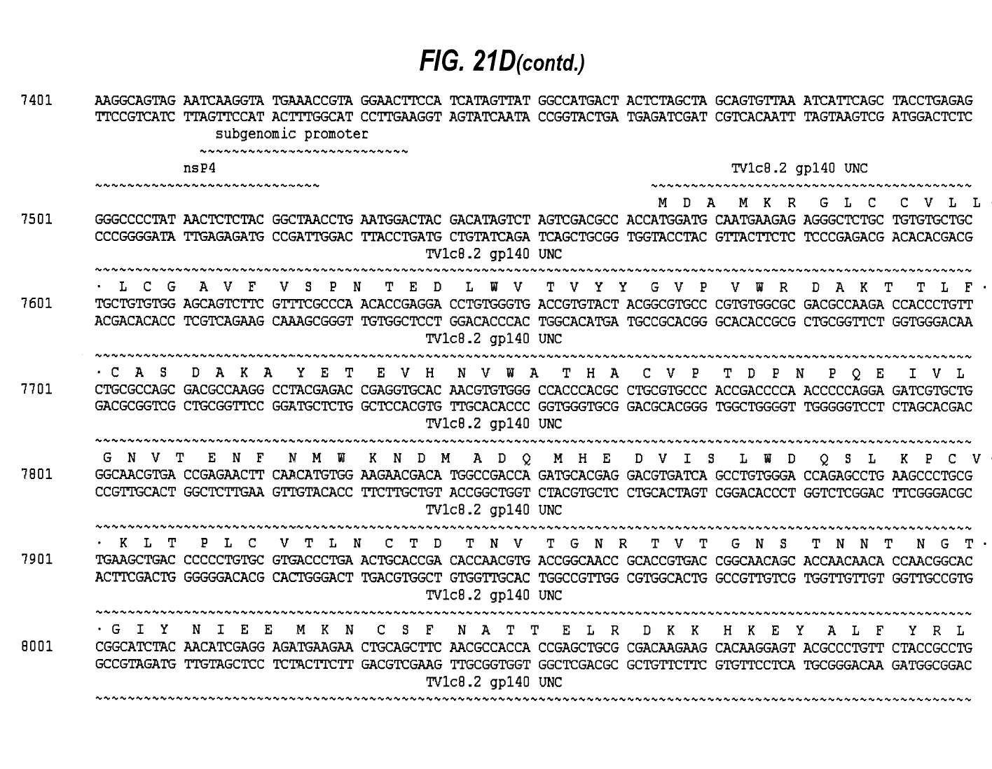

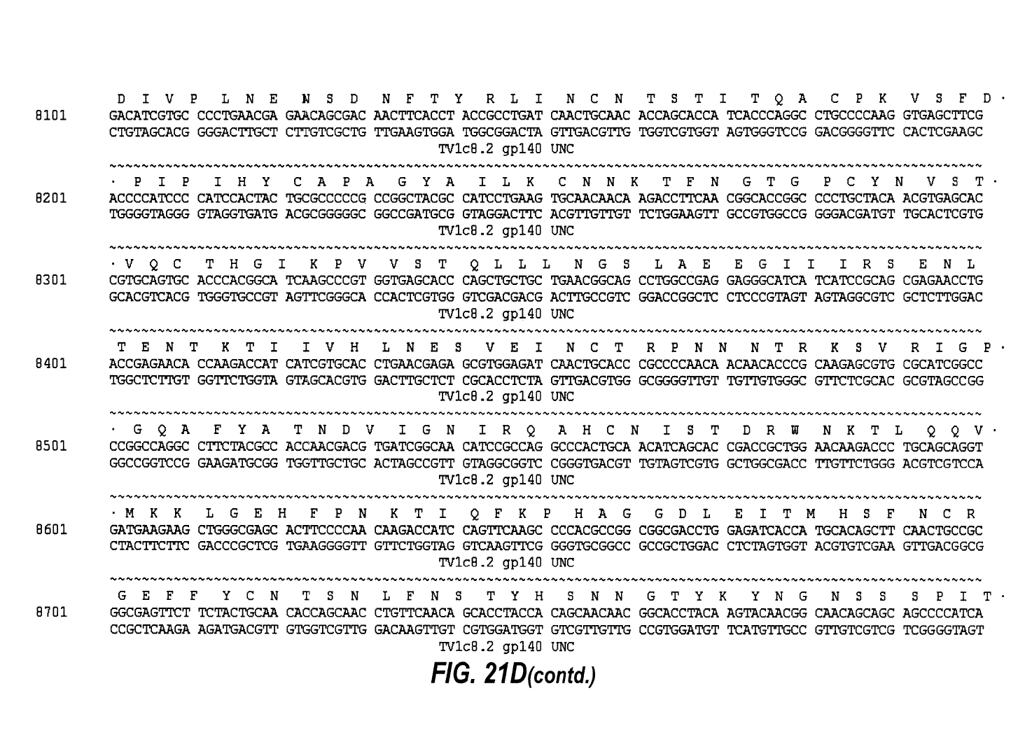

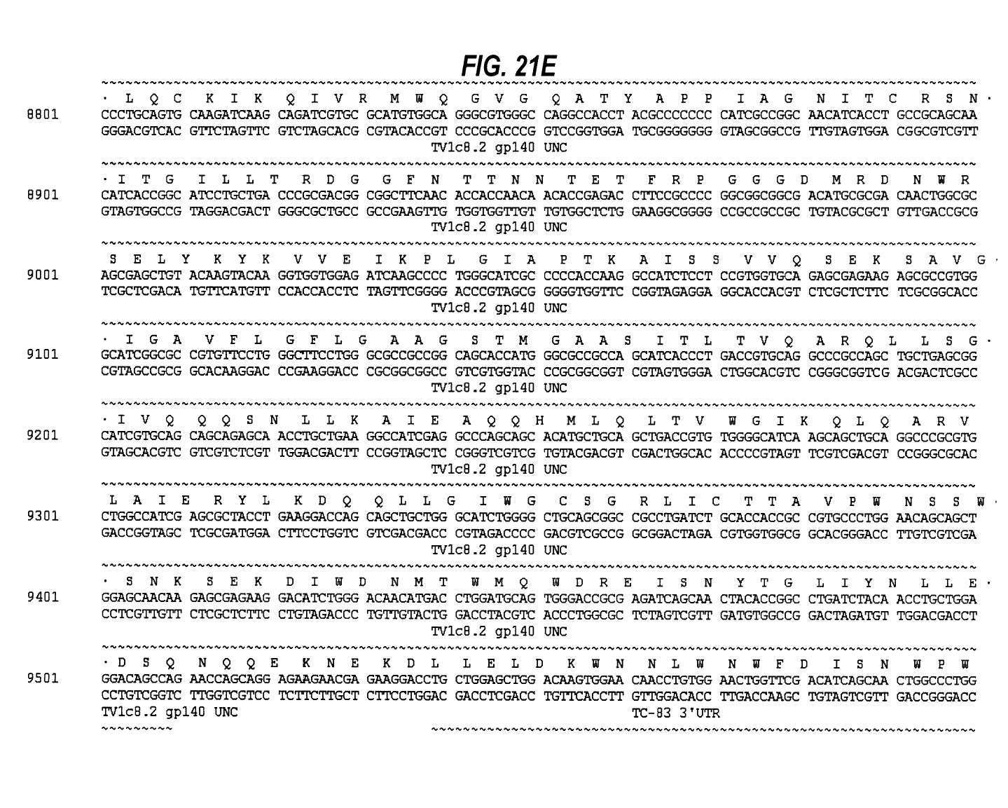

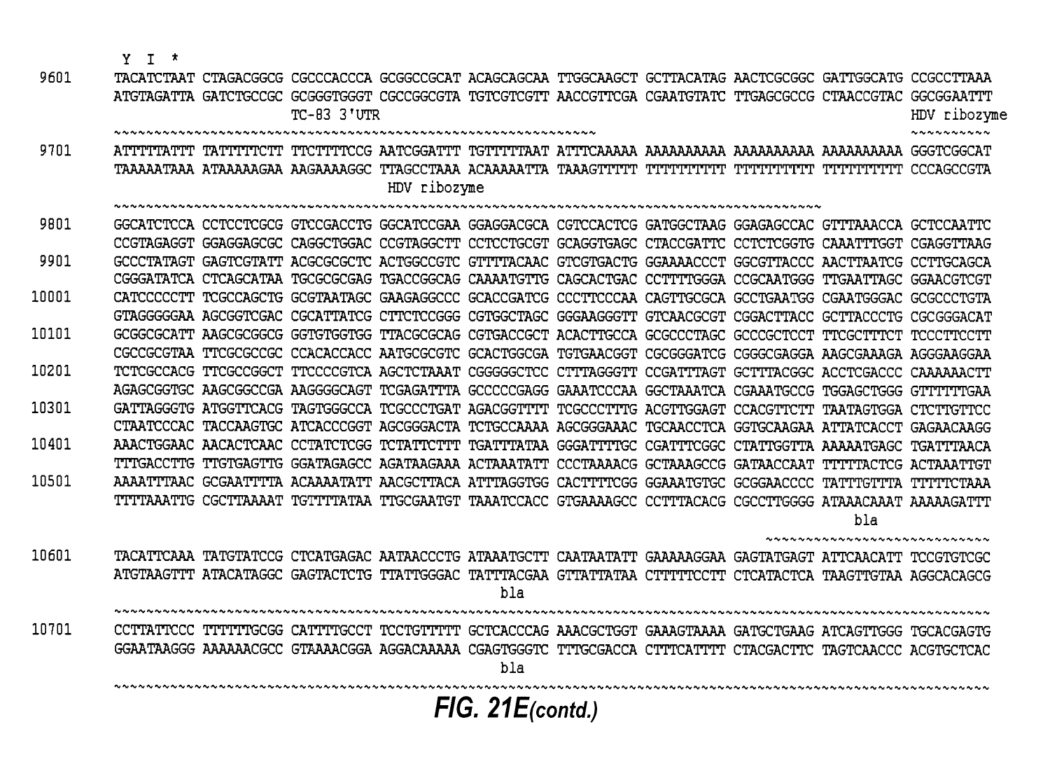

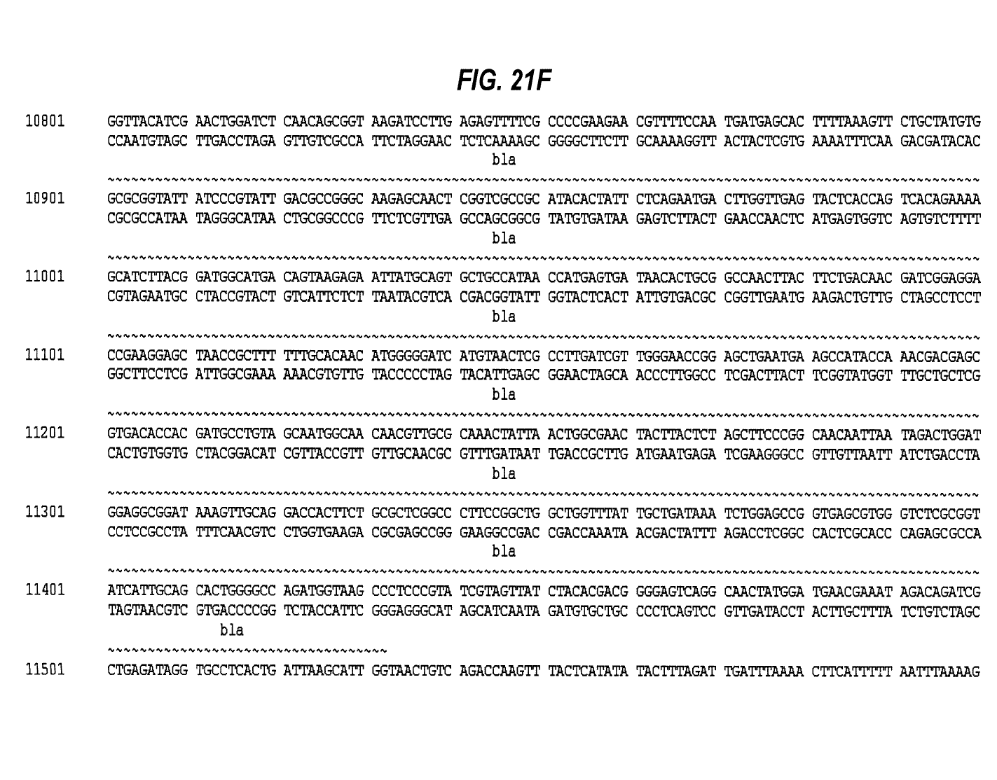

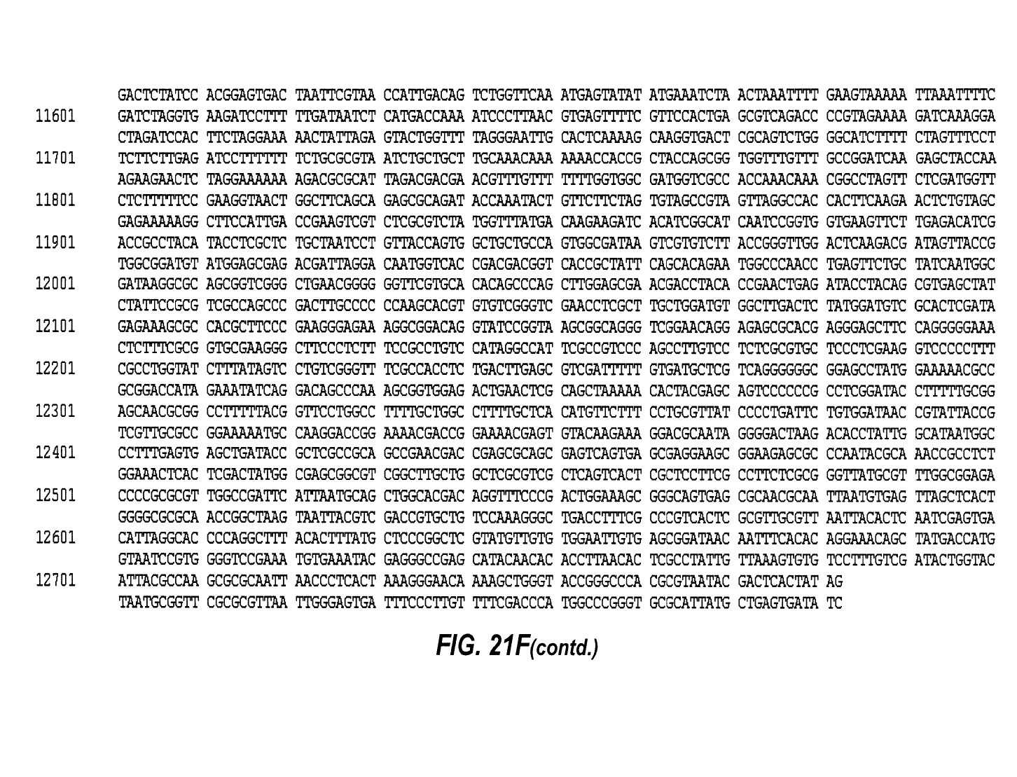

The following RNA replicon was used
H354-T7G-TV1c8.2 gp140mod unc—this RNA replicon expresses the uncleavable gp140 envelope protein from the Clade C TV1c8.2 strain. The vector used to transcribe the RNA, the annotated sequence of the vector and the insert are shown in FIG. 21.
   RNA was produced and purified as described in Example I.

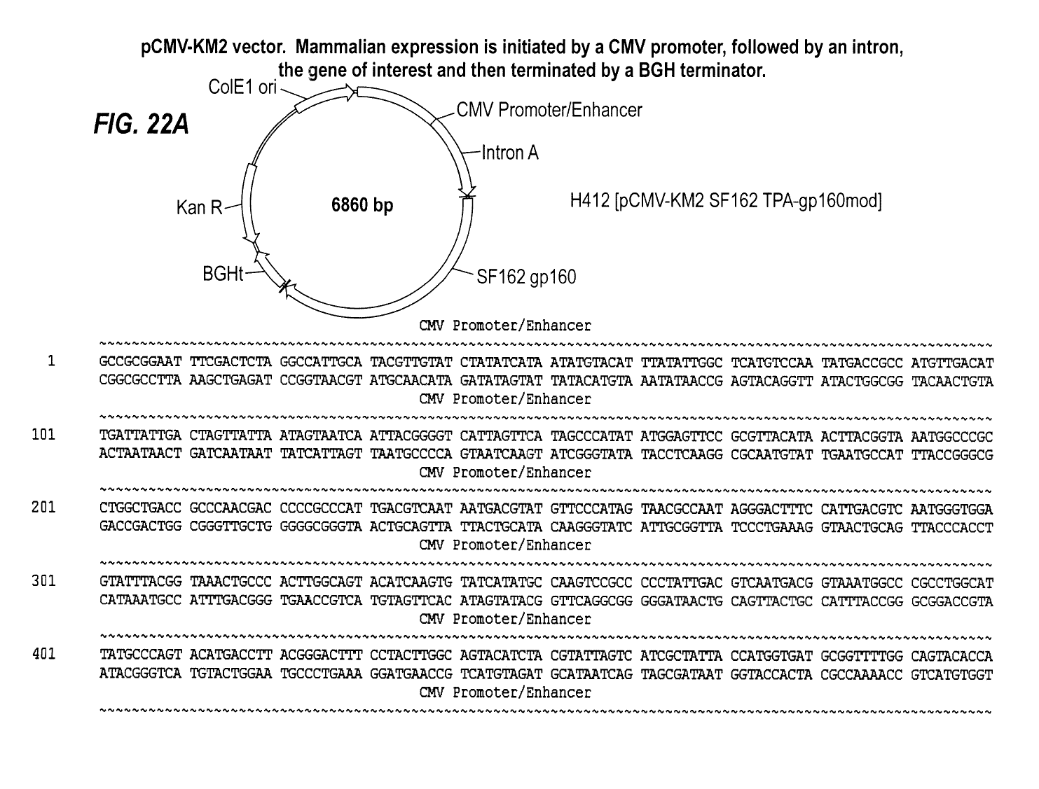

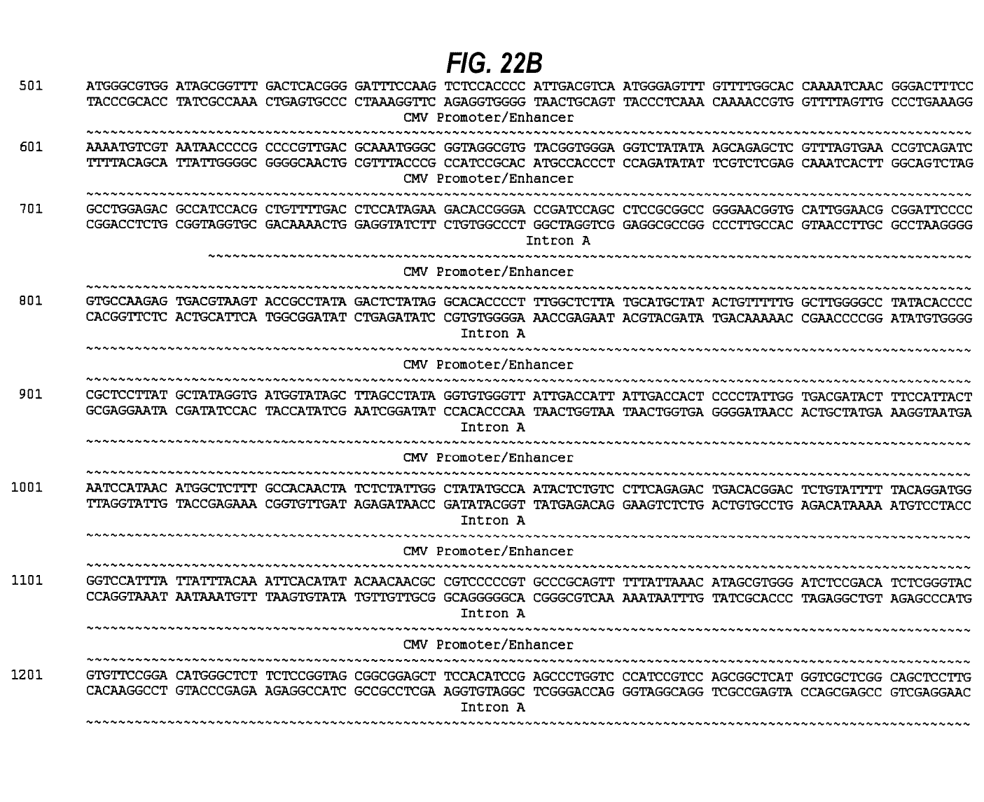

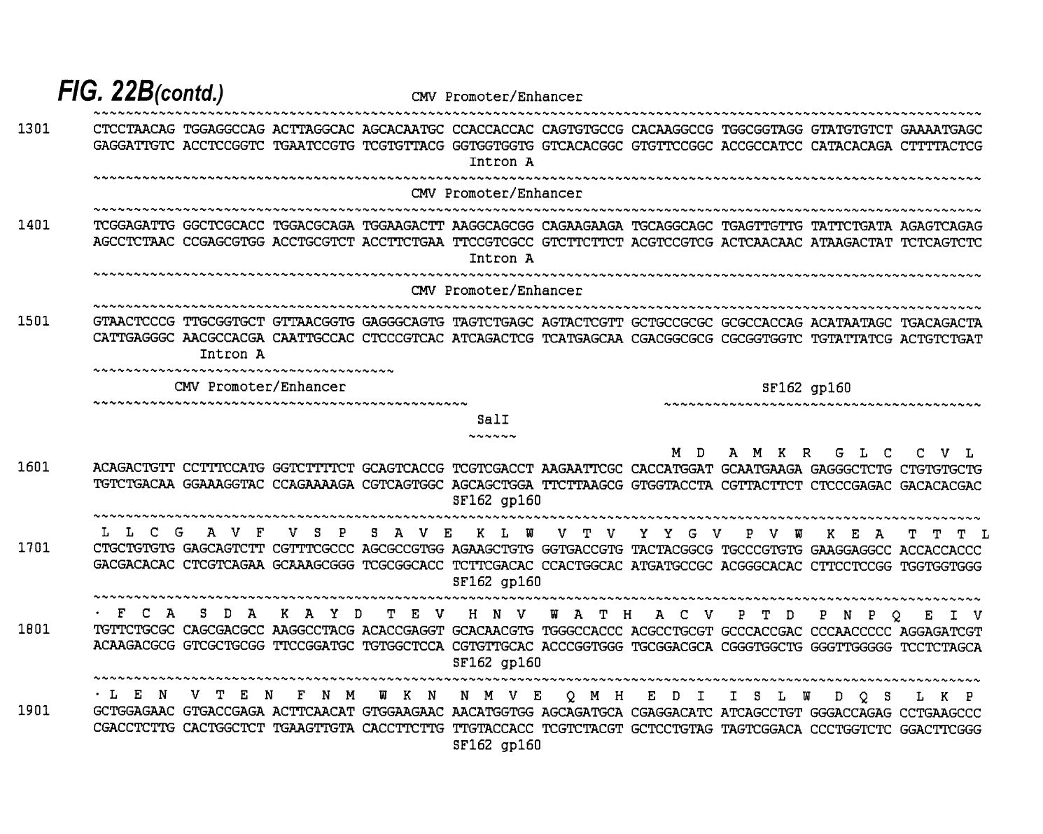

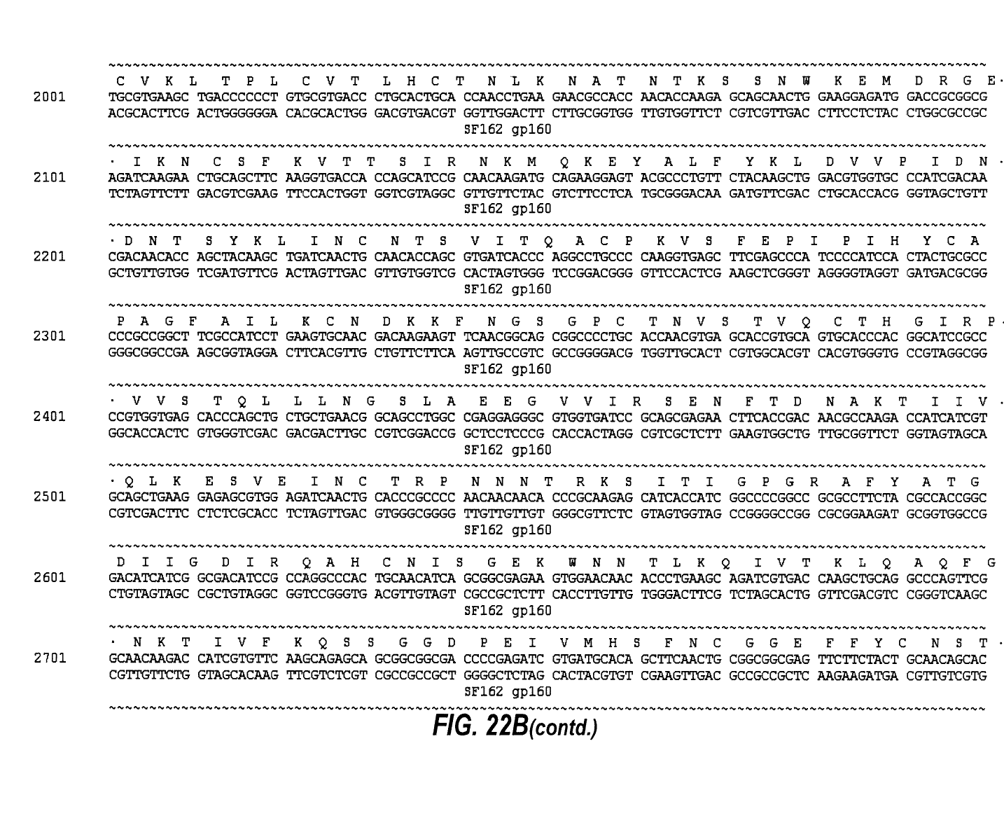

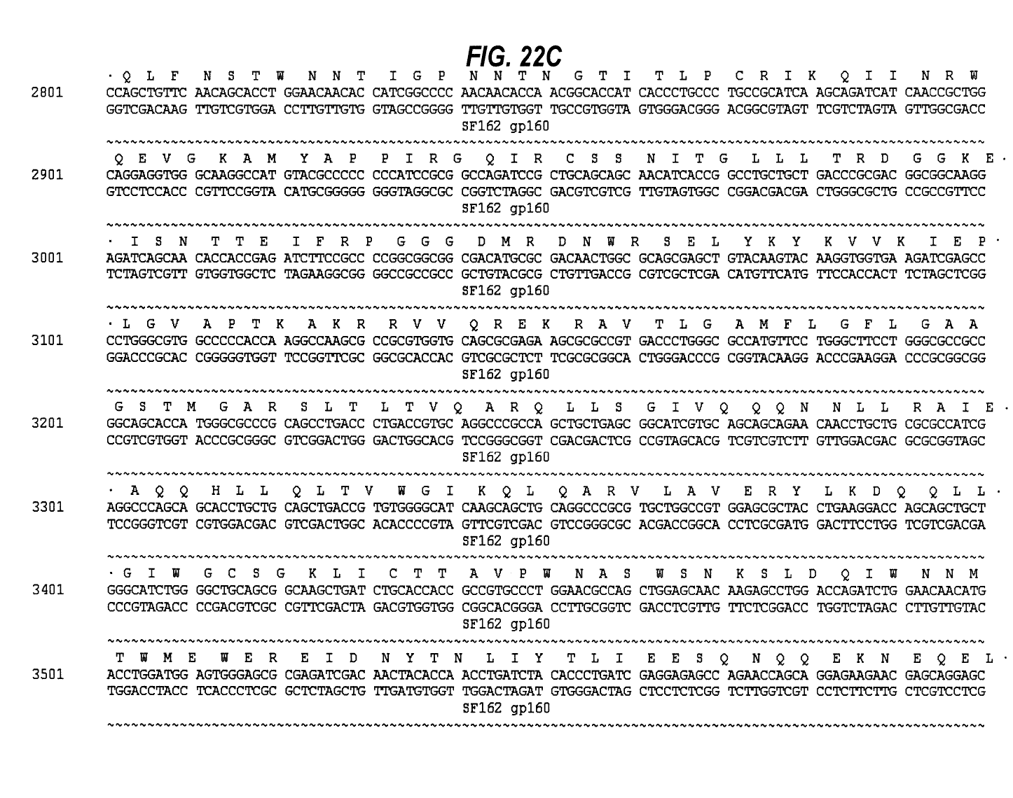

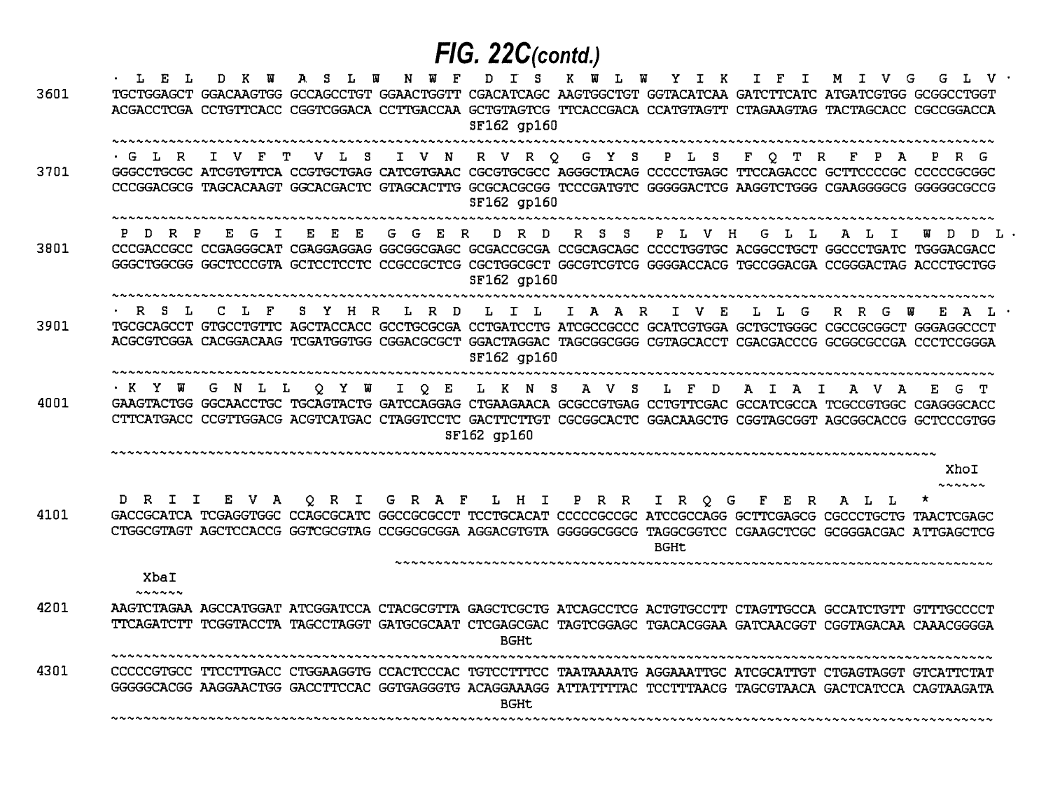

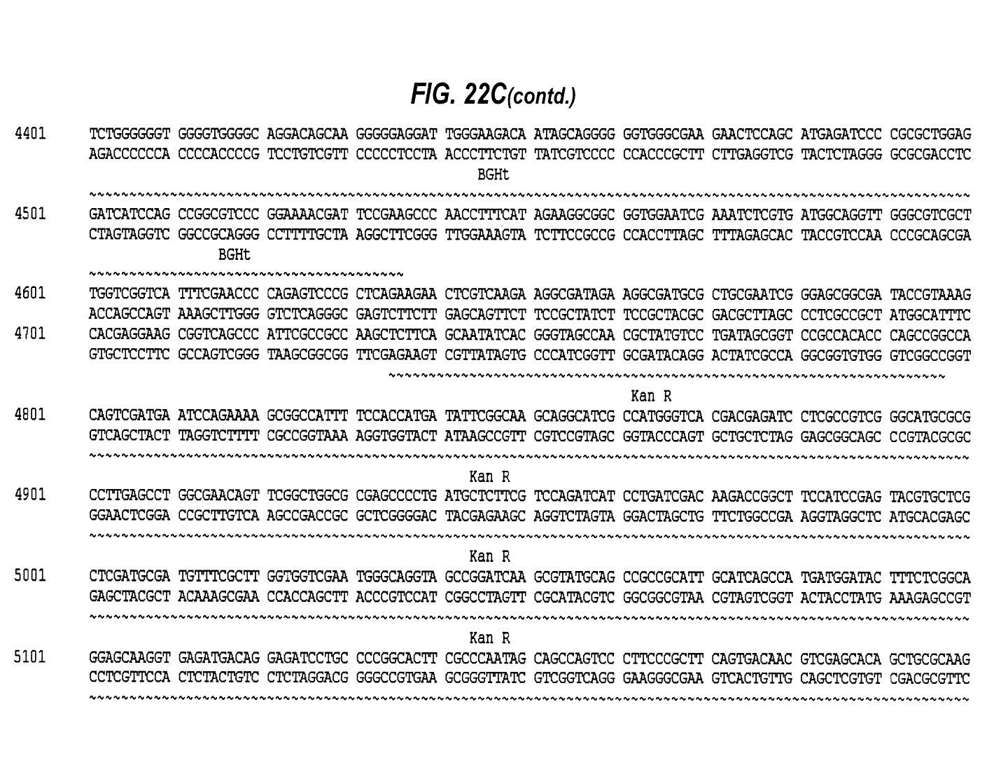

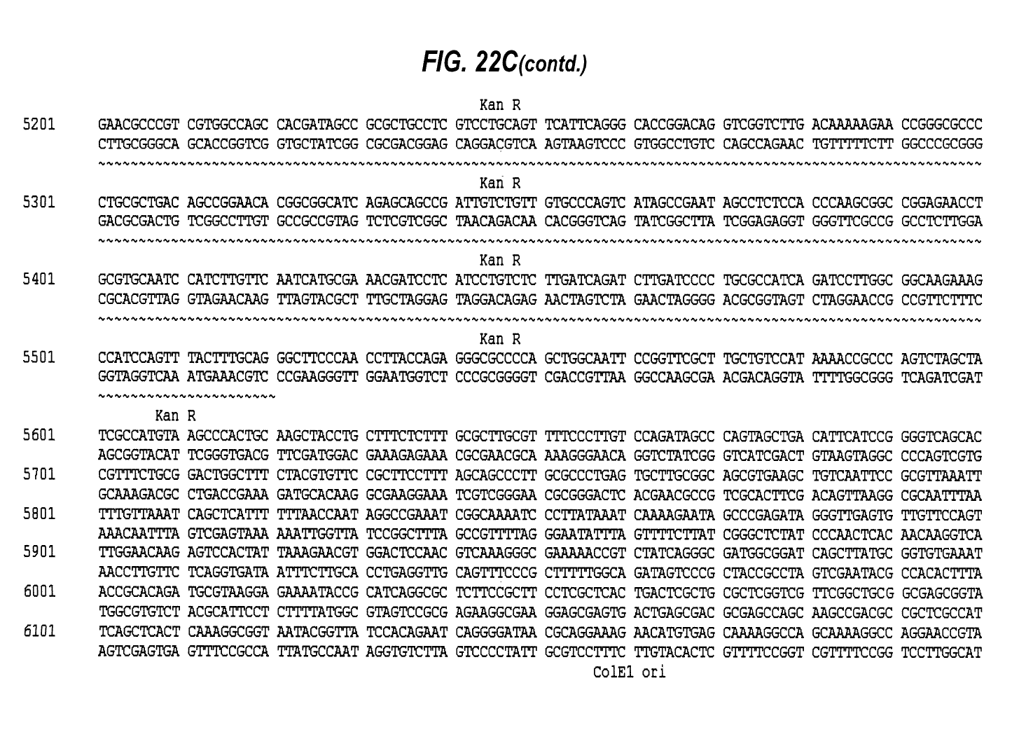

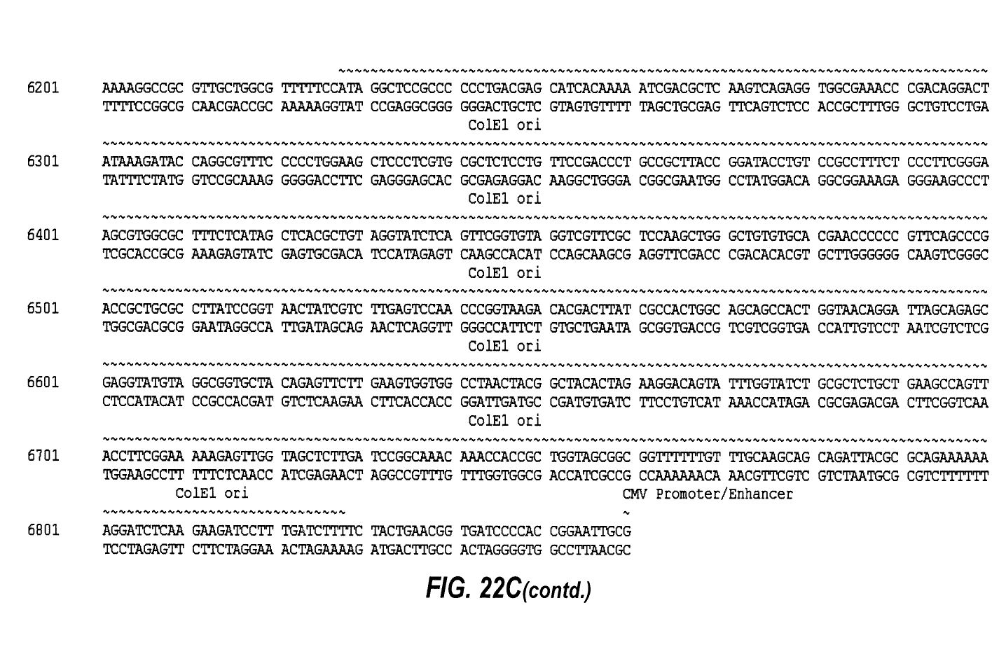

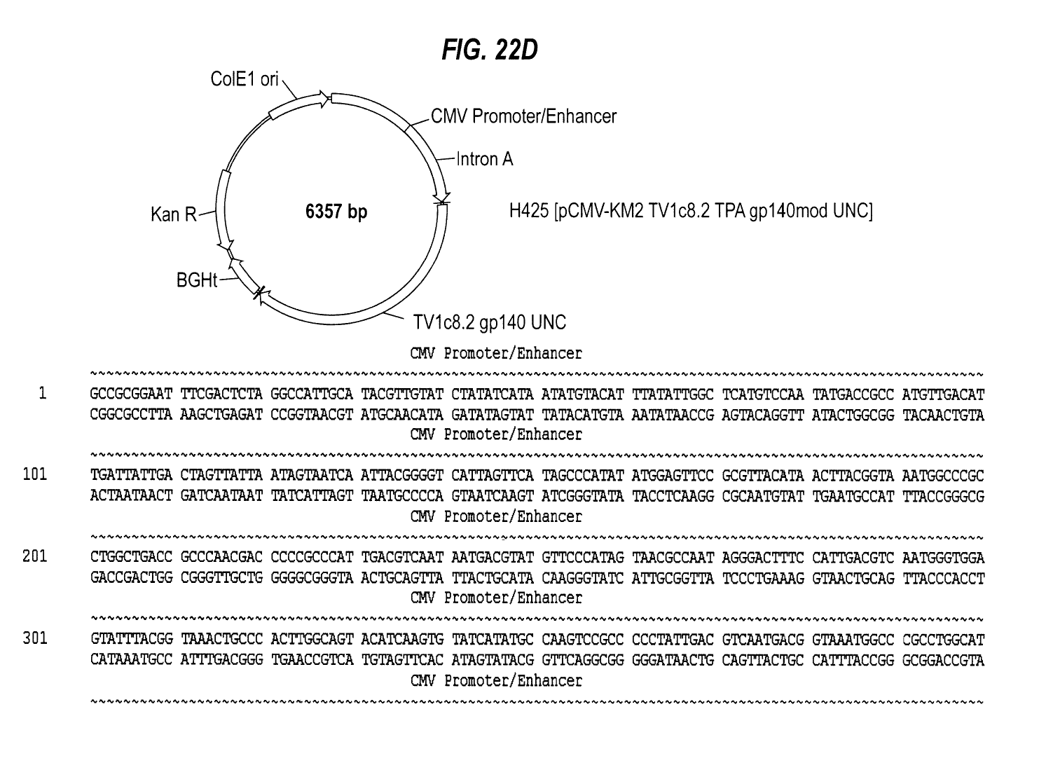

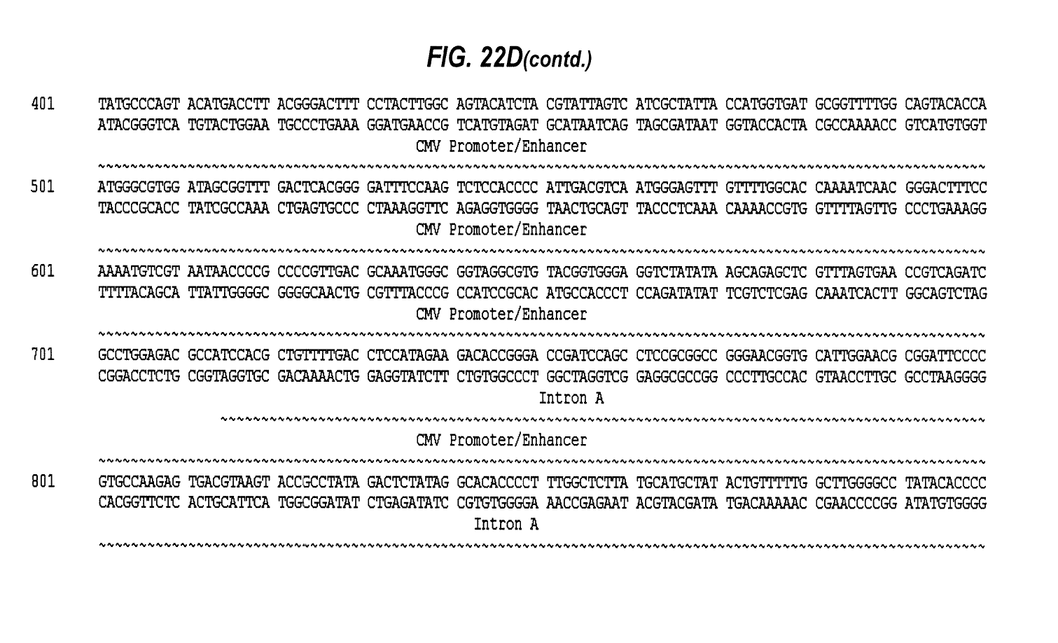

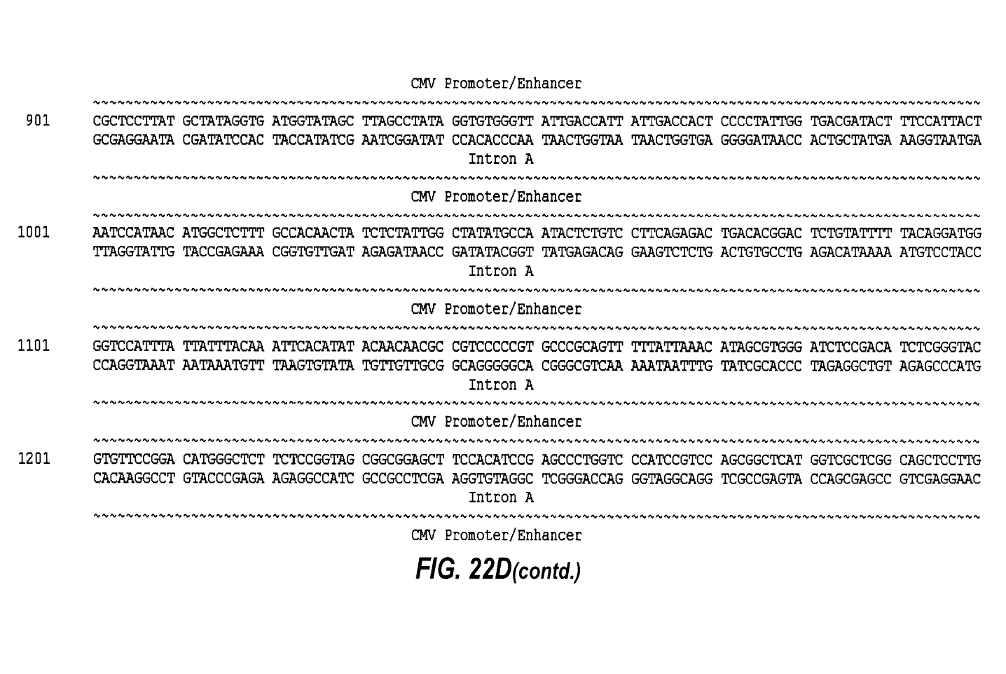

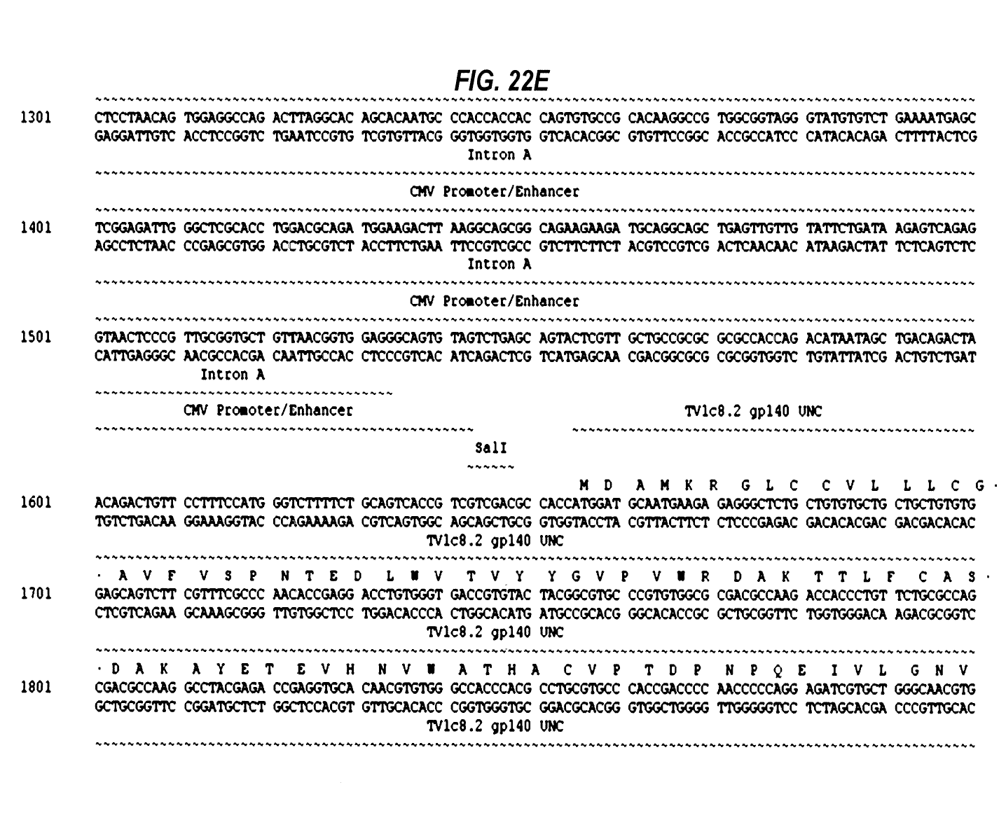

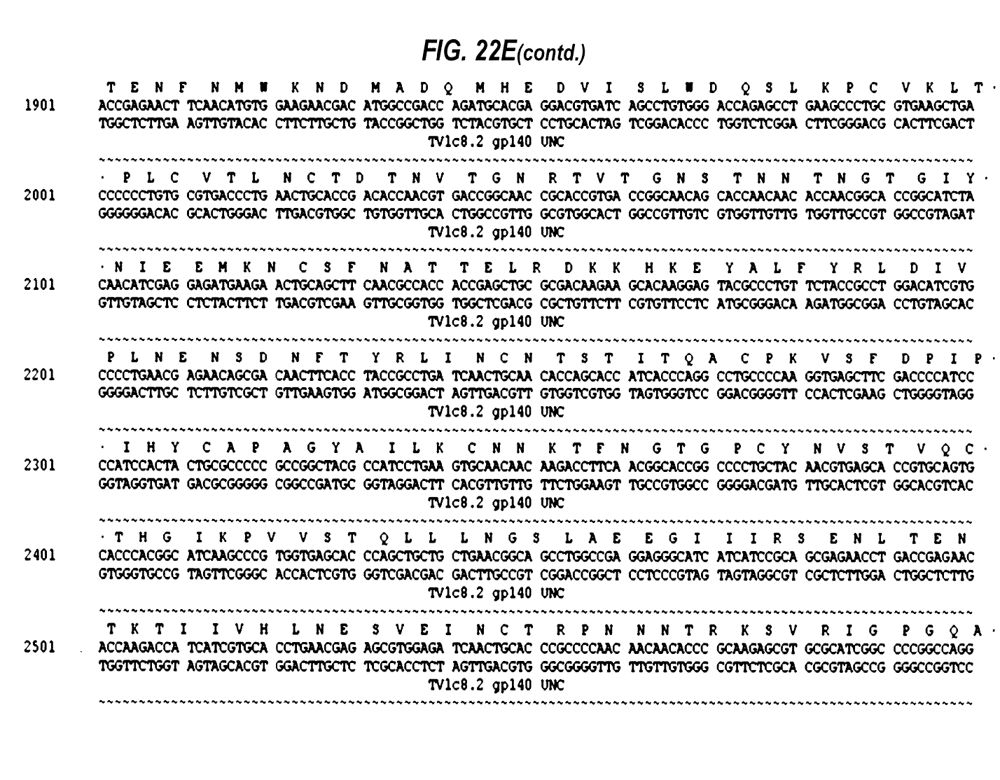

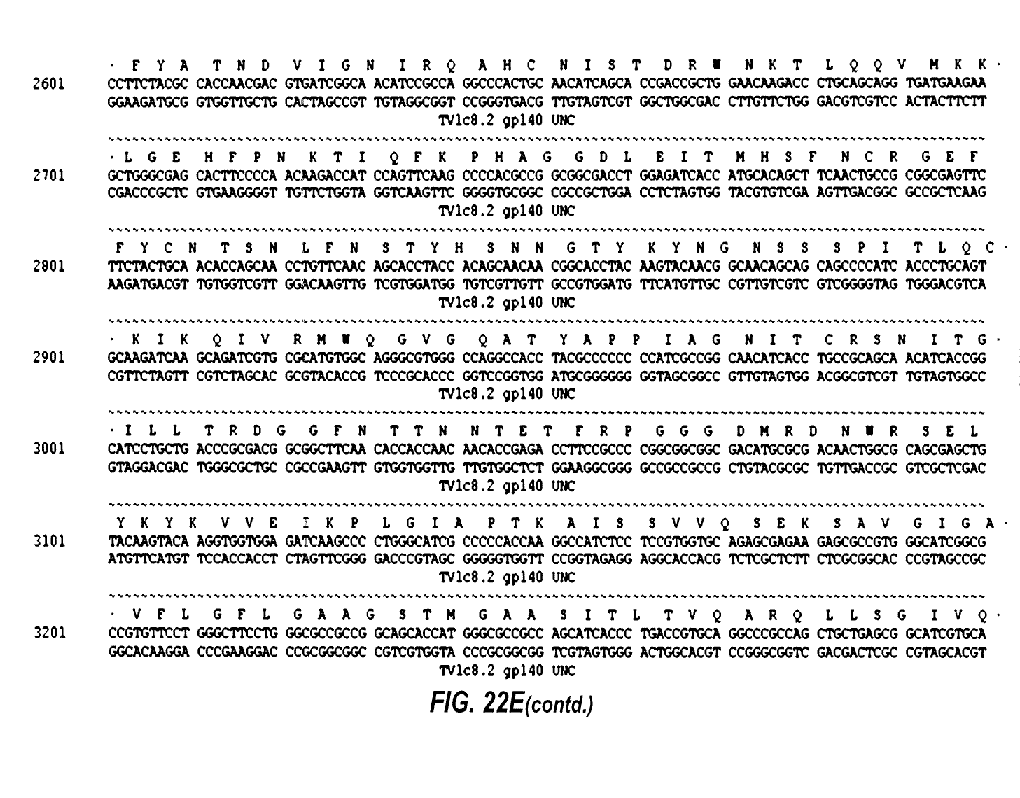

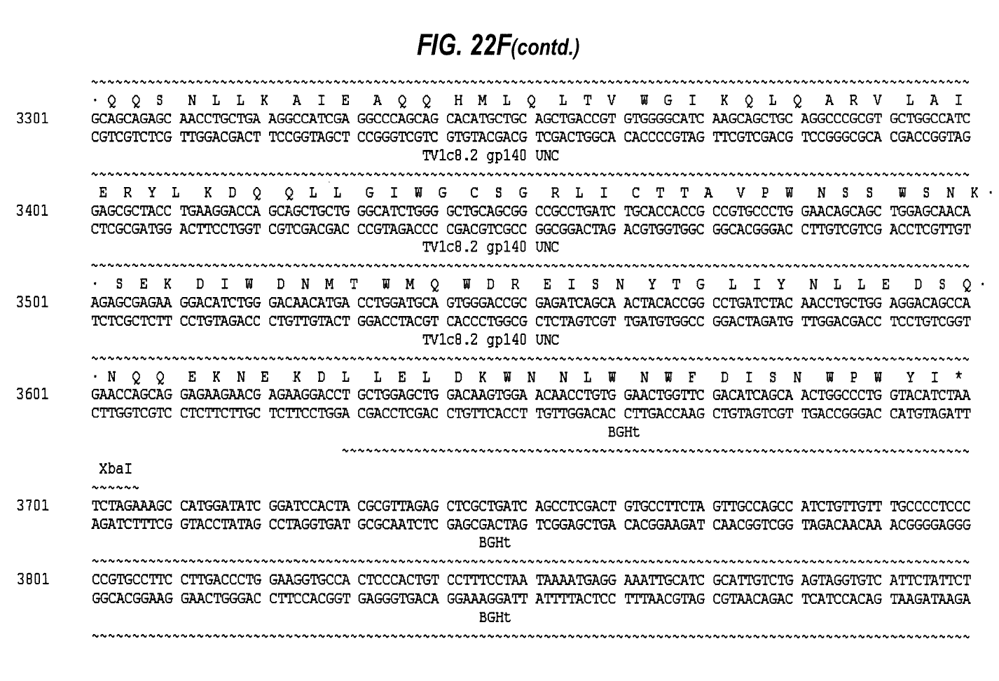

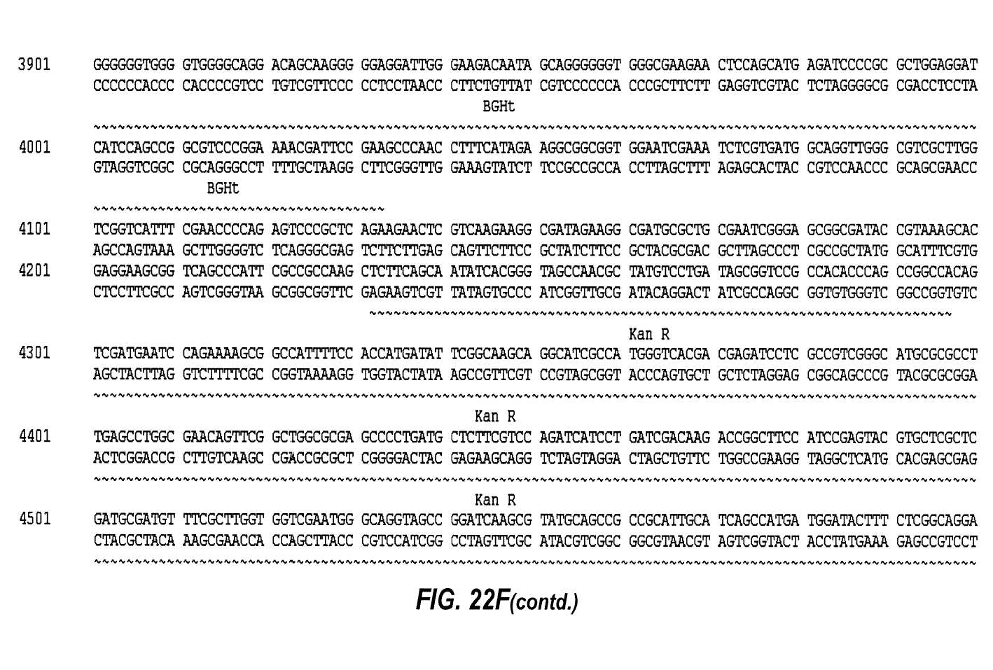

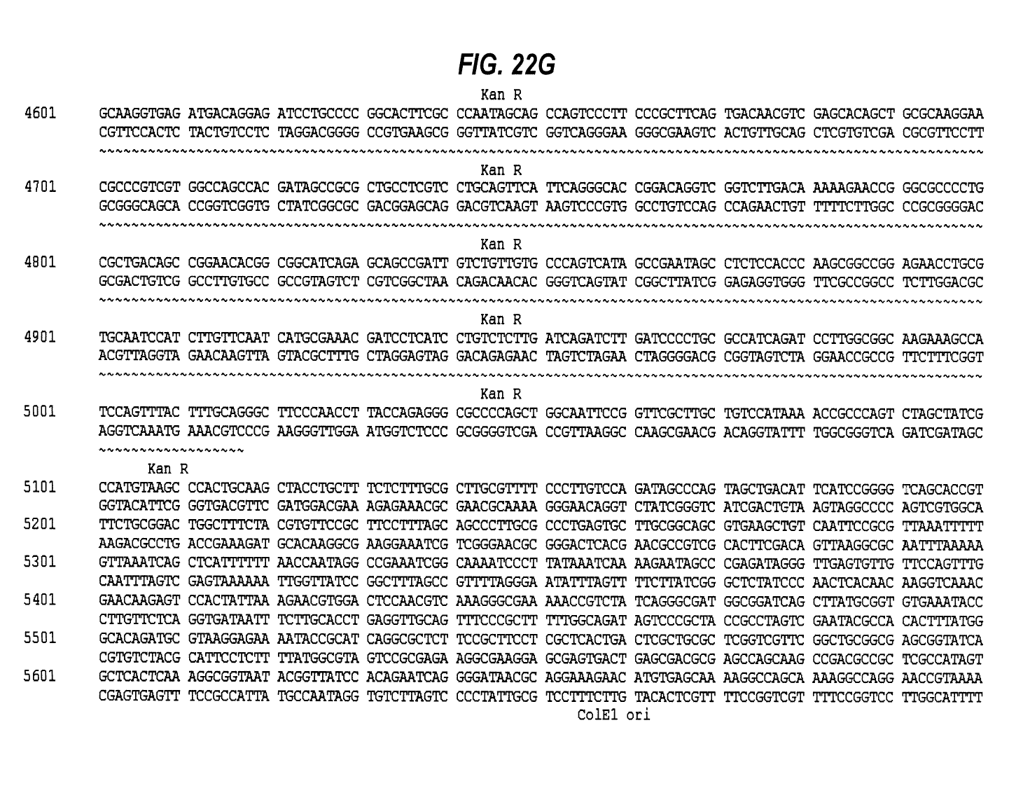

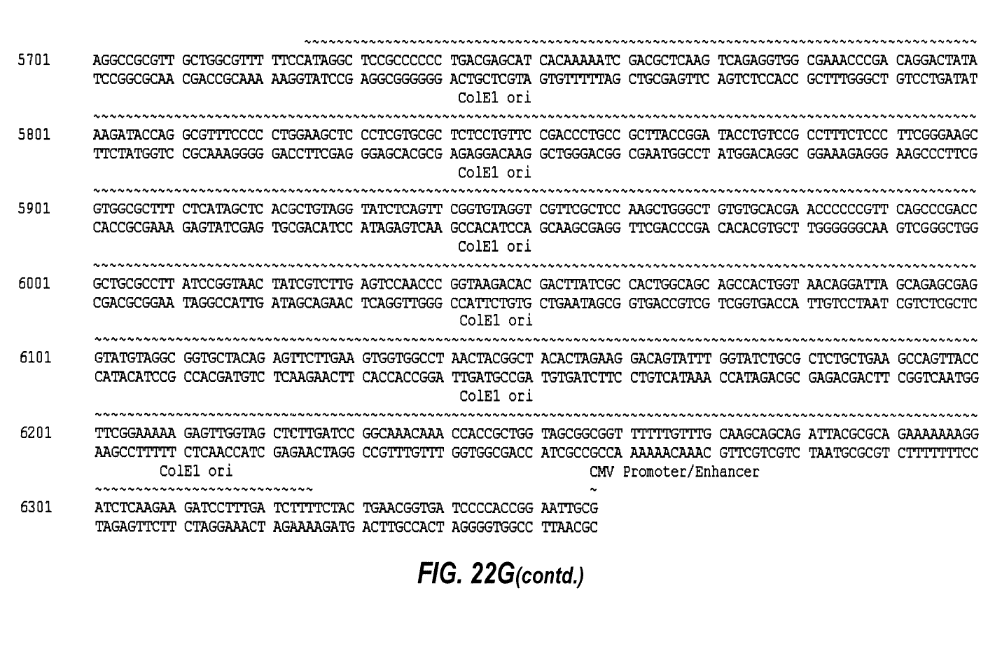

The following DNA vector was used to prime mice
H425—pCMV-KM2-TV1c8.2 gp140mod unc—this DNA vector expresses the uncleavable gp140 envelope protein from the Clade C TV1c8.2 strain. The expression vector, the annotated sequence of vector and insert are shown in FIG. 22.
   DNA was produced as described in Example I.

The following viral replicon particle (VRP) was used to prime mice
VRP gp140.TV1c8.2—this VRP expresses the gp140 envelope protein from the Clade C TV1c8.2 strain. See, e.g., Perri et al. (2003). J. Virol. 77(19): 10394-10403 regarding production and characterization of VRPs.

gp140 protein from Clade C TV1c8.2 strain was produced as described for gp140 from Clade B SF162 in Example I.

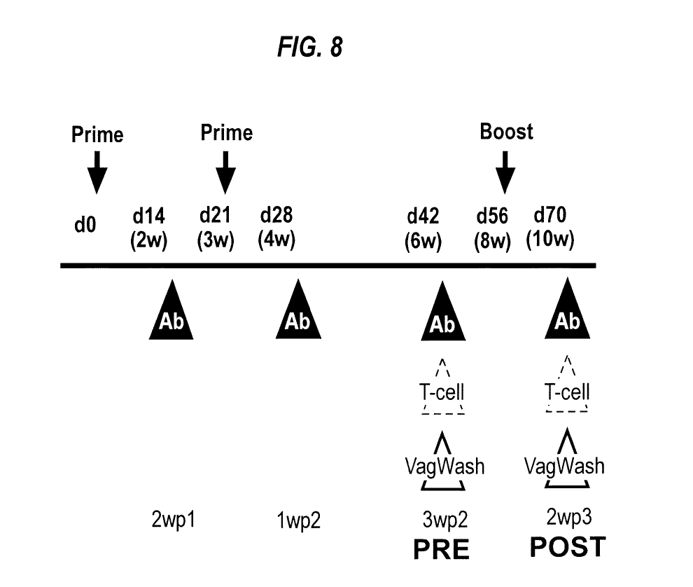

Various HIV gp140 formulations were administered to mice according to the schedule depicted in FIG. 8. Groups of BALB/c mice receiving the gp140 formulations are summarized in Table II-1 below.

TABLE II-1

| | No. of | | | | | |
|---|---|---|---|---|---|---|
| Group | mice | HIV gp140 formulation | | Priming (2X) | | Boost |
| Gp1 | 8 | RNA TV1 gp140 | Naked RNA | 1 | μg | 10 μg gp140. |
| Gp2 | 8 | RNA TV1 gp140 | Naked RNA | 10 | μg | TV1 (with |
| Gp3 | 8 | RNA TV1 gp140 − | RNA in liposome | 1 | μg | MF59) |
| | | Liposome | | | | |
| Gp4 | 8 | RNA TV1 gp140 − | RNA in liposome | 10 | μg | |
| | | Liposome | | | | |
| Gp5 | 8 | DNA TV1 gp140 | Naked DNA | 1 | μg | |
| Gp6 | 8 | DNA TV1 gp140 | Naked DNA | 10 | μg | |
| Gp7 | 8 | DNA TV1 gp140 − | DNA in liposome | 1 | μg | |
| | | Liposome | | | | |
| Gp8 | 8 | DNA TV1 gp140 − | DNA in liposome | 10 | μg | |
| | | Liposome | | | | |
| Gp9 | 8 | DNA TV1 gp140 | Naked DNA delivered | 10 | μg | |
| | | (EP) | by electroporation | | | |
| Gp10 | 8 | VRP TV1 gp140 | virus replicon particles | 10⁷ | IU | |
| Gp11 | 8 | TV1 Protein | | 10 | μg (with | |
| | | | | | MF59) | |

| | | | | | |
|---|---|---|---|---|---|
| Gp12 | 8 | RNA TV1 gp140 + | RNA in liposome + | 1 μg RNA + | |
| | | TV1 Protein | protein | 10 μg protein | |
| Gp13 | 4 | naive | — | — | — |

### 2 Co-Administering RNA and its Encoded Protein Induced a Robust and Balanced Immune Response.

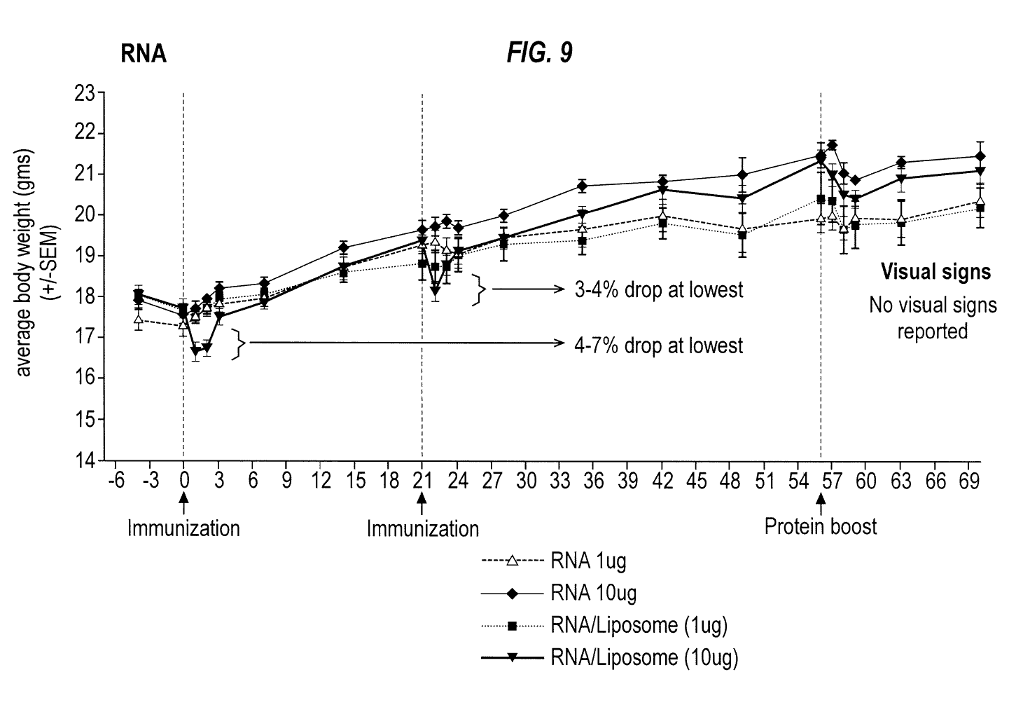

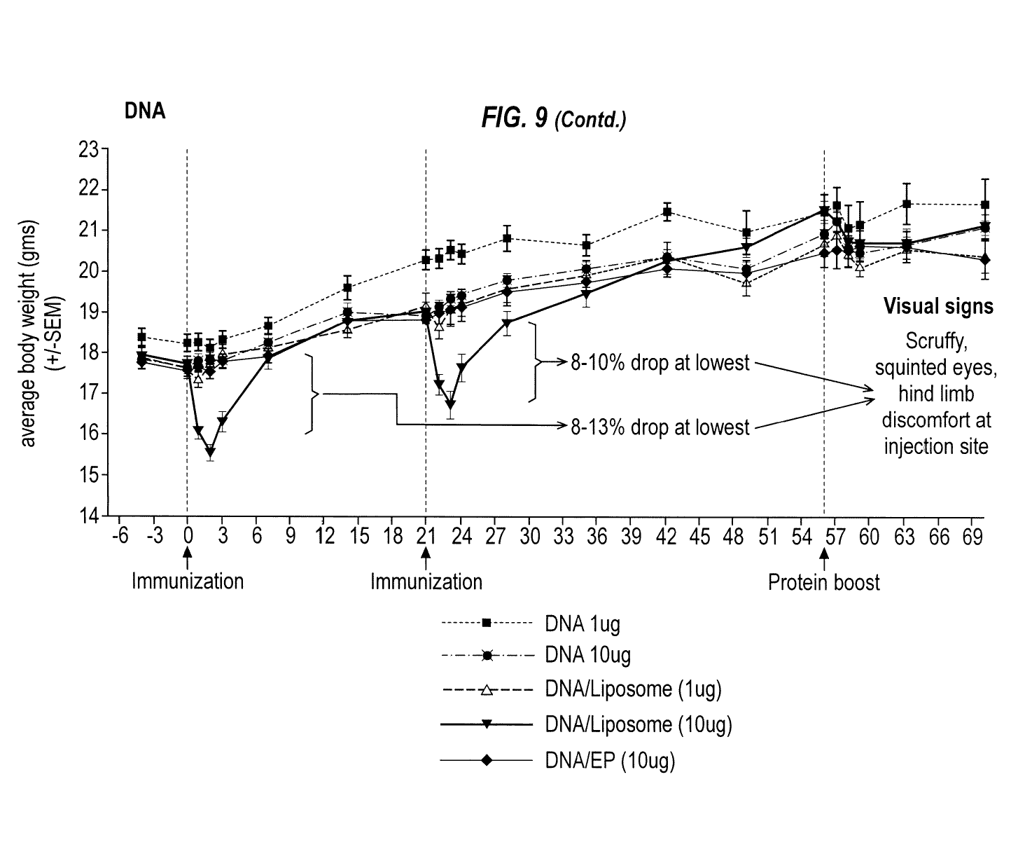

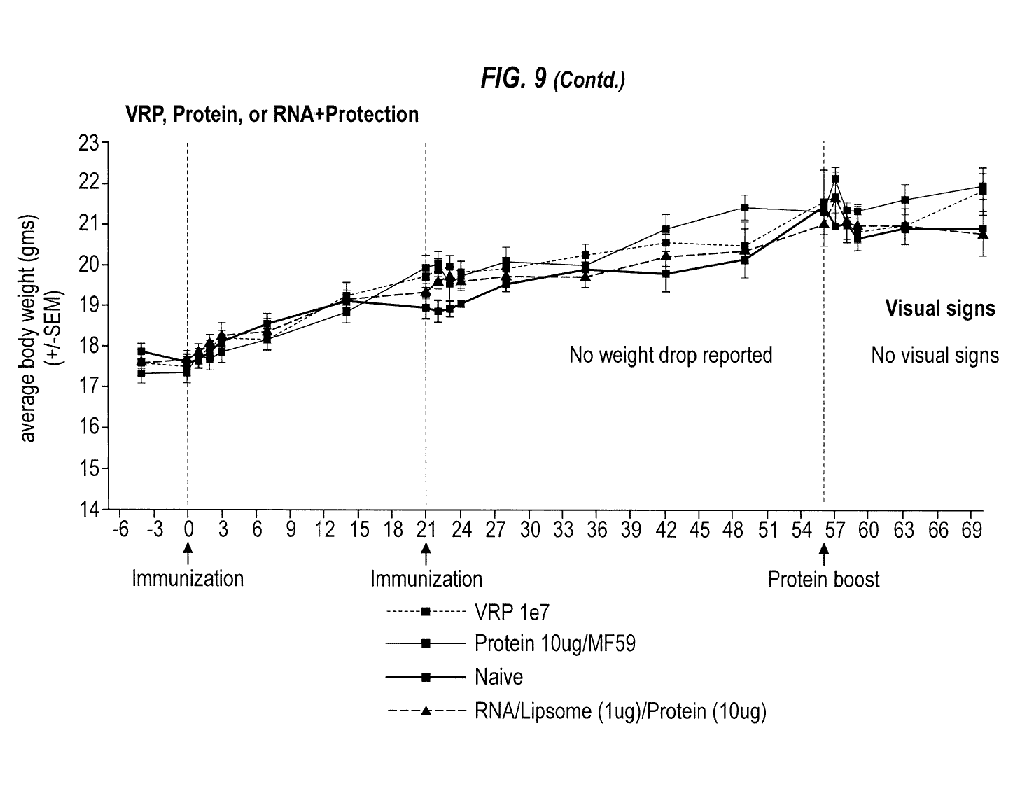

First, the HIV gp140 formations described in Table II-1 were evaluated for potential adverse effects. FIG. 9 shows that co-delivery of RNA replicon and its encoded protein antigen showed no adverse effect. Transient loss of body weight and other visual signs of distress were observed after high dose (10 μg) Liposome encapsulated RNA and DNA formulations were administered, but there was no evidence of adverse effects with 1 μg RNA/Liposome, or 1 μg RNA/Liposome/Protein. In addition, weight loss and other visual signs of adverse effects were lower with 10 μg of RNA/Liposome, as compared to 10 μg of DNA/Liposome.

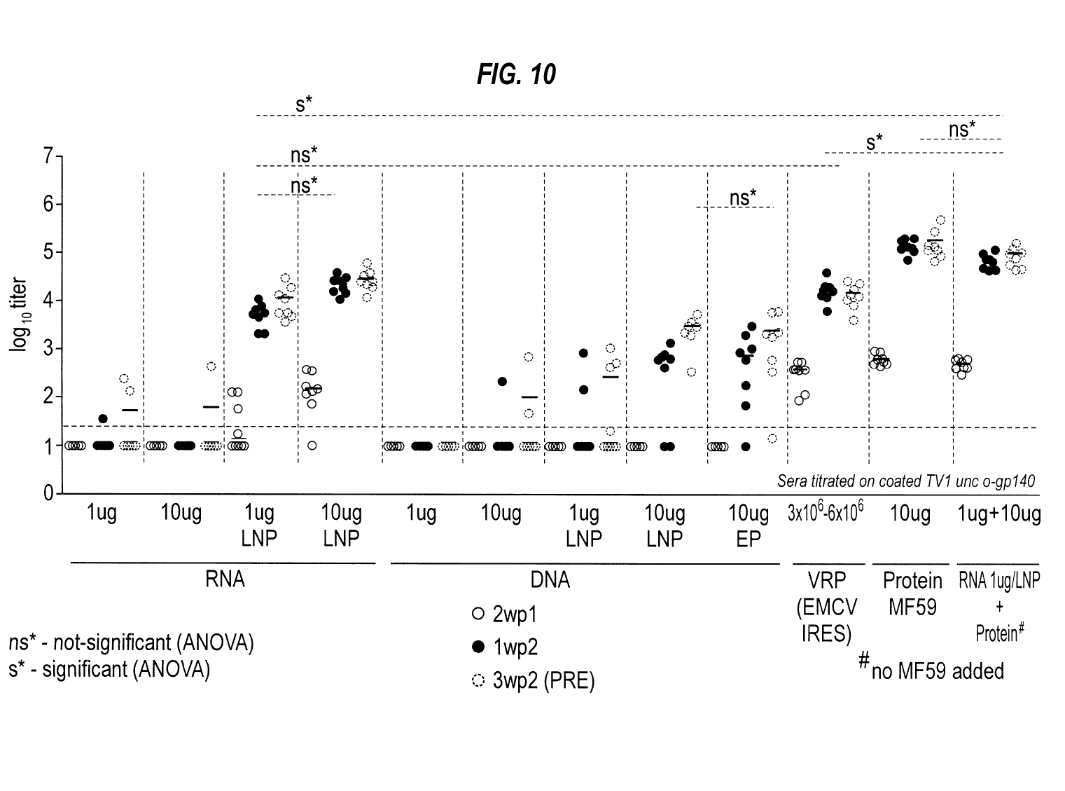

Second, anti-gp140 IgG antibody titers were measured to evaluate the immune response induced by the HIV gp140 formations described in Table II-1. As shown in FIG. 10, prior to a protein boost, 1 μg dose of liposome encapsulated RNA replicon (RNA/Liposome) induced a strong immune response, with gp140-specific IgG titers comparable to that of virus replicon particles (VRP) at 3wp2. In addition, at 3wp2, there was no significant difference in IgG titers between 1 μg RNA/Liposome and 10 μg RNA/Liposome. IgG titers induced by 1 μg RNA/Liposome and 10 μg RNA/Liposome were superior to 1 μg DNA/Liposome and 10 μg DNA/Liposome, and were also superior to electroporated 10 μg DNA.

Combining the RNA replicon with gp140 protein (RNA/Liposome/Protein) induces an even stronger immune response as compared to RNA/Liposome. As shown in FIG. 10, anti-gp140 IgG titers induced by 1 μg RNA/Liposome/Protein was significantly higher than that of 1 μg RNA/Liposome, and was also significantly higher than that of VRP. There was no significant difference in anti-gp140 IgG titers between the 1 μg RNA/Liposome/Protein group and Protein/MF59 group.

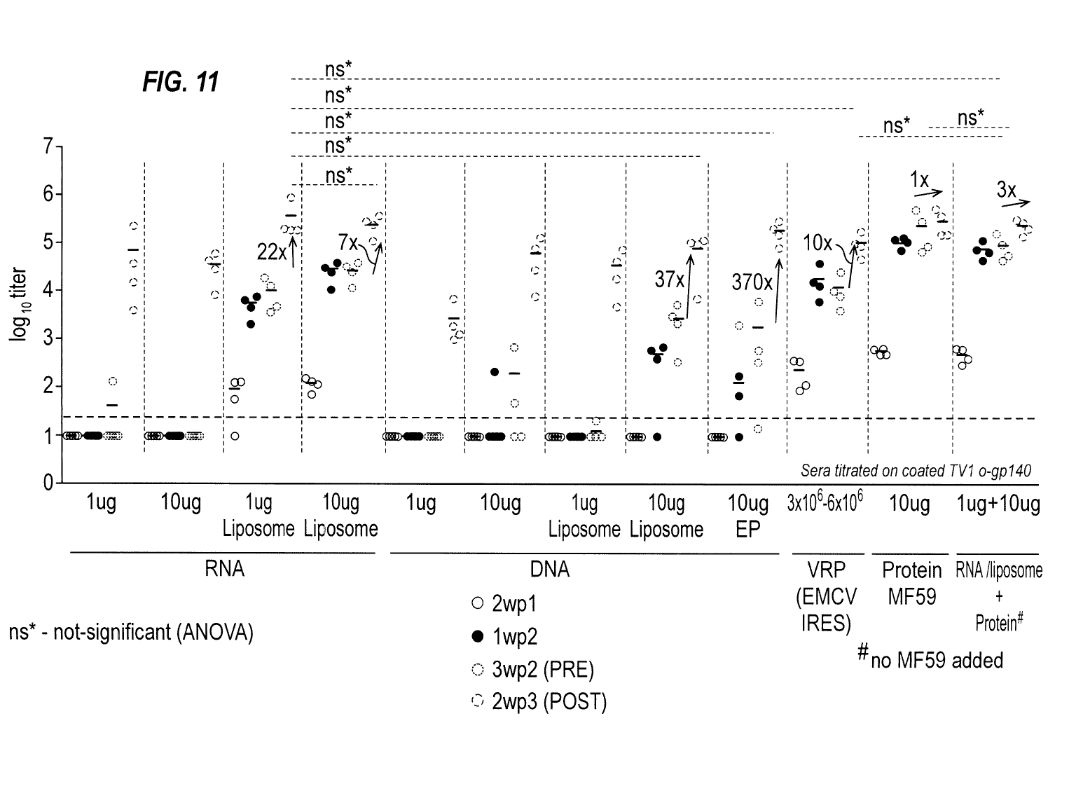

FIG. 11 shows the anti-gp140 IgG titers measured after a boost (10 μg protein/MF59, see Table II-1) was administered. The IgG titers of the 1 μg RNA/Liposome primed group did not differ significantly from that of 10 μg RNA/Liposome primed group, 1 μg DNA/Liposome primed group, 10 μg DNA/Liposome primed group, or VRP primed group.

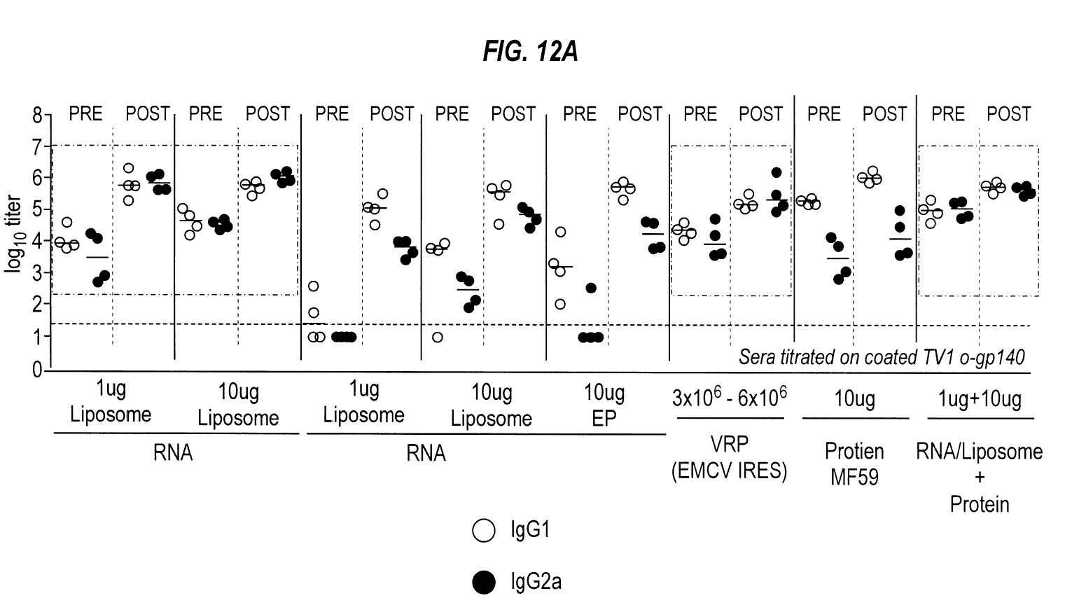

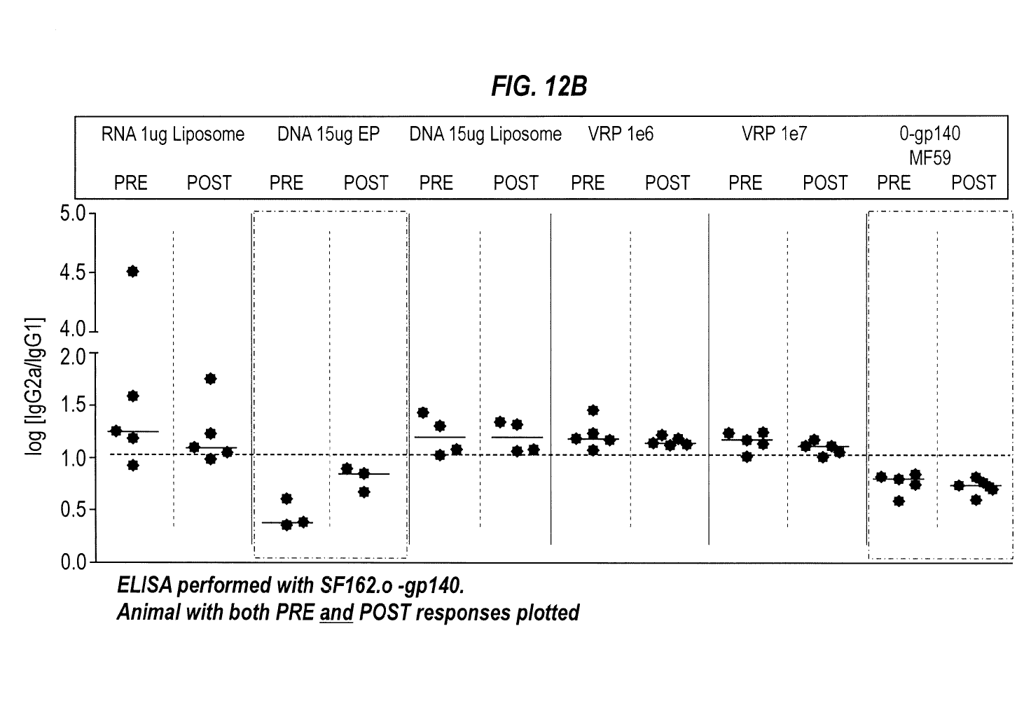

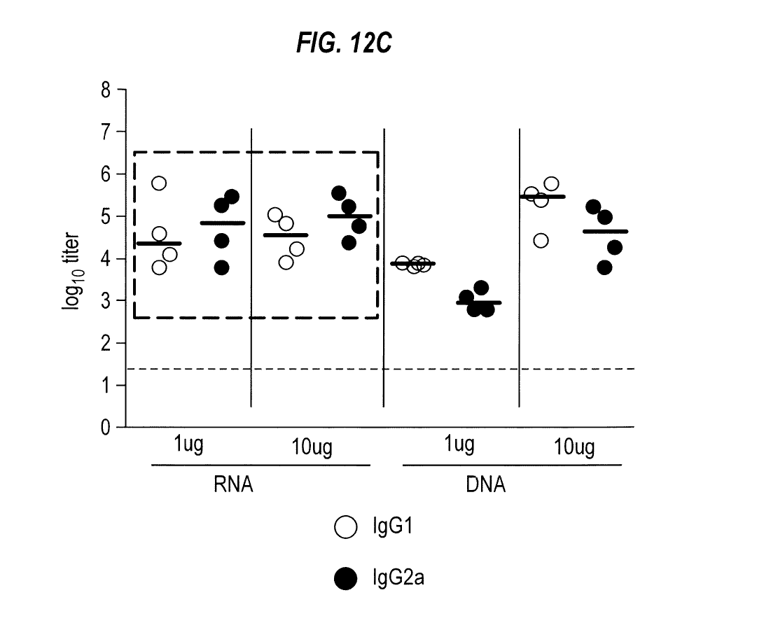

FIGS. 12A and 12B show that RNA/Liposome and RNA/Liposome/Protein formulations induced a balanced IgG1:IgG2a subtype profile, similar to that of VRP. Naked RNA immunized groups, in which titers were not detectable before the protein/MF59 boost, also showed a balanced IgG1:IgG2a profile after the protein boost (FIG. 12C). In contrast, in the Protein/MF59 primed group, the IgG1 titers were significantly higher than IgG2a titers. IgG2a is considered as a surrogate of Th1 response, and IgG1 is considered as a surrogate of Th2 response. A balanced Th1:Th2 response is desirable.

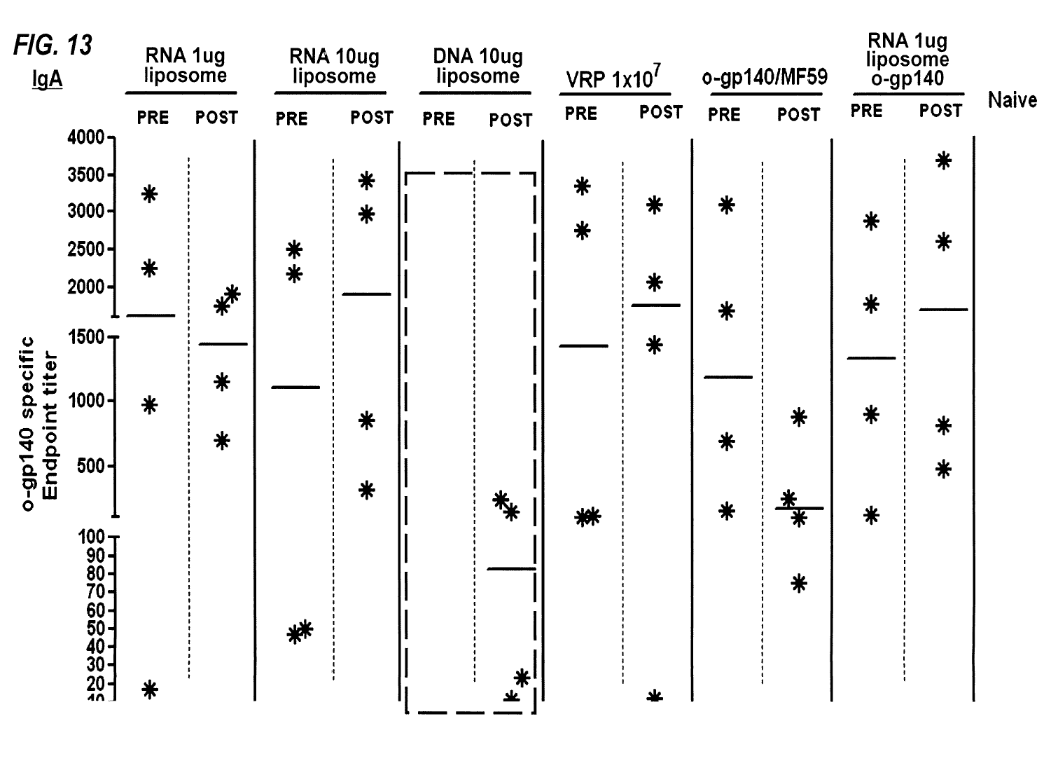

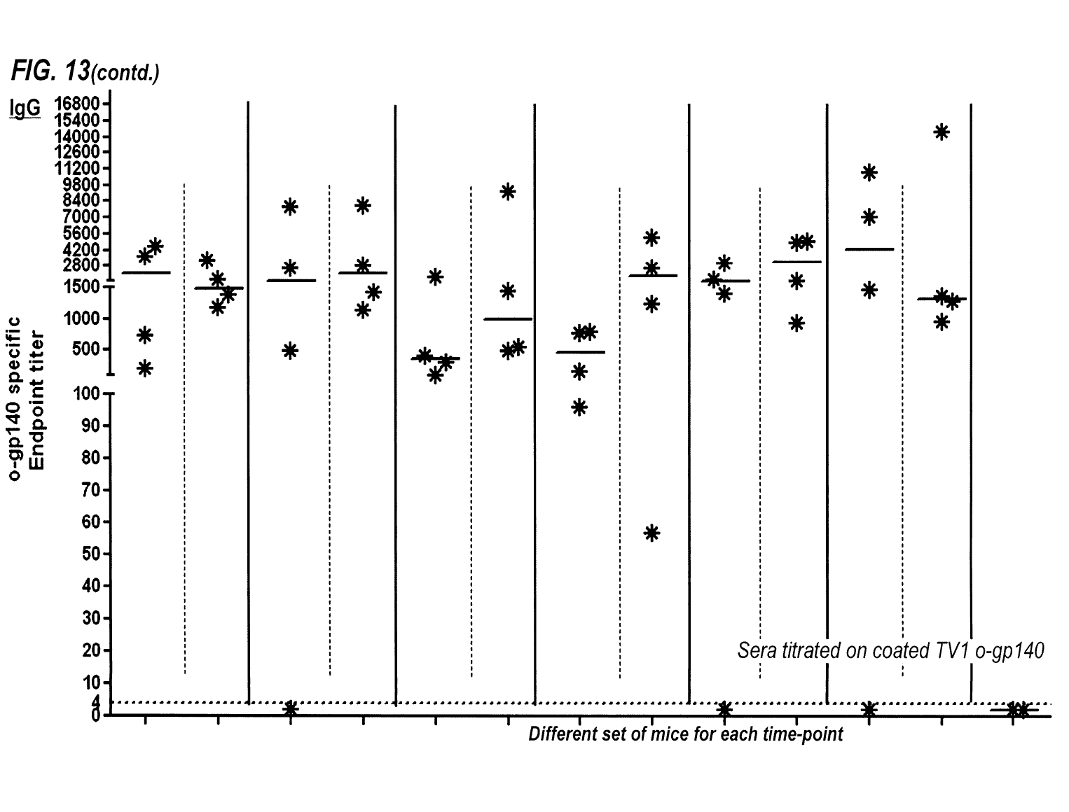

FIG. 13 shows the titers of gp140-specific IgA in vaginal washes of the mice administered with gp140 DNA or RNA vaccines. In this study, no protein/MF59 boost was administered. Notably, very low level of IgA was detected in group administered with DNA/Liposome.

### Example III

### Potency of an HIV-SAM™ Vaccine in a Heterologous Prime-Boost Vaccination Regimen

Recombinant alphavirus replicon particles (VRP), carrying self-amplifying RNA, protected rhesus macaques against SHIVSF162P4 challenge when used in a prime-boost regimen. A SAM™ vaccine platform, which is based on synthetic self-amplifying RNA that avoids limitations of cell culture production and employs synthetic non-viral vaccine delivery systems, was used.

Systemic and mucosal immune responses in mice and rabbits were evaluated using the SAM™ platform expressing HIV-1 gp140 (HIV-SAM™ vaccine) prime, protein/MF59 vaccine boost regimen for both HIV-1 Clade B and C Env antigens. In mice, the primed Env-specific IgG response to 1 μg of the HIV-SAM™ vaccine was comparable to a 10 μg dose of an identically formulated DNA vaccine, 10⁷ IU of VRP, and 10 μg protein/MF59 vaccines. The HIV-SAM™ vaccine primed response could be boosted robustly by a protein/MF59 vaccine and resulted in a balanced IgG1, IgG2a subclass response, similar to that seen with the VRP vaccine, but unlike the dominant IgG1 response to protein/MF59 only vaccinations. Both Env-specific CD4⁺ and CD8⁺ T-cell responses were detectable after two HIV-SAM™ vaccinations. A TH1 type (IFNγ⁺, IL-5⁻) profile was demonstrable for the HIV-SAM™ vaccine primed, protein boosted CD4⁺ T-cell response, similar to that seen with the DNA or VRP primed protein boosted responses, in contrast to a TH2 type (IFNγ^{low}, IL-5⁺) response seen with protein/MF59 vaccination. In rabbits, priming with the 25 or 50 μg of the formulated HIV-SAM™ vaccine induced robust and avid Env-binding IgG and HIV neutralizing antibodies that were superior to 500 μg of an unformulated DNA vaccine and comparable to VRP and protein/MF59 vaccines. In addition, protein/MF59 boostable Env-specific vaginal wash Ig was consistently demonstrable in both mice and rabbits immunized with the HIV-SAM™ vaccine.

Together, these results show that HIV-SAM™ vaccine is potent and versatile and offers a novel immune priming strategy.

### Example IV

### Dosing Studies in Rabbits Using 5, 25 and 50 μg Dose of CNE-RNA Vaccine

Dosing studies in rabbits using 5, 25 and 50 μg dose of the CNE-RNA vaccine (delivering a Clade C TV1.0 oligomeric gp140 protein) were conducted and the immunogenicity was compared to that induced by LNP-RNA, VRP and MF59-adjuvanted-o-gp140 vaccines. A prime-boost vaccination regimen was used with the rabbits primed at 0 and 4 weeks and boosted at 12 and 24 weeks.

TABLE IV-1

| | | | Boost I/M |
|---|---|---|---|
| | | Prime I/M | (12, 24 w) - 0.5 ml |
| Group | n | (0, 4 w) - 0.5 ml (single site) | (single site |
| Gp1 | 5 | RNA-LNP (5 μg) | o-gp140/MF59 (25 μg) |
| Gp2 | 5 | RNA-LNP (25 μg) | o-gp140/MF59 (25 μg) |
| Gp3 | 5 | RNA-LNP (50 μg) | o-gp140/MF59 (25 μg) |
| Gp4 | 5 | RNA-CMF34 (5 μg) | o-gp140/MF59 (25 μg) |
| Gp5 | 5 | RNA-CMF34 (25 μg) | o-gp140/MF59 (25 μg) |
| Gp6 | 5 | RNA-CMF34 (50 μg) | o-gp140/MF59 (25 μg) |
| Gp7 | 5 | RNA-(naked) (50 μg) | o-gp140/MF59 (25 μg) |
| Gp8 | 5 | DNA (500 μg) - no electroporation | o-gp140/MF59 (25 μg) |
| Gp9 | 5 | VRP-Env (10⁸) | o-gp140/MF59 (25 μg) |
| Gp10 | 5 | o-gp140/MF59 (25 μg) | o-gp140/MF59 (25 μg) |
| Gp11 | 5 | RNA-LNP (5 μg) intradermal | o-gp140/MF59 (25 μg) |
| | | injection (100 μl × 5 sites) | |

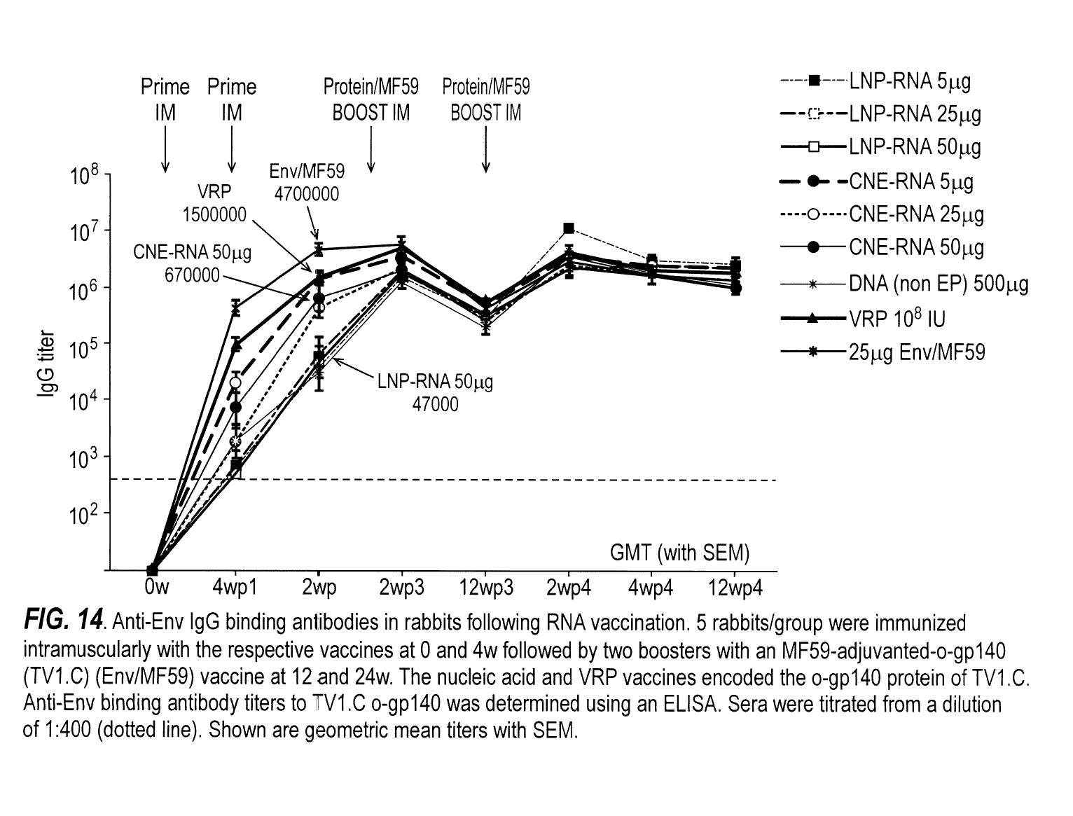

Robust Env-specific binding antibody titers were induced by both the CNE- and the LNP-RNA vaccines upon immunization in rabbits (FIG. 14). Two weeks after 2 primes (2wp2) the CNE-RNA vaccines induced titers that were, on average, ˜15-20-fold higher than the LNP-RNA vaccine, ˜10-40-fold higher than a 500 μg dose of a naked DNA vaccine and comparable to that seen with the VRP vaccine (FIG. 14). The response seen with the MF59-adjuvanted-o-gp140 vaccine was ˜4-10-fold higher than that seen with the CNE-RNA vaccine (FIG. 14). Upon boosting twice with a MF59-adjuvanted-o-gp140 vaccine a similar magnitude of response was achieved, for all vaccines (FIG. 14).

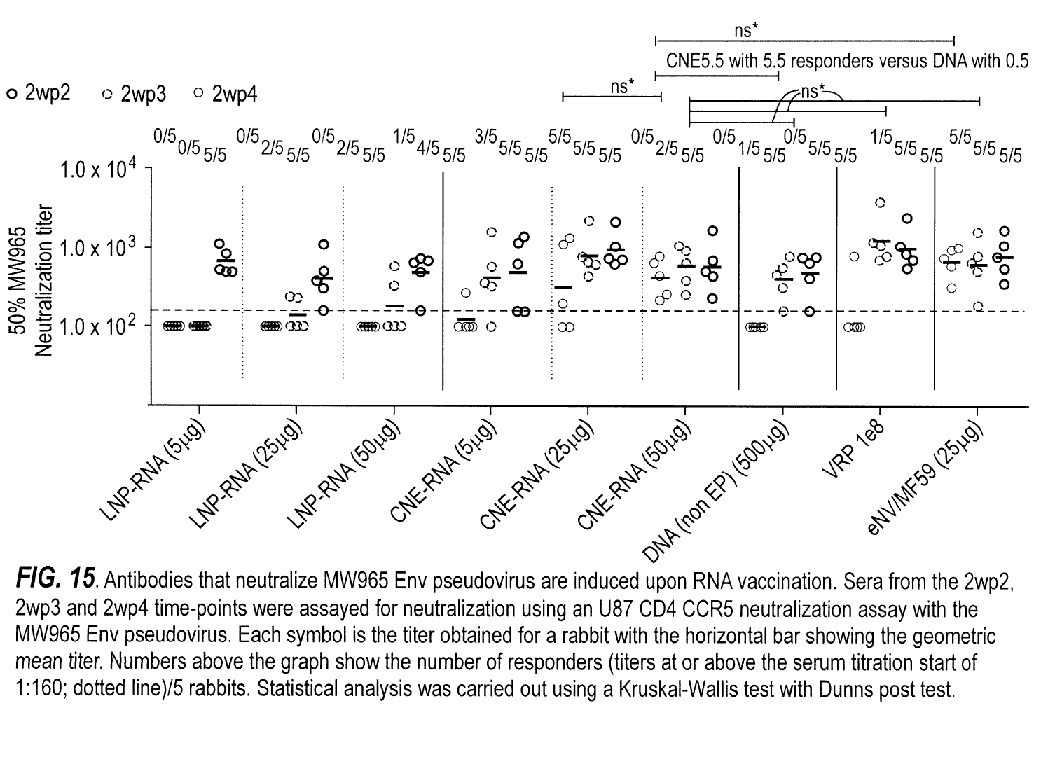

The CMF-34 vaccine, consistent to that that seen with binding antibodies, induced superior neutralizing antibodies after priming, in comparison to the LNP-RNA and DNA vaccines (FIG. 15). Thus, while sera from 5/5 animals immunized twice (2wp2) with the 50 μg dose of the CNE-RNA vaccine neutralized the Clade C virus MW965, no animals in the LNP-RNA high dose or the 500 μg DNA groups demonstrate neutralizing antibodies (FIG. 15). Upon boosting the LNP-RNA primed animals once with the MF59-adjuvanted-o-gp140 vaccine (2wp3), only 2/5 animals demonstrated neutralization and an additional boost (2wp4) was required to increase the number of responders to 100% (FIG. 15). Thus the CNE-RNA vaccine induced neutralizing antibodies earlier than that induced by an LNP-RNA vaccine in rabbits. The neutralizing response seen with the CNE-RNA vaccine was also comparable to the VRP and MF59-adjuvanted-o-gp140 vaccines (FIG. 15).

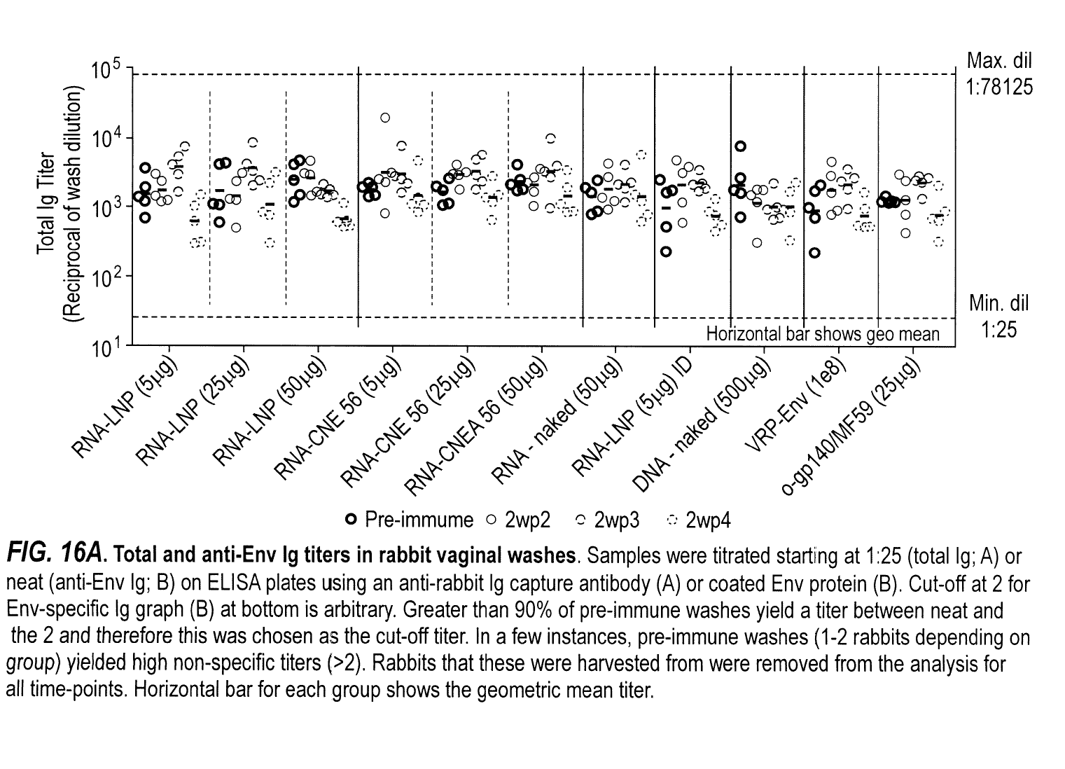

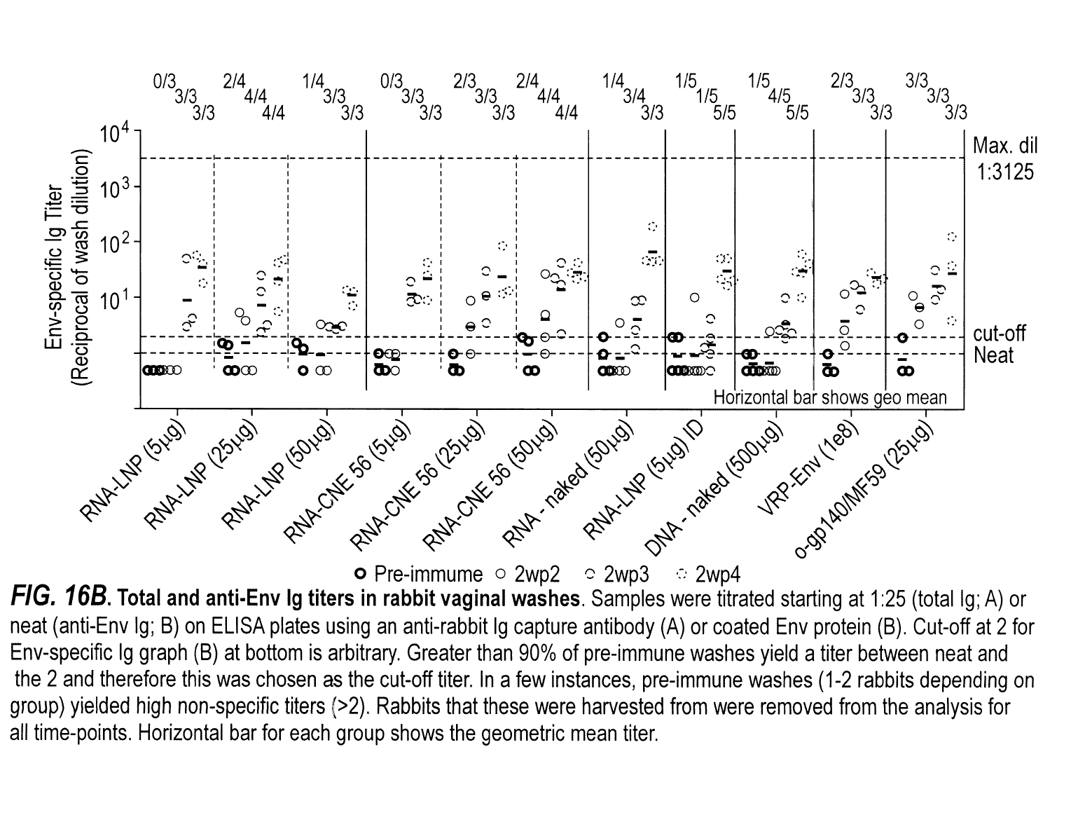

Vaginal washes from rabbits were also collected at 2wp2, 2wp3 and 2wp4. Washes were assayed for Env-specific Ig using an anti-rabbit Ig (H+L) antibody. Low level Env-specific Ig titers were demonstrated in 50-100% of the CNE-RNA and LNP-RNA vaccinated groups, with evidence of Env protein boosting of the primed responses such that 100% of the animals showed vaginal responses (FIG. 16).

### Example V

### Immunogenecity Profile of Vaccines in Rhesus Macaques

A prime-boost vaccination regimen was used in a study of rhesus macaques whereby the primates were primed at 0, 4 and 12 weeks followed by boosting at 24, 36 and 54 weeks. Challenge can be effected using SHIV1157ipd3N4.

TABLE V-1

| | | | | Challenge (IR) - |
|---|---|---|---|---|
| | | | | low dose |
| | | | Boost 24, | SHIV1157ipd3N4 |
| Group | n | Prime 0, 4, 12 w | 36, 54 w | Repeat (5x) 62 w |
| 1 | 6 | VRP | Env/MF59 | ✓ |
| | | 10⁸ IU | 100 μg | |
| 2 | 6 | HIV-SAM vaccine/LNP | Env/MF59 | ✓ |
| | | 50 μg | 100 μg | |
| 3 | 6 | HIV-SAM vaccine/CNE | Env/MF59 | ✓ |
| | | 50 μg | 100 μg | |
| 4 | 6 | Env/MF59 | Env/MF59 | ✓ |
| | | 100 μg | 100 μg | |
| 5a | 3 | VRP gH/gL_{(CMV)} | gH/gL_{(CMV)}/ | ✓ |
| | | 10⁸ IU | MF59 | |
| | | | 100 μg | |
| 5b | 3 | HIV-SAM vaccine/LNP | gH/gL/MF59 | ✓ |
| | | (gH/gL) | 100 μg | |
| | | 50 μg | | |

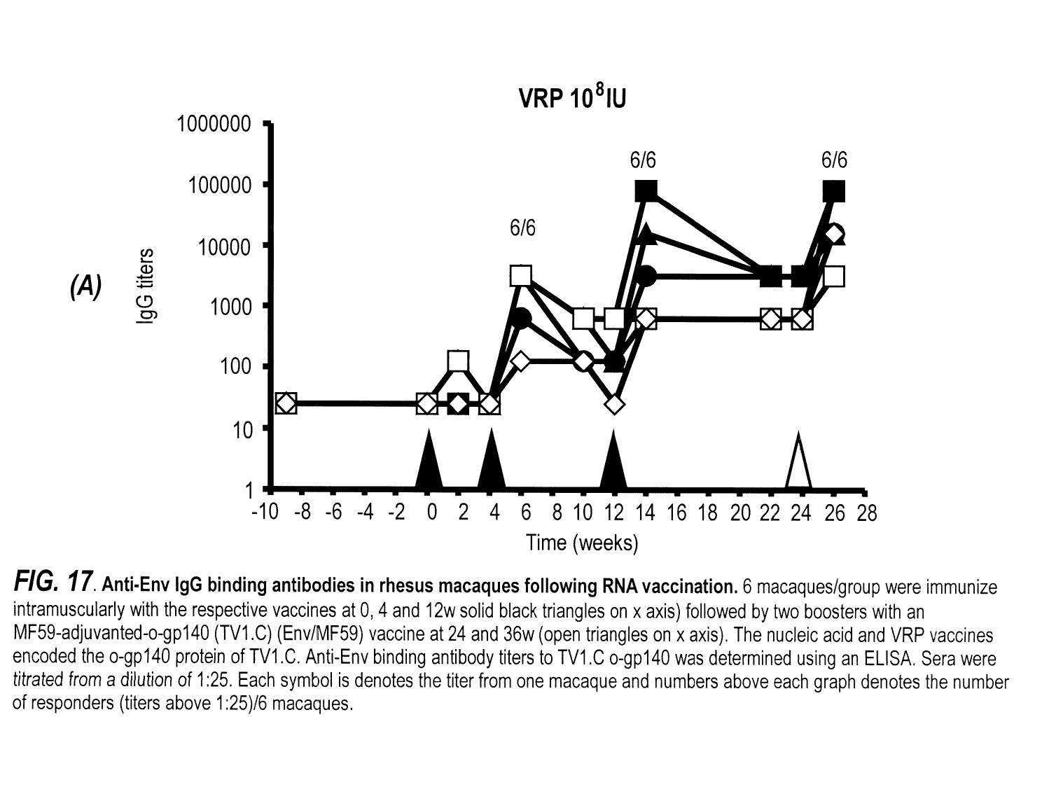

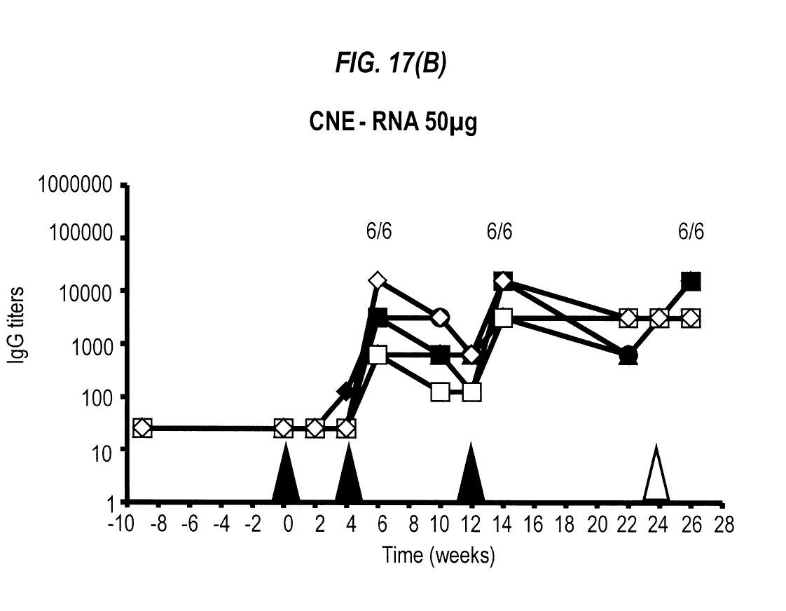

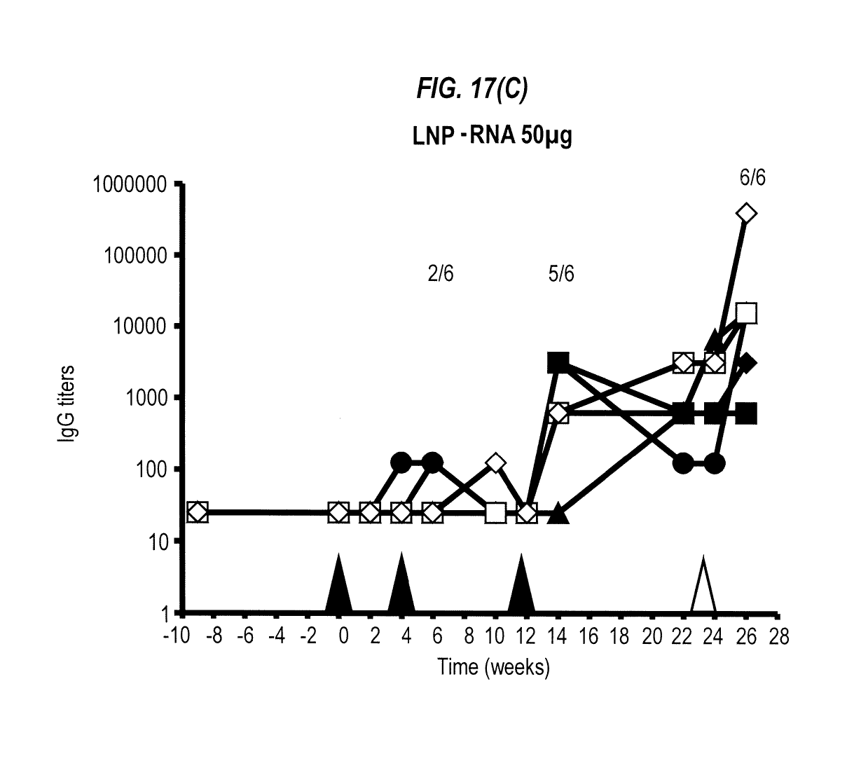

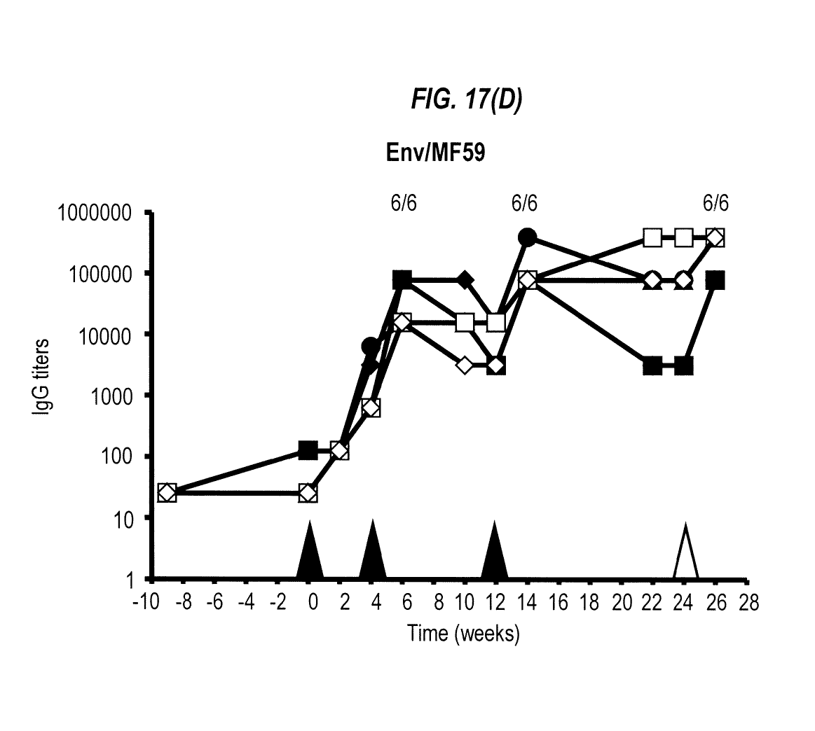

The immunogenicity profile of the vaccines in rhesus macaques was similar to that seen in rabbits. In these studies, primates were primed thrice followed by two MF59-adjuvanted-o-gp140 boosts. After two priming immunizations with the CNE-RNA vaccine all 6 animals responded with anti-Env binding titers in the range of ˜1000-10000 (2wp2, week 6) (FIG. 17). This response was comparable to that seen with the VRP vaccine at the same time-point, which induced ˜10-fold lower titers (titer range ˜100-1000) (FIG. 17). In contrast, only 2/6 animals responded to the LNP-RNA vaccine with anti-Env titers (titers of ˜100) (FIG. 17). An additional immunization of the LNP-RNA vaccine was required to induce a response in 5/6 animals (2wp3, week 14) (FIG. 17). Boosting the LNP-RNA primed response with the MF59-adjuvanted-o-gp140 vaccine resulted in anti-Env titers demonstrable in all 6 animals (2wp4 week 26), but with a wide titer range (range ˜1000-500000) (FIG. 17). In contrast, the CNE-RNA primed MF59-adjuvanted-o-gp140 vaccine boosted anti-Env titers were tightly clustered (range ˜5000-10000) and comparable to that induced by the VRP vaccine (FIG. 17).

Mean titers of binding IgG antibodies against TV1 gp140 were measured. Five of the six animals responded to the LNP-RNA vaccine with anti-TV1 titers. An additional immunization of the LNP-RNA vaccine was required to induce a response in all six animals. In contrast, the CNE-RNA primed vaccine boosted anti-TV1 titers were tightly clustered and titers were even higher than those induced by the VRP vaccine.

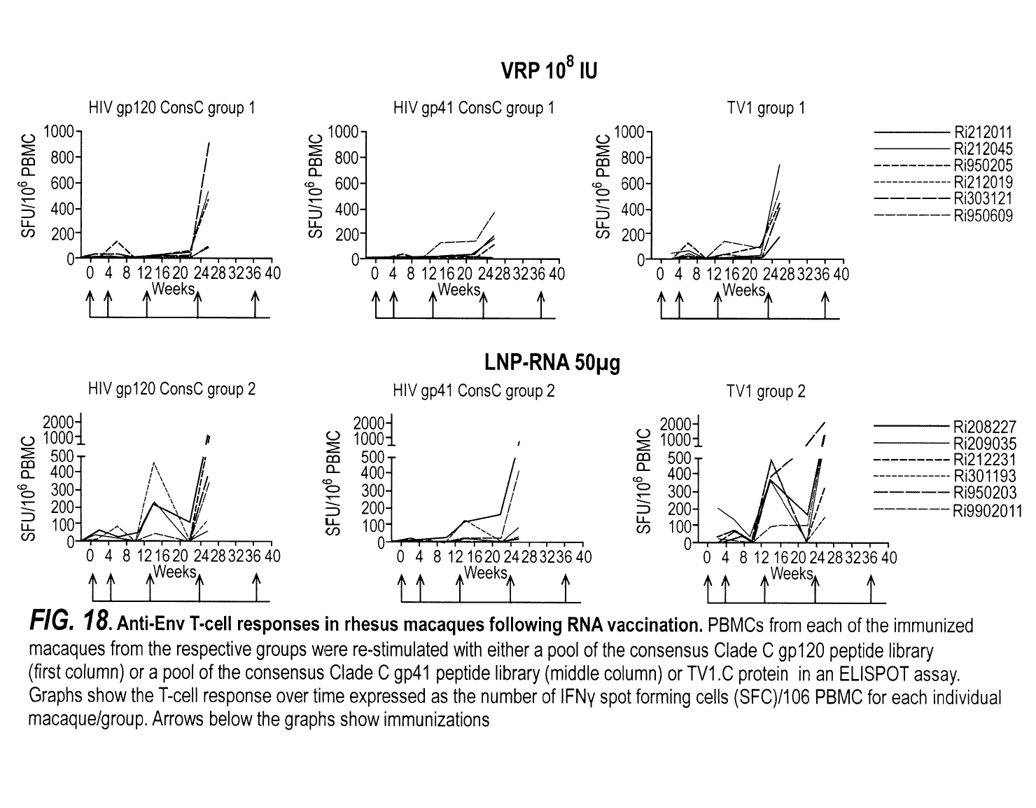

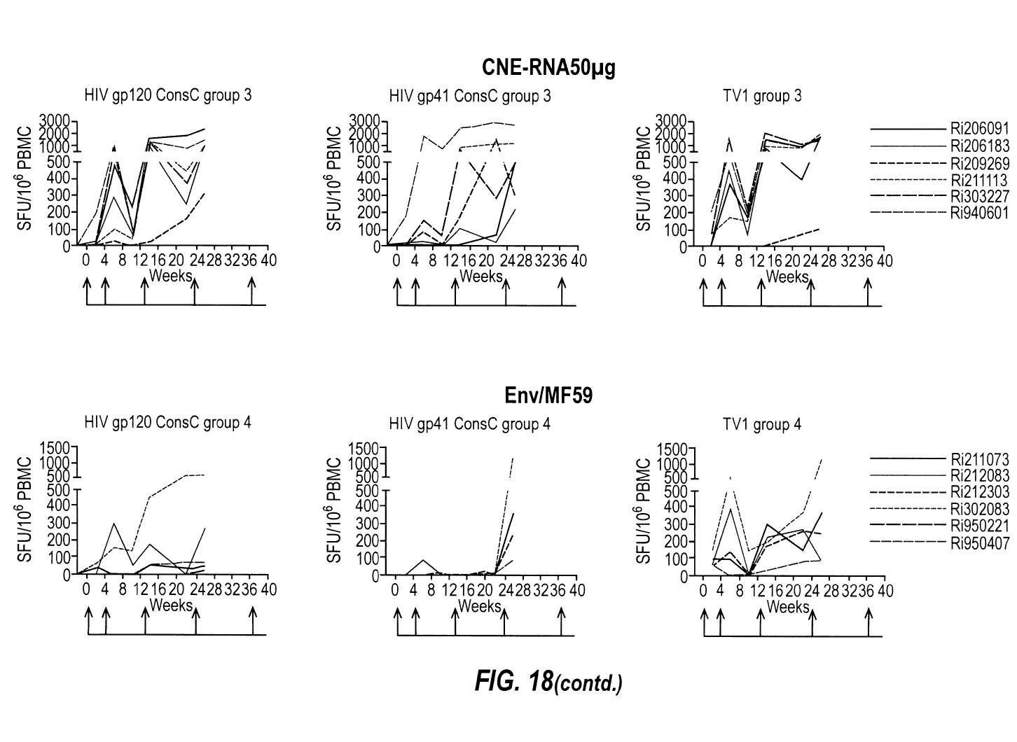

Both the CNE-RNA and the LNP-RNA vaccines induced T-cell responses (as measured by ex vivo peptide and protein re-stimulation ELISPOT assays). Two weeks after the 1st MF59-adjuvanted-o-gp140 boost (2wp4, week 26), 6/6 animals demonstrated Env-specific T-cell IFNγ responses with one low responder (˜300 SFC/10⁶ PBMC) and 5 high responders (˜1000-2000 SFC/10⁶ PBMC) (FIG. 18). In contrast, a scattered T-cell response was demonstrable upon boosting the with the LNP-RNA primed responses with a range of ˜50-1000 (FIG. 18). Weak T-cell responses were seen when priming with the VRP vaccine and a scattered response was achieved after boosting (range ˜100-900 SFC/10⁶ PBMC) (FIG. 18). Weak T-cell responses were also observed with the MF59-adjuvanted-o-gp140 vaccine (FIG. 18). Both nasal and rectal washes have been collected from various time-points after priming with the RNA vaccines and boosting with MF59-adjuvanted o-gp140. Env-specific IgA binding titers are being assessed, using an ELISA.

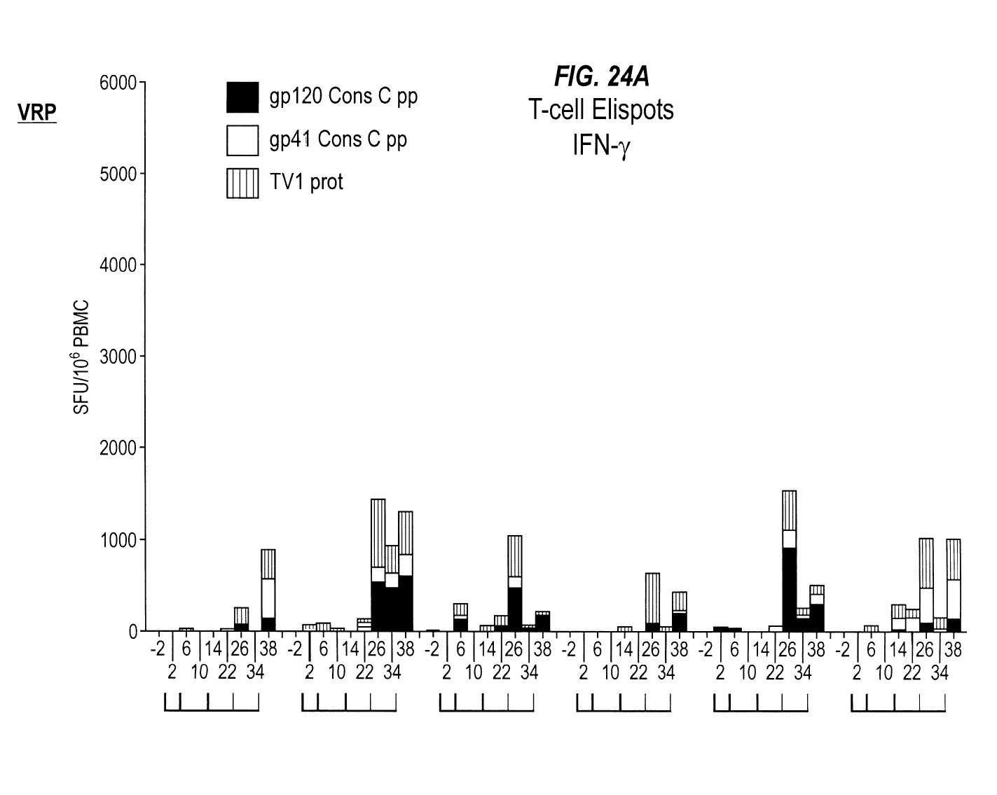

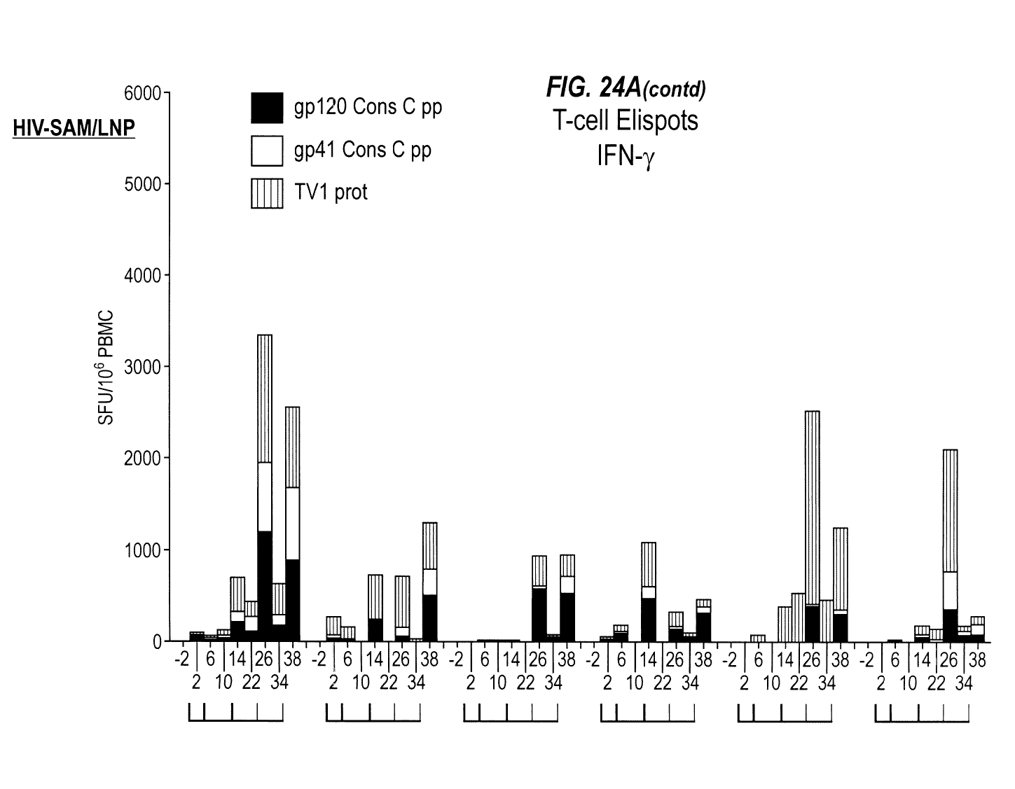

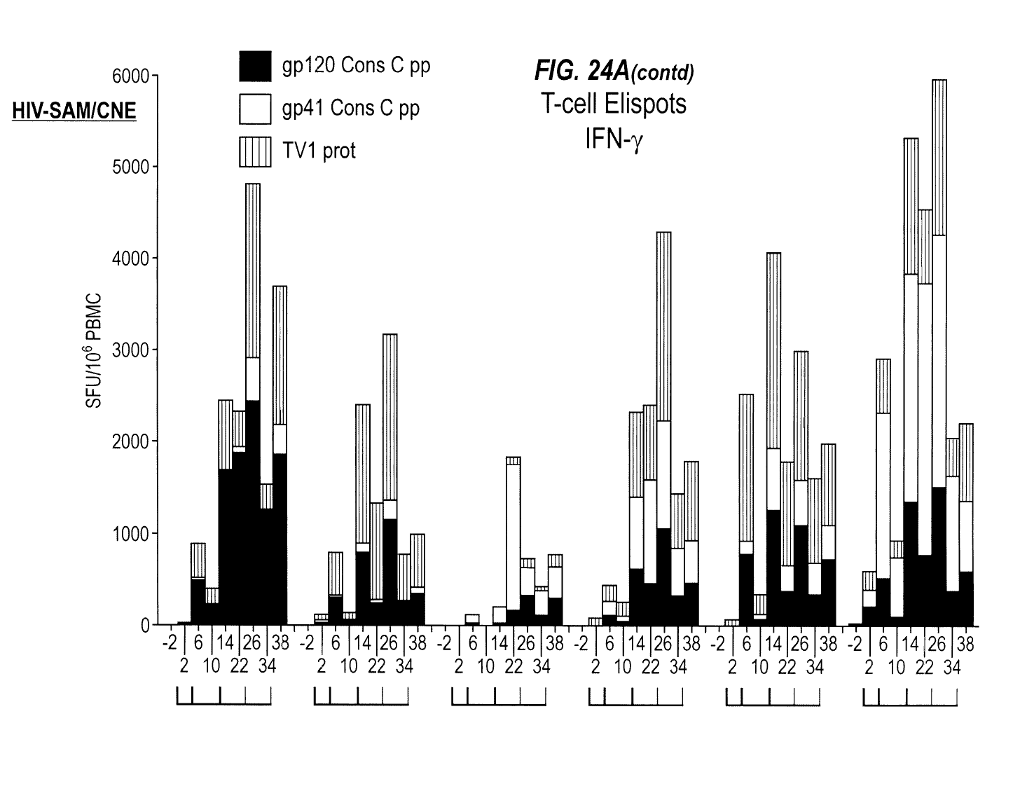

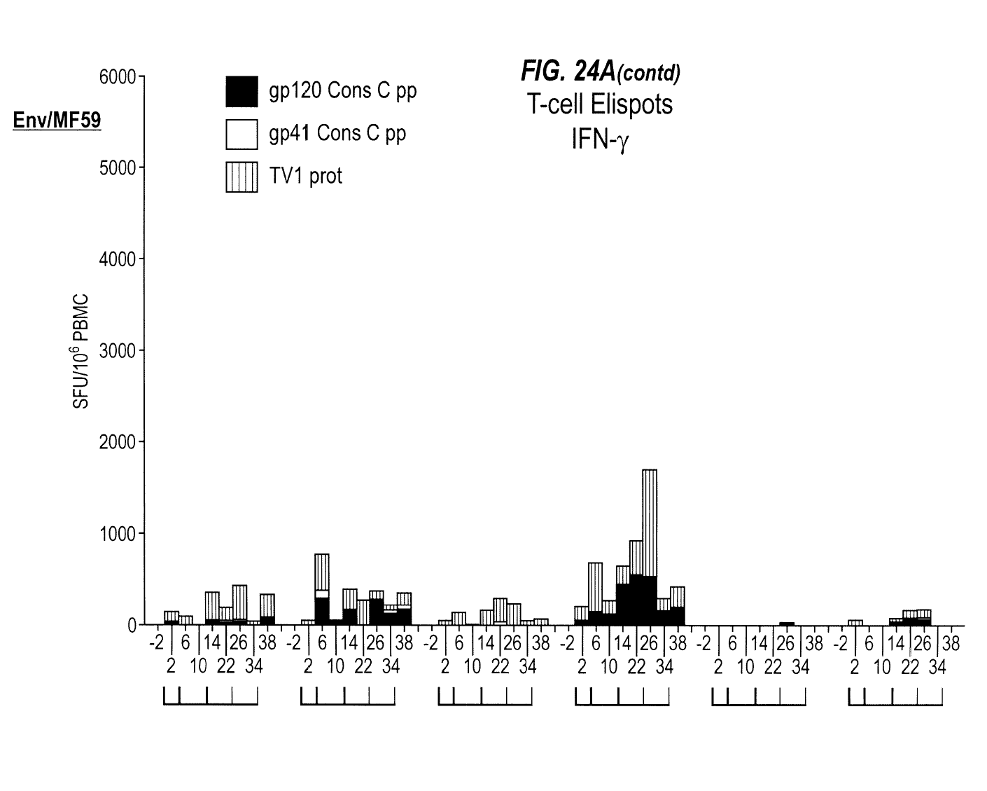

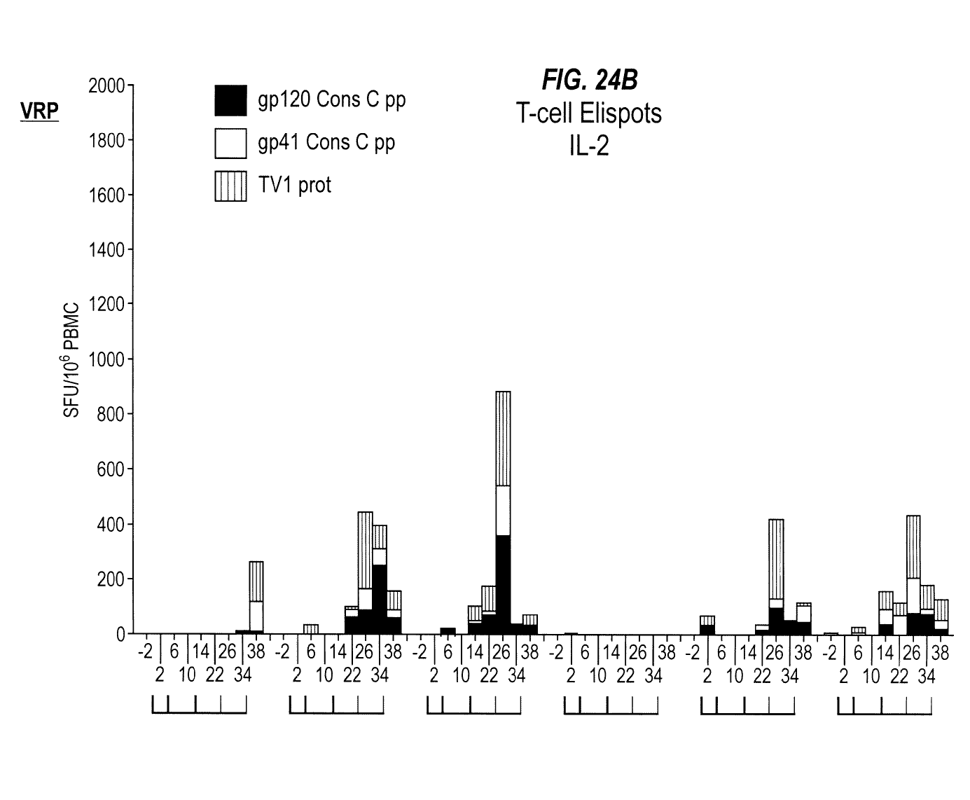

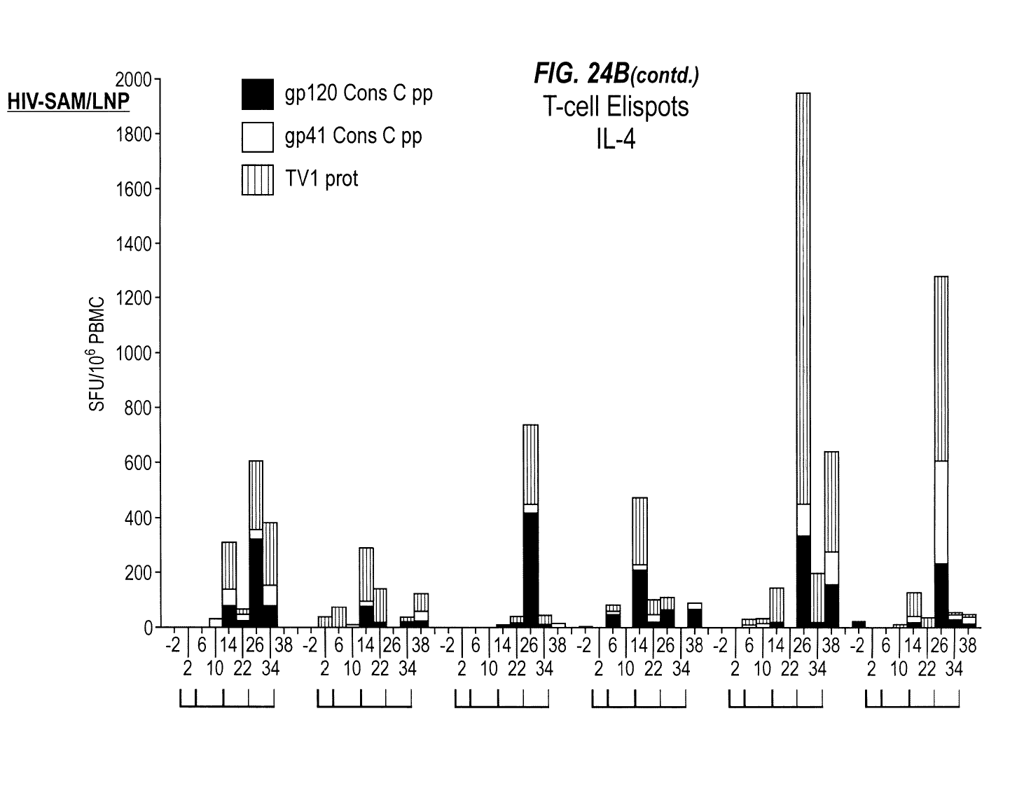

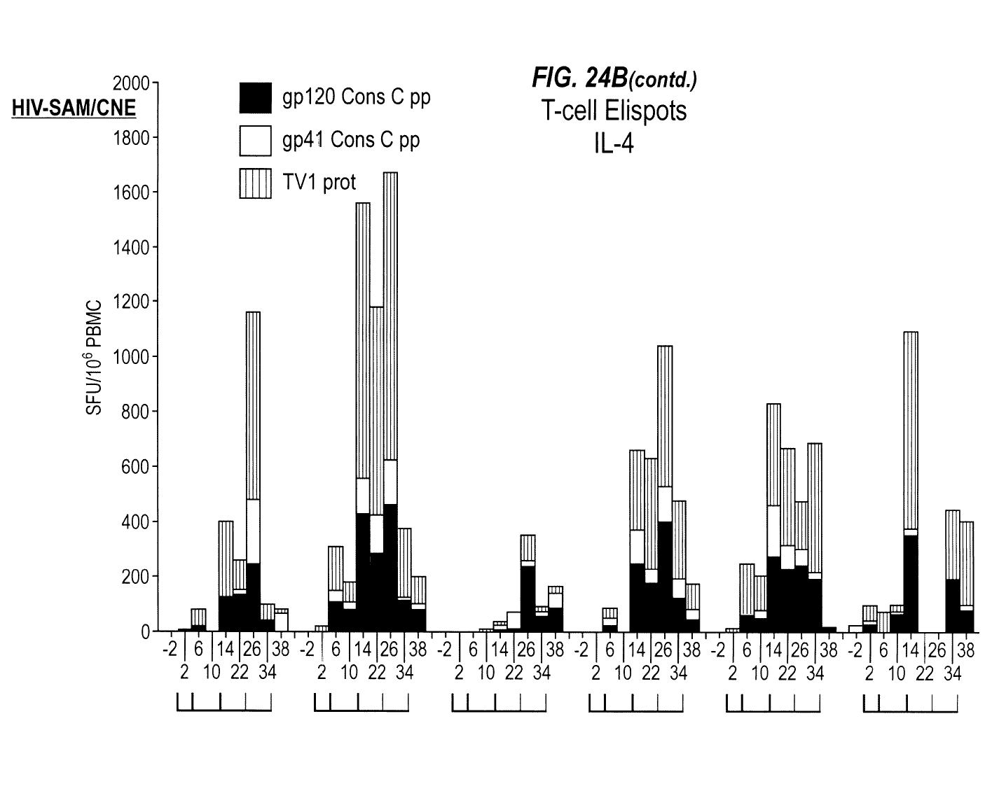

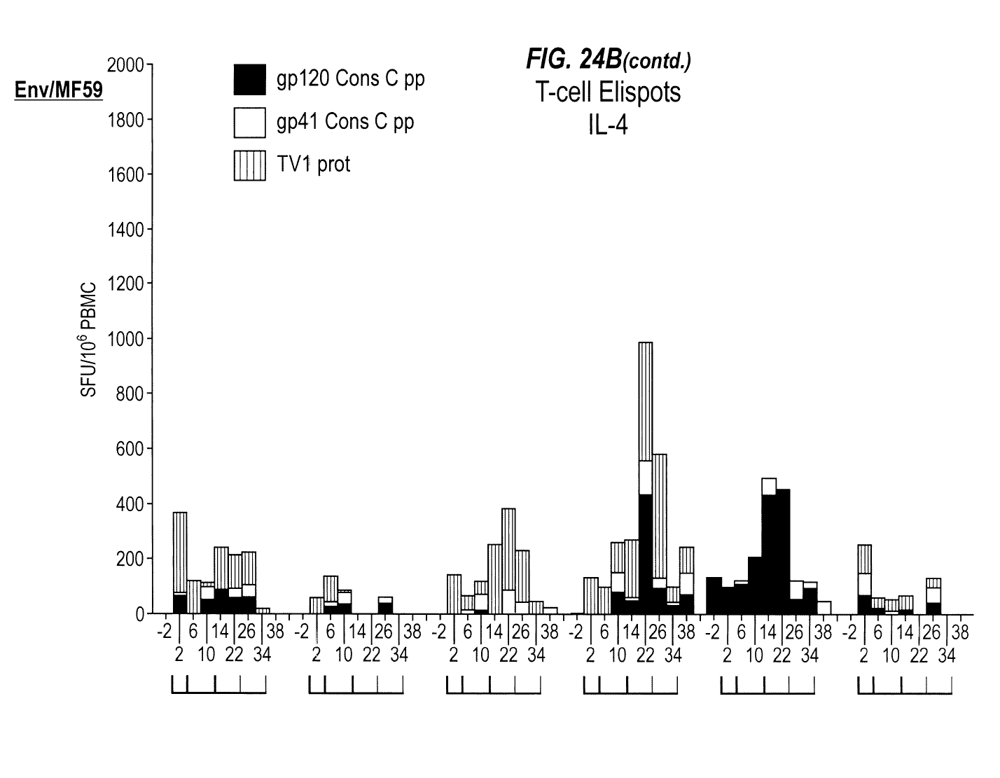

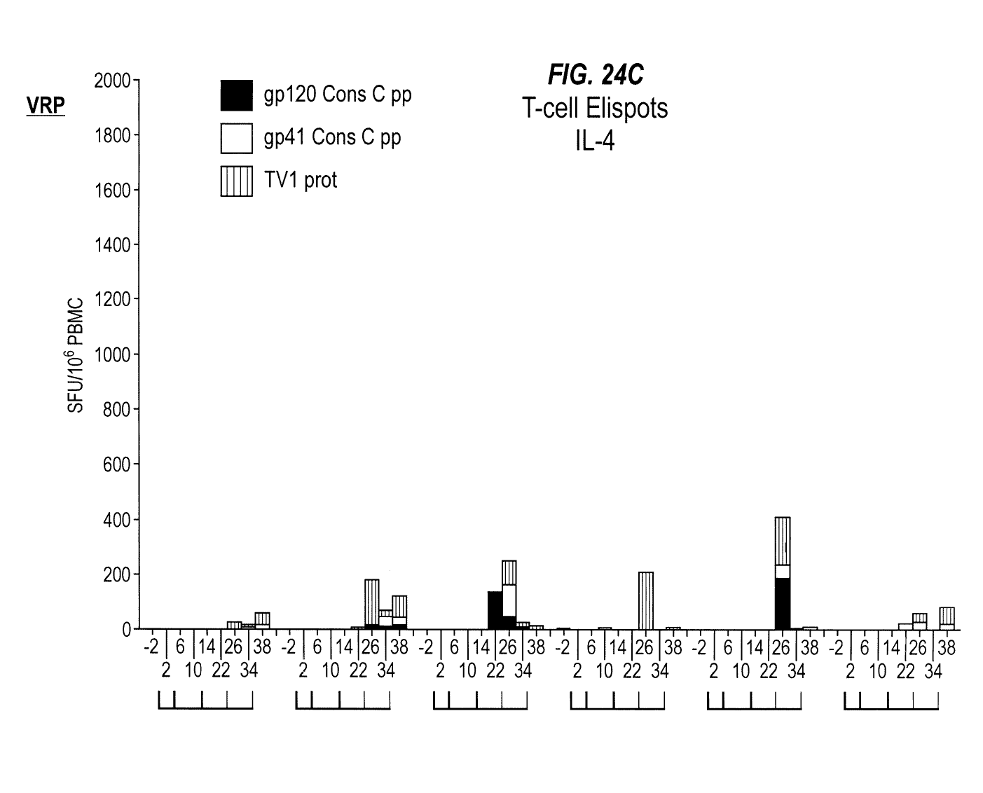

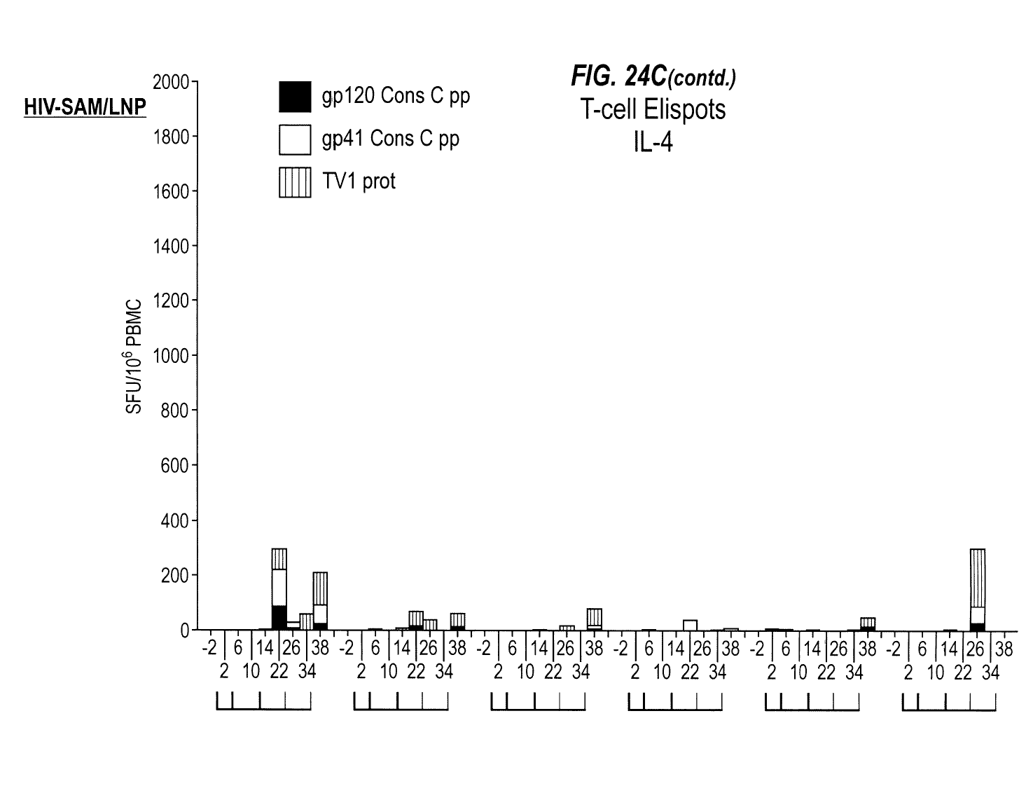

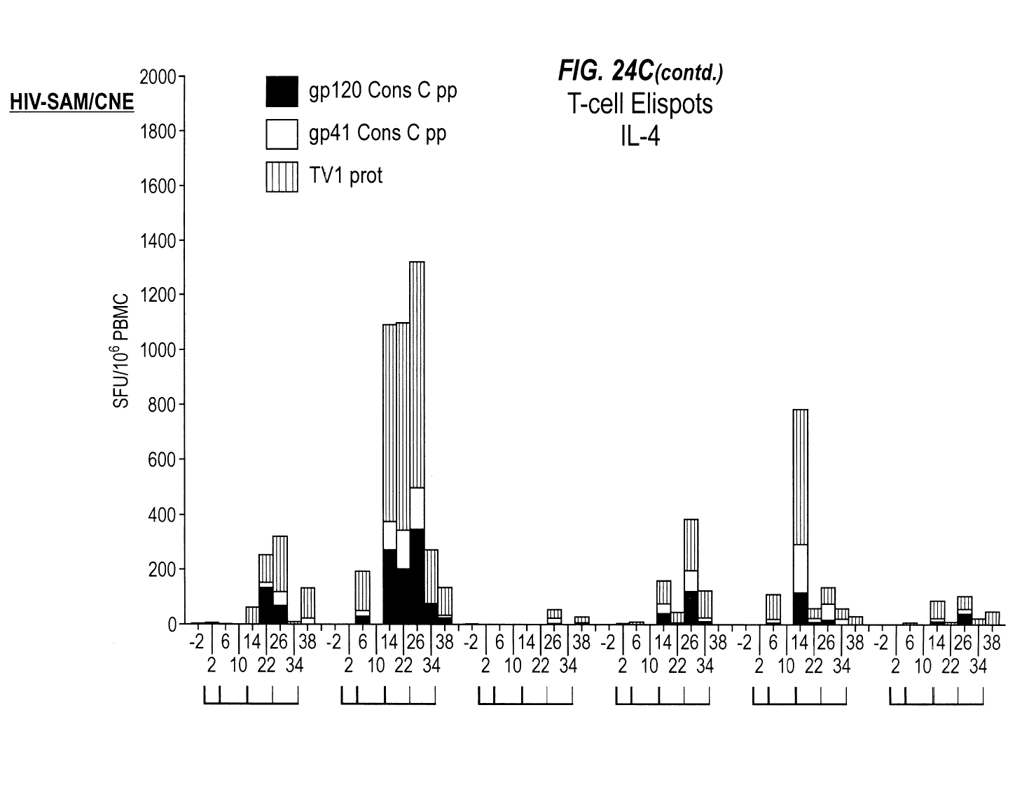

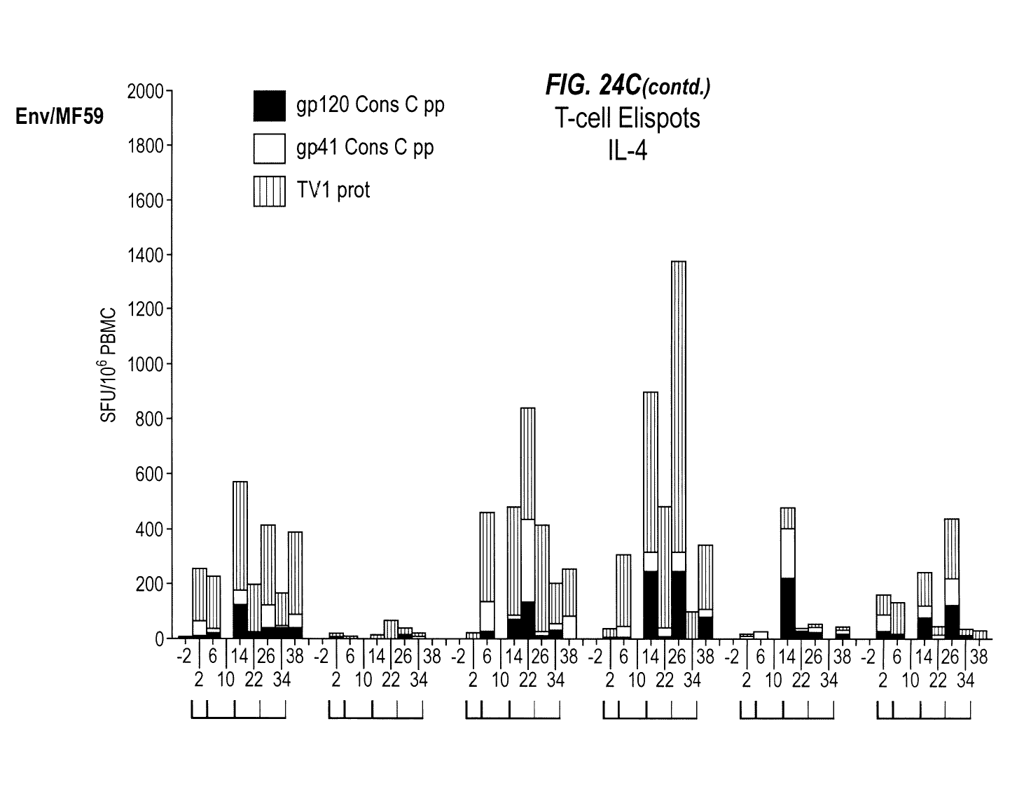

Vaccine induced antigen-specific T cell responses for IFNγ, IL2 and IL4 responses were measured in time. IFNγ, IL2, and IL4 secretion by PBMC of all individual animals per group towards gp120 Consensus C peptide pool (pp), gp41 Cons C pp, or recombinant TV 1 gp140 were measured by ELISpot assay (FIGS. 24A-C). Strong responses were seen for RNA-CNE and RNA-LNP when IFN-γ was measured (FIG. 24A). A scattered response was seen for IL-2 (FIG. 24B) and IL4 (FIG. 24C).

Both the CNE-RNA and LNP-RNA vaccines induced B-cell responses (as measured by ELISpot assays). Two weeks after the 1st MF59-adjuvanted-o-gp140 boost (2wp4, week 26), 6/6 animals demonstrated antigen specific-specific B-cell responses with one low responder (˜300 SFC/10⁶ PBMC) and 5 high responders (˜1000-2000 SFC/10⁶ PBMC) (FIG. 18).

Similar results were seen with CNE-RNA and LNP-RNA vaccines induced B-cell responses (as measured by ELISpot assays that were TV1-gp140 specific). Two weeks after the first MF59-adjuvanted-o-gp140 boost (2wp4, week 26), 4/6 animals primed with LNP-RNA demonstrated antigen specific B-cell responses, and 6/6 animals primed with CNE-RNA demonstrated antigen specific B-cell responses.

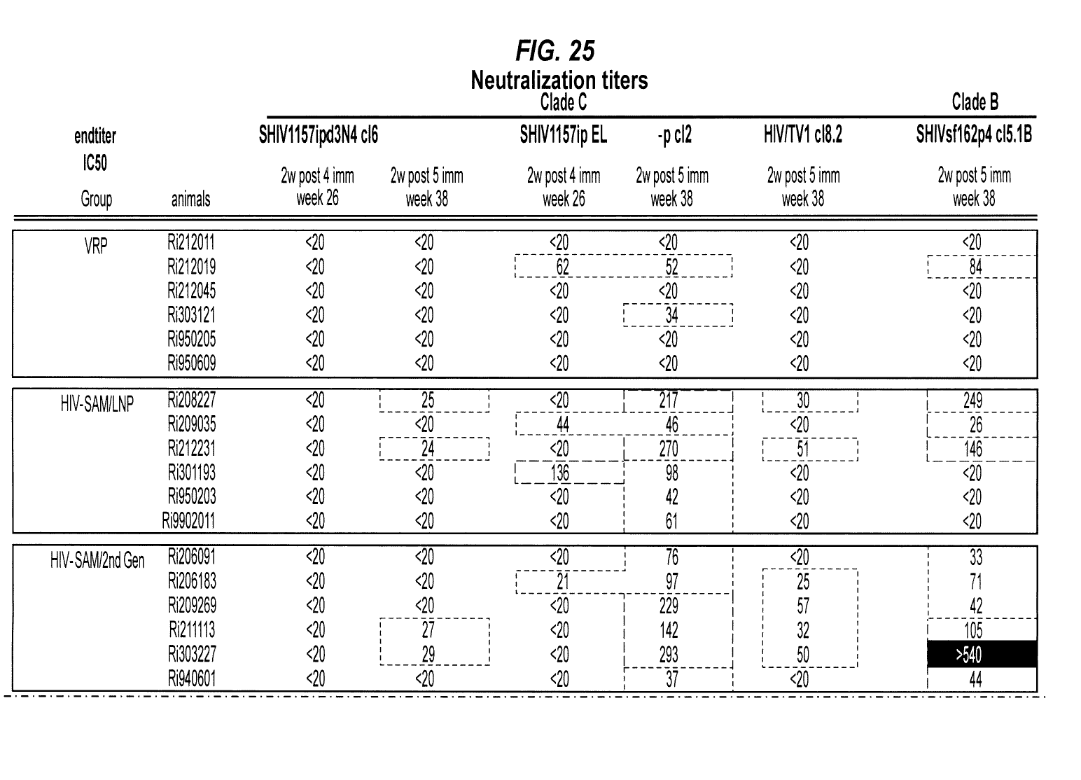

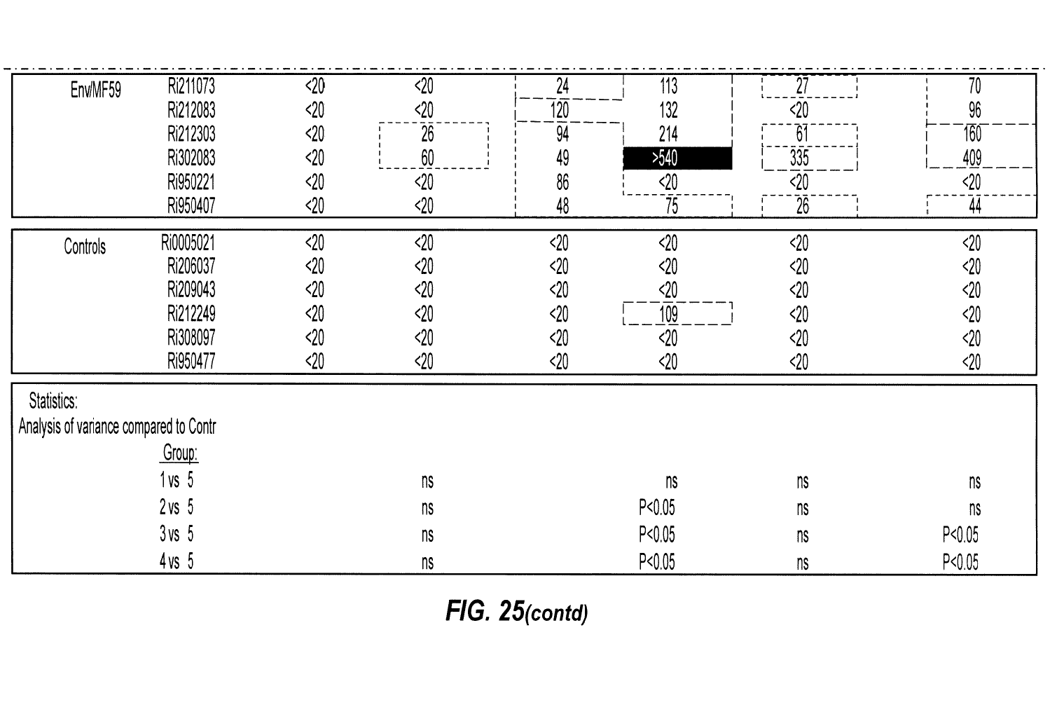

Neutralization (IC50) assays were performed on sera taken at two weeks post 4th (wk 26) and two weeks post 5th (wk 38) immunization (FIG. 25). Sera were evaluated against a clade C Tier 2 (SHIV1157ipd3N4) Pseudovirus, a Tier 1 (SHIV1157ipEL-p) PV, a Tier 1 HIV-1/TV1 PV and against a Tier 1 Clade B PV (SHIV SF162P4). Large neutralization titers were seen in 2/6 RNA-LNP primed animals, 3/6 CNE-RNA primed animals and 4/6 protein/MF59 primed animals in sera evaluated against the Tier 1 (SHIV1157ipEL-p) at week 38.

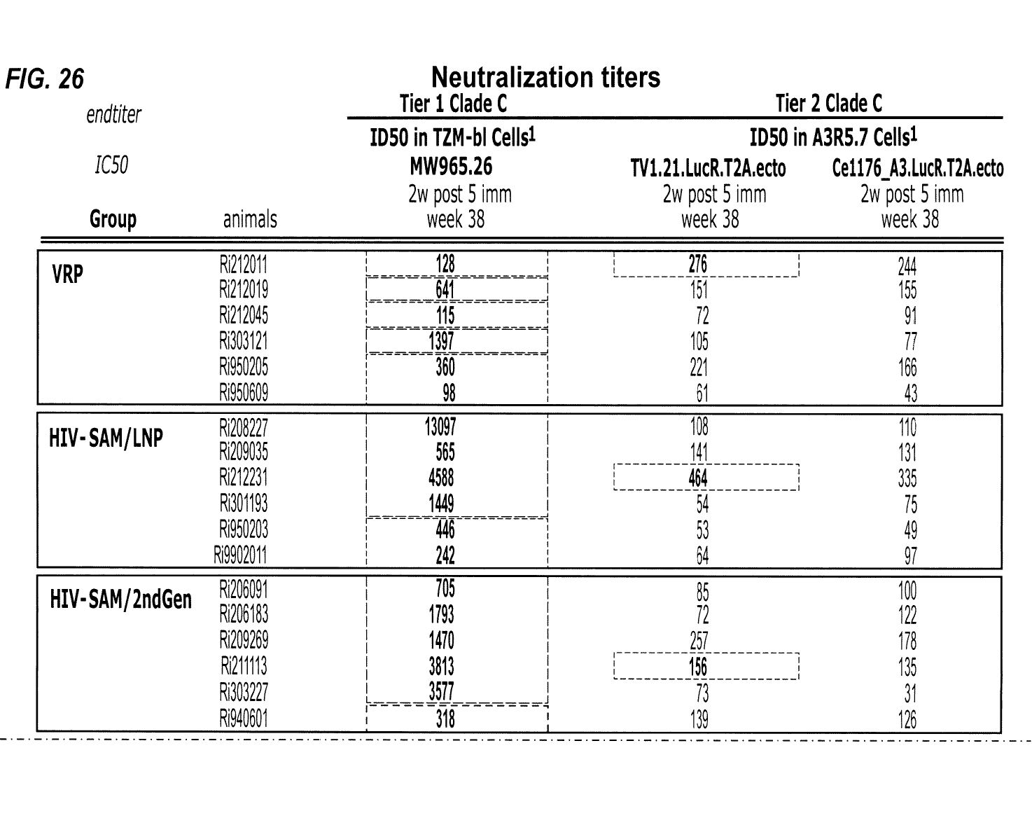

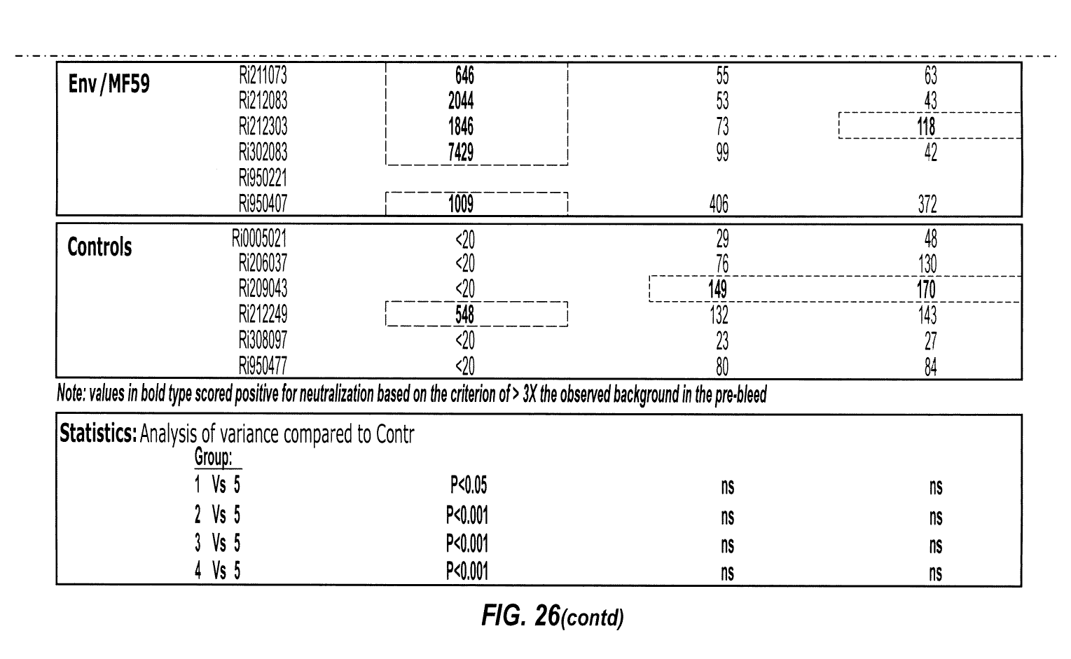

Additional neutralization (IC50) assays were performed on sera taken at two weeks post 5th (wk 38) immunization. These sera were evaluated against a clade C Tier 1 (MW965.26) in TZM-bl cells and Tier 2 viruses (TV1.21.LucR.T2A.ecto and Cel 176_A3.LucR.T2A.ecto) in A3R5.7 cells (FIG. 26). 6/6 animals for each of VRP, RNA-LNP, RNA-CNE, and Protein/MF59 scored positive for neutralization based on the criterior on >3× the observed background in the pre-bleed for sera tested against Tier 1 (MW965.26).

For most antigens, binding responses peaked at week 38 (post 2^{nd} boost). It was also shown that CNE-RNA primes elicited higher binding responses to both V1/V2 and envelope antigens. Further, after protein boosts (week 38 and week 56), LNP-RNA and CNE-RNA groups developed significantly higher binding antibodies against the o-gp140 groups than the VRP and Env groups.

These studies provide the first evidence in nonhuman primates that vaccination with formulated self-amplifying RNA is safe and immunogenic, eliciting both humoral and cellular immune responses.

### Example VI

### HIV Prime-Boost v. Concurrent Administration of HIV-SAM_{gp140} Vaccine/CMF34 and Env Protein (TV1 gp140)

In this example, antibody responses to HIV Env using prime boost was compared against concurrent administration. The study also compared the use of MF59 against alum for prime boost versus concurrent, and compared use of no adjuvant or alum against MF59 for concurrent administration. An objective of this example was to benchmark prime boost and concurrent administration with single modality immunizations.

Various HIV-SAMgp140/CMF34 and Env protein (TV1 pg140) formulations were administered to rabbits according to Table VI-1 below

TABLE VI-1

| Group | Rabbits | Injections | Antigen | Dose | |
|---|---|---|---|---|---|
| 1 | 6 | 4 | HIV-SAM_{gp140}/CNE | 25 | μg |
| 2 | 6 | 4 | HIV-SAM_{gp140}/CNE prime (2) + TV1 gp140 + MF59 | 25 + 25 | μg |
| | | | boost (2) | | |
| 3 | 6 | 4 | HIV-SAM_{gp140}/CNE prime (2) + TV1 gp140 + Alum | 25 + 25 | μg |
| | | | boost (2) | | |
| 4 | 6 | 4 | HIV-SAM_{gp140}/CNE + TV1 gp140 (same side, two | 25 + 25 | μg |
| | | | sites) | | |
| 5 | 6 | 4 | HIV-SAM_{gp140}/CNE + TV1 gp140 + MF59 (same side, | 25 + 25 | μg |
| | | | two sites) | | |
| 6 | 6 | 4 | HIV-SAM_{gp140}/CNE + TV1 gp140 + Alum (same side, | 25 + 25 | μg |
| | | | two sites) | | |
| 7 | 6 | 4 | HIV-SAM_{gp140}/CNE + TV1 gp140 + MF59 (opposite | 25 + 25 | μg |
| | | | sides) | | |
| 8 | 6 | 4 | HIV-SAM_{gp140}/CNE + TV1 gp140 + Alum (opposite | 25 + 25 | μg |
| | | | sides) | | |
| 9 | 6 | 4 | TV1 gp140 + MF59 | 25 | μg |
| 10 | 6 | 4 | TV1 gp140 + Alum | 25 | μg |

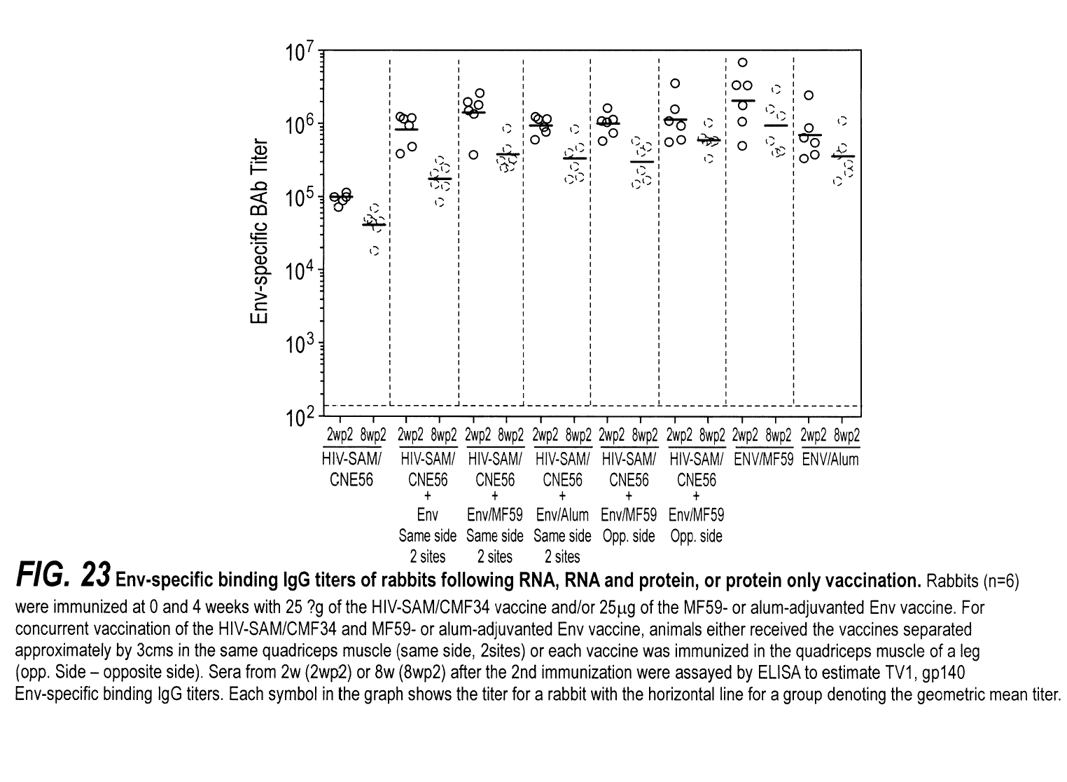

A prime-boost vaccination regimen was used with the rabbits primed at 0 and 4 weeks and boosted at 12 and 24 weeks. Serum as well as vaginal wash and fecal pellet samples were collected at various time points. Env-specific binding IgG titers are shown in FIG. 23. Antibody responses to HIV Env were comparable between prime boost and concurrent administration subjects. No significant difference was observed between rabbits receiving vaccine with no adjuvant, alum or MF59, in prime boost or concurrent administrations.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. An immunogenic composition comprising:
(i) a self-replicating RNA molecule that encodes a first polypeptide antigen comprising a first epitope; and
(ii) a second polypeptide antigen comprising a second epitope;
wherein said first epitope and second epitope are epitopes from human immunodeficiency virus (HIV).

2. The immunogenic composition of claim 1, wherein said first epitope and second epitope are the same epitope.

3. The immunogenic composition of claim 1, wherein said first epitope and second epitope are different epitopes.

4. The immunogenic composition of claim 1, wherein said first polypeptide antigen and second polypeptide antigen are substantially the same.

5. The immunogenic composition of claim 1, wherein said first polypeptide antigen is a soluble or membrane anchored polypeptide, and said second polypeptide antigen is a soluble polypeptide.

6-8. (canceled)

9. The immunogenic composition of claim 1, wherein the self-replicating RNA is an alphavirus-derived RNA replicon.

10. (canceled)

11. The immunogenic composition of claim 1, further comprising a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, or a cationic nanoemulsion.

12. (canceled)

13. The immunogenic composition of claim 1, wherein the HIV antigens are independently selected from the group consisting of gp 160, gp140 and gp 120.

14. The immunogenic composition of claim 13, wherein the HIV antigens comprise an amino acid sequence selected from the group consisting of SEQ ID NOs: 4, 6, and 8.

15. The immunogenic composition of claim 1, further comprising an adjuvant.

16-17. (canceled)

18. A method for inducing an immune response against human immunodeficiency virus (HIV) in a subject, comprising administering to a subject in need thereof a therapeutically effective amount of a composition of claim 1.

19. (canceled)

20. A kit comprising:
(i) a priming composition comprising a self-replicating RNA molecule that encodes a first polypeptide antigen that comprises a first epitope from HIV; and
(ii) a boosting composition comprising a second polypeptide antigen that comprises a second epitope from HIV;
wherein said first epitope and second epitope are the same epitope.

21. The kit of claim 20, wherein said first polypeptide antigen and second polypeptide antigen are substantially the same.

22. The kit of claim 20, wherein said first polypeptide antigen is a soluble or membrane anchored polypeptide, and said second polypeptide antigen is a soluble polypeptide.

23-24. (canceled)

25. The kit of claim 20, wherein the self-replicating RNA is an alphavirus-derived RNA replicon.

26. (canceled)

27. The kit of claim 20, wherein the priming composition further comprises a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, and a polycationic peptide, or a cationic nanoemulsion.

28. (canceled)

29. The kit of claim 20, wherein the HIV antigens are independently selected from the group consisting of gp 160, gp140 and gp 120.

30. The kit of claim 29, wherein the HIV antigens comprise an amino acid sequence selected from the group consisting of SEQ ID NOs: 4, 6, and 8.

31. The kit of claim 20, wherein the priming composition, the boosting composition, or both, comprise(s) an adjuvant.

32-33. (canceled)

34. A method of raising an immune response against human immunodeficiency virus (HIV) in a subject comprising:
(i) administering to a subject in need thereof at least once a therapeutically effective amount of a priming composition comprising a self-replicating RNA molecule that encodes a first polypeptide antigen that comprises a first epitope from HIV; and
(ii) subsequently administering the subject at least once a therapeutically effective amount of a boosting composition comprising a second polypeptide antigen that comprises a second epitope from HIV;
wherein said first epitope and second epitope are the same epitope.

35-46. (canceled)
